(19) United States
(12) Patent Application Publication
(10) Pub. No.: US 2024/0376469 A1
(43) Pub. Date: Nov. 14, 2024

(54) COMPOUNDS AND METHODS FOR MODULATING HUNTINGTIN

(71) Applicant: Ionis Pharmaceuticals, Inc., Carlsbad, CA (US)

(72) Inventors: Bethany Fitzsimmons, Durham, NC (US); Susan M. Freier, San Diego, CA (US); Holly Kordasiewicz, San Diego, CA (US)

(73) Assignee: Ionis Pharmaceuticals, Inc., Carlsbad, CA (US)

(21) Appl. No.: 18/263,194

(22) PCT Filed: Jan. 28, 2022
(86) PCT No.: PCT/US2022/014231

§ 371 Date: Jul. 27, 2023

Related U.S. Application Data
(60) Provisional application No. 63/143,686, filed on Jan. 29, 2021.

(51) Int. Cl. C12N 15/113; A61K 31/7125

(52) U.S. Cl. CPC C12N 15/113; A61K 31/7125; C12N 2310/315; C12N 2310/3341

(57) ABSTRACT

Provided are compounds, methods, and pharmaceutical compositions for reducing the amount or activity of HTT RNA in a cell or subject, and in certain instances reducing the amount of HTT protein in a cell or subject. Such compounds, methods, and pharmaceutical compositions are useful to prevent, treat, or ameliorate at least one symptom or hallmark of a repeat expansion disease. Such repeat expansion diseases include myotonic dystrophy, amyotrophic lateral sclerosis, frontotemporal dementia, Huntington's disease, polyglutamine disorders, Fragile X syndrome, and spinocerebellar ataxia. Such symptoms or hallmarks include brain atrophy, muscle atrophy, nerve degeneration, uncontrolled movement, seizure, tremor, anxiety, and depression.

### SEQUENCE LISTING

The present application is being filed along with a Sequence Listing in electronic format. The Sequence Listing is provided as a file entitled BIOL0413WOSEQ_ST25.txt, created on Jan. 24, 2022, which is 1.204 MB in size. The information in the electronic format of the sequence listing is incorporated herein by reference in its entirety.

### FIELD

Provided herein are compounds, pharmaceutical compositions, and methods of use for reducing an amount of HTT RNA in a cell or a subject, and in certain instances reducing the amount of HTT protein in a cell or a subject. In certain embodiments, compounds, methods, and pharmaceutical compositions are useful for ameliorating at least one symptom or hallmark of a repeat expansion disease, e.g., Huntington's Disease. Such symptoms or hallmarks of Huntington's disease include, but are not limited to, brain atrophy, muscle atrophy, nerve degeneration, uncontrolled movement, seizure, tremor, anxiety, and depression.

### BACKGROUND

Repeat expansion diseases are a group of diseases characterized by a pathological number of consecutive repeat units in a region of a gene, each repeat unit consisting of several linked nucleosides having the same nucleobase sequence as the other repeat units. In general, repeat expansion diseases are genetically inherited. In some cases, an individual is born with a pathological number of repeat units and is symptomatic from a young age. In other cases, an individual is born with a normal, or near normal, number of repeats. However, due to a genetic mutation and genetic instability, the number of repeats increases with cell division, time, and age. The expanded region of the gene ultimately has pathological effects, most often observed as neurological symptoms. Many repeat expansion diseases are classified as a spinocerebellar ataxia, a neurodegenerative disease, or a neuromuscular disease. Non-limiting examples of repeat expansion diseases are myotonic dystrophy (DM1 and DM2); Huntington's disease (HD) and many autosomal dominant spinocerebellar ataxias; some forms of amyotrophic lateral sclerosis and/or frontotemporal dementia; various polyglutamine disorders (including spinal and bulbar muscular atrophy); Fragile X syndrome; and Friedrich's ataxia.

HD is caused by the expansion of a cytosine-adenine-guanine (CAG) trinucleotide repeat region in IT15, the gene that encodes huntingtin protein (HTT protein). The resulting expanded CAG repeat region encodes an abnormally long polyglutamine (PolyQ) tract in HTT protein, resulting in the expression of a mutant HTT (mHTT) protein. As a result of excessive polyglutamine length, mHTT protein forms aggregates in the cytoplasm and nucleus of CNS neurons (Davies et al., Cell 1997, 90:537-548). Due to its genomic instability, the expanded CAG repeat region can further expand with age and during meiotic transmission to include additional CAG repeats. Individuals with 27 to 35 CAG repeats typically do not develop HD, but their children are at risk of developing HD. Individuals with 35 to 60 CAG repeats typically experience adult-onset HD. Individuals with greater than 60 CAG repeats generally develop juvenile HD, experiencing symptoms of HD before the age of 20 years. Individuals with a normal number of CAG repeats (<27) are not considered to be at risk of developing HD.

### SUMMARY OF THE INVENTION

Provided herein are compounds, pharmaceutical compositions, and methods of use for reducing the amount of HTT RNA, and in certain embodiments reducing the amount or activity of HTT protein in a cell or a subject. In certain embodiments, the HTT RNA is mHTT RNA. In certain embodiments, the HTT protein is mHTT protein. In certain embodiments, the subject has or is at risk of having a neurodegenerative disease. In certain embodiments, the neurodegenerative disease is a repeat expansion disease. In certain embodiments, the repeat expansion disease is myotonic dystrophy (DM1 and DM2), amyotrophic lateral sclerosis, frontotemporal dementia, Huntington's disease, various polyglutamine disorders, Fragile X syndrome, or a spinocerebellar ataxia (SCA1, SCA2, SCA3, SCA6, SCA7, SCA8, SCA10, SCA17, or Friedrich's ataxia). In certain embodiments, compounds useful for reducing the amount of HTT RNA and/or HTT protein are oligomeric compounds. In certain embodiments, oligomeric compounds comprise modified oligonucleotides.

Also provided herein are methods useful for ameliorating a symptom or hallmark of a neurodegenerative disease. In certain embodiments, the neurodegenerative disease is a repeat expansion disease. In certain embodiments, the repeat expansion disease is myotonic dystrophy (DM1 and DM2), amyotrophic lateral sclerosis, frontotemporal dementia, Huntington's disease, various polyglutamine disorders, Fragile X syndrome, or a spinocerebellar ataxia (SCA1, SCA2, SCA3, SCA6, SCA7, SCA8, SCA0, SCA17, or Friedrich's ataxia). In certain embodiments, the symptom or hallmark is brain atrophy, reduced brain activity, reduced brain connectivity, muscle atrophy, muscle weakness, muscle cramping, disordered muscle movement, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” or “2′-MOE sugar moiety” or “2′-O-methoxyethylribose modified sugar” means a 2′-OCH₂CH₂OCH₃ group in place of the 2′-OH group of a ribosyl sugar moiety. “MOE” means methoxyethyl. Unless otherwise indicated, a 2′-MOE has the β-D stereochemical configuration.

As used herein, “2′-MOE nucleoside” means a nucleoside comprising a 2′-MOE sugar moiety.

As used herein, “2′-OMe” or “2′-O-methyl sugar moiety” means a 2′-OCH₃ group in place of the 2′—OH group of a ribosyl sugar moiety. Unless otherwise indicated, a 2′-OMe has the β-D stereochemical configuration.

As used herein, “2′-OMe nucleoside” means a nucleoside comprising a 2′-OMe sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark, or the delayed onset of or slowing of progression in the severity or frequency of a symptom or hallmark. In certain embodiments, the symptom or hallmark is brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, or suicidal behavior. The progression or severity of indicators may be determined by subjective or objective measures, which are known to those skilled in the art.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an oligomeric compound capable of achieving at least one antisense activity. In certain embodiments, an antisense compound further comprises one or more additional features, such as a conjugate group.

As used herein, “sense compound” means a sense oligonucleotide and optionally one or more additional features, such as a conjugate group. As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “CAG repeat” means one of multiple contiguous trinucleotide units, wherein each trinucleotide unit consists of three contiguous nucleosides having a nucleobase sequence from 5′ to 3′ of cytosine (C), adenine (A), and guanine (G).

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid. aCSF is a buffered solution that closely matches the electrolyte concentrations of CSF.

As used herein, “cleavable moiety” means a bond or group of atoms that is cleaved under physiological conditions, for example, inside a cell, a subject, or a human.

As used herein, “cell-targeting moiety” means a conjugate moiety that interacts with a cell or a portion thereof. In certain embodiments, the cell-targeting moiety binds a receptor on a surface of a cell.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “complementary” in reference to an oligonucleotide means that at least 70% of the nucleobases of the oligonucleotide or one or more portions thereof and the nucleobases of a target nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Certain modified nucleobases that pair with natural nucleobases or with other modified nucleobases are known in the art. For example, inosine can pair with adenosine, cytosine, or uracil. Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly attached to an oligonucleotide. Conjugate groups include a conjugate moiety and a conjugate linker that attaches the conjugate moiety to the oligonucleotide. In certain embodiments, the conjugate moiety comprises a cell-targeting moiety.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “diluent” means an ingredient in a composition that lacks pharmacological activity, but is pharmaceutically necessary or desirable. For example, the diluent in an injected composition can be a liquid, e.g. aCSF, PBS, or saline solution.

As used herein, “gapmer” means an oligonucleotide having a central region comprising a plurality of nucleosides that support RNase H1 cleavage positioned between a 5′-region and a 3′-region. Herein, the nucleosides of the 5′-region and 3′-region each comprise a 2′-modified furanosyl sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings” or “wing segments.” In certain embodiments, the central region is a deoxy region. Unless otherwise indicated, a gapmer may comprise one or more modified internucleoside linkages and/or modified nucleobases and such modifications do not necessarily follow the gapmer pattern of the sugar modifications.

As used herein, the term “MOE gapmer” indicates a gapmer having a gap comprising 2′-β-D-deoxynucleosides and wings comprising 2′-MOE nucleosides.

As used herein, the term “mixed wing gapmer” indicates a gapmer having wings comprising modified nucleosides comprising at least two different sugar modifications.

As used herein, “hotspot region” is a range of nucleobases on a target nucleic acid that is amenable to oligomeric agent or oligomeric compound-mediated reduction of the amount or activity of the target nucleic acid.

As used herein, “HTT RNA” is the RNA expression product of the human gene, IT15. “mHTT RNA” is the RNA expression product of the human gene, IT15, that contains 27 or more contiguous CAG repeats.

As used herein, “HTT protein” is the protein expression product of HTT RNA. “mHTT protein” is the protein expression product of mHTT.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “IT15 gene” refers to a genomic sequence encoding an HTT RNA. In general, a human has two IT15 genes which may have the same or different nucleobase sequences.

As used herein, “identifying a subject at risk for developing a repeat expansion disease” means identifying a subject having been diagnosed with a repeat expansion disease or identifying a subject that has a risk factor for developing a repeat expansion disease.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a target nucleic acid or oligonucleotide independent of any sugar or internucleoside linkage modification.

As used herein, “nucleoside” means a compound, or a fragment of a compound, comprising a nucleobase and a sugar moiety. The nucleobase and sugar moiety are each, independently, unmodified or modified.

As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by an animal. In certain embodiments, a pharmaceutically acceptable carrier or diluent is sterile water, sterile saline, sterile buffer solution, or sterile artificial cerebrospinal fluid. In certain embodiments, a pharmaceutically acceptable carrier or diluent is phosphate buffered saline. In certain embodiments, a pharmaceutically acceptable carrier or diluent is artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto. As used herein, “potassium salt” means a salt of a modified oligonucleotide, wherein the cation of the salt is potassium.

As used herein, “sodium salt” means a salt of a modified oligonucleotide, wherein the cation of the salt is sodium.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prevent” or “preventing” refers to delaying or forestalling the onset or development of a neurodegenerative disease, or a symptom or hallmark thereof, for a period of time or indefinitely.

As used herein, “prodrug” means a therapeutic agent in a first form outside the body that is converted to a second form within a subject or cells thereof. Typically, conversion of a prodrug within the subject is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing the amount or activity,” or “inhibiting the amount or activity,” in connection with a gene transcript (RNA) refers to a reduction or blockade of the transcriptional expression or activity relative to the transcriptional expression or activity in an untreated or control sample and does not necessarily indicate a total elimination of transcriptional expression or activity.

As used herein, “reducing the amount or activity,” or “inhibiting the amount or activity,” in connection with a protein refers to a reduction or blockade of the protein's expression or activity relative to the protein expression or activity in an untreated or control sample and does not necessarily indicate a total elimination of protein expression or activity.

As used herein, “repeat expansion disease” means a disease associated with an increased number of contiguous repeat units in a region of a gene of a subject, each repeat unit consisting of 3-10 linked nucleosides having the same nucleobase sequence as the other repeat units, relative to a control subject without the disease. In certain embodiments, the subject experiences a symptom or hallmark of the repeat expansion disease.

As used herein, “repeat region” means a region of a gene comprising three or more contiguous repeat units, each repeat unit consisting of 3-10 linked nucleosides having the same nucleobase sequence as the other repeat units. Non-limiting examples of repeat units are CTG, CUG, CAG, CUG, GGGGCC, CAG, GAA, CAA and ATTCT.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNAi), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay” means the assay described in Examples 1, 2, and 3, and reasonable variations thereof.

As used herein, “standard in vivo assay” means the assay described in Example 9 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human subject. A “subject in need thereof,” is a subject who would benefit from administration of a modified oligonucleotide disclosed herein. In certain embodiments, the subject in need thereof has HD.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent that provides a therapeutic benefit to a subject. For example, a therapeutically effective amount improves a symptom or hallmark of a disease.

### CERTAIN EMBODIMENTS

The present disclosure provides the following non-limiting numbered embodiments:

Embodiment 1. An oligomeric compound comprising a modified oligonucleotide consisting of 12 to 30 linked nucleosides, wherein the nucleobase sequence of the modified oligonucleotide is at least 80% complementary to an equal length portion of an HTT RNA, and wherein the modified oligonucleotide comprises at least one modification selected from a modified sugar moiety and a modified internucleoside linkage.

Embodiment 2. An oligomeric compound comprising a modified oligonucleotide consisting of 12 to 30 linked nucleosides and having a nucleobase sequence comprising at least 12, at least 13, at least 14, at least 15, at least 16, at least 17, at least 18, at least 19, or 20 contiguous nucleobases of any of SEQ ID NOs: 26-3707.

Embodiment 3. An oligomeric compound comprising a modified oligonucleotide consisting of 12 to 30 linked nucleosides and having a nucleobase sequence comprising:
a) at least 12, at least 13, at least 14, at least 15, at least 16, or 17 contiguous nucleobases of any of SEQ ID NOs: 26-857 or 3552-3590;
b) at least 12, at least 13, at least 14, at least 15, at least 16, at least 17, at least 18, at least 19, or 20 contiguous nucleobases of any of SEQ ID NOs: 858-3551 or 3591-3682; or
c) at least 12, at least 13, at least 14, at least 15, at least 16, at least 17, or 18 contiguous nucleobases of any of SEQ ID NOs: 3683-3707.

Embodiment 4. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases complementary to:
an equal length portion of nucleobases 56443-56500 of SEQ ID NO: 2;
an equal length portion of nucleobases 62833-62860 of SEQ ID NO: 2;
an equal length portion of nucleobases 69788-69824 of SEQ ID NO: 2;
an equal length portion of nucleobases 9984-10008 of SEQ ID NO: 2;
an equal length portion of nucleobases 106863-106924 of SEQ ID NO: 2;
an equal length portion of nucleobases 115297-115337 of SEQ ID NO: 2;
an equal length portion of nucleobases 117868-117902 of SEQ ID NO: 2;
an equal length portion of nucleobases 119644-119670 of SEQ ID NO: 2;
an equal length portion of nucleobases 126780-126829 of SEQ ID NO: 2; or
an equal length portion of nucleobases 130388-130418 of SEQ ID NO: 2;
wherein the modified oligonucleotide comprises at least one modification selected from a modified sugar moiety and a modified internucleoside linkage.

Embodiment 5. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of a sequence selected from:
SEQ ID NOs: 1184, 1274, 1331, 1455, 1481, 1562, and 1684;
SEQ ID NOs: 3029, 3104, 3557, 3562, 3569, 3586, 3610, 3620, 3622, 3623, 3627, 3634, 3664, 3687, 3692, and 3697;
SEQ ID NOs: 2750, 2814, 2887, 2991, and 3090;
SEQ ID NOs: 2581, 2640, 2736, 2857, and 2917;
SEQ ID NOs: 1105, 1232, 1249, 3330, 3342, 3344, 3362, 3382, 3411, 3419, 3439, 3456, 3475, 3488, 3491, 3511, 3531, 3550, and 3551;
SEQ ID NOs: 2208, 2310, 2335, 2471, 3553, 3559, 3563, 3565, 3570, 3580, 3581, 3583, 3585, 3589, 3593, 3594, 3597, 3600, 3601, 3604, 3614, 3616, 3630, 3631, 3637, 3648, 3650, 3652, 3653, 3660, 3661, 3662, 3684, and 3689;
SEQ ID NOs: 2644, 2770, 2786, 2916, 3574, 3576, 3578, 3587, 3665, 3666, 3667, 3668, 3669, 3670, 3671, 3672, 3673, 3674, 3675, 3676, 3686, 3694, and 3698;
SEQ ID NOs: 919, 940, 3677, 3678, 3679, 3680, 3681, 3682, 3705, 3706, and 3707;
SEQ ID NOs: 1576, 1652, 1771, 1813, 1933, 1987, 2078, 3552, 3555, 3558, 3560, 3572, 3573, 3575, 3579, 3584, 3598, 3608, 3611, 3613, 3619, 3628, 3635, 3644, 3646, 3649, 3663, 3683, 3688, 3690, 3700, 3701, 3702, 3703, and 3704; and
SEQ ID NOs: 1343, 1406, 1539, 3335, 3401, 3472, 3477, 3519, and 3591;

wherein the modified oligonucleotide comprises at least one modification selected from a modified sugar moiety and a modified internucleoside linkage.

Embodiment 6. The oligomeric compound of any of embodiments 1-5, wherein the modified oligonucleotide has a nucleobase sequence that is at least 85%, at least 90%, at least 95%, or 100% complementary to the nucleobase sequence of any one of SEQ ID NOS: 1-5 when measured across the entire nucleobase sequence of the modified oligonucleotide.

Embodiment 7. The oligomeric compound of any of embodiments 1-6, wherein the modified oligonucleotide consists of 10 to 25, 10 to 30, 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 25, 18 to 30, 19 to 20, 19 to 25, 19 to 30, 20 to 25, 20 to 30, 21 to 25, 21 to 30, 22 to 25, 22 to 30, 23 to 25, or 23 to 30 linked nucleosides.

Embodiment 8. The oligomeric compound of any of embodiments 1-7, wherein the modified oligonucleotide comprises at least one modified nucleoside.

Embodiment 9. The oligomeric compound of embodiment 8, wherein the modified oligonucleotide comprises at least one modified nucleoside comprising a modified sugar moiety.

Embodiment 10. The oligomeric compound of embodiment 9, wherein the modified oligonucleotide comprises at least one modified nucleoside comprising a bicyclic sugar moiety.

Embodiment 11. The oligomeric compound of embodiment 10, wherein the bicyclic sugar moiety has a 2′-4′ bridge selected from —O—CH₂—; and —O—CH(CH₃)—.

Embodiment 12. The oligomeric compound of any of embodiments 8-11, wherein the modified oligonucleotide comprises at least one modified nucleoside comprising a non-bicyclic modified sugar moiety.

Embodiment 13. The oligomeric compound of embodiment 12, wherein the non-bicyclic modified sugar moiety is a 2′-MOE modified sugar moiety or a 2′-cEt modified sugar moiety.

Embodiment 14. The oligomeric compound of any of embodiments 8-13, wherein the modified oligonucleotide comprises at least one modified nucleoside comprising a sugar surrogate.

Embodiment 15. The oligomeric compound of embodiment 14, wherein the sugar surrogate is any of morpholino, modified morpholino, PNA, THP, and F-HNA.

Embodiment 16. The oligomeric compound of any of embodiments 1-15, wherein the modified oligonucleotide is a gapmer.

Embodiment 17. The oligomeric compound of any of embodiments 1-16, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides,
wherein each of the 5′-region nucleosides and each of the 3′-region nucleosides comprises a modified sugar moiety and each of the central region nucleosides comprises a 2′-β-D-deoxyribosyl sugar moiety.

Embodiment 18. The oligomeric compound of embodiment 17, wherein
the 5′-region consists of 5 linked 5′-region nucleosides;
the central region consists of 10 linked central region nucleosides; and
the 3′-region consists of 5 linked 3′-region nucleosides, and
wherein each of the 5′-region nucleosides and each of the 3′-region nucleosides comprises a 2′-MOE sugar moiety and each of the central region nucleosides comprises a 2′-β-D-deoxyribosyl sugar moiety.

Embodiment 19. The oligomeric compound of embodiment 17, wherein
the 5′-region consists of 6 linked 5′-region nucleosides;
the central region consists of 10 linked central region nucleosides; and
the 3′-region consists of 4 linked 3′-region nucleosides, and
wherein each of the 5′-region nucleosides and each of the 3′-region nucleosides comprises a 2′-MOE sugar moiety and each of the central region nucleosides comprises a 2′-β-D-deoxyribosyl sugar moiety.

Embodiment 20. The oligomeric compound of embodiment 17, wherein
the 5′-region consists of 5 linked 5′-region nucleosides;
the central region consists of 8 linked central region nucleosides; and
the 3′-region consists of 5 linked 3′-region nucleosides, and
wherein each of the 5′-region nucleosides and each of the 3′-region nucleosides comprises a 2′-MOE sugar moiety and each of the central region nucleosides comprises a 2′-β-D-deoxyribosyl sugar moiety.

Embodiment 21. The oligomeric compound of embodiment 17, wherein
the 5′-region consists of 4 linked 5′-region nucleosides;
the central region consists of 8 linked central region nucleosides; and
the 3′-region consists of 5 linked 3′-region nucleosides, and
wherein each of the 5′-region nucleosides and each of the 3′-region nucleosides comprises a modified sugar moiety selected from a 2′-MOE sugar moiety and a 2′-cEt sugar moiety and each of the central region nucleosides comprises a 2′-β-D-deoxyribosyl sugar moiety.

Embodiment 22. The oligomeric compound of any of embodiments 1-21, wherein the modified oligonucleotide comprises at least one modified internucleoside linkage.

Embodiment 23. The oligomeric compound of embodiment 22, wherein each internucleoside linkage of the modified oligonucleotide is a modified internucleoside linkage.

Embodiment 24. The oligomeric compound of embodiment 22 or embodiment 23, wherein each internucleoside linkage is a phosphorothioate internucleoside linkage.

Embodiment 25. The oligomeric compound of embodiment 22, wherein the modified oligonucleotide comprises at least one phosphodiester internucleoside linkage.

Embodiment 26. The oligomeric compound of any of embodiments 22, 23, or 25, wherein each internucleoside linkage is independently selected from a phosphodiester internucleoside linkage and a phosphorothioate internucleoside linkage.

Embodiment 27. The oligomeric compound of any of embodiments 22, 23, or 25-26, wherein at least 4, at least 5, at least 6, at least 7, at least 8, at least 9, at least 10, at least 11, at least 12, at least 13, at least 14, at least 15, at least 16, at least 17, at least 18, or 19 internucleoside linkages of the modified oligonucleotide are phosphorothioate internucleoside linkages.

Embodiment 28. The oligomeric compound of any of embodiments 1-23 or 25-27, wherein the internucleoside linkage motif of the modified oligonucleotide is selected from: 5′-soosssssssssooss-3′, 5′-sooosssssssssssooss-3′, 5′-sooosssssssssssooos-3′, 5′-sossssssssssssssoss-3′, 5′-ssoosssssssssssooss-3′, 5′-sooosssssssssooss-3′, 5′-sossssssssssssoss-3′, and 5′-sooooossssssssssoss-3′, wherein each “s” represents a phosphorothioate internucleoside linkage and each “o” represents a phosphodiester internucleoside linkage.

Embodiment 29. The oligomeric compound of any of embodiments 1-28, wherein the modified oligonucleotide comprises a modified nucleobase.

Embodiment 30. The oligomeric compound of embodiment 29, wherein the modified nucleobase is a 5-methylcytosine.

Embodiment 31. The oligomeric compound of any of embodiments 1-30, wherein the modified oligonucleotide comprises a deoxy region.

Embodiment 32. The oligomeric compound of embodiment 31, wherein each nucleoside of the deoxy region is a 2′-β-D-deoxynucleoside.

Embodiment 33. The oligomeric compound of embodiment 31 or embodiment 32, wherein the deoxy region consists of 6, 7, 8, 9, 10, or 6-10 linked nucleosides.

Embodiment 34. The oligomeric compound of any of embodiments 31-33, wherein each nucleoside immediately adjacent to the deoxy region comprises a modified sugar moiety.

Embodiment 35. The oligomeric compound of any of embodiments 31-34, wherein the deoxy region is flanked on the 5′-side by a 5′-region consisting of 1-6 linked 5′-region nucleosides and on the 3′-side by a 3′-region consisting of 1-6 linked 3′-region nucleosides; wherein at least one nucleoside of the 5′-region comprises a modified sugar moiety; and at least one nucleoside of the 3′-region comprises a modified sugar moiety.

Embodiment 36. The oligomeric compound of embodiment 35, wherein each nucleoside of the 5′-region comprises a modified sugar moiety.

Embodiment 37. The oligomeric compound of embodiment 35 or embodiment 36, wherein each nucleoside of the 3′-region comprises a modified sugar moiety.

Embodiment 38. The oligomeric compound of any of embodiments 1-37, wherein the modified oligonucleotide consists of 12-30, 12-22, 12-20,14-18, 14-20, 15-17, 15-25, 16-20, 18-22, or 18-20 linked nucleosides.

Embodiment 39. The oligomeric compound of any of embodiments 1-38, wherein the modified oligonucleotide consists of 17 linked nucleosides.

Embodiment 40. The oligomeric compound of any of embodiments 1-38, wherein the modified oligonucleotide consists of 18 linked nucleosides.

Embodiment 41. The oligomeric compound of any of embodiments 1-38, wherein the modified oligonucleotide consists of 20 linked nucleosides.

Embodiment 42. The oligomeric compound of any of embodiments 1-41, wherein the modified oligonucleotide is a pharmaceutically acceptable salt.

Embodiment 43. The oligomeric compound of embodiment 42, which is a pharmaceutically acceptable salt comprising one or more cations selected from sodium, potassium, calcium, and magnesium.

Embodiment 44. The oligomeric compound of any of embodiments 1-43, consisting of the modified oligonucleotide.

Embodiment 45. The oligomeric compound of any of embodiments 1-44, wherein the oligomeric compound comprises a conjugate group.

Embodiment 46. The oligomeric compound of embodiment 45, wherein the conjugate group comprises a conjugate moiety and a conjugate linker.

Embodiment 47. The oligomeric compound of embodiment 46, wherein the conjugate linker consists of a single bond.

Embodiment 48. The oligomeric compound of embodiment 46, wherein the conjugate linker is cleavable.

Embodiment 49. The oligomeric compound of embodiment 46, wherein the conjugate linker comprises 1-3 linker-nucleosides.

Embodiment 50. The oligomeric compound of embodiment 46, wherein the oligomeric compound does not comprise a linker-nucleoside.

Embodiment 51. The oligomeric compound of any of embodiments 45-50, wherein the conjugate group is attached to the modified oligonucleotide at the 5′-end of the modified oligonucleotide.

Embodiment 52. The oligomeric compound of any of embodiments 45-50, wherein the conjugate group is attached to the modified oligonucleotide at the 3′-end of the modified oligonucleotide.

Embodiment 53. The oligomeric compound of any of embodiments 1-52, comprising a terminal group.

Embodiment 54. The oligomeric compound of embodiment 53, wherein the terminal group is an abasic sugar moiety.

Embodiment 55. The oligomeric compound of any of embodiments 1-54, wherein the modified oligonucleotide is a single-stranded modified oligonucleotide.

Embodiment 56. The oligomeric compound of any of embodiments 1-55, wherein the oligomeric compound is an RNase H agent comprising the oligomeric compound.

Embodiment 57. An oligomeric compound comprising a modified oligonucleotide according to the following chemical notation:

(SEQ ID NO: 3619)
^{m}Cₑₛ Aₑₒ ^{m}Cₑₒ Aₑₒ Gₑₒ ^{m}Cₑₒ T_{ds} T_{ds} T_{ds} T_{ds} A_{ds} T_{ds} T_{ds} T_{ds} ^{m}C_{ds}
^{m}C_{ds} Aₑₒ Tₑₛ Aₑₛ ^{m}Cₑ,

wherein
A=an adenine,
^{m}C=a 5-methylcytosine,
G=a guanine,
T=a thymine,
e=a 2′-O-methoxyethylribose modified sugar,
d=a 2′-deoxyribose sugar moiety,
s=a phosphorothioate internucleoside linkage, and
o=a phosphodiester internucleoside linkage.

Embodiment 58. An oligomeric compound comprising a modified oligonucleotide according to the following chemical notation:

(SEQ ID NO: 1502)
Gₑₛ ^{m}Cₑₒ Tₑₒ Tₑₒ Tₑₛ G_{ds} A_{ds} T_{ds} T_{ds} T_{ds} T_{ds} T_{ds} A_{ds} ^{m}C_{ds} ^{m}C_{ds}
Aₑₒ Gₑₒ Tₑₛ Tₑₛ Aₑ,

wherein
A=an adenine,
^{m}C=a 5-methylcytosine,
G=a guanine,
T=a thymine,
e=a 2′-O-methoxyethylribose modified sugar,
d=a 2′-deoxyribose sugar moiety,
s=a phosphorothioate internucleoside linkage, and
o=a phosphodiester internucleoside linkage.

Embodiment 59. An oligomeric compound comprising a modified oligonucleotide according to the following chemical notation:

(SEQ ID NO: 3646)
Aₑₛ ^{m}Cₑₒ Aₑₒ Gₑₒ ^{m}Cₑₒ Tₑₒ T_{ds} T_{ds} T_{ds} A_{ds} T_{ds} T_{ds} T_{ds} ^{m}C_{ds} ^{m}C_{ds}
A_{ds} Tₑₒ Aₑₛ ^{m}Cₑₛ Aₑ,

wherein
A=an adenine,
^{m}C=a 5-methylcytosine,
G=a guanine,
T=a thymine,
e=a 2′-O-methoxyethylribose modified sugar,
d=a 2′-deoxyribose sugar moiety,
s=a phosphorothioate internucleoside linkage, and
o=a phosphodiester internucleoside linkage.

Embodiment 60. The oligomeric compound of any of embodiments 57-59, wherein the modified oligonucleotide is a pharmaceutically acceptable salt.

Embodiment 61. The oligomeric compound of embodiment 60, which is a pharmaceutically acceptable salt comprising one or more cations selected from sodium, potassium, calcium, and magnesium.

Embodiment 62. The oligomeric compound of any of embodiments 57-61, wherein the oligomeric compound is a singled-stranded oligomeric compound.

Embodiment 63. A modified oligonucleotide according to the following chemical structure:

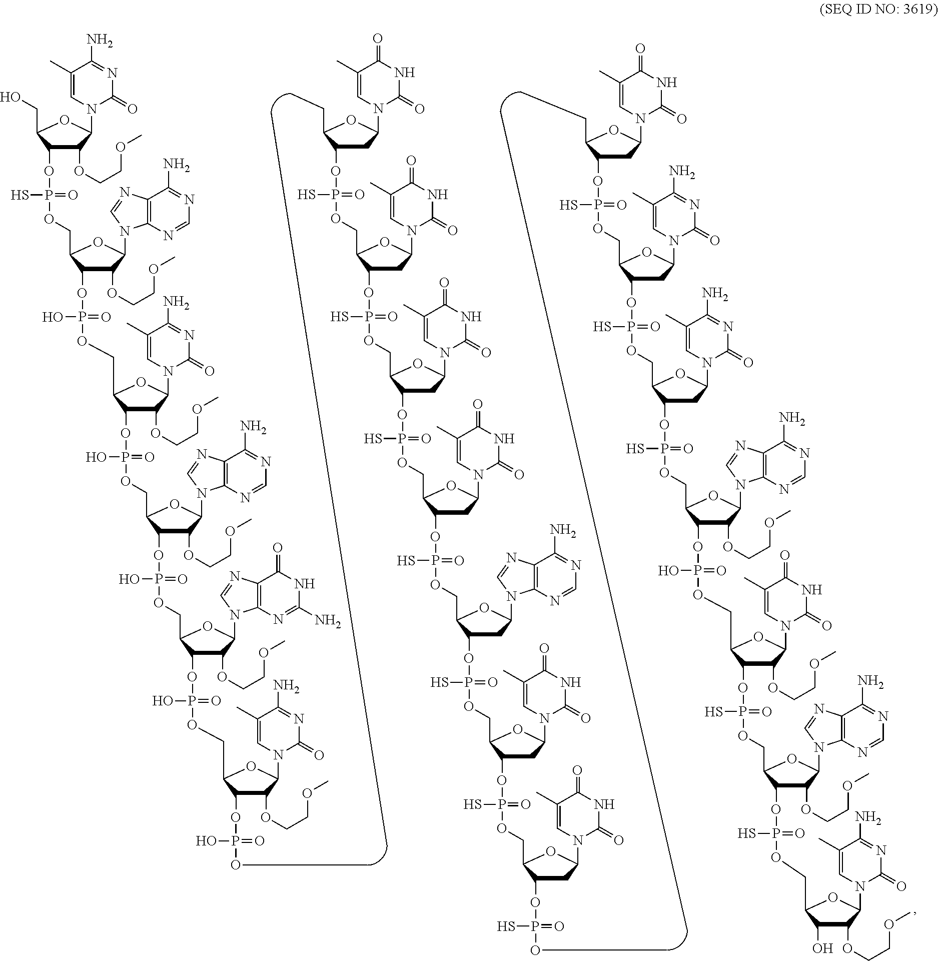

or a pharmaceutically acceptable salt thereof.

Embodiment 64. The modified oligonucleotide of embodiment 63, which is a pharmaceutically acceptable salt comprising one or more cations selected from sodium, potassium, calcium, and magnesium.

Embodiment 65. A modified oligonucleotide according to the following chemical structure:

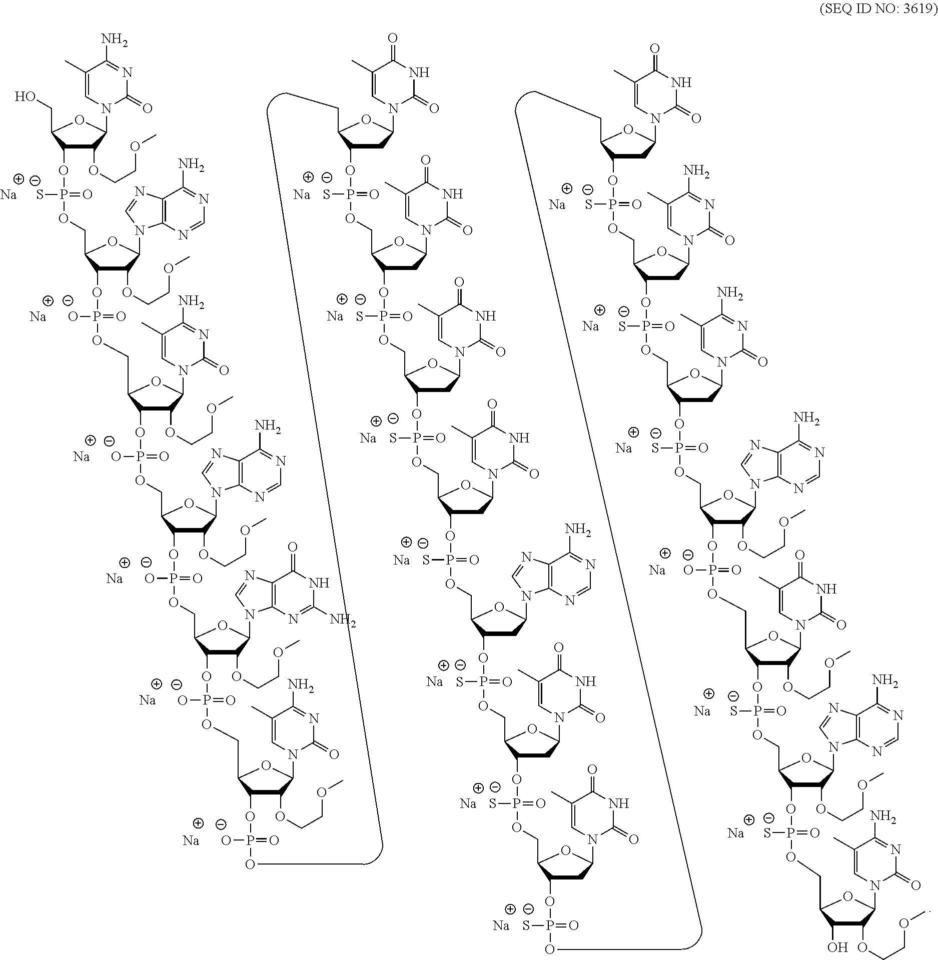

Embodiment 66. A modified oligonucleotide according to the following chemical structure:

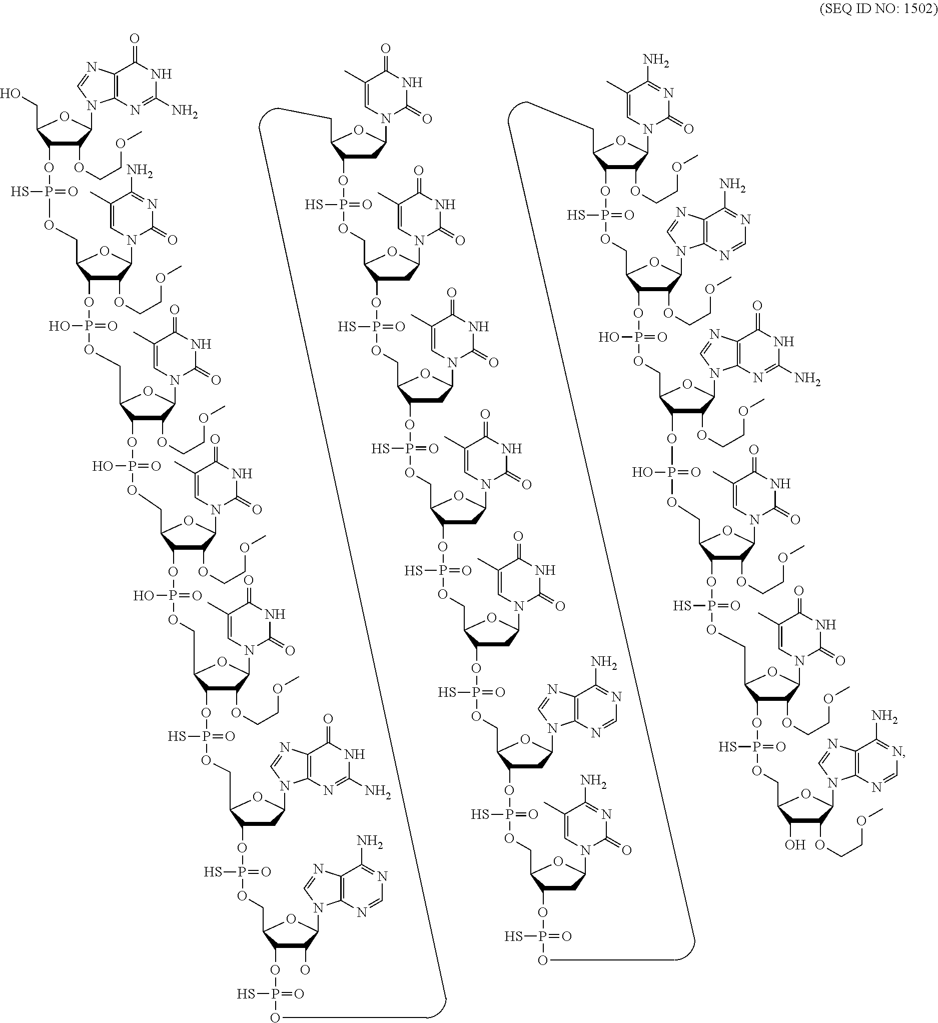

or a pharmaceutically acceptable salt thereof.

Embodiment 67. The modified oligonucleotide of embodiment 66, which is a pharmaceutically acceptable salt comprising one or more cations selected from sodium, potassium, calcium, and magnesium.

Embodiment 68. A modified oligonucleotide according to the following chemical structure:

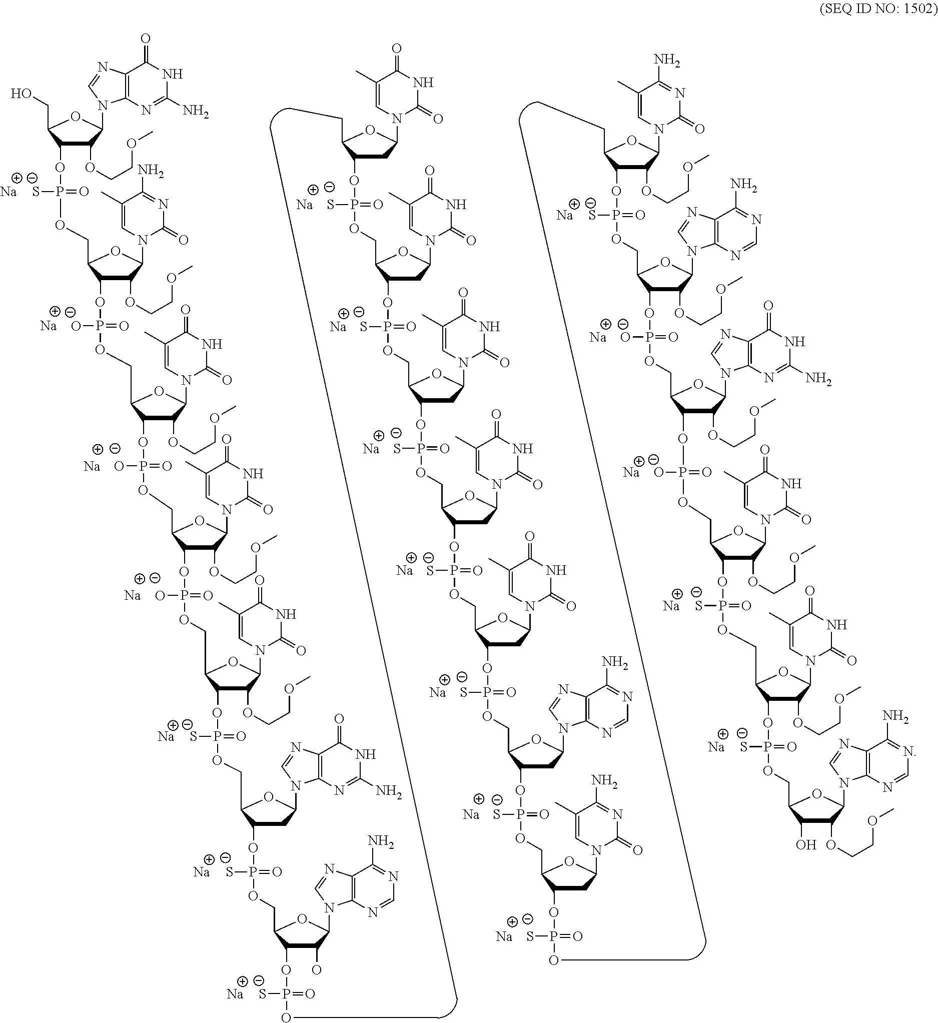

Embodiment 69. A modified oligonucleotide according to the following chemical structure:

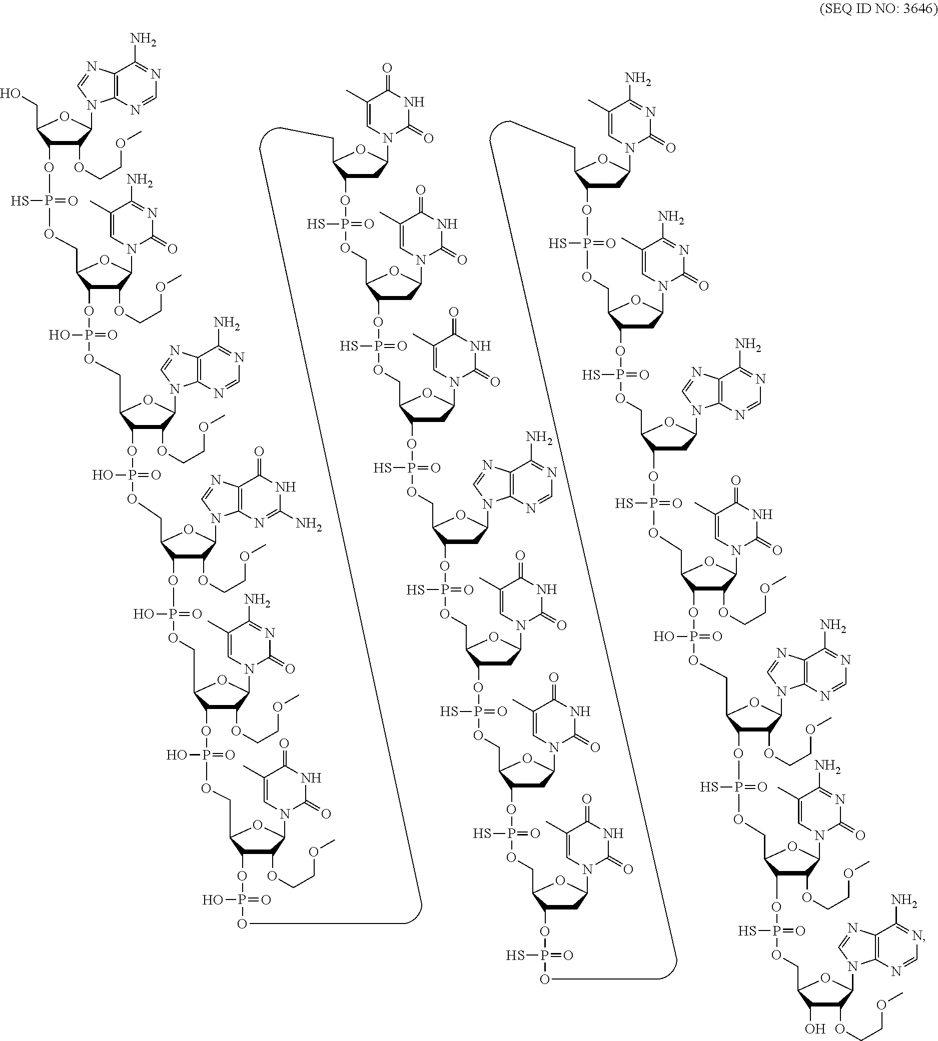

or a pharmaceutically acceptable salt thereof.

Embodiment 70. The modified oligonucleotide of embodiment 69, which is a pharmaceutically acceptable salt comprising one or more cations selected from sodium, potassium, calcium, and magnesium.

Embodiment 71. A modified oligonucleotide according to the following chemical structure:

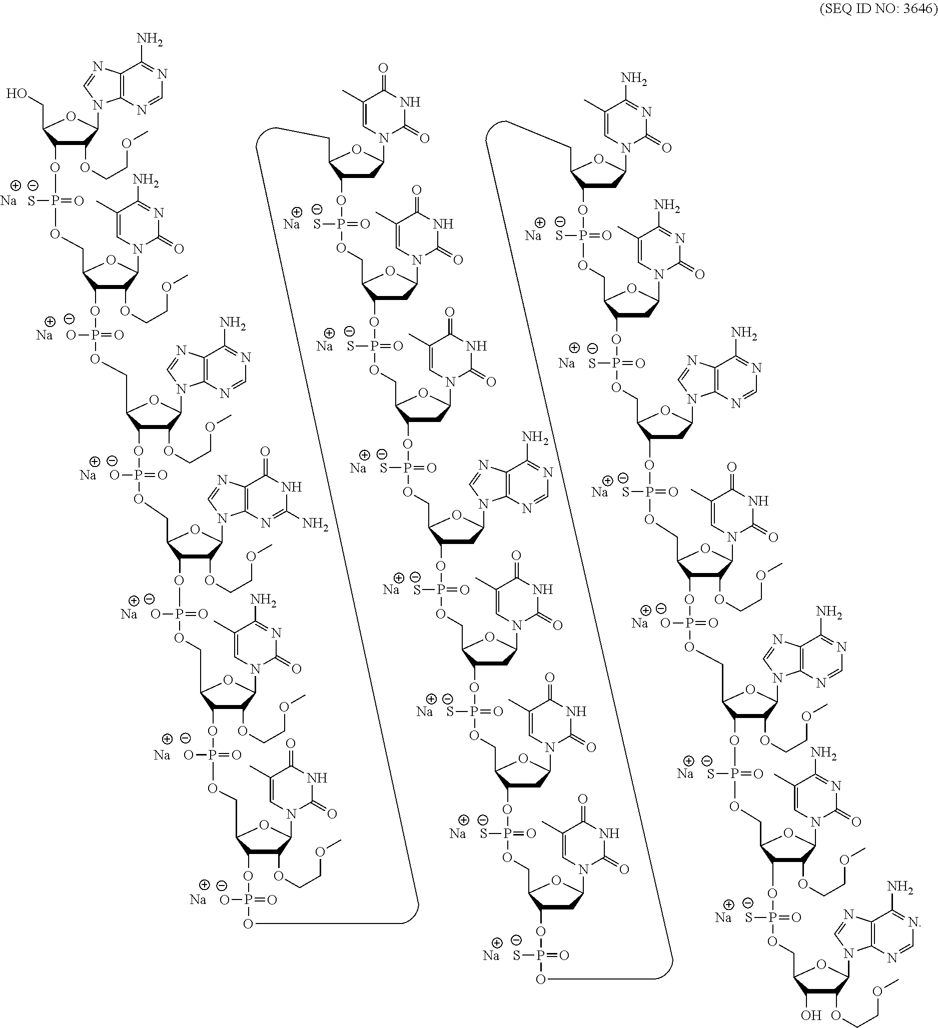

Embodiment 72. A chirally enriched population of oligomeric compounds of any of embodiments 1-62 or modified oligonucleotides of any of embodiments 63-71, wherein the population is enriched for modified oligonucleotides comprising at least one particular phosphorothioate internucleoside linkage having a particular stereochemical configuration.

Embodiment 73. The chirally enriched population of embodiment 72, wherein the population is enriched for modified oligonucleotides comprising at least one particular phosphorothioate internucleoside linkage having the (Sp) configuration.

Embodiment 74. The chirally enriched population of embodiment 72, wherein the population is enriched for modified oligonucleotides comprising at least one particular phosphorothioate internucleoside linkage having the (Rp) configuration.

Embodiment 75. The chirally enriched population of embodiment 72, wherein the population is enriched for modified oligonucleotides having a particular, independently selected stereochemical configuration at each phosphorothioate internucleoside linkage.

Embodiment 76. The chirally enriched population of embodiment 72, wherein the population is enriched for modified oligonucleotides having the (Sp) configuration at each phosphorothioate internucleoside linkage.

Embodiment 77. The chirally enriched population of embodiment 72, wherein the population is enriched for modified oligonucleotides having the (Rp) configuration at each phosphorothioate internucleoside linkage.

Embodiment 78. The chirally enriched population of embodiment 72, wherein the population is enriched for modified oligonucleotides having the (Rp) configuration at one particular phosphorothioate internucleoside linkage and the (Sp) configuration at each of the remaining phosphorothioate internucleoside linkages.

Embodiment 79. The chirally enriched population of embodiment 72, wherein the population is enriched for modified oligonucleotides having at least 3 contiguous phosphorothioate internucleoside linkages in the Sp, Sp, and Rp configurations, in the 5′ to 3′ direction.

Embodiment 80. A population of oligomeric compounds of any of embodiments 1-62 or modified oligonucleotides of any of embodiments 63-71, wherein all of the phosphorothioate internucleoside linkages of the modified oligonucleotide are stereorandom.

Embodiment 81. An oligomeric duplex, comprising a first oligomeric compound and a second oligomeric compound comprising a second modified oligonucleotide, wherein the first oligomeric compound is an oligomeric compound of any of embodiments 1-62.

Embodiment 82. The oligomeric duplex of embodiment 81, wherein the second modified oligonucleotide consists of 12 to 30 linked nucleosides, and wherein the nucleobase sequence of the second modified oligonucleotide comprises a complementary region of at least 8 nucleobases that is at least 90% complementary to an equal length portion of the first modified oligonucleotide.

Embodiment 83. The oligomeric duplex of embodiment 81 or embodiment 82, wherein the modified oligonucleotide of the first oligomeric compound comprises a 5′-stabilized phosphate group.

Embodiment 84. The oligomeric duplex of embodiment 83, wherein the stabilized phosphate group comprises a cyclopropyl phosphonate or a vinyl phosphonate.

Embodiment 85. The oligomeric duplex of any of embodiments 81-84, wherein at least one nucleoside of the second modified oligonucleotide comprises a modified sugar moiety.

Embodiment 86. The oligomeric duplex of embodiment 85, wherein the modified sugar moiety of the second modified oligonucleotide comprises a bicyclic sugar moiety.

Embodiment 87. The oligomeric duplex of embodiment 86, wherein the bicyclic sugar moiety of the second modified oligonucleotide comprises a 2′-4′ bridge selected from —O—CH₂—; and —O—CH(CH₃)—.

Embodiment 88. The oligomeric duplex of embodiment 85, wherein the modified sugar moiety of the second modified oligonucleotide comprises a non-bicyclic modified sugar moiety.

Embodiment 89. The oligomeric duplex of embodiment 88, wherein the non-bicyclic modified sugar moiety of the second modified oligonucleotide is a 2′-MOE sugar moiety, a 2′-cEt sugar moiety, a 2′-F sugar moiety, or a 2′-OMe sugar moiety.

Embodiment 90. The oligomeric duplex of any of embodiments 81-89, wherein at least one internucleoside linkage of the second modified oligonucleotide is a modified internucleoside linkage.

Embodiment 91. The oligomeric duplex of embodiment 90, wherein at least one modified internucleoside linkage of the second modified oligonucleotide is a phosphorothioate internucleoside linkage.

Embodiment 92. The oligomeric duplex of any of embodiments 81-91, wherein at least one internucleoside linkage of the second modified oligonucleotide is a phosphodiester internucleoside linkage.

Embodiment 93. The oligomeric duplex of any of embodiments 81-92, wherein each internucleoside linkage of the second modified oligonucleotide is independently selected from a phosphodiester or a phosphorothioate internucleoside linkage.

Embodiment 94. The oligomeric duplex of any of embodiments 81-93, wherein the second modified oligonucleotide comprises at least one modified nucleobase.

Embodiment 95. The oligomeric duplex of embodiment 94, wherein the at least one modified nucleobase of the second modified oligonucleotide is 5-methylcytosine.

Embodiment 96. The oligomeric duplex of any of embodiments 81-95, wherein the second modified oligonucleotide comprises a conjugate group.

Embodiment 97. The oligomeric duplex of embodiment 96, wherein the conjugate group comprises a conjugate linker and a conjugate moiety.

Embodiment 98. The oligomeric duplex of embodiment 96 or embodiment 97, wherein the conjugate group is attached to the 5′-end of the second modified oligonucleotide.

Embodiment 99. The oligomeric duplex of embodiment 96 or embodiment 97, wherein the conjugate group is attached to the 3′-end of the second modified oligonucleotide.

Embodiment 100. The oligomeric duplex of any of embodiments 96-99, wherein the conjugate group comprises a lipid.

Embodiment 101. The oligomeric duplex of any of embodiments 81-100, wherein the second modified oligonucleotide comprises a terminal group.

Embodiment 102. The oligomeric duplex of embodiment 101, wherein the terminal group is an abasic sugar moiety.

Embodiment 103. The oligomeric duplex of any of embodiments 81-102, wherein the second modified oligonucleotide consists of 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 22, 18 to 25, 18 to 30, 19 to 20, 19 to 25, 19 to 30, 20 to 25, 20 to 30, 21 to 25, 21 to 30, 22 to 25, 22 to 30, 23 to 25, or 23 to 30 linked nucleosides.

Embodiment 104. An antisense agent comprising an antisense compound, wherein the antisense compound is an oligomeric compound of any of embodiments 1-62.

Embodiment 105. An antisense agent comprising an antisense compound, wherein the antisense compound is an oligomeric duplex of any of embodiments 81-103.

Embodiment 106. The antisense agent of embodiment 104 or embodiment 105, wherein the antisense agent is:
a) an RNase H agent capable of reducing the amount of HTT nucleic acid through the activation of RNase H; or
b) an RNAi agent capable of reducing the amount of HTT nucleic acid through the activation of RISC/Ago2.

Embodiment 107. A pharmaceutical composition comprising an oligomeric compound of any of embodiments 1-62, a modified oligonucleotide of any of embodiments 63-71, a population of any of embodiments 72-79, an oligomeric duplex of any of embodiments 81-103, or an antisense agent of any of embodiments 104-106; and a pharmaceutically acceptable carrier or diluent.

Embodiment 108. The pharmaceutical composition of embodiment 107, wherein the pharmaceutically acceptable diluent comprises phosphate buffered saline (PBS) or artificial cerebrospinal fluid (aCSF).

Embodiment 109. The pharmaceutical composition of embodiment 108, wherein the pharmaceutical composition consists essentially of the oligomeric compound of any of embodiments 1-62, the modified oligonucleotide of any of embodiments 63-71, the population of any of embodiments 72-79, the oligomeric duplex of any of embodiments 81-103, or the antisense agent of any of embodiments 104-106, and PBS.

Embodiment 110. The pharmaceutical composition of embodiment 108, wherein the pharmaceutical composition consists essentially of the oligomeric compound of any of embodiments 1-62, the modified oligonucleotide of any of embodiments 63-71, the population of any of embodiments 72-79, the oligomeric duplex of any of embodiments 81-103, or the antisense agent of any of embodiments 104-106, and aCSF.

Embodiment 111. A method comprising administering to a subject an oligomeric compound of any of embodiments 1-62, a modified oligonucleotide of any of embodiments 63-71, a population of any of embodiments 72-79, an oligomeric duplex of any of embodiments 81-103, an antisense agent of any of embodiments 104-106, or a pharmaceutical composition of any of embodiments 107-110.

Embodiment 112. A method of ameliorating or preventing a repeat expansion disease, comprising administering to a subject in need thereof a therapeutically effective amount of an oligomeric compound of any of embodiments 1-62, a modified oligonucleotide of any of embodiments 63-71, a population of any of embodiments 72-79, an oligomeric duplex of any of embodiments 81-103, an antisense agent of any of embodiments 104-106, or a pharmaceutical composition of any of embodiments 107-110.

Embodiment 113. The method of embodiment 112, wherein the repeat expansion disease is selected from myotonic dystrophy (DM1 and DM2), amyotrophic lateral sclerosis, frontotemporal dementia, Huntington's disease, a polyglutamine disorder, Fragile X syndrome, and a spinocerebellar ataxia.

Embodiment 114. The method of embodiment 112, wherein the repeat expansion disease is Huntington's disease.

Embodiment 115. The method of embodiment 112, wherein the repeat expansion disease is a spinocerebellar ataxia.

Embodiment 116. The method of any of embodiments 112-115, wherein at least one symptom or hallmark of the repeat expansion disease is ameliorated.

Embodiment 117. The method of embodiment 116, wherein the symptom or hallmark is selected from brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, and a combination thereof.

Embodiment 118. The method of any of embodiments 116-117, wherein administering the oligomeric compound of any of embodiments 1-62, the modified oligonucleotide of any of embodiments 63-71, the population of any of embodiments 72-79, the oligomeric duplex of any of embodiments 81-103, the antisense agent of any of embodiments 104-106, or the pharmaceutical composition of any of embodiments 107-110 reduces or delays the onset or progression of brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, or suicidal behavior.

Embodiment 119. The method of any of embodiments 111-118, comprising identifying the subject as having the repeat expansion disease or at risk for having the repeat expansion disease.

Embodiment 120. The method of any of embodiments 111-119, wherein the oligomeric compound of any of embodiments 1-62, the modified oligonucleotide of any of embodiments 63-71, the population of any of embodiments 72-79, the oligomeric duplex of any of embodiments 81-103, the antisense agent of any of embodiments 104-106, or the pharmaceutical composition of any of embodiments 107-110 is administered to the central nervous system or systemically.

Embodiment 121. The method of any of embodiments 111-119, wherein the oligomeric compound of any of embodiments 1-62, the modified oligonucleotide of any of embodiments 63-71, the population of any of embodiments 72-79, the oligomeric duplex of any of embodiments 81-103, the antisense agent of any of embodiments 104-106, or the pharmaceutical composition of any of embodiments 107-110 is administered intrathecally.

Embodiment 122. The method of any of embodiments 111-121, wherein the subject is a human.

Embodiment 123. A method of reducing expression of HTT in a cell comprising contacting the cell with an oligomeric compound of any of embodiments 1-62, a modified oligonucleotide of any of embodiments 63-71, a population of any of embodiments 72-79, an oligomeric duplex of any of embodiments 81-103, an antisense agent of any of embodiments 104-106, or a pharmaceutical composition of any of embodiments 107-110.

Embodiment 124. The method of embodiment 123, wherein the cell is from a brain tissue, optionally from the cortex, substantia nigra, striatum, midbrain, brainstem, or spinal cord.

Embodiment 125. The method of embodiment 123 or embodiment 124, wherein the cell is a human cell.

Embodiment 126. Use of an oligomeric compound of any of embodiments 1-62, a modified oligonucleotide of any of embodiments 63-71, a population of any of embodiments 72-79, an oligomeric duplex of any of embodiments 81-103, an antisense agent of any of embodiments 104-106, or a pharmaceutical composition of any of embodiments 107-110 for ameliorating or preventing a repeat expansion disease.

Embodiment 127. Use of an oligomeric compound of any of embodiments 1-62, a modified oligonucleotide of any of embodiments 63-71, a population of any of embodiments 72-79, an oligomeric duplex of any of embodiments 81-103, an antisense agent of any of embodiments 104-106, or a pharmaceutical composition of any of embodiments 107-110 in the manufacture of a medicament for ameliorating or preventing a repeat expansion disease.

Embodiment 128. The use of embodiment 126 or embodiment 127, wherein the repeat expansion disease is Huntington's disease.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: —F, —OCH₃ (“OMe” or “O-methyl”), and —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂(“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include but are not limited to vinyl, alkoxy (e.g., methoxy), alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

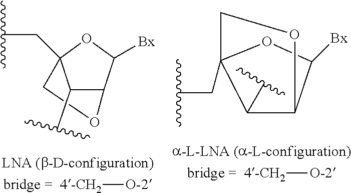

α-L-methyleneoxy (4′-CH₂—O—2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

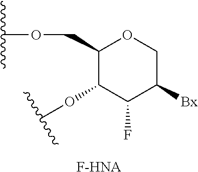

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

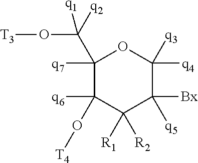

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

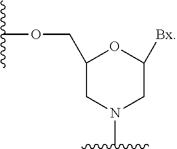

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

### (S)-GNA

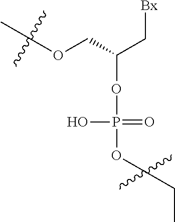

where Bx represents any nucleobase.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

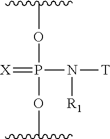

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

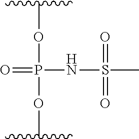 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

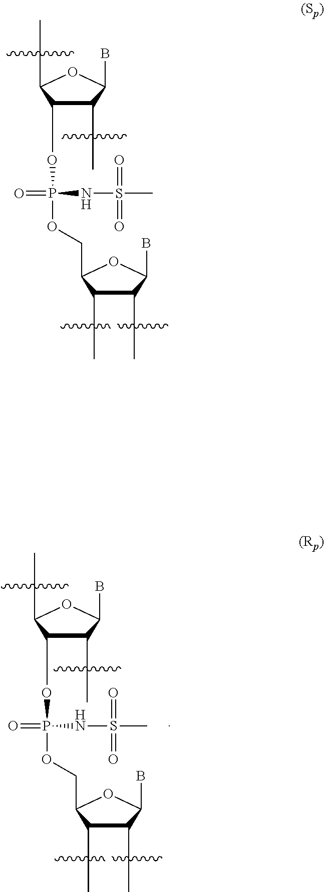

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

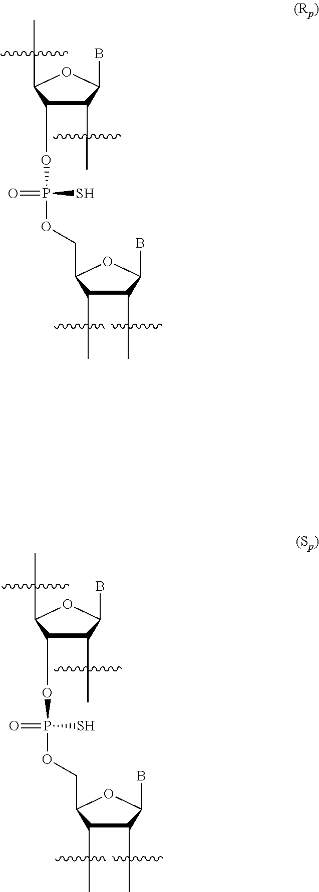

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O—5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

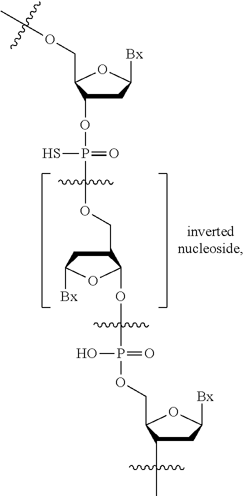

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

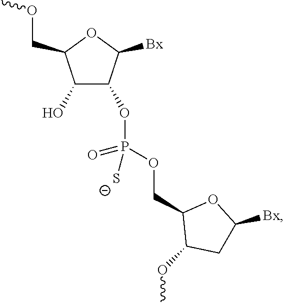

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-β-D-deoxyribosyl sugar moieties and the nucleosides on the wing sides of each wing/gap junction comprise modified sugar moieties. In certain embodiments, each nucleoside of the gap comprise 2′-β-D-deoxyribosyl sugar moieties. In certain embodiments, each nucleoside of each wing of a gapmer comprises a modified sugar moiety. In certain embodiments, at least one nucleoside of the gap of a gapmer comprises a modified sugar moiety. In certain embodiments, at least one nucleoside of the gap of a gapmer comprises a 2′-OMe or a 2′-cEt sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments, modified oligonucleotides are 5-8-5 MOE gapmers. In certain embodiments, modified oligonucleotides are 6-10-4 MOE gapmers.

A mixed wing gapmer has at least two different modified sugars in the 5′ and/or 3′ wing. In certain embodiments, modified oligonucleotides are 4-8-5 mixed wing gapmers which has at least two different sugar moieties in the 5′- and/or the 3′-wing. In certain embodiments, the at least two different sugar moieties are selected from 2′-MOE and 2′-cEt sugar moieties.

In certain embodiments, modified oligonucleotides have a sugar motif selected from the following: 5′-eekkddddddddkkeee-3′, 5′-ekekddddddddkekee-3′, 5′-eeeeeddddddddddeeeee-3′, 5′-eeeeddddddddkkeee-3′, 5′-eeeeeddddddddeeeee-3′, and 5′-eeeeeeddddddddddeeee-3′, wherein each “d” represents a 2′-β-D-deoxyribosyl sugar moiety, each “e” represents a 2′-MOE sugar moiety, and each “k” represents a cEt sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-β-D-deoxyribosyl sugar moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, or Rp motif. In certain embodiments, populations of modified oligonucleotides are enriched for modified oligonucleotides comprising such internucleoside linkage motifs.

In certain embodiments, modified oligonucleotides have an internucleoside linkage motif selected from: 5′-soosssssssssooss-3′, 5′-sooosssssssssssooss-3′, 5′-sooosssssssssssooos-3′, 5′-sossssssssssssssoss-3′, 5′-ssoosssssssssssooss-3′, 5′-sooosssssssssooss-3′, 5′-sossssssssssssoss-3′, and 5′-sooooossssssssssoss-3′. In certain embodiments, each “s” represents a phosphorothioate internucleoside linkage and each “o” represents a phosphodiester internucleoside linkage.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidates linking group.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

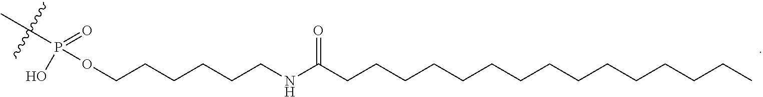 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates, antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

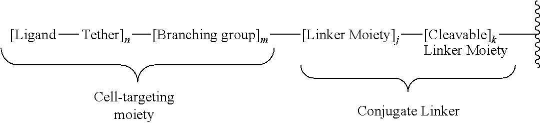

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

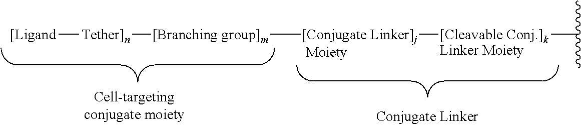

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)2, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA or dsRNAi) or single-stranded (ssRNAi).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### A. Complementarity/Mismatches to the Target Nucleic Acid

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. HTT

In certain embodiments, oligomeric compounds comprise or consist of an oligonucleotide comprising a region that is complementary to an HTT nucleic acid. In certain embodiments, the HTT nucleic acid has the sequence set forth as GENBANK Accession No. NM_002111.6 (incorporated herein as SEQ ID NO: 1), GENBANK Accession No. NT_006081.17 truncated from nucleotides 462000 to 634000 (incorporated herein as SEQ ID NO: 2), GENBANK Accession No. NM_010414.1 (incorporated herein as SEQ ID NO: 3), the complement of GENBANK Accession No. NW_001109716.1 truncated at nucleotides 698000 to 866000 (incorporated herein as SEQ ID NO: 4), and GENBANK Accession No. NM_024357.2 (incorporated herein as SEQ ID NO: 5).

In certain embodiments an oligomeric compound complementary to any one of SEQ ID NOs: 1-5 is capable of reducing HTT RNA in a cell. In certain embodiments an oligomeric compound complementary to any one of SEQ ID NOs: 1-5 is capable of reducing HTT protein in a cell. In certain embodiments, the cell is in vitro. In certain embodiments, the cell is in a subject. In certain embodiments, the oligomeric compound consists of a modified oligonucleotide. In certain embodiments, an oligomeric compound complementary to any one of SEQ ID NOs: 1-5 is capable of ameliorating one or more symptom or hallmark of a neurodegenerative disease when it is introduced to a cell in a subject. In certain embodiments, the neurodegenerative disease is a repeat expansion disease. In certain embodiments, the repeat expansion disease is selected from myotonic dystrophy (DM1 and DM2), amyotrophic lateral sclerosis, frontotemporal dementia, Huntington's disease, various polyglutamine disorders, Fragile X syndrome, and a spinocerebellar ataxia (SCA1, SCA2, SCA3, SCA6, SCA7, SCA8, SCA10, SCA17, or Friedrich's ataxia). In certain embodiments, the symptom or hallmark is brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

In certain embodiments, an oligomeric compound complementary to any one of SEQ ID NOs: 1-5 is capable of reducing a detectable amount of HTT RNA in the CSF of a subject after the oligomeric compound is administered to the subject. The detectable amount of HTT RNA may be reduced by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing a detectable amount of HTT protein in the CSF of the subject when the oligomeric compound is administered to the subject. The detectable amount of HTT protein may be reduced by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing a detectable amount of mHTT protein in the CSF of the subject when the oligomeric compound is administered to the subject. The detectable amount of mHTT protein may be reduced by at least 10%, at least 20%, at least 30%, at least 40%, at least 50%, at least 60%, at least 70%, at least 80%, or at least 90%. In certain embodiments, the oligomeric compound is administered into the CSF of the subject.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, cortex, substantia nigra, striatum, midbrain, and brainstem and spinal cord.

### VI. Certain Methods and Uses

Certain embodiments provided herein relate to methods of reducing or inhibiting HTT expression or activity, which can be useful for treating, preventing, or ameliorating a repeat expansion disease. In certain embodiments, the repeat expansion disease is myotonic dystrophy (DM1 and DM2), amyotrophic lateral sclerosis, frontotemporal dementia, Huntington's disease, a polyglutamine disorder, Fragile X syndrome, or a spinocerebellar ataxia. In certain embodiments, the repeat expansion disease is Huntington's disease. In certain embodiments, the repeat expansion disease is a spinocerebellar ataxia.

In certain embodiments, a method comprises administering to a subject an oligomeric compound, a modified oligonucleotide, an oligomeric duplex, or an antisense agent, any of which having a nucleobase sequence complementary to a HTT nucleic acid. In certain embodiments, the subject has or is at risk for developing a repeat expansion disease. In certain embodiments, the subject has or is at risk for developing myotonic dystrophy (DM1 and DM2), amyotrophic lateral sclerosis, frontotemporal dementia, Huntington's disease, a polyglutamine disorder, Fragile X syndrome, or a spinocerebellar ataxia. In certain embodiments, the subject has or is at risk for developing Huntington's disease. In certain embodiments, the subject has or is at risk for developing a spinocerebellar ataxia.

In certain embodiments, a method of ameliorating, treating, or preventing a repeat expansion disease comprises administering to a subject an oligomeric compound, a modified oligonucleotide, an oligomeric duplex, or an antisense agent, any of which having a nucleobase sequence complementary to a HTT nucleic acid. In certain embodiments, a method of ameliorating or preventing a repeat expansion disease comprises administering to a subject an oligomeric compound, a modified oligonucleotide, an oligomeric duplex, or an antisense agent, any of which having a nucleobase sequence complementary to a HTT nucleic acid. In certain embodiments, the subject has or is at risk for developing a repeat expansion disease. In certain embodiments, the subject has myotonic dystrophy (DM1 and DM2), amyotrophic lateral sclerosis, frontotemporal dementia, Huntington's disease, a polyglutamine disorder, Fragile X syndrome, or a spinocerebellar ataxia. In certain embodiments, the subject has Huntington's disease. In certain embodiments, the subject has a spinocerebellar ataxia. In certain embodiments, at least one symptom or hallmark of the repeat expansion disease is ameliorated. In certain embodiments, the symptom or hallmark is selected from brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, and a combination thereof. In certain embodiments, administration of the oligomeric compound, the modified oligonucleotide, the oligomeric duplex, or the antisense agent reduces or delays the onset or progression of brain atrophy, reduced brain activity, reduced brain connectivity, muscle atrophy, muscle weakness, muscle cramping, difficulty swallowing, seizure, tremor, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, or suicidal behavior.

In certain embodiments, a method of ameliorating, treating, or preventing Huntington's disease comprises administering to a subject an oligomeric compound, a modified oligonucleotide, an oligomeric duplex, or an antisense agent, any of which having a nucleobase sequence complementary to a HTT nucleic acid. In certain embodiments, a method of ameliorating or preventing Huntington's disease comprises administering to a subject an oligomeric compound, a modified oligonucleotide, an oligomeric duplex, or an antisense agent, any of which having a nucleobase sequence complementary to a HTT nucleic acid. In certain embodiments, at least one symptom or hallmark of Huntington's disease is ameliorated. In certain embodiments, the symptom or hallmark is brain atrophy, reduced brain activity, reduced brain connectivity, muscle atrophy, muscle weakness, muscle cramping, difficulty swallowing, seizure, tremor, nerve degeneration, impaired glucose tolerance, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired mental concentration, impaired speech, depression, irritability, impaired mobility, impaired self-care, anxiety, suicidal ideation, or suicidal behavior. In certain embodiments, administration of the oligomeric compound, the modified oligonucleotide, the oligomeric duplex, or the antisense agent reduces or delays the onset or progression of brain atrophy, reduced brain activity, reduced brain connectivity, muscle atrophy, muscle weakness, muscle cramping, difficulty swallowing, seizure, tremor, nerve degeneration, impaired glucose tolerance, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired mental concentration, impaired speech, depression, irritability, impaired mobility, impaired self-care, anxiety, suicidal ideation, or suicidal behavior.

In certain embodiments, a method of reducing expression of HTT nucleic acid, for example RNA, or reducing expression of HTT protein in a cell comprises contacting the cell with an oligomeric compound, a modified oligonucleotide, an oligomeric duplex, or an antisense agent, any of which having a nucleobase sequence complementary to a HTT nucleic acid. In certain embodiments, the cell is from a subject. In certain embodiments, the subject has or is at risk for developing a repeat expansion disease. In certain embodiments, the subject has or is at risk for developing Huntington's disease. In certain embodiments, the cell is from a brain tissue, optionally from the cortex, substantia nigra, striatum, midbrain, brainstem, or spinal cord. In certain embodiments, the cell is a human cell.

Certain embodiments are drawn to an oligomeric compound, a modified oligonucleotide, an oligomeric duplex, or an antisense agent, any of which having a nucleobase sequence complementary to a HTT nucleic acid, for use in ameliorating, treating, or preventing a repeat expansion disease or for use in the manufacture of a medicament for ameliorating, treating, or preventing a repeat expansion disease. In certain embodiments, an oligomeric compound, a modified oligonucleotide, an oligomeric duplex, or an antisense agent, any of which having a nucleobase sequence complementary to a HTT nucleic acid, is for use in ameliorating or preventing a repeat expansion disease or for use in the manufacture of a medicament for ameliorating or preventing a repeat expansion disease. In certain embodiments, the repeat expansion disease is selected from myotonic dystrophy (DM1 and DM2), amyotrophic lateral sclerosis, frontotemporal dementia, Huntington's disease, a polyglutamine disorder, Fragile X syndrome, and a spinocerebellar ataxia. In certain embodiments, the repeat expansion disease is Huntington's disease. In certain embodiments, the repeat expansion disease is a spinocerebellar ataxia.

Certain embodiments are drawn to an oligomeric compound, a modified oligonucleotide, an oligomeric duplex, or an antisense agent, any of which having a nucleobase sequence complementary to a HTT nucleic acid, for use in ameliorating, treating, or preventing Huntington's disease or for use in the manufacture of a medicament for ameliorating, treating, or preventing Huntington's disease. In certain embodiments, an oligomeric compound, a modified oligonucleotide, an oligomeric duplex, or an antisense agent, any of which having a nucleobase sequence complementary to a HTT nucleic acid, is for use in ameliorating or preventing Huntington's disease or for use in the manufacture of a medicament for ameliorating or preventing Huntington's disease.

In any of the methods or uses described herein, the oligomeric compound, the modified oligonucleotide, the oligomeric duplex, or the antisense agent can be any described herein.

### VII. Certain Compositions

### Compound No. 1394371

In certain embodiments, Compound No. 1394371 is characterized as a 6-10-4 MOE gapmer, having a sequence of (from 5′ to 3′) CACAGCTTTTATTTCCATAC (incorporated herein as SEQ ID NO: 3619), wherein each of nucleosides 1-6 and 17-20 are 2′-O-methoxyethyl nucleosides, and each of nucleosides 7-16 are β-D-deoxyribonucleosides, wherein the internucleoside linkages between nucleosides 2 to 3, 3 to 4, 4 to 5, 5 to 6, 6 to 7, and 17 to 18 are phosphodiester linkages and the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate linkages, and wherein each cytosine is a 5-methylcytosine.

In certain embodiments, Compound No. 1394371 is described by the following chemical notation: ^{m}Cₑₛ Aₑₒ ^{m}Cₑₒ Aₑₒ Gₑₒ ^{m}Cₑₒ T_{ds} T_{ds} T_{ds} T_{ds} A_{ds} T_{ds} T_{ds} T_{ds} ^{m}C_{ds} ^{m}C_{ds} Aₑₒ Tₑₛ Aₑₛ ^{m}Cₑ (SEQ ID NO: 3619); wherein, A=an adenine, ^{m}C=a 5-methylcytosine, G=a guanine, T=a thymine, e=a 2′-O-methoxyethylribose modified sugar, d=a 2′-deoxyribose sugar moiety, s=a phosphorothioate internucleoside linkage, and o=a phosphodiester internucleoside linkage.

In certain embodiments, Compound No. 1394371 is described by the following chemical structure, or a pharmaceutically acceptable salt thereof:

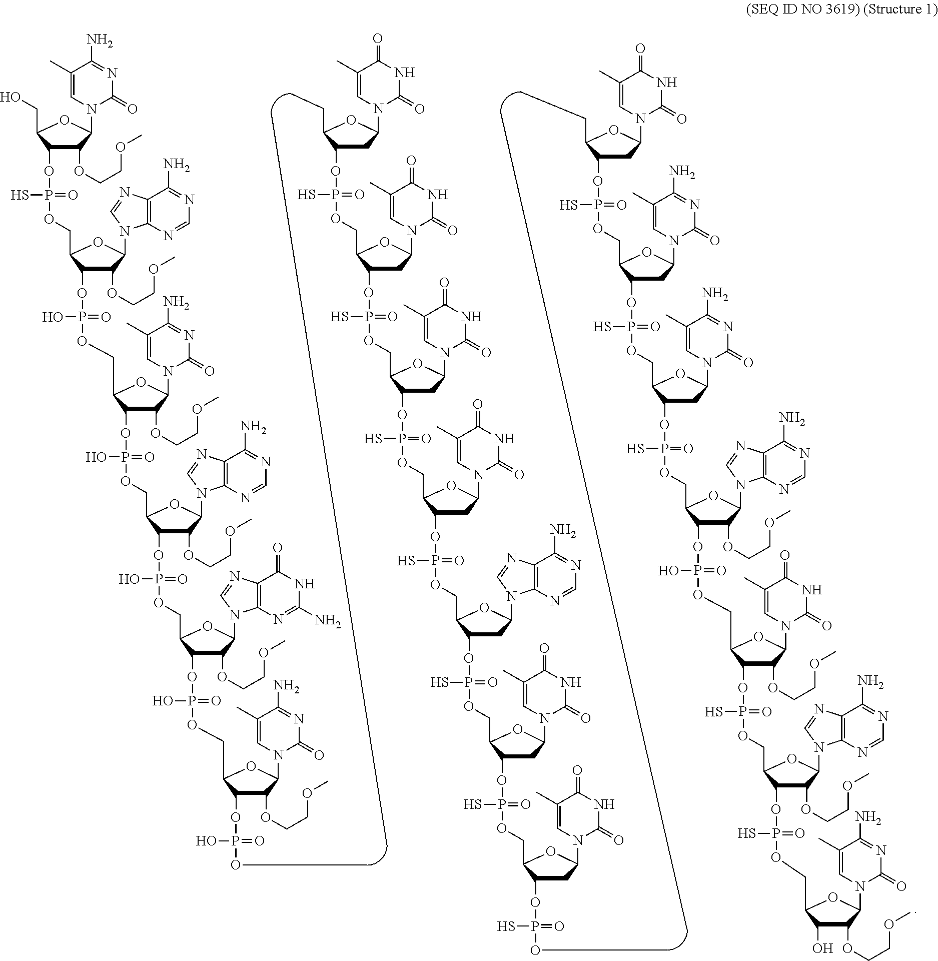

In certain embodiments, a pharmaceutically acceptable salt of Compound No. 1394371 described by Structure 1 comprises one or more cations selected from sodium, potassium, calcium, and magnesium.

In certain embodiments, the sodium salt of Compound No. 1394371 is described by the following chemical structure:

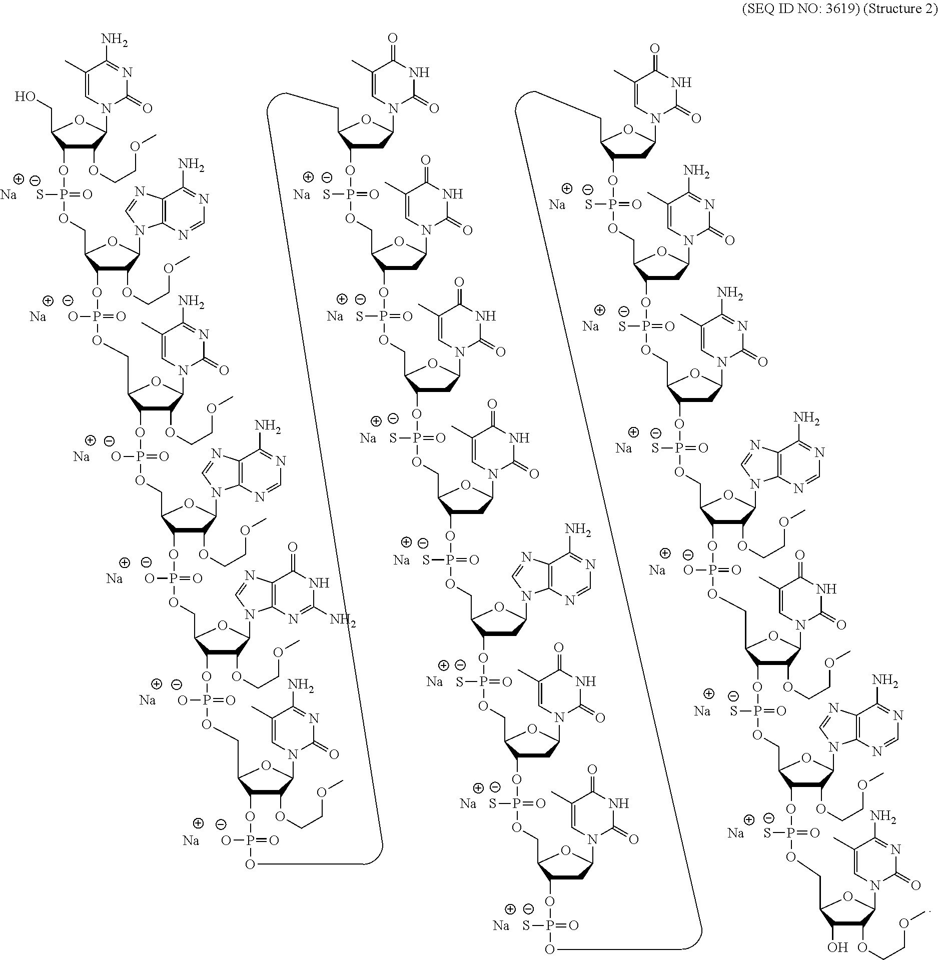

### Compound No. 1315578

In certain embodiments, Compound No. 1315578 is characterized as a 5-10-5 MOE gapmer, having a sequence of (from 5′ to 3′) GCTTTGATTTTTACCAGTTA (incorporated herein as SEQ ID NO: 1502), wherein each of nucleosides 1-5 and 16-20 are 2′-O-methoxyethyl nucleosides, and each of nucleosides 6-15 are β-D-deoxyribonucleosides, wherein the internucleoside linkages between nucleosides 2 to 3, 3 to 4, 4 to 5, 16 to 17, and 17 to 18 are phosphodiester linkages and the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate linkages, and wherein each cytosine is a 5-methylcytosine.

In certain embodiments, Compound No. 1315578 is described by the following chemical notation: Gₑₛ ^{m}Cₑₒ Tₑₒ Tₑₒ Tₑₛ G_{ds} A_{ds} T_{ds} T_{ds} T_{ds} T_{ds} T_{ds} A_{ds} ^{m}C_{ds} ^{m}C_{ds} Aₑₒ Gₑₒ Tₑₛ Tₑₛ Aₑ (SEQ ID NO: 1502); wherein, A=an adenine, ^{m}C=a 5-methylcytosine, G=a guanine, T=a thymine, e=a 2′-O-methoxyethylribose modified sugar, d=a 2′-deoxyribose sugar moiety, s=a phosphorothioate internucleoside linkage, and o=a phosphodiester internucleoside linkage.

In certain embodiments, Compound No. 1315578 is described by the following chemical structure, or a pharmaceutically acceptable salt thereof:

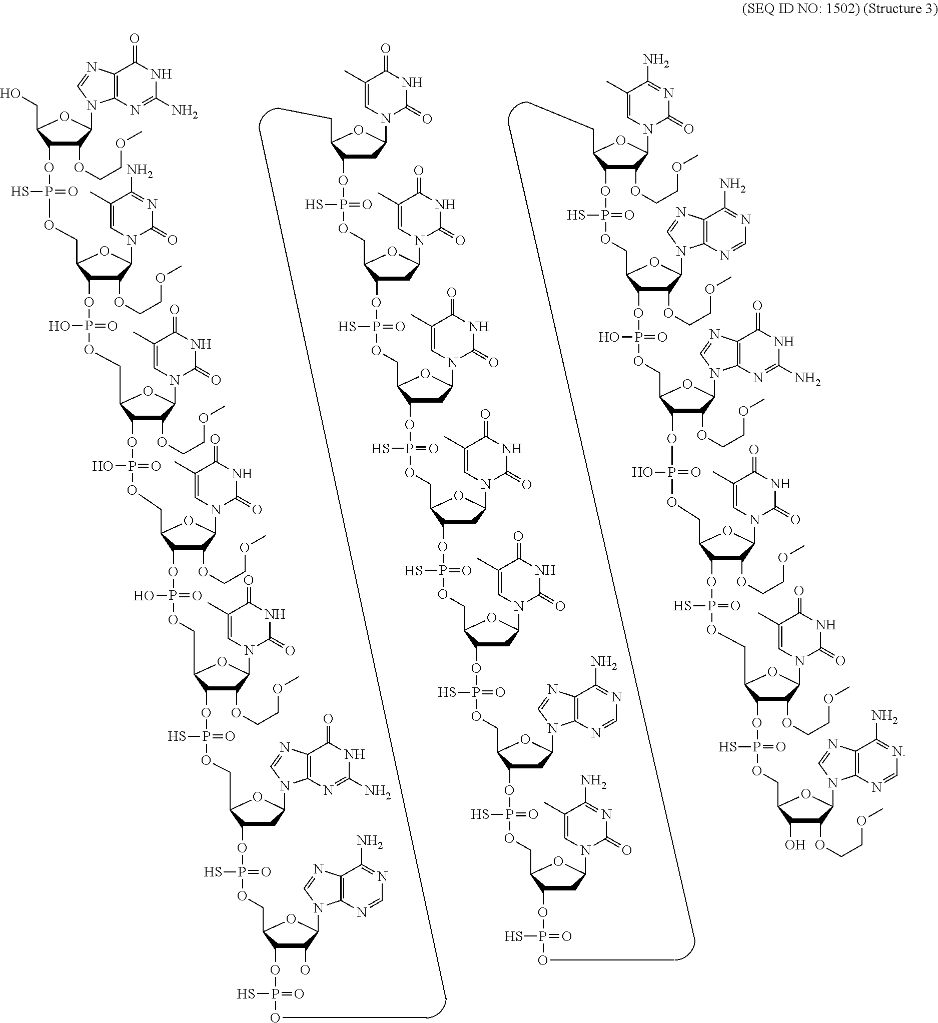

In certain embodiments, a pharmaceutically acceptable salt of Compound No. 1315578 described by Structure 3 comprises one or more cations selected from sodium, potassium, calcium, and magnesium.

In certain embodiments, the sodium salt of Compound No. 1315578 is described by the following chemical structure:

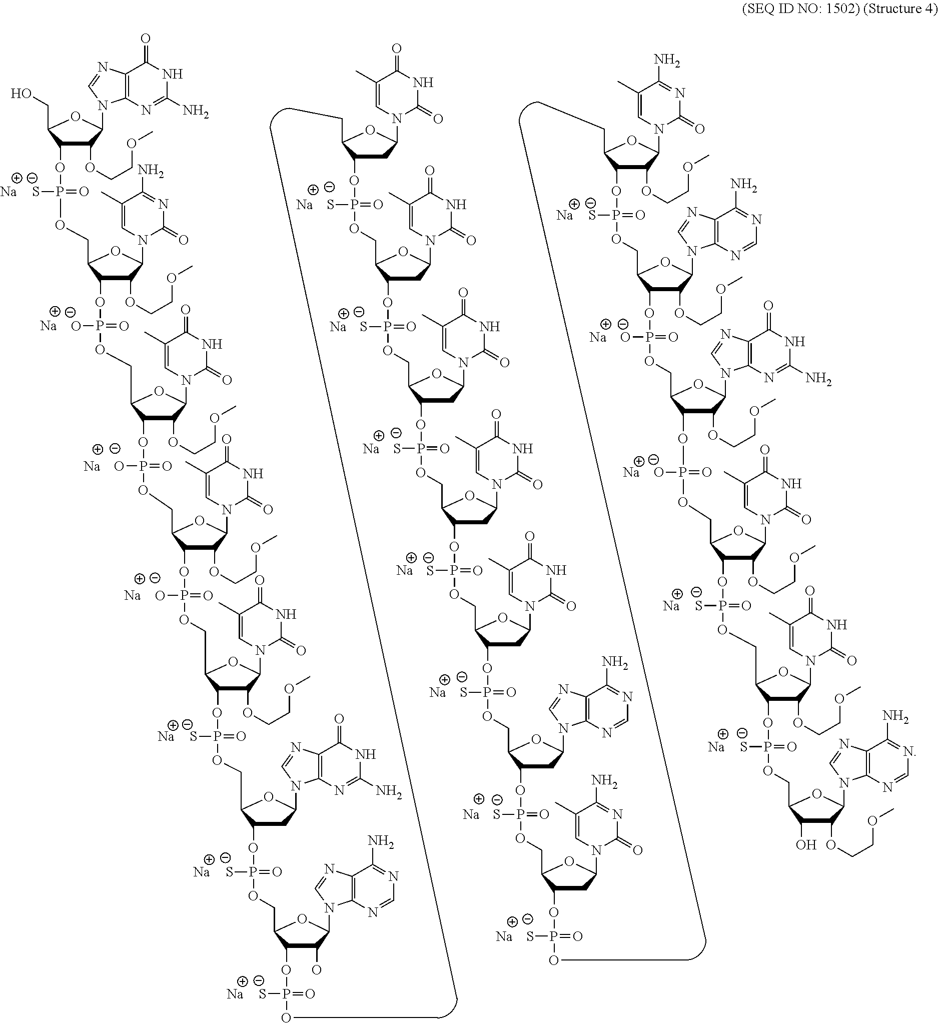

### Compound No. 1394378

In certain embodiments, Compound No. 1394378 is characterized as a 6-10-4 MOE gapmer, having a sequence of (from 5′ to 3′) ACAGCTTTTATTTCCATACA (incorporated herein as SEQ ID NO: 3646), wherein each of nucleosides 1-6 and 17-20 are 2′-O-methoxyethyl nucleosides, and each of nucleosides 7-16 are β-D-deoxyribonucleosides, wherein the internucleoside linkages between nucleosides 2 to 3, 3 to 4, 4 to 5, 5 to 6, 6 to 7, and 17 to 18 are phosphodiester linkages and the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate linkages, and wherein each cytosine is a 5-methylcytosine.

In certain embodiments, Compound No. 1394378 is described by the following chemical notation: Aₑₛ ^{m}Cₑₒ Aₑₒ Gₑₒ ^{m}Cₑₒ Tₑₒ T_{ds} T_{ds} T_{ds} A_{ds} T_{ds} T_{ds} T_{ds} ^{m}C_{ds} ^{m}C_{ds} A_{ds} Tₑₒ Aₑₛ ^{m}Cₑₛ Aₑ (SEQ ID NO: 3646); wherein, A=an adenine, ^{m}C=a 5-methylcytosine, G=a guanine, T=a thymine, e=a 2′-O-methoxyethylribose modified sugar, d=a 2′-deoxyribose sugar moiety, s=a phosphorothioate internucleoside linkage, and o=a phosphodiester internucleoside linkage.

In certain embodiments, Compound No. 1394378 is described by the following chemical structure, or a pharmaceutically acceptable salt thereof:

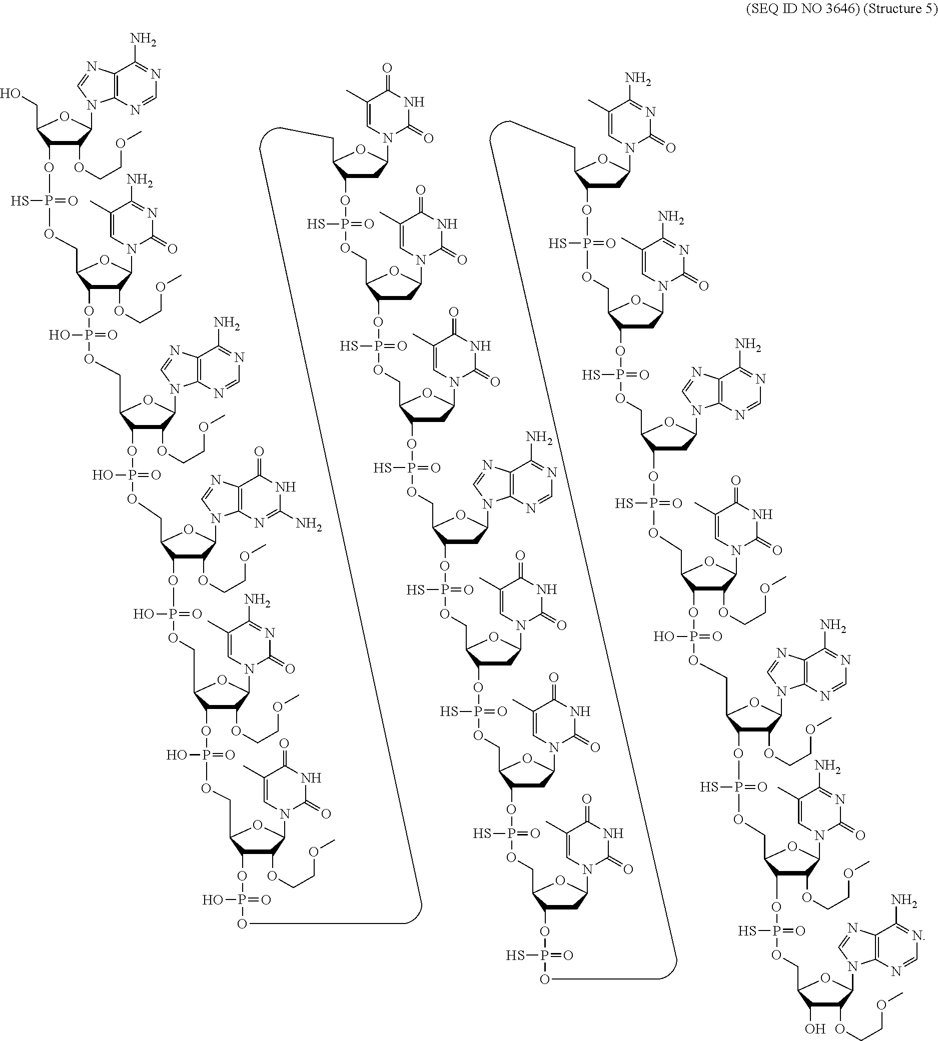

In certain embodiments, a pharmaceutically acceptable salt of Compound No. 1394378 described by Structure 5 comprises one or more cations selected from sodium, potassium, calcium, and magnesium.

In certain embodiments, the sodium salt of Compound No. 1394378 is described by the following chemical structure:

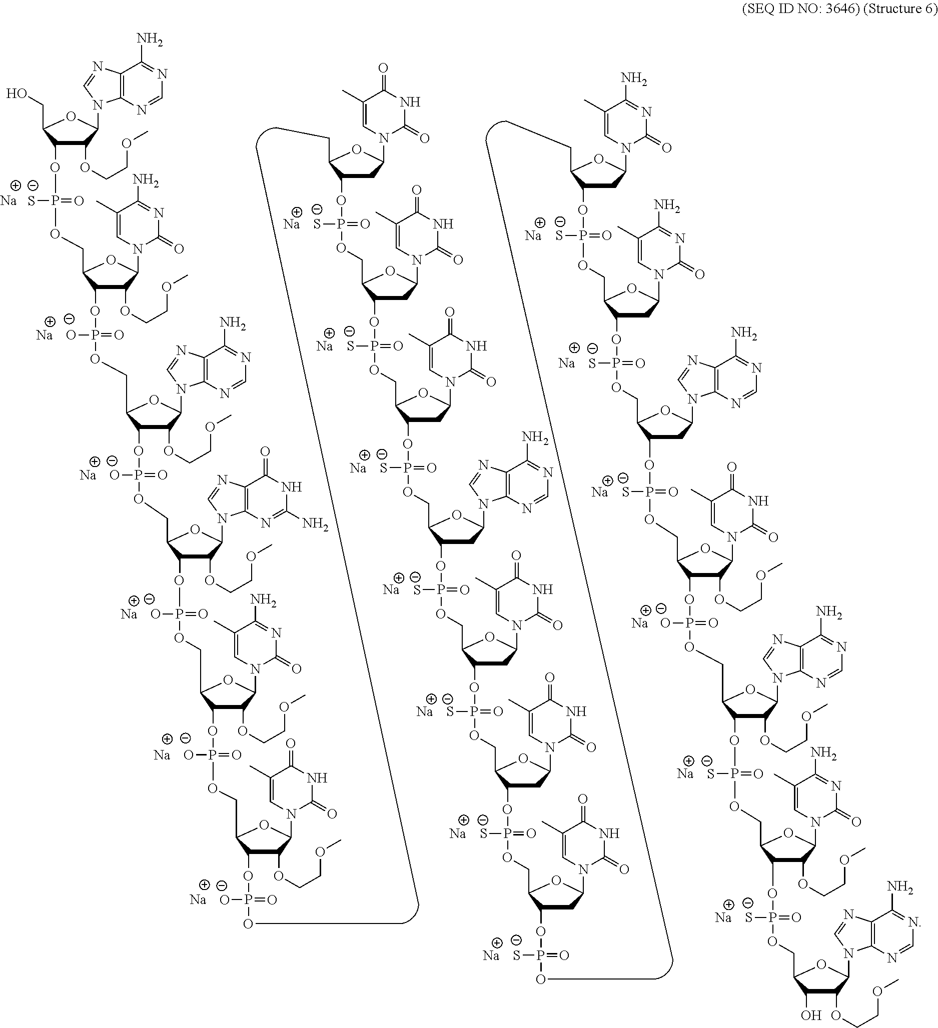

### VIII. Certain Comparator Compositions

In certain embodiments, Compound No: 388241, a 5-10-5 MOE gapmer, having a sequence of (from 5′ to 3′) CTCAGTAACATTGACACCAC (incorporated herein as SEQ ID NO: 858), wherein each internucleoside linkage is a phosphorothioate internucleoside linkage, each cytosine is a 5-methylcytosine, and each of nucleosides 1-5 and 16-20 (from 5′ to 3′) comprise a 2′-MOE modified sugar, which was previously described in WO 2011/032045, incorporated herein by reference, is a comparator compound. Compound No. 388241 was selected as a comparator compound because it was one of the most potent compounds described in WO 2011/032045. Compound No. 388241 and Compound No. 443139, also one of the most potent compounds described in WO 2011/032045, were tested in comparative studies and achieved similar results, as described in WO 2011/032045.

In certain embodiments, compounds described herein, including Compound Nos. 1394371, 1315578, and 1394378, are superior relative to Compound No. 388241 because they demonstrate one or more improved properties, such as, activity and tolerability. Compound Nos. 1394371, 1315578, and 1394378 may be dosed at lower amounts than Compound No. 388241 to achieve a comparable therapeutic effect. Compound Nos. 1394371, 1315578, and 1394378 may be dosed less frequently than Compound No. 388241 to achieve a comparable therapeutic effect.

For example, as provided in Example 8 (hereinbelow), Compound Nos. 1394371, 1315578, and 1394378 demonstrated improved tolerability in rats relative to that of Compound No. 388241. Compound Nos. 1394371, 1315578, and 1394378 demonstrated 3 h FOB scores of 0.00, 0.50, and 0.00, respectively. In contrast, Compound No. 388241 demonstrated a 3 h FOB score of 2.75. See Tables 77 and 78. Therefore, Compound Nos. 1394371, 1315578, and 1394378 are demonstrably more tolerable than Compound No. 388241 in rats.

For example, as provided in Example 9 (hereinbelow), Compound Nos. 1394371, 1315578, and 1394378 demonstrated improved activity in human HTT transgenic mice relative to that of Compound No. 388241. Compound Nos. 1394371, 1315578, and 1394378 reduced HTT RNA levels in spinal cords of mice to 26%, 46% and 28% of HTT RNA levels in untreated control mice, respectively, whereas as Compound No. 388241 only reduced HTT RNA levels in spinal cords of mice to 73% of HTT RNA levels in untreated control mice. Compound Nos. 1394371, 1315578, and 1394378 reduced HTT RNA levels in the cortex of mice to 10% of HTT RNA levels in untreated control mice, respectively, whereas as Compound No. 388241 only reduced HTT RNA levels in the cortex of mice to 56% of HTT RNA levels in untreated control mice.

### IX. Certain Hotspot Regions

In certain embodiments, the ranges described in the Table below comprise hotspot regions. Each hotspot region begins with the nucleobase of SEQ ID NO: 2 identified in the “Start Site SEQ ID NO: 2” column and ends with the nucleobase of SEQ ID NO: 2 identified in the “Stop Site SEQ ID NO: 2” column. In certain embodiments, oligomeric compounds comprise modified oligonucleotides that are complementary within any of the hotspot regions 1-10, as defined in the table below. In certain embodiments, modified oligonucleotides are 16 nucleobases in length. In certain embodiments, modified oligonucleotides are 17 nucleobases in length. In certain embodiments, modified oligonucleotides are 18 nucleobases in length. In certain embodiments, modified oligonucleotides are 20 nucleobases in length.

In certain embodiments, oligomeric compounds comprise modified oligonucleotides that are gapmers. In certain embodiments, modified oligonucleotides are 5-10-5 MOE gapmers. In certain embodiments, modified oligonucleotides are 6-10-4 MOE gapmers. In certain embodiments, modified oligonucleotides are 5-8-5 MOE gapmers. In certain embodiments, modified oligonucleotides are gapmers, wherein the wings have a cEt/MOE sugar motif. In certain embodiments, modified oligonucleotides have a sugar motif selected from: eekkddddddddkkeee, ekekddddddddkekee, eeeeeddddddddddeeeee, eeeeddddddddkkeee, and eeeeeeddddddddeeee, wherein each “e” is a nucleoside comprising a 2′-MOE sugar moiety, each “k” is a nucleoside comprising a cEt sugar moiety, and each “d” is a nucleoside comprising a 2′-β-D-deoxyribosyl sugar moiety.

In certain embodiments, oligomeric compounds comprise a modified oligonucleotide comprising a modified internucleoside linkage. In certain embodiments, the modified internucleotides linkage is a phosphorothioate linkage. In certain embodiments, modified oligonucleotides have a internucleoside linkage motif selected from: soosssssssssooss, sooosssssssssssooss, sssssssssssssssssss, sooosssssssssssooos, sossssssssssssssoss, ssoosssssssssssooss, sooosssssssssooss, sooooossssssssssoss, and sossssssssssssoss, wherein each “s” is a phosphorothioate linkage an each “o” is a phosphodiester linkage.

The nucleobase sequences of the oligomeric compounds in the table below are complementary to SEQ ID NO: 2 within the specified hotspot region. In certain embodiments, compounds comprising a modified oligonucleotide complementary to nucleobases within the hotspot region achieve at least “Min. % Red.” (minimum 0 reduction, relative to untreated control cells) of HTT RNA in the standard in vitro assay, as indicated in the table below. In certain embodiments, modified oligonucleotides complementary to nucleobases within the hotspot region achieve an average of “Avg. % Red.” (average % reduction, relative to untreated control cells) of HTT RNA in the standard in vitro assay, as indicated in the table below. In certain embodiments, modified oligonucleotides complementary to nucleobases within the hotspot region achieve a maximum “Max. % Red.” (maximum % reduction, relative to untreated control cells) of HTT RNA in the standard in vitro assay, as indicated in the table below.

TABLE 1
HTT Hotspots

| | Start | Stop | Min | Max | | | |
|---|---|---|---|---|---|---|---|
| | Site | Site | % | % | Avg % | | |
| | SEQ | SEQ | Red. | Red. | Red. | | SEQ ID |
| Hotspot | ID | ID | In | In | In | Compound No. | NOs in |
| ID | NO: 2 | NO: 2 | vitro | vitro | vitro | in range | range |
| 1 | 56443 | 56500 | 38 | 88 | 72.1 | 1314697, 1314867, | 1184, 1274, |
| | | | | | | 1314999, 1315294, | 1331, 1455, |
| | | | | | | 1315702, 1316563, | 1481, 1562, |
| | | | | | | 1316677 | 1684 |
| 2 | 62833 | 62860 | 83 | 92 | 87.5 | 1391321, 1314907, | 3029, 3104, |
| | | | | | | 1314967, 1391310, | 3557, 3562, |
| | | | | | | 1391323, 1391324, | 3569, 3586, |
| | | | | | | 1391328, 1391336, | 3610, 3620, |
| | | | | | | 1391368, 1394346, | 3622, 3623, |
| | | | | | | 1394375, 1394381, | 3627, 3634, |
| | | | | | | 1394384, 1394385, | 3664, 3687, |
| | | | | | | 1394393, 1394395, | 3692, 3697 |
| | | | | | | 1394422, 1394428, | |
| | | | | | | 1394433, 1394440, | |
| | | | | | | 1394451, 1419391, | |
| | | | | | | 1419396, 1419403, | |
| | | | | | | 1419421 | |
| 3 | 69788 | 69824 | 52 | 86 | 65.8 | 1315412, 1315885, | 2750, 2814, |
| | | | | | | 1315950, 1316385, | 2887, 2991, |
| | | | | | | 1317102 | 3090 |
| 4 | 9984 | 10008 | 52 | 84 | 73.3 | 1314829, 1315347, | 2581, 2640, |
| | | | | | | 1315586, 1316745, | 2736, 2857, |
| | | | | | | 1317017 | 2917 |
| 5 | 106863 | 106924 | 25 | 75 | 57.8 | 1314696, 1315159, | 1105, 1232, |
| | | | | | | 1316117, 1316972, | 1249, 3330, |
| | | | | | | 1317030, 1318565, | 3342, 3344, |
| | | | | | | 1318566, 1318569, | 3362, 3382, |
| | | | | | | 1318573, 1318577, | 3411, 3419, |
| | | | | | | 1318632, 1318634, | 3439, 3456, |
| | | | | | | 1318636, 1318690, | 3475, 3488, |
| | | | | | | 1318691, 1318695, | 3491, 3511, |
| | | | | | | 1318741, 1318745, | 3531, 3550, |
| | | | | | | 1318747 | 3551 |
| 6 | 115297 | 115337 | 65 | 74 | 69.2 | 1315073, 1316061, | 2208, 2310, |
| | | | | | | 1316776, 1317084, | 2335, 2471, |
| | | | | | | 1391292, 1391293, | 3553, 3559, |
| | | | | | | 1391296, 1391299, | 3563, 3565, |
| | | | | | | 1391301, 1391304, | 3570, 3580, |
| | | | | | | 1391314, 1391316, | 3581, 3583, |
| | | | | | | 1391332, 1391333, | 3585, 3589, |
| | | | | | | 1391339, 1391351, | 3593, 3594, |
| | | | | | | 1391353, 1391356, | 3597, 3600, |
| | | | | | | 1391357, 1391364, | 3601, 3604, |
| | | | | | | 1391365, 1391366, | 3614, 3616, |
| | | | | | | 1394343, 1394373, | 3630, 3631, |
| | | | | | | 1394377, 1394382, | 3637, 3648, |
| | | | | | | 1394386, 1394392, | 3650, 3652, |
| | | | | | | 1394394, 1394419, | 3653, 3660, |
| | | | | | | 1394424, 1394441, | 3661, 3662, |
| | | | | | | 1394449, 1419387, | 3684, 3689 |
| | | | | | | 1419393, 1419397, | |
| | | | | | | 1419399, 1419404, | |
| | | | | | | 1419415, 1419416, | |
| | | | | | | 1419418, 1419420, | |
| | | | | | | 1419424 | |
| 7 | 117868 | 117902 | 52 | 86 | 73.7 | 1314540, 1315083, | 2644, 2770, |
| | | | | | | 1316639, 1316648, | 2786, 2916, |
| | | | | | | 1394315, 1394316, | 3574, 3576, |
| | | | | | | 1394317, 1394318, | 3578, 3587, |
| | | | | | | 1394320, 1394321, | 3665, 3666, |
| | | | | | | 1394323, 1394325, | 3667, 3668, |
| | | | | | | 1394326, 1394331, | 3669, 3670, |
| | | | | | | 1394335, 1394336, | 3671, 3672, |
| | | | | | | 1394349, 1394397, | 3673, 3674, |
| | | | | | | 1394398, 1394399, | 3675, 3676, |
| | | | | | | 1394400, 1394401, | 3686, 3694, |
| | | | | | | 1394402, 1394421, | 3698 |
| | | | | | | 1394430, 1394434, | |
| | | | | | | 1394439, 1394447, | |
| | | | | | | 1419408, 1419410, | |
| | | | | | | 1419413, 1419422 | |
| 8 | 119644 | 119670 | 64 | 81 | 72.5 | 1314723, 1316689, | 919, 940, |
| | | | | | | 1456397, 1456398, | 3677, 3678, |
| | | | | | | 1456399, 1456400, | 3679, 3680, |
| | | | | | | 1456401, 1456402, | 3681, 3682, |
| | | | | | | 1456403, 1456404, | 3705, 3706, |
| | | | | | | 1456405, 1456406, | 3707 |
| | | | | | | 1456407, 1456408, | |
| | | | | | | 1456409, 1456410, | |
| | | | | | | 1456411, 1456412, | |
| | | | | | | 1456413 | |
| 9 | 126780 | 126829 | 60 | 87 | 72.1 | 1315214, 1315343, | 1576, 1652, |
| | | | | | | 1315670, 1316310, | 1771, 1813, |
| | | | | | | 1316802, 1316996, | 1933, 1987, |
| | | | | | | 1317163, 1391297, | 2078, 3552, |
| | | | | | | 1391308, 1391311, | 3555, 3558, |
| | | | | | | 1391313, 1391320, | 3560, 3572, |
| | | | | | | 1391329, 1391337, | 3573, 3575, |
| | | | | | | 1391346, 1391348, | 3579, 3584, |
| | | | | | | 1391352, 1391367, | 3598, 3608, |
| | | | | | | 1394345, 1394367, | 3611, 3613, |
| | | | | | | 1394368, 1394371, | 3619, 3628, |
| | | | | | | 1394378, 1394379, | 3635, 3644, |
| | | | | | | 1394387, 1394418, | 3646, 3649, |
| | | | | | | 1394423, 1394425, | 3663, 3683, |
| | | | | | | 1394444, 1394450, | 3688, 3690, |
| | | | | | | 1419360, 1419361, | 3700, 3701, |
| | | | | | | 1419362, 1419365, | 3702, 3703, |
| | | | | | | 1419370, 1419374, | 3704 |
| | | | | | | 1419375, 1419376, | |
| | | | | | | 1419379, 1419380, | |
| | | | | | | 1419383, 1419384, | |
| | | | | | | 1419386, 1419389, | |
| | | | | | | 1419392, 1419394, | |
| | | | | | | 1419406, 1419407, | |
| | | | | | | 1419409, 1419414, | |
| | | | | | | 1419419 | |
| 10 | 130388 | 130418 | 52 | 90 | 68.6 | 444652, 1231536, | 1343, 1406, |
| | | | | | | 1314810, 1315556, | 1539, 3335, |
| | | | | | | 1315878, 1316545, | 3401, 3472, |
| | | | | | | 1316803, 1318652, | 3477, 3519, |
| | | | | | | 1318742 | 3591 |

### X. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for administration by intrathecal injection. In certain embodiments, pharmaceutical compositions are prepared for administration by intracerebroventricular injection. In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in aqueous solution with phosphate buffered saline (PBS). In certain embodiments, modified oligonucleotides or oligomeric compounds are in aqueous solution with water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of Compound No. 1127954, equals the number of fully protonated molecules that weighs 10 mg. This would be equivalent to 10.47 mg of solvent-free, sodium acetate-free, anhydrous sodiated Compound No. 1127954.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or f such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ²C, ¹⁵N in place of ¹⁴N, ¹⁷O or ¹⁸O in place of ¹⁶O, and ³³S, ³⁴S, ³⁵S or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Effect of Mixed MOE and cEt, Mixed Backbone Modified Oligonucleotides on Human HTT RNA In Vitro, Single Dose

Modified oligonucleotides complementary to human HTT nucleic acid were designed and tested for their single dose effects on HTT RNA in vitro. The modified oligonucleotides were tested in a series of experiments under the culture conditions indicated in the tables below.

The modified oligonucleotides in the tables below are 17 nucleosides in length, wherein the sugar motif for the gapmers is (from 5′ to 3′): eekkddddddddkkeee; wherein each “d” represents a 2′-β-D-deoxyribosyl sugar, each “e” represents a 2′-MOE sugar moiety, and each “k” represents a cEt sugar moiety. The internucleoside linkage motif for the gapmers is (from 5′ to 3′): soosssssssssooss; wherein each “o” represents a phosphodiester internucleoside linkage, and each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NM_002111.6), to SEQ ID NO: 2 (GENBANK Accession No. NT_006081.17, truncated from 462000 to 634000), or to both. ‘N/A’ indicates that the modified oligonucleotide is not 100% complementary to that particular target nucleic acid sequence.

Cultured HepG2 or Hep3B cells were treated with modified oligonucleotide at a concentration of 2000 nM by electroporation at a density of 20,000 cells per well. After a treatment period of approximately 24 hours, total RNA was isolated from the cells and HTT RNA levels were measured by quantitative real-time RTPCR. HTT RNA levels were measured by human primer probe set RTS2617 (forward sequence CTCCGTCCGGTAGACATGCT, designated herein as SEQ ID NO: 11; reverse sequence GGAAATCAGAACCCTCAAAATGG, designated herein as SEQ ID NO: 12; probe sequence TGAGCACTGTTCAACTGTGGATATCGGGA, designated herein as SEQ ID NO: 13). HTT RNA levels were normalized to total RNA content, as measured by RIBOGREEN®. Reduction of HTT RNA is presented in the tables below as percent HTT RNA relative to the amount of the HTT RNA in untreated control cells (% UTC). Each table represents results from an individual assay plate.

TABLE 2
Reduction of HTT RNA by modified oligonucleotides with a mixed MOE/cEt sugar motif
and mixed PO/PS internucleoside linkages in HepG2 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646201 | N/A | N/A | 896 | 912 | GGCCAGCATGATTGACA | 113 | 26 |
| 646202 | N/A | N/A | 900 | 916 | CGCCGGCCAGCATGATT | 94 | 27 |
| 646203 | N/A | N/A | 904 | 920 | GCCACGCCGGCCAGCAT | 77 | 28 |
| 646204 | N/A | N/A | 921 | 937 | GCGCCGGCGGAGGCGGG | 75 | 29 |
| | | | 941 | 957 | | | |
| 646205 | N/A | N/A | 922 | 938 | CGCGCCGGCGGAGGCGG | 89 | 30 |
| 646206 | N/A | N/A | 926 | 942 | GGGCCGCGCCGGCGGAG | 91 | 31 |
| 646207 | N/A | N/A | 944 | 960 | GCTGCGCCGGCGGAGGC | 70 | 32 |
| 646208 | 47 | 63 | 1032 | 1048 | GCTCTGGGCCGCAGGTA | 56 | 33 |
| 646209 | 181 | 197 | 1166 | 1182 | GGAAGGACTTGAGGGAC | 67 | 34 |
| 646210 | 185 | 201 | 1170 | 1186 | TGCTGGAAGGACTTGAG | 34 | 35 |
| 646211 | 189 | 205 | 1174 | 1190 | CTGCTGCTGGAAGGACT | 23 | 36 |
| 646212 | 254 | 270 | 1233 | 1249 | GGCGGCTGTTGCTGCTG | 46 | 37 |
| 646213 | 262 | 278 | 1241 | 1257 | GCGGCGGTGGCGGCTGT | 81 | 38 |
| 646214 | 284 | 300 | 1263 | 1279 | TGAGGAGGCGGCGGCGG | 40 | 39 |
| 646215 | 288 | 304 | 1267 | 1283 | AAGCTGAGGAGGCGGCG | 45 | 40 |
| 646216 | 292 | 308 | 1271 | 1287 | GAGGAAGCTGAGGAGGC | 54 | 41 |
| 646217 | 296 | 312 | 1275 | 1291 | GGCTGAGGAAGCTGAGG | 52 | 42 |
| 646218 | 300 | 316 | 1279 | 1295 | CGGCGGCTGAGGAAGCT | 24 | 43 |
| 646219 | 304 | 320 | 1283 | 1299 | GCGGCGGCGGCTGAGGA | 26 | 44 |
| 646220 | 308 | 324 | 1287 | 1303 | GCCTGCGGCGGCGGCTG | 61 | 45 |
| 646221 | 312 | 328 | 1291 | 1307 | CTGTGCCTGCGGCGGCG | 31 | 46 |
| 646222 | 316 | 332 | 1295 | 1311 | GCGGCTGTGCCTGCGGC | 36 | 47 |
| 646223 | 320 | 336 | 1299 | 1315 | AGCAGCGGCTGTGCCTG | 64 | 48 |
| 646224 | 324 | 340 | 1303 | 1319 | AGGCAGCAGCGGCTGTG | 76 | 49 |
| 646225 | 328 | 344 | 1307 | 1323 | GCTGAGGCAGCAGCGGC | 63 | 50 |
| 646226 | 332 | 348 | 1311 | 1327 | TGCGGCTGAGGCAGCAG | 82 | 51 |
| 646227 | 336 | 352 | 1315 | 1331 | CGGCTGCGGCTGAGGCA | 48 | 52 |
| 646228 | 378 | 394 | 1357 | 1373 | AGCCACAGCCGGGCCGG | 108 | 53 |
| 646229 | 382 | 398 | 1361 | 1377 | CCTCAGCCACAGCCGGG | 87 | 54 |
| 646230 | N/A | N/A | 1402 | 1418 | GGAGCTGCAGCGGGCCC | 90 | 55 |
| 646231 | N/A | N/A | 1424 | 1440 | AGCCTGGGACCCGCCGG | 67 | 56 |
| 646232 | N/A | N/A | 1425 | 1441 | TAGCCTGGGACCCGCCG | 76 | 57 |
| 646233 | N/A | N/A | 1426 | 1442 | GTAGCCTGGGACCCGCC | 68 | 58 |
| 646234 | N/A | N/A | 1427 | 1443 | CGTAGCCTGGGACCCGC | 59 | 59 |
| 646235 | N/A | N/A | 1428 | 1444 | CCGTAGCCTGGGACCCG | 74 | 60 |
| 646236 | N/A | N/A | 1429 | 1445 | GCCGTAGCCTGGGACCC | 83 | 61 |
| 646237 | N/A | N/A | 1430 | 1446 | CGCCGTAGCCTGGGACC | 80 | 62 |
| 646238 | N/A | N/A | 1431 | 1447 | CCGCCGTAGCCTGGGAC | 71 | 63 |
| 646239 | N/A | N/A | 1432 | 1448 | CCCGCCGTAGCCTGGGA | 95 | 64 |
| 646240 | N/A | N/A | 1433 | 1449 | CCCCGCCGTAGCCTGGG | 96 | 65 |
| 646241 | N/A | N/A | 1434 | 1450 | TCCCCGCCGTAGCCTGG | 78 | 66 |
| 646242 | N/A | N/A | 1435 | 1451 | ATCCCCGCCGTAGCCTG | 88 | 67 |
| 646243 | N/A | N/A | 1436 | 1452 | CATCCCCGCCGTAGCCT | 72 | 68 |
| 646244 | N/A | N/A | 1438 | 1454 | GCCATCCCCGCCGTAGC | 65 | 69 |
| 646245 | N/A | N/A | 1439 | 1455 | CGCCATCCCCGCCGTAG | 101 | 70 |
| 646246 | N/A | N/A | 1440 | 1456 | CCGCCATCCCCGCCGTA | 110 | 71 |
| 646247 | N/A | N/A | 1441 | 1457 | ACCGCCATCCCCGCCGT | 97 | 72 |
| 646248 | N/A | N/A | 1442 | 1458 | TACCGCCATCCCCGCCG | 77 | 73 |
| 646249 | N/A | N/A | 1443 | 1459 | TTACCGCCATCCCCGCC | 72 | 74 |
| 646250 | N/A | N/A | 1444 | 1460 | GTTACCGCCATCCCCGC | 91 | 75 |
| 646251 | N/A | N/A | 1445 | 1461 | GGTTACCGCCATCCCCG | 72 | 76 |
| 646252 | N/A | N/A | 1446 | 1462 | GGGTTACCGCCATCCCC | 83 | 77 |
| 646253 | N/A | N/A | 1447 | 1463 | AGGGTTACCGCCATCCC | 86 | 78 |
| 646254 | N/A | N/A | 1448 | 1464 | CAGGGTTACCGCCATCC | 53 | 79 |
| 646255 | N/A | N/A | 1449 | 1465 | GCAGGGTTACCGCCATC | 96 | 80 |
| 646256 | N/A | N/A | 1450 | 1466 | TGCAGGGTTACCGCCAT | 91 | 81 |
| 646257 | N/A | N/A | 1452 | 1468 | GCTGCAGGGTTACCGCC | 81 | 82 |
| 646258 | N/A | N/A | 1453 | 1469 | GGCTGCAGGGTTACCGC | 71 | 83 |
| 646259 | N/A | N/A | 1454 | 1470 | AGGCTGCAGGGTTACCG | 35 | 84 |
| 646260 | N/A | N/A | 1531 | 1547 | GCGGGTGTCCCTCATGG | 98 | 85 |
| 646261 | N/A | N/A | 1634 | 1650 | GGACAGGCCCCAACAAG | 92 | 86 |
| 646262 | N/A | N/A | 1637 | 1653 | TCAGGACAGGCCCCAAC | 63 | 87 |
| 646263 | N/A | N/A | 1640 | 1656 | AATTCAGGACAGGCCCC | 61 | 88 |
| 646264 | N/A | N/A | 1643 | 1659 | GTGAATTCAGGACAGGC | 41 | 89 |
| 646265 | N/A | N/A | 1646 | 1662 | TCGGTGAATTCAGGACA | 30 | 90 |
| 646266 | N/A | N/A | 1649 | 1665 | CCCTCGGTGAATTCAGG | 51 | 91 |
| 646267 | N/A | N/A | 1652 | 1668 | CTCCCCTCGGTGAATTC | 69 | 92 |
| 646268 | N/A | N/A | 1655 | 1671 | TGACTCCCCTCGGTGAA | 89 | 93 |
| 646269 | N/A | N/A | 1658 | 1674 | CCGTGACTCCCCTCGGT | 70 | 94 |
| 646270 | N/A | N/A | 1661 | 1677 | AGGCCGTGACTCCCCTC | 87 | 95 |
| 646271 | N/A | N/A | 1664 | 1680 | CTGAGGCCGTGACTCCC | 69 | 96 |
| 646272 | N/A | N/A | 1667 | 1683 | GGGCTGAGGCCGTGACT | 56 | 97 |
| 646273 | N/A | N/A | 1670 | 1686 | AGAGGGCTGAGGCCGTG | 80 | 98 |
| 646274 | N/A | N/A | 1673 | 1689 | GCGAGAGGGCTGAGGCC | 78 | 99 |
| 646275 | N/A | N/A | 1682 | 1698 | CTGCGAAGGGCGAGAGG | 97 | 100 |
| 646276 | N/A | N/A | 1685 | 1701 | ATCCTGCGAAGGGCGAG | 76 | 101 |
| 646277 | N/A | N/A | 1688 | 1704 | CGCATCCTGCGAAGGGC | 88 | 102 |
| 646278 | N/A | N/A | 1691 | 1707 | CTTCGCATCCTGCGAAG | 107 | 103 |

TABLE 3
Reduction of HTT RNA by modified oligonucleotides with a mixed MOE/cEt sugar motif
and mixed PO/PS internucleoside linkages in Hep3B cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 78 | 78 |
| 646279 | N/A | N/A | 1694 | 1710 | ACTCTTCGCATCCTGCG | 67 | 104 |
| 646280 | N/A | N/A | 1697 | 1713 | CCAACTCTTCGCATCCT | 87 | 105 |
| 646281 | N/A | N/A | 1700 | 1716 | GCCCCAACTCTTCGCAT | 93 | 106 |
| 646282 | N/A | N/A | 1703 | 1719 | CTCGCCCCAACTCTTCG | 86 | 107 |
| 646283 | N/A | N/A | 1706 | 1722 | GTTCTCGCCCCAACTCT | 65 | 108 |
| 646284 | N/A | N/A | 1709 | 1725 | CAAGTTCTCGCCCCAAC | 38 | 109 |
| 646285 | N/A | N/A | 1712 | 1728 | AAACAAGTTCTCGCCCC | 65 | 110 |
| 646286 | N/A | N/A | 1715 | 1731 | AAGAAACAAGTTCTCGC | 75 | 111 |
| 646287 | N/A | N/A | 1725 | 1741 | CGCAAATAAAAAGAAAC | 47 | 112 |
| 646288 | N/A | N/A | 1728 | 1744 | TCTCGCAAATAAAAAGA | 70 | 113 |
| 646289 | N/A | N/A | 1731 | 1747 | GTTTCTCGCAAATAAAA | 59 | 114 |
| 646290 | N/A | N/A | 1734 | 1750 | CTGGTTTCTCGCAAATA | 59 | 115 |
| 646291 | N/A | N/A | 1737 | 1753 | GCCCTGGTTTCTCGCAA | 71 | 116 |
| 646292 | N/A | N/A | 1740 | 1756 | CCCGCCCTGGTTTCTCG | 64 | 117 |
| 646293 | N/A | N/A | 1743 | 1759 | ACCCCCGCCCTGGTTTC | 81 | 118 |
| 646294 | N/A | N/A | 1746 | 1762 | AGAACCCCCGCCCTGGT | 74 | 119 |
| 646295 | N/A | N/A | 1749 | 1765 | AAAAGAACCCCCGCCCT | 112 | 120 |
| 646296 | N/A | N/A | 1752 | 1768 | GTTAAAAGAACCCCCGC | 124 | 121 |
| 646297 | N/A | N/A | 1755 | 1771 | GCAGTTAAAAGAACCCC | 54 | 122 |
| 646298 | N/A | N/A | 1758 | 1774 | AACGCAGTTAAAAGAAC | 90 | 123 |
| 646299 | N/A | N/A | 1761 | 1777 | CACAACGCAGTTAAAAG | 90 | 124 |
| 646300 | N/A | N/A | 1764 | 1780 | CTTCACAACGCAGTTAA | 74 | 125 |
| 646301 | N/A | N/A | 1767 | 1783 | TCTCTTCACAACGCAGT | 43 | 126 |
| 646302 | N/A | N/A | 1770 | 1786 | AGTTCTCTTCACAACGC | 82 | 127 |
| 646303 | N/A | N/A | 1773 | 1789 | CCAAGTTCTCTTCACAA | 59 | 128 |
| 646304 | N/A | N/A | 1776 | 1792 | CCTCCAAGTTCTCTTCA | 85 | 129 |
| 646305 | N/A | N/A | 1779 | 1795 | GCTCCTCCAAGTTCTCT | 75 | 130 |
| 646306 | N/A | N/A | 1782 | 1798 | TCGGCTCCTCCAAGTTC | 84 | 131 |
| 646307 | N/A | N/A | 1785 | 1801 | ATCTCGGCTCCTCCAAG | 53 | 132 |
| 646308 | N/A | N/A | 1788 | 1804 | CAAATCTCGGCTCCTCC | 71 | 133 |
| 646309 | N/A | N/A | 1791 | 1807 | GAGCAAATCTCGGCTCC | 78 | 134 |
| 646310 | N/A | N/A | 1794 | 1810 | ACTGAGCAAATCTCGGC | 79 | 135 |
| 646311 | N/A | N/A | 1797 | 1813 | GGCACTGAGCAAATCTC | 39 | 136 |
| 646312 | N/A | N/A | 1800 | 1816 | AGTGGCACTGAGCAAAT | 58 | 137 |
| 646313 | N/A | N/A | 1803 | 1819 | GGAAGTGGCACTGAGCA | 46 | 138 |
| 646314 | N/A | N/A | 1806 | 1822 | GAGGGAAGTGGCACTGA | 82 | 139 |
| 646315 | N/A | N/A | 1809 | 1825 | GAAGAGGGAAGTGGCAC | 81 | 140 |
| 646316 | N/A | N/A | 1812 | 1828 | CTAGAAGAGGGAAGTGG | 100 | 141 |
| 646317 | N/A | N/A | 1815 | 1831 | AGACTAGAAGAGGGAAG | 103 | 142 |
| 646318 | N/A | N/A | 1818 | 1834 | CTCAGACTAGAAGAGGG | 108 | 143 |
| 646319 | N/A | N/A | 1821 | 1837 | CCTCTCAGACTAGAAGA | 104 | 144 |
| 646320 | N/A | N/A | 1824 | 1840 | TTCCCTCTCAGACTAGA | 81 | 145 |
| 646321 | N/A | N/A | 1827 | 1843 | CTCTTCCCTCTCAGACT | 103 | 146 |
| 646322 | N/A | N/A | 1830 | 1846 | GCCCTCTTCCCTCTCAG | 85 | 147 |
| 646323 | N/A | N/A | 1838 | 1854 | CGCCCCCAGCCCTCTTC | 93 | 148 |
| 646324 | N/A | N/A | 1841 | 1857 | CCGCGCCCCCAGCCCTC | 82 | 149 |
| 646325 | N/A | N/A | 1844 | 1860 | GTCCCGCGCCCCCAGCC | 92 | 150 |
| 646326 | N/A | N/A | 1847 | 1863 | AGTGTCCCGCGCCCCCA | 58 | 151 |
| 646327 | N/A | N/A | 1850 | 1866 | CGAAGTGTCCCGCGCCC | 59 | 152 |
| 646328 | N/A | N/A | 1853 | 1869 | TCTCGAAGTGTCCCGCG | 95 | 153 |
| 646329 | N/A | N/A | 1856 | 1872 | TCCTCTCGAAGTGTCCC | 64 | 154 |
| 646330 | N/A | N/A | 1859 | 1875 | GCCTCCTCTCGAAGTGT | 69 | 155 |
| 646331 | N/A | N/A | 1862 | 1878 | CCCGCCTCCTCTCGAAG | 75 | 156 |
| 646332 | N/A | N/A | 1865 | 1881 | AACCCCGCCTCCTCTCG | 65 | 157 |
| 646333 | N/A | N/A | 1868 | 1884 | CCAAACCCCGCCTCCTC | 71 | 158 |
| 646334 | N/A | N/A | 1871 | 1887 | GCTCCAAACCCCGCCTC | 76 | 159 |
| 646335 | N/A | N/A | 1874 | 1890 | CCAGCTCCAAACCCCGC | 59 | 160 |
| 646336 | N/A | N/A | 1877 | 1893 | TCTCCAGCTCCAAACCC | 101 | 161 |
| 646337 | N/A | N/A | 1880 | 1896 | ATCTCTCCAGCTCCAAA | 67 | 162 |
| 646338 | N/A | N/A | 1883 | 1899 | CACATCTCTCCAGCTCC | 66 | 163 |
| 646339 | N/A | N/A | 1889 | 1905 | TGCCCCCACATCTCTCC | 71 | 164 |
| 646340 | N/A | N/A | 1892 | 1908 | CACTGCCCCCACATCTC | 67 | 165 |
| 646341 | N/A | N/A | 1895 | 1911 | ATCCACTGCCCCCACAT | 89 | 166 |
| 646342 | N/A | N/A | 1898 | 1914 | GTCATCCACTGCCCCCA | 44 | 167 |
| 646343 | N/A | N/A | 1901 | 1917 | TATGTCATCCACTGCCC | 54 | 168 |
| 646344 | N/A | N/A | 1907 | 1923 | AAGCATTATGTCATCCA | 29 | 169 |
| 646345 | N/A | N/A | 1910 | 1926 | TAAAAGCATTATGTCAT | 67 | 170 |
| 646346 | N/A | N/A | 1913 | 1929 | TCCTAAAAGCATTATGT | 97 | 171 |
| 646347 | N/A | N/A | 1930 | 1946 | CCACTCCCGCCGAGGCG | 74 | 172 |
| 646348 | N/A | N/A | 1933 | 1949 | CCGCCACTCCCGCCGAG | 71 | 173 |
| 646349 | N/A | N/A | 1936 | 1952 | GCCCCGCCACTCCCGCC | 80 | 174 |
| 646350 | N/A | N/A | 1939 | 1955 | CCTGCCCCGCCACTCCC | 78 | 175 |
| 646351 | N/A | N/A | 1947 | 1963 | GCCCCCCCCCTGCCCCG | 82 | 176 |
| 646352 | N/A | N/A | 1952 | 1968 | TCCCCGCCCCCCCCCTG | 109 | 177 |
| 646353 | N/A | N/A | 1955 | 1971 | CACTCCCCGCCCCCCCC | 68 | 178 |
| 646354 | N/A | N/A | 1960 | 1976 | GCCCTCACTCCCCGCCC | 79 | 179 |
| 646355 | N/A | N/A | 1963 | 1979 | CGCGCCCTCACTCCCCG | 92 | 180 |

TABLE 4
Reduction of HTT RNA by modified oligonucleotides with a mixed MOE/cEt sugar motif
and mixed PO/PS internucleoside linkages in HepG2 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 65 | 78 |
| 646356 | N/A | N/A | 1969 | 1985 | ATTGGACGCGCCCTCAC | 38 | 181 |
| 646357 | N/A | N/A | 1972 | 1988 | CCCATTGGACGCGCCCT | 54 | 182 |
| 646358 | N/A | N/A | 1975 | 1991 | TCTCCCATTGGACGCGC | 69 | 183 |
| 646359 | N/A | N/A | 1981 | 1997 | AAGAAATCTCCCATTGG | 55 | 184 |
| 646360 | N/A | N/A | 1984 | 2000 | GAAAAGAAATCTCCCAT | 58 | 185 |
| 646361 | N/A | N/A | 1987 | 2003 | TAGGAAAAGAAATCTCC | 87 | 186 |
| 646362 | N/A | N/A | 1990 | 2006 | CACTAGGAAAAGAAATC | 113 | 187 |
| 646363 | N/A | N/A | 1993 | 2009 | TGCCACTAGGAAAAGAA | 90 | 188 |
| 646364 | N/A | N/A | 1996 | 2012 | AAGTGCCACTAGGAAAA | 33 | 189 |
| 646365 | N/A | N/A | 1999 | 2015 | TTTAAGTGCCACTAGGA | 52 | 190 |
| 646366 | N/A | N/A | 2002 | 2018 | TGTTTTAAGTGCCACTA | 39 | 191 |
| 646367 | N/A | N/A | 2005 | 2021 | GGCTGTTTTAAGTGCCA | 68 | 192 |
| 646368 | N/A | N/A | 2008 | 2024 | TCAGGCTGTTTTAAGTG | 69 | 193 |
| 646369 | N/A | N/A | 2011 | 2027 | ATCTCAGGCTGTTTTAA | 70 | 194 |
| 646370 | N/A | N/A | 2014 | 2030 | CAAATCTCAGGCTGTTT | 28 | 195 |
| 646371 | N/A | N/A | 2017 | 2033 | CCTCAAATCTCAGGCTG | 59 | 196 |
| 646372 | N/A | N/A | 2020 | 2036 | GAGCCTCAAATCTCAGG | 53 | 197 |
| 646373 | N/A | N/A | 2023 | 2039 | GAAGAGCCTCAAATCTC | 58 | 198 |
| 646374 | N/A | N/A | 2026 | 2042 | TAGGAAGAGCCTCAAAT | 64 | 199 |
| 646375 | N/A | N/A | 2029 | 2045 | ATGTAGGAAGAGCCTCA | 50 | 200 |
| 646376 | N/A | N/A | 2032 | 2048 | ACAATGTAGGAAGAGCC | 31 | 201 |
| 646377 | N/A | N/A | 2035 | 2051 | CTGACAATGTAGGAAGA | 44 | 202 |
| 646378 | N/A | N/A | 2038 | 2054 | GTCCTGACAATGTAGGA | 77 | 203 |
| 646379 | N/A | N/A | 2041 | 2057 | AATGTCCTGACAATGTA | 44 | 204 |
| 646380 | N/A | N/A | 2044 | 2060 | TGAAATGTCCTGACAAT | 56 | 205 |
| 646381 | N/A | N/A | 2047 | 2063 | AAATGAAATGTCCTGAC | 47 | 206 |
| 646382 | N/A | N/A | 2050 | 2066 | ACTAAATGAAATGTCCT | 48 | 207 |
| 646383 | N/A | N/A | 2057 | 2073 | ATCATGAACTAAATGAA | 84 | 208 |
| 646384 | N/A | N/A | 2060 | 2076 | GTGATCATGAACTAAAT | 37 | 209 |
| 646385 | N/A | N/A | 2063 | 2079 | ACCGTGATCATGAACTA | 43 | 210 |
| 646386 | N/A | N/A | 2066 | 2082 | ACCACCGTGATCATGAA | 46 | 211 |
| 646387 | N/A | N/A | 2069 | 2085 | ACTACCACCGTGATCAT | 66 | 212 |
| 646388 | N/A | N/A | 2072 | 2088 | GTTACTACCACCGTGAT | 86 | 213 |
| 646389 | N/A | N/A | 2075 | 2091 | CGTGTTACTACCACCGT | 50 | 214 |
| 646390 | N/A | N/A | 2090 | 2106 | GGTGGTGCTTAAAATCG | 74 | 215 |
| 646391 | N/A | N/A | 2093 | 2109 | TTAGGTGGTGCTTAAAA | 85 | 216 |
| 646392 | N/A | N/A | 2096 | 2112 | CTCTTAGGTGGTGCTTA | 60 | 217 |
| 646393 | N/A | N/A | 2099 | 2115 | GATCTCTTAGGTGGTGC | 53 | 218 |
| 646394 | N/A | N/A | 2102 | 2118 | GCAGATCTCTTAGGTGG | 53 | 219 |
| 646395 | N/A | N/A | 2105 | 2121 | TGAGCAGATCTCTTAGG | 58 | 220 |
| 646396 | N/A | N/A | 2111 | 2127 | CTTAGATGAGCAGATCT | 55 | 221 |
| 646397 | N/A | N/A | 2114 | 2130 | AGGCTTAGATGAGCAGA | 68 | 222 |
| 646398 | N/A | N/A | 2120 | 2136 | CAACTTAGGCTTAGATG | 35 | 223 |
| 646399 | N/A | N/A | 2126 | 2142 | GCAGACCAACTTAGGCT | 53 | 224 |
| 646400 | N/A | N/A | 2129 | 2145 | CCTGCAGACCAACTTAG | 67 | 225 |
| 646401 | N/A | N/A | 2132 | 2148 | ACGCCTGCAGACCAACT | 31 | 226 |
| 646402 | N/A | N/A | 2145 | 2161 | ACAACTCATTCAAACGC | 62 | 227 |
| 646403 | N/A | N/A | 2148 | 2164 | ACCACAACTCATTCAAA | 67 | 228 |
| 646404 | N/A | N/A | 2151 | 2167 | GCAACCACAACTCATTC | 40 | 229 |
| 646405 | N/A | N/A | 2154 | 2170 | TTGGCAACCACAACTCA | 63 | 230 |
| 646406 | N/A | N/A | 2157 | 2173 | TACTTGGCAACCACAAC | 53 | 231 |
| 646407 | N/A | N/A | 2160 | 2176 | CTTTACTTGGCAACCAC | 44 | 232 |
| 646408 | N/A | N/A | 2166 | 2182 | TCACCACTTTACTTGGC | 48 | 233 |
| 646409 | N/A | N/A | 2169 | 2185 | AGTTCACCACTTTACTT | 99 | 234 |
| 646410 | N/A | N/A | 2172 | 2188 | GTAAGTTCACCACTTTA | 75 | 235 |
| 646411 | N/A | N/A | 2188 | 2204 | TTTCATTAATCACCACG | 21 | 236 |
| 646412 | N/A | N/A | 2191 | 2207 | TAATTTCATTAATCACC | 30 | 237 |
| 646413 | N/A | N/A | 2194 | 2210 | AGATAATTTCATTAATC | 78 | 238 |
| 646414 | N/A | N/A | 2197 | 2213 | TTAAGATAATTTCATTA | 81 | 239 |
| 646415 | N/A | N/A | 2205 | 2221 | CCTAATATTTAAGATAA | 82 | 240 |
| 646416 | N/A | N/A | 2208 | 2224 | CTTCCTAATATTTAAGA | 88 | 241 |
| 646417 | N/A | N/A | 2211 | 2227 | ACTCTTCCTAATATTTA | 80 | 242 |
| 646418 | N/A | N/A | 2214 | 2230 | TCAACTCTTCCTAATAT | 72 | 243 |
| 646419 | N/A | N/A | 2217 | 2233 | CAATCAACTCTTCCTAA | 85 | 244 |
| 646420 | N/A | N/A | 2220 | 2236 | CTTCAATCAACTCTTCC | 47 | 245 |
| 646421 | N/A | N/A | 2223 | 2239 | AAACTTCAATCAACTCT | 50 | 246 |
| 646422 | N/A | N/A | 2226 | 2242 | AAAAAACTTCAATCAAC | 58 | 247 |
| 646423 | N/A | N/A | 2229 | 2245 | GGCAAAAAACTTCAATC | 46 | 248 |
| 646424 | N/A | N/A | 2232 | 2248 | ATAGGCAAAAAACTTCA | 66 | 249 |
| 646425 | N/A | N/A | 2235 | 2251 | CACATAGGCAAAAAACT | 82 | 250 |
| 646426 | N/A | N/A | 2239 | 2255 | AACACACATAGGCAAAA | 75 | 251 |
| 646427 | N/A | N/A | 2242 | 2258 | CCCAACACACATAGGCA | 44 | 252 |
| 646428 | N/A | N/A | 2245 | 2261 | ATTCCCAACACACATAG | 87 | 253 |
| 646429 | N/A | N/A | 2248 | 2264 | TTTATTCCCAACACACA | 81 | 254 |
| 646430 | N/A | N/A | 2251 | 2267 | GGTTTTATTCCCAACAC | 95 | 255 |
| 646431 | N/A | N/A | 2254 | 2270 | GTTGGTTTTATTCCCAA | 91 | 256 |
| 646432 | N/A | N/A | 2257 | 2273 | CGTGTTGGTTTTATTCC | 85 | 257 |

### Example 2: Effect of Mixed MOE and cEt, Mixed Backbone Modified Oligonucleotides on Human HTT RNA In Vitro, Single Dose

Modified oligonucleotides complementary to human HTT nucleic acid were designed and tested for their single dose effects on HTT RNA in vitro. The modified oligonucleotides were tested in a series of experiments that had the same culture conditions.

The modified oligonucleotides in the tables below are 17 nucleosides in length, wherein the sugar motif for the gapmers is (from 5′ to 3′): ekekddddddddkekee; wherein each “d” represents a 2′-β-D-deoxyribosyl sugar, each “e” represents a 2′-MOE sugar moiety, and each “k” represents a cEt sugar moiety. The internucleoside linkage motif for the gapmers is (from 5′ to 3′): soosssssssssooss; wherein each “o” represents a phosphodiester internucleoside linkage, and each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

Cultured A431 cells were treated with modified oligonucleotide at a concentration of 2000 nM by free uptake at a density of 10,000 cells per well. After a treatment period of approximately 48 hours, total RNA was isolated from the cells and HTT RNA levels were measured by quantitative real-time RTPCR. HTT RNA levels were measured by human primer probe set RTS36485 (forward sequence GAGGAAGTAGATCCAAACACACA, designated herein as SEQ ID NO: 14; reverse sequence GCCGCTATTCCTTTTATGACC, designated herein as SEQ ID NO: 15; probe sequence TCCACCATTTCTGCCACCATCTCA, designated herein as SEQ ID NO: 16). HTT RNA levels were normalized to total RNA content, as measured by RIBOGREEN®. Reduction of HTT RNA is presented in the tables below as percent HTT RNA relative to the amount of the HTT RNA in untreated control cells (% UTC). Each table represents results from an individual assay plate.

In the tables below, Compound 646253 (described herein above) was used as an inter-experimental control.

TABLE 5
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 140 | 78 |
| 953797 | 33 | 49 | 1018 | 1034 | GTAAAAGCAGAACCTGA | 93 | 258 |
| 953805 | N/A | N/A | 1584 | 1600 | ACCCAAGTGAGGGAGCG | 115 | 259 |
| 953813 | N/A | N/A | 1712 | 1728 | AAACAAGTTCTCGCCCC | 85 | 110 |
| 953821 | N/A | N/A | 1805 | 1821 | AGGGAAGTGGCACTGAG | 108 | 260 |
| 953829 | N/A | N/A | 1906 | 1922 | AGCATTATGTCATCCAC | 107 | 261 |
| 953837 | N/A | N/A | 1984 | 2000 | GAAAAGAAATCTCCCAT | 95 | 185 |
| 953845 | N/A | N/A | 2045 | 2061 | ATGAAATGTCCTGACAA | 76 | 262 |
| 953853 | N/A | N/A | 2147 | 2163 | CCACAACTCATTCAAAC | 146 | 263 |
| 953861 | N/A | N/A | 2225 | 2241 | AAAAACTTCAATCAACT | 137 | 264 |
| 953869 | N/A | N/A | 2240 | 2256 | CAACACACATAGGCAAA | 91 | 265 |
| 953877 | N/A | N/A | 2397 | 2413 | AGCAAACAGAAACCCTT | 103 | 266 |
| 953885 | N/A | N/A | 2426 | 2442 | TCCAAAAAGTAACAAGC | 87 | 267 |
| 953893 | N/A | N/A | 2468 | 2484 | TCAAAAATTCTCCCCAA | 102 | 268 |
| 953901 | N/A | N/A | 2570 | 2586 | CACCAGAGTACTTCCTA | 85 | 269 |
| 953909 | N/A | N/A | 2617 | 2633 | CCAGATCGGAAATAGAA | 147 | 270 |
| 953917 | N/A | N/A | 2660 | 2676 | GTCTTAACAGAGATATT | 93 | 271 |
| 953925 | N/A | N/A | 2714 | 2730 | AGACAAGGATCATAACA | 121 | 272 |
| 953933 | N/A | N/A | 2736 | 2752 | TTCTAATTAAACTTTGA | 94 | 273 |
| 953941 | N/A | N/A | 2784 | 2800 | CCTAAATTTTATTCCAA | 63 | 274 |
| 953949 | N/A | N/A | 2827 | 2843 | CCAAATATCCAAAAAGT | 107 | 275 |
| 953957 | N/A | N/A | 2893 | 2909 | CTACAGTAAATTTCTGG | 79 | 276 |
| 953965 | N/A | N/A | 2906 | 2922 | TCCTAGAAAAGCACTAC | 93 | 277 |
| 953973 | N/A | N/A | 2952 | 2968 | GTTTACACAAGATACAT | 102 | 278 |
| 953981 | N/A | N/A | 3026 | 3042 | TATTTTTAGAATGATCA | 97 | 279 |
| 953989 | N/A | N/A | 3055 | 3071 | TGTCAAATCAGCATAAG | 83 | 280 |
| 953997 | N/A | N/A | 3088 | 3104 | ACCCATATTTTGAAGAT | 120 | 281 |
| 954005 | N/A | N/A | 3134 | 3150 | ACAAAATCTATCTCCTT | 82 | 282 |
| 954013 | N/A | N/A | 3161 | 3177 | AATCATATATTACCTTA | 107 | 283 |
| 954021 | N/A | N/A | 3515 | 3531 | GTCAATGTATTACTTAT | 60 | 284 |
| 954029 | N/A | N/A | 3570 | 3586 | GTGGAAGGAAGATATTT | 88 | 285 |
| 954037 | N/A | N/A | 3647 | 3663 | TTATATCTGGCAGGATA | 102 | 286 |
| 954045 | N/A | N/A | 3674 | 3690 | TTAAATCTATCTTACAA | 113 | 287 |
| 954053 | N/A | N/A | 3748 | 3764 | TGACAATGTGAGACTAC | 114 | 288 |
| 954061 | N/A | N/A | 4068 | 4084 | ATAAATAAACCTAATGC | 92 | 289 |
| 954069 | N/A | N/A | 4151 | 4167 | AATTAGTGAATACCTCC | 98 | 290 |
| 954077 | N/A | N/A | 4188 | 4204 | CAAAATCCAAACTTTGT | 89 | 291 |
| 954085 | N/A | N/A | 4272 | 4288 | ACAGAACAGATACTTTC | 78 | 292 |
| 954093 | N/A | N/A | 4338 | 4354 | TCATAAACTGAGGCCCA | 117 | 293 |
| 954101 | N/A | N/A | 4441 | 4457 | CTAATTACTGTGAGGAC | 59 | 294 |
| 954109 | N/A | N/A | 4475 | 4491 | TAAGAAACTGCCAGAAG | 92 | 295 |
| 954117 | N/A | N/A | 4511 | 4527 | AGTCAGGGAAACACCAG | 103 | 296 |
| 954125 | N/A | N/A | 4548 | 4564 | GTACATGTTTAGTAAAT | 102 | 297 |
| 954133 | N/A | N/A | 4622 | 4638 | ATGAAAGCTTCACGAGG | 80 | 298 |
| 954141 | N/A | N/A | 4839 | 4855 | CAACACTTAGTGCTACA | 96 | 299 |
| 954149 | N/A | N/A | 5500 | 5516 | TTAAAATATCAACACTG | 88 | 300 |
| 954157 | N/A | N/A | 5615 | 5631 | TACGATAGTGATACTGC | 102 | 301 |
| 954165 | N/A | N/A | 5763 | 5779 | CCTTACACTAGACTCTT | 84 | 302 |
| 954173 | N/A | N/A | 5822 | 5838 | CTGAAACAGGAAGATTT | 91 | 303 |
| 954181 | N/A | N/A | 5876 | 5892 | TACAAATGTCCCCCTCA | 117 | 304 |
| 954189 | N/A | N/A | 5959 | 5975 | GCCAATATCTTTATTCC | 102 | 305 |
| 954197 | N/A | N/A | 6032 | 6048 | TAAGATTGAAATTCAGA | 89 | 306 |
| 954205 | N/A | N/A | 6187 | 6203 | TCAAAATCCAGTCAGTC | 46 | 307 |
| 954213 | N/A | N/A | 6238 | 6254 | TGCTAAATTGTTATACT | 84 | 308 |
| 954221 | N/A | N/A | 6300 | 6316 | AGCTATTGCAGCAGGAT | 86 | 309 |
| 954229 | N/A | N/A | 6705 | 6721 | TATAAGAAAGAGCCTGG | 95 | 310 |
| 954237 | N/A | N/A | 6727 | 6743 | GAACAAGGGTTTGCAAG | 109 | 311 |
| 954245 | N/A | N/A | 6843 | 6859 | GACAATCCAAAGGAAGG | 116 | 312 |
| 954253 | N/A | N/A | 7036 | 7052 | GATCAGACTTAAATGTA | 99 | 313 |
| 954261 | N/A | N/A | 7163 | 7179 | ACTAAGATAATTCCATG | 91 | 314 |
| 954269 | N/A | N/A | 7193 | 7209 | AATTAAGGACGATTAAT | 135 | 315 |
| 954277 | N/A | N/A | 7202 | 7218 | AATTTATACAATTAAGG | 88 | 316 |
| 954285 | N/A | N/A | 7254 | 7270 | TGGCACAGGATCGACAT | 111 | 317 |
| 954293 | N/A | N/A | 7373 | 7389 | CGGAAATGCAAGGGACC | 126 | 318 |
| 954301 | N/A | N/A | 7523 | 7539 | TTACACAAAGTTTGCCC | 132 | 319 |
| 954309 | N/A | N/A | 7597 | 7613 | TACAATGGCAGAGCCGC | 115 | 320 |
| 954317 | N/A | N/A | 7712 | 7728 | TCAAATACTATCTCATG | 74 | 321 |
| 954325 | N/A | N/A | 7791 | 7807 | AAAACTATAACCTGCAT | 142 | 322 |
| 954333 | N/A | N/A | 8161 | 8177 | AAAGAAATGCTTTCCAG | 115 | 323 |
| 954341 | N/A | N/A | 8292 | 8308 | AGACAAAGATGACAGAC | 83 | 324 |
| 954349 | N/A | N/A | 8332 | 8348 | GGGAAGAAGACACAAGA | 82 | 325 |
| 954357 | N/A | N/A | 8365 | 8381 | CATTTAAATGCTTGTAT | 82 | 326 |
| 954365 | N/A | N/A | 8442 | 8458 | AACAATATCTATGACAC | 56 | 327 |
| 954373 | N/A | N/A | 8467 | 8483 | ATAAGAATTACAGGTAC | 83 | 328 |
| 954381 | N/A | N/A | 8490 | 8506 | TCTAAAATATTACATAG | 80 | 329 |
| 954389 | N/A | N/A | 8551 | 8567 | AGCCACAAAAATGTGAG | 116 | 330 |
| 954397 | N/A | N/A | 8624 | 8640 | ATTTACAGGAAATGGAG | 80 | 331 |
| 954405 | N/A | N/A | 8679 | 8695 | ACTAAATGTTTTGAGTG | 138 | 332 |

TABLE 6
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 140 | 78 |
| 953798 | 34 | 50 | 1019 | 1035 | GGTAAAAGCAGAACCTG | 142 | 333 |
| 953806 | N/A | N/A | 1595 | 1611 | GACAAGGGAAGACCCAA | 122 | 334 |
| 953814 | N/A | N/A | 1716 | 1732 | AAAGAAACAAGTTCTCG | 125 | 335 |
| 953822 | N/A | N/A | 1809 | 1825 | GAAGAGGGAAGTGGCAC | 105 | 140 |
| 953830 | N/A | N/A | 1911 | 1927 | CTAAAAGCATTATGTCA | 83 | 336 |
| 953838 | N/A | N/A | 1985 | 2001 | GGAAAAGAAATCTCCCA | 88 | 337 |
| 953846 | N/A | N/A | 2046 | 2062 | AATGAAATGTCCTGACA | 85 | 338 |
| 953854 | N/A | N/A | 2163 | 2179 | CCACTTTACTTGGCAAC | 105 | 339 |
| 953862 | N/A | N/A | 2226 | 2242 | AAAAAACTTCAATCAAC | 106 | 247 |
| 953870 | N/A | N/A | 2282 | 2298 | CGGCAATTAACCTCCCC | 94 | 340 |
| 953878 | N/A | N/A | 2398 | 2414 | AAGCAAACAGAAACCCT | 125 | 341 |
| 953886 | N/A | N/A | 2427 | 2443 | TTCCAAAAAGTAACAAG | 105 | 342 |
| 953894 | N/A | N/A | 2469 | 2485 | TTCAAAAATTCTCCCCA | 94 | 343 |
| 953902 | N/A | N/A | 2590 | 2606 | TAAGAGAAAGGCCTGAA | 84 | 344 |
| 953910 | N/A | N/A | 2619 | 2635 | ATCCAGATCGGAAATAG | 122 | 345 |
| 953918 | N/A | N/A | 2669 | 2685 | AATTAATCAGTCTTAAC | 86 | 346 |
| 953926 | N/A | N/A | 2718 | 2734 | GACGAGACAAGGATCAT | 120 | 347 |
| 953934 | N/A | N/A | 2747 | 2763 | TCCGAATCATTTTCTAA | 113 | 348 |
| 953942 | N/A | N/A | 2785 | 2801 | TCCTAAATTTTATTCCA | 88 | 349 |
| 953950 | N/A | N/A | 2828 | 2844 | TCCAAATATCCAAAAAG | 122 | 350 |
| 953958 | N/A | N/A | 2895 | 2911 | CACTACAGTAAATTTCT | 84 | 351 |
| 953966 | N/A | N/A | 2936 | 2952 | TGCAATGTGTGAACTTT | 60 | 352 |
| 953974 | N/A | N/A | 2959 | 2975 | GTTTAGTGTTTACACAA | 114 | 353 |
| 953982 | N/A | N/A | 3029 | 3045 | GCATATTTTTAGAATGA | 159 | 354 |
| 953990 | N/A | N/A | 3069 | 3085 | GCAAATGCAGGTTCTGT | 103 | 355 |
| 953998 | N/A | N/A | 3103 | 3119 | AGGAAATTTCTTGATAC | 124 | 356 |
| 954006 | N/A | N/A | 3138 | 3154 | TGAAACAAAATCTATCT | 89 | 357 |
| 954014 | N/A | N/A | 3169 | 3185 | AATAAGGTAATCATATA | 117 | 358 |
| 954022 | N/A | N/A | 3516 | 3532 | TGTCAATGTATTACTTA | 77 | 359 |
| 954030 | N/A | N/A | 3597 | 3613 | ACAAAGTTAGGTACTCA | 50 | 360 |
| 954038 | N/A | N/A | 3654 | 3670 | GAACAGGTTATATCTGG | 84 | 361 |
| 954046 | N/A | N/A | 3675 | 3691 | TTTAAATCTATCTTACA | 119 | 362 |
| 954054 | N/A | N/A | 4018 | 4034 | AGTTATTATTCTAGCCA | 84 | 363 |
| 954062 | N/A | N/A | 4069 | 4085 | TATAAATAAACCTAATG | 85 | 364 |
| 954070 | N/A | N/A | 4152 | 4168 | AAATTAGTGAATACCTC | 90 | 365 |
| 954078 | N/A | N/A | 4189 | 4205 | CCAAAATCCAAACTTTG | 118 | 366 |
| 954086 | N/A | N/A | 4274 | 4290 | AAACAGAACAGATACTT | 92 | 367 |
| 954094 | N/A | N/A | 4344 | 4360 | AAACACTCATAAACTGA | 91 | 368 |
| 954102 | N/A | N/A | 4443 | 4459 | TCCTAATTACTGTGAGG | 101 | 369 |
| 954110 | N/A | N/A | 4478 | 4494 | GAATAAGAAACTGCCAG | 89 | 370 |
| 954118 | N/A | N/A | 4516 | 4532 | AATAAAGTCAGGGAAAC | 71 | 371 |
| 954126 | N/A | N/A | 4553 | 4569 | ACATAGTACATGTTTAG | 87 | 372 |
| 954134 | N/A | N/A | 4623 | 4639 | AATGAAAGCTTCACGAG | 84 | 373 |
| 954142 | N/A | N/A | 4897 | 4913 | TGATAGAGCAAGAACTG | 87 | 374 |
| 954150 | N/A | N/A | 5504 | 5520 | GTATTTAAAATATCAAC | 88 | 375 |
| 954158 | N/A | N/A | 5616 | 5632 | CTACGATAGTGATACTG | 101 | 376 |
| 954166 | N/A | N/A | 5781 | 5797 | TACCACTACTTCATACA | 94 | 377 |
| 954174 | N/A | N/A | 5823 | 5839 | CCTGAAACAGGAAGATT | 92 | 378 |
| 954182 | N/A | N/A | 5877 | 5893 | CTACAAATGTCCCCCTC | 97 | 379 |
| 954190 | N/A | N/A | 5965 | 5981 | ATAAATGCCAATATCTT | 107 | 380 |
| 954198 | N/A | N/A | 6034 | 6050 | CTTAAGATTGAAATTCA | 84 | 381 |
| 954206 | N/A | N/A | 6193 | 6209 | CCGCATTCAAAATCCAG | 124 | 382 |
| 954214 | N/A | N/A | 6244 | 6260 | AGCTACTGCTAAATTGT | 89 | 383 |
| 954222 | N/A | N/A | 6305 | 6321 | GTTTAAGCTATTGCAGC | 101 | 384 |
| 954230 | N/A | N/A | 6706 | 6722 | GTATAAGAAAGAGCCTG | 115 | 385 |
| 954238 | N/A | N/A | 6732 | 6748 | AATGAGAACAAGGGTTT | 64 | 386 |
| 954246 | N/A | N/A | 6844 | 6860 | AGACAATCCAAAGGAAG | 128 | 387 |
| 954254 | N/A | N/A | 7043 | 7059 | CAAAATGGATCAGACTT | 85 | 388 |
| 954262 | N/A | N/A | 7164 | 7180 | TACTAAGATAATTCCAT | 134 | 389 |
| 954270 | N/A | N/A | 7194 | 7210 | CAATTAAGGACGATTAA | 88 | 390 |
| 954278 | N/A | N/A | 7231 | 7247 | CAACAGGTAGAACTGAC | 118 | 391 |
| 954286 | N/A | N/A | 7286 | 7302 | CACAAACTTCAGTAATC | 120 | 392 |
| 954294 | N/A | N/A | 7374 | 7390 | ACGGAAATGCAAGGGAC | 73 | 393 |
| 954302 | N/A | N/A | 7525 | 7541 | ATTTACACAAAGTTTGC | 84 | 394 |
| 954310 | N/A | N/A | 7598 | 7614 | CTACAATGGCAGAGCCG | 132 | 395 |
| 954318 | N/A | N/A | 7723 | 7739 | GACCAAAATAATCAAAT | 113 | 396 |
| 954326 | N/A | N/A | 7792 | 7808 | GAAAACTATAACCTGCA | 107 | 397 |
| 954334 | N/A | N/A | 8167 | 8183 | GCCAAAAAAGAAATGCT | 131 | 398 |
| 954342 | N/A | N/A | 8296 | 8312 | AGTTAGACAAAGATGAC | 102 | 399 |
| 954350 | N/A | N/A | 8340 | 8356 | TAAAATCTGGGAAGAAG | 87 | 400 |
| 954358 | N/A | N/A | 8368 | 8384 | ACACATTTAAATGCTTG | 92 | 401 |
| 954366 | N/A | N/A | 8443 | 8459 | GAACAATATCTATGACA | 77 | 402 |
| 954374 | N/A | N/A | 8469 | 8485 | TAATAAGAATTACAGGT | 78 | 403 |
| 954382 | N/A | N/A | 8516 | 8532 | CCAAAAGTCCTCTTTTA | 95 | 404 |
| 954390 | N/A | N/A | 8577 | 8593 | GAGAATATGTCACCAGC | 102 | 405 |
| 954398 | N/A | N/A | 8626 | 8642 | TTATTTACAGGAAATGG | 87 | 406 |
| 954406 | N/A | N/A | 8680 | 8696 | GACTAAATGTTTTGAGT | 81 | 407 |

TABLE 7
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 130 | 78 |
| 953799 | 39 | 55 | 1024 | 1040 | CCGCAGGTAAAAGCAGA | 66 | 408 |
| 953807 | N/A | N/A | 1596 | 1612 | GGACAAGGGAAGACCCA | 104 | 409 |
| 953815 | N/A | N/A | 1725 | 1741 | CGCAAATAAAAAGAAAC | 122 | 112 |
| 953823 | N/A | N/A | 1812 | 1828 | CTAGAAGAGGGAAGTGG | 145 | 141 |
| 953831 | N/A | N/A | 1912 | 1928 | CCTAAAAGCATTATGTC | 83 | 410 |
| 953839 | N/A | N/A | 1986 | 2002 | AGGAAAAGAAATCTCCC | 133 | 411 |
| 953847 | N/A | N/A | 2051 | 2067 | AACTAAATGAAATGTCC | 132 | 412 |
| 953855 | N/A | N/A | 2165 | 2181 | CACCACTTTACTTGGCA | 102 | 413 |
| 953863 | N/A | N/A | 2227 | 2243 | CAAAAAACTTCAATCAA | 117 | 414 |
| 953871 | N/A | N/A | 2308 | 2324 | GTAAAATGTACACCTCA | 74 | 415 |
| 953879 | N/A | N/A | 2403 | 2419 | CAATGAAGCAAACAGAA | 106 | 416 |
| 953887 | N/A | N/A | 2446 | 2462 | AGCAACAGAAACCCCTA | 115 | 417 |
| 953895 | N/A | N/A | 2470 | 2486 | TTTCAAAAATTCTCCCC | 136 | 418 |
| 953903 | N/A | N/A | 2592 | 2608 | GGTAAGAGAAAGGCCTG | 121 | 419 |
| 953911 | N/A | N/A | 2623 | 2639 | ACACATCCAGATCGGAA | 115 | 420 |
| 953919 | N/A | N/A | 2670 | 2686 | AAATTAATCAGTCTTAA | 90 | 421 |
| 953927 | N/A | N/A | 2723 | 2739 | TTGAAGACGAGACAAGG | 110 | 422 |
| 953935 | N/A | N/A | 2766 | 2782 | AAATAAGCTAACACTGC | 110 | 423 |
| 953943 | N/A | N/A | 2797 | 2813 | AGAATAATTTATTCCTA | 123 | 424 |
| 953951 | N/A | N/A | 2855 | 2871 | AGATAAGCCAAATTGTT | 129 | 425 |
| 953959 | N/A | N/A | 2897 | 2913 | AGCACTACAGTAAATTT | 105 | 426 |
| 953967 | N/A | N/A | 2941 | 2957 | ATACATGCAATGTGTGA | 111 | 427 |
| 953975 | N/A | N/A | 2990 | 3006 | CAGAAAGGTCTTCCTTC | 131 | 428 |
| 953983 | N/A | N/A | 3031 | 3047 | AGGCATATTTTTAGAAT | 116 | 429 |
| 953991 | N/A | N/A | 3070 | 3086 | AGCAAATGCAGGTTCTG | 49 | 430 |
| 953999 | N/A | N/A | 3104 | 3120 | AAGGAAATTTCTTGATA | 123 | 431 |
| 954007 | N/A | N/A | 3139 | 3155 | ATGAAACAAAATCTATC | 103 | 432 |
| 954015 | N/A | N/A | 3170 | 3186 | AAATAAGGTAATCATAT | 89 | 433 |
| 954023 | N/A | N/A | 3531 | 3547 | ACAGAATTTGTATCATG | 47 | 434 |
| 954031 | N/A | N/A | 3598 | 3614 | GACAAAGTTAGGTACTC | 81 | 435 |
| 954039 | N/A | N/A | 3656 | 3672 | ATGAACAGGTTATATCT | 101 | 436 |
| 954047 | N/A | N/A | 3679 | 3695 | GCATTTTAAATCTATCT | 28 | 437 |
| 954055 | N/A | N/A | 4032 | 4048 | TAAGAACCTTTAAAAGT | 109 | 438 |
| 954063 | N/A | N/A | 4070 | 4086 | CTATAAATAAACCTAAT | 104 | 439 |
| 954071 | N/A | N/A | 4153 | 4169 | AAAATTAGTGAATACCT | 80 | 440 |
| 954079 | N/A | N/A | 4190 | 4206 | GCCAAAATCCAAACTTT | 118 | 441 |
| 954087 | N/A | N/A | 4280 | 4296 | CCATAAAAACAGAACAG | 93 | 442 |
| 954095 | N/A | N/A | 4348 | 4364 | GCACAAACACTCATAAA | 100 | 443 |
| 954103 | N/A | N/A | 4454 | 4470 | TATTAATTGCCTCCTAA | 81 | 444 |
| 954111 | N/A | N/A | 4479 | 4495 | AGAATAAGAAACTGCCA | 119 | 445 |
| 954119 | N/A | N/A | 4517 | 4533 | GAATAAAGTCAGGGAAA | 79 | 446 |
| 954127 | N/A | N/A | 4555 | 4571 | GCACATAGTACATGTTT | 107 | 447 |
| 954135 | N/A | N/A | 4629 | 4645 | CATTAGAATGAAAGCTT | 74 | 448 |
| 954143 | N/A | N/A | 4899 | 4915 | GCTGATAGAGCAAGAAC | 116 | 449 |
| 954151 | N/A | N/A | 5505 | 5521 | CGTATTTAAAATATCAA | 101 | 450 |
| 954159 | N/A | N/A | 5618 | 5634 | AGCTACGATAGTGATAC | 108 | 451 |
| 954167 | N/A | N/A | 5786 | 5802 | TGGAATACCACTACTTC | 110 | 452 |
| 954175 | N/A | N/A | 5844 | 5860 | TAAAATGACATCCTTGC | 123 | 453 |
| 954183 | N/A | N/A | 5882 | 5898 | TCTTACTACAAATGTCC | 125 | 454 |
| 954191 | N/A | N/A | 5966 | 5982 | CATAAATGCCAATATCT | 120 | 455 |
| 954199 | N/A | N/A | 6036 | 6052 | CACTTAAGATTGAAATT | 122 | 456 |
| 954207 | N/A | N/A | 6211 | 6227 | CAACAAGTGCAGTTGGT | 104 | 457 |
| 954215 | N/A | N/A | 6249 | 6265 | ACGCAAGCTACTGCTAA | 115 | 458 |
| 954223 | N/A | N/A | 6328 | 6344 | TGATAAGCTCTTGCTTG | 107 | 459 |
| 954231 | N/A | N/A | 6707 | 6723 | AGTATAAGAAAGAGCCT | 101 | 460 |
| 954239 | N/A | N/A | 6802 | 6818 | TGATAAGAATCCAGTAT | 95 | 461 |
| 954247 | N/A | N/A | 6913 | 6929 | ATAGATAAACTCGACTT | 86 | 462 |
| 954255 | N/A | N/A | 7044 | 7060 | TCAAAATGGATCAGACT | 97 | 463 |
| 954263 | N/A | N/A | 7167 | 7183 | GGGTACTAAGATAATTC | 100 | 464 |
| 954271 | N/A | N/A | 7195 | 7211 | ACAATTAAGGACGATTA | 93 | 465 |
| 954279 | N/A | N/A | 7234 | 7250 | GACCAACAGGTAGAACT | 109 | 466 |
| 954287 | N/A | N/A | 7287 | 7303 | ACACAAACTTCAGTAAT | 128 | 467 |
| 954295 | N/A | N/A | 7423 | 7439 | TCCTACTTGGCTTCTTT | 131 | 468 |
| 954303 | N/A | N/A | 7528 | 7544 | ACCATTTACACAAAGTT | 86 | 469 |
| 954311 | N/A | N/A | 7618 | 7634 | TGCTTATGGCTACTTTC | 108 | 470 |
| 954319 | N/A | N/A | 7733 | 7749 | TTAAATGGTTGACCAAA | 84 | 471 |
| 954327 | N/A | N/A | 7793 | 7809 | GGAAAACTATAACCTGC | 120 | 472 |
| 954335 | N/A | N/A | 8222 | 8238 | GTATATGTGTAACTTTT | 64 | 473 |
| 954343 | N/A | N/A | 8301 | 8317 | TAGGAAGTTAGACAAAG | 56 | 474 |
| 954351 | N/A | N/A | 8343 | 8359 | TATTAAAATCTGGGAAG | 105 | 475 |
| 954359 | N/A | N/A | 8370 | 8386 | TGACACATTTAAATGCT | 126 | 476 |
| 954367 | N/A | N/A | 8445 | 8461 | TGGAACAATATCTATGA | 85 | 477 |
| 954375 | N/A | N/A | 8470 | 8486 | TTAATAAGAATTACAGG | 90 | 478 |
| 954383 | N/A | N/A | 8517 | 8533 | TCCAAAAGTCCTCTTTT | 122 | 479 |
| 954391 | N/A | N/A | 8578 | 8594 | AGAGAATATGTCACCAG | 67 | 480 |
| 954399 | N/A | N/A | 8629 | 8645 | GACTTATTTACAGGAAA | 77 | 481 |
| 954407 | N/A | N/A | 8683 | 8699 | TAAGACTAAATGTTTTG | 117 | 482 |

TABLE 8
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 93 | 78 |
| 953800 | 287 | 303 | 1266 | 1282 | AGCTGAGGAGGCGGCGG | 129 | 483 |
| 953808 | N/A | N/A | 1603 | 1619 | CGCGAGAGGACAAGGGA | 114 | 484 |
| 953816 | N/A | N/A | 1753 | 1769 | AGTTAAAAGAACCCCCG | 116 | 485 |
| 953824 | N/A | N/A | 1814 | 1830 | GACTAGAAGAGGGAAGT | 110 | 486 |
| 953832 | N/A | N/A | 1913 | 1929 | TCCTAAAAGCATTATGT | 141 | 171 |
| 953840 | N/A | N/A | 1987 | 2003 | TAGGAAAAGAAATCTCC | 115 | 186 |
| 953848 | N/A | N/A | 2096 | 2112 | CTCTTAGGTGGTGCTTA | 104 | 217 |
| 953856 | N/A | N/A | 2188 | 2204 | TTTCATTAATCACCACG | 115 | 487 |
| 953864 | N/A | N/A | 2229 | 2245 | GGCAAAAAACTTCAATC | 81 | 248 |
| 953872 | N/A | N/A | 2309 | 2325 | GGTAAAATGTACACCTC | 109 | 487 |
| 953880 | N/A | N/A | 2405 | 2421 | AGCAATGAAGCAAACAG | 96 | 488 |
| 953888 | N/A | N/A | 2447 | 2463 | AAGCAACAGAAACCCCT | 112 | 489 |
| 953896 | N/A | N/A | 2494 | 2510 | GTTTTAATGGTCTCTCT | 59 | 490 |
| 953904 | N/A | N/A | 2603 | 2619 | GAATACTGAGAGGTAAG | 101 | 491 |
| 953912 | N/A | N/A | 2634 | 2650 | TGCCATCTGGGACACAT | 117 | 492 |
| 953920 | N/A | N/A | 2671 | 2687 | AAAATTAATCAGTCTTA | 88 | 493 |
| 953928 | N/A | N/A | 2730 | 2746 | TTAAACTTTGAAGACGA | 86 | 494 |
| 953936 | N/A | N/A | 2767 | 2783 | CAAATAAGCTAACACTG | 112 | 495 |
| 953944 | N/A | N/A | 2803 | 2819 | TCCTTTAGAATAATTTA | 111 | 496 |
| 953952 | N/A | N/A | 2859 | 2875 | GAAGAGATAAGCCAAAT | 101 | 497 |
| 953960 | N/A | N/A | 2898 | 2914 | AAGCACTACAGTAAATT | 111 | 498 |
| 953968 | N/A | N/A | 2945 | 2961 | CAAGATACATGCAATGT | 121 | 499 |
| 953976 | N/A | N/A | 2991 | 3007 | GCAGAAAGGTCTTCCTT | 114 | 500 |
| 953984 | N/A | N/A | 3035 | 3051 | GAGAAGGCATATTTTTA | 103 | 501 |
| 953992 | N/A | N/A | 3075 | 3091 | AGATAAGCAAATGCAGG | 97 | 502 |
| 954000 | N/A | N/A | 3108 | 3124 | AGCAAAGGAAATTTCTT | 121 | 503 |
| 954008 | N/A | N/A | 3148 | 3164 | CTTAAAGTAATGAAACA | 132 | 504 |
| 954016 | N/A | N/A | 3171 | 3187 | TAAATAAGGTAATCATA | 125 | 505 |
| 954024 | N/A | N/A | 3533 | 3549 | TTACAGAATTTGTATCA | 138 | 506 |
| 954032 | N/A | N/A | 3620 | 3636 | TGAAATAGTGCTTGTTC | 91 | 507 |
| 954040 | N/A | N/A | 3657 | 3673 | TATGAACAGGTTATATC | 120 | 508 |
| 954048 | N/A | N/A | 3701 | 3717 | CTAAACTACTTTTGTTT | 95 | 509 |
| 954056 | N/A | N/A | 4033 | 4049 | CTAAGAACCTTTAAAAG | 135 | 510 |
| 954064 | N/A | N/A | 4071 | 4087 | ACTATAAATAAACCTAA | 117 | 511 |
| 954072 | N/A | N/A | 4154 | 4170 | CAAAATTAGTGAATACC | 93 | 512 |
| 954080 | N/A | N/A | 4199 | 4215 | AACAGAACTGCCAAAAT | 117 | 513 |
| 954088 | N/A | N/A | 4288 | 4304 | AGCAAGAGCCATAAAAA | 122 | 514 |
| 954096 | N/A | N/A | 4355 | 4371 | GAGCAGAGCACAAACAC | 102 | 515 |
| 954104 | N/A | N/A | 4455 | 4471 | GTATTAATTGCCTCCTA | 110 | 516 |
| 954112 | N/A | N/A | 4480 | 4496 | GAGAATAAGAAACTGCC | 108 | 517 |
| 954120 | N/A | N/A | 4519 | 4535 | ATGAATAAAGTCAGGGA | 105 | 518 |
| 954128 | N/A | N/A | 4557 | 4573 | AGGCACATAGTACATGT | 144 | 519 |
| 954136 | N/A | N/A | 4632 | 4648 | CTTCATTAGAATGAAAG | 123 | 520 |
| 954144 | N/A | N/A | 4900 | 4916 | GGCTGATAGAGCAAGAA | 118 | 521 |
| 954152 | N/A | N/A | 5506 | 5522 | CCGTATTTAAAATATCA | 101 | 522 |
| 954160 | N/A | N/A | 5679 | 5695 | CAACACATTGTGAGGTT | 110 | 523 |
| 954168 | N/A | N/A | 5815 | 5831 | AGGAAGATTTTGGACCT | 108 | 524 |
| 954176 | N/A | N/A | 5845 | 5861 | CTAAAATGACATCCTTG | 96 | 525 |
| 954184 | N/A | N/A | 5934 | 5950 | GGCAAGTAGTATTGGTC | 102 | 526 |
| 954192 | N/A | N/A | 5967 | 5983 | ACATAAATGCCAATATC | 115 | 527 |
| 954200 | N/A | N/A | 6046 | 6062 | AGCCAGAGTTCACTTAA | 98 | 528 |
| 954208 | N/A | N/A | 6221 | 6237 | TAGCAGAGTTCAACAAG | 106 | 529 |
| 954216 | N/A | N/A | 6253 | 6269 | GATAACGCAAGCTACTG | 107 | 530 |
| 954224 | N/A | N/A | 6345 | 6361 | GACTTATTTTTGTCTTC | 129 | 531 |
| 954232 | N/A | N/A | 6709 | 6725 | TAAGTATAAGAAAGAGC | 99 | 532 |
| 954240 | N/A | N/A | 6823 | 6839 | AGTTTTTACAAATCATC | 91 | 533 |
| 954248 | N/A | N/A | 6914 | 6930 | AATAGATAAACTCGACT | 100 | 534 |
| 954256 | N/A | N/A | 7086 | 7102 | AATAAAGTTGGACTTCT | 121 | 535 |
| 954264 | N/A | N/A | 7172 | 7188 | AACAAGGGTACTAAGAT | 80 | 536 |
| 954272 | N/A | N/A | 7196 | 7212 | TACAATTAAGGACGATT | 63 | 537 |
| 954280 | N/A | N/A | 7239 | 7255 | ATAAAGACCAACAGGTA | 118 | 538 |
| 954288 | N/A | N/A | 7297 | 7313 | TTAAACTGTGACACAAA | 71 | 539 |
| 954296 | N/A | N/A | 7441 | 7457 | ACACAATCCCTCTCAGA | 100 | 540 |
| 954304 | N/A | N/A | 7543 | 7559 | AATACTTACAATCTGAC | 54 | 541 |
| 954312 | N/A | N/A | 7626 | 7642 | ATACATATTGCTTATGG | 113 | 542 |
| 954320 | N/A | N/A | 7734 | 7750 | TTTAAATGGTTGACCAA | 94 | 543 |
| 954328 | N/A | N/A | 7794 | 7810 | GGGAAAACTATAACCTG | 121 | 544 |
| 954336 | N/A | N/A | 8272 | 8288 | CAAGAAGGGCTTTGTTT | 116 | 545 |
| 954344 | N/A | N/A | 8305 | 8321 | TTCTTAGGAAGTTAGAC | 107 | 546 |
| 954352 | N/A | N/A | 8344 | 8360 | ATATTAAAATCTGGGAA | 78 | 547 |
| 954360 | N/A | N/A | 8407 | 8423 | ACATATGTAATGTGATC | 111 | 548 |
| 954368 | N/A | N/A | 8450 | 8466 | TGCTATGGAACAATATC | 135 | 549 |
| 954376 | N/A | N/A | 8473 | 8489 | CAATTAATAAGAATTAC | 110 | 550 |
| 954384 | N/A | N/A | 8545 | 8561 | AAAAATGTGAGACCTTT | 103 | 551 |
| 954392 | N/A | N/A | 8582 | 8598 | GTAGAGAGAATATGTCA | 66 | 552 |
| 954400 | N/A | N/A | 8640 | 8656 | ATCAAGTGGTTGACTTA | 114 | 553 |
| 954408 | N/A | N/A | 8688 | 8704 | CTGAATAAGACTAAATG | 103 | 554 |

TABLE 9
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 114 | 78 |
| 953801 | 329 | 345 | 1308 | 1324 | GGCTGAGGCAGCAGCGG | 129 | 555 |
| 953809 | N/A | N/A | 1624 | 1640 | CAACAAGGCTCTGCCTC | 119 | 556 |
| 953817 | N/A | N/A | 1757 | 1773 | ACGCAGTTAAAAGAACC | 88 | 557 |
| 953825 | N/A | N/A | 1817 | 1833 | TCAGACTAGAAGAGGGA | 123 | 558 |
| 953833 | N/A | N/A | 1942 | 1958 | CCCCCTGCCCCGCCACT | 91 | 559 |
| 953841 | N/A | N/A | 1993 | 2009 | TGCCACTAGGAAAAGAA | 125 | 188 |
| 953849 | N/A | N/A | 2108 | 2124 | AGATGAGCAGATCTCTT | 99 | 560 |
| 953857 | N/A | N/A | 2193 | 2209 | GATAATTTCATTAATCA | 92 | 561 |
| 953865 | N/A | N/A | 2230 | 2246 | AGGCAAAAAACTTCAAT | 103 | 562 |
| 953873 | N/A | N/A | 2310 | 2326 | TGGTAAAATGTACACCT | 88 | 563 |
| 953881 | N/A | N/A | 2406 | 2422 | CAGCAATGAAGCAAACA | 118 | 564 |
| 953889 | N/A | N/A | 2451 | 2467 | GAACAAGCAACAGAAAC | 99 | 565 |
| 953897 | N/A | N/A | 2502 | 2518 | CGCTAGATGTTTTAATG | 87 | 566 |
| 953905 | N/A | N/A | 2608 | 2624 | AAATAGAATACTGAGAG | 67 | 567 |
| 953913 | N/A | N/A | 2654 | 2670 | ACAGAGATATTCTTACC | 104 | 568 |
| 953921 | N/A | N/A | 2674 | 2690 | CTAAAAATTAATCAGTC | 73 | 569 |
| 953929 | N/A | N/A | 2731 | 2747 | ATTAAACTTTGAAGACG | 110 | 570 |
| 953937 | N/A | N/A | 2768 | 2784 | ACAAATAAGCTAACACT | 105 | 571 |
| 953945 | N/A | N/A | 2818 | 2834 | CAAAAAGTTTTTCCATC | 90 | 572 |
| 953953 | N/A | N/A | 2866 | 2882 | ACTTACTGAAGAGATAA | 83 | 573 |
| 953961 | N/A | N/A | 2902 | 2918 | AGAAAAGCACTACAGTA | 105 | 574 |
| 953969 | N/A | N/A | 2946 | 2962 | ACAAGATACATGCAATG | 100 | 575 |
| 953977 | N/A | N/A | 2993 | 3009 | CAGCAGAAAGGTCTTCC | 128 | 576 |
| 953985 | N/A | N/A | 3036 | 3052 | AGAGAAGGCATATTTTT | 89 | 577 |
| 953993 | N/A | N/A | 3077 | 3093 | GAAGATAAGCAAATGCA | 70 | 578 |
| 954001 | N/A | N/A | 3109 | 3125 | CAGCAAAGGAAATTTCT | 92 | 579 |
| 954009 | N/A | N/A | 3149 | 3165 | CCTTAAAGTAATGAAAC | 112 | 580 |
| 954017 | N/A | N/A | 3172 | 3188 | TTAAATAAGGTAATCAT | 93 | 581 |
| 954025 | N/A | N/A | 3537 | 3553 | GTAATTACAGAATTTGT | 104 | 582 |
| 954033 | N/A | N/A | 3622 | 3638 | ACTGAAATAGTGCTTGT | 68 | 583 |
| 954041 | N/A | N/A | 3658 | 3674 | ATATGAACAGGTTATAT | 91 | 584 |
| 954049 | N/A | N/A | 3703 | 3719 | TTCTAAACTACTTTTGT | 99 | 585 |
| 954057 | N/A | N/A | 4035 | 4051 | TGCTAAGAACCTTTAAA | 88 | 586 |
| 954065 | N/A | N/A | 4072 | 4088 | AACTATAAATAAACCTA | 107 | 587 |
| 954073 | N/A | N/A | 4155 | 4171 | TCAAAATTAGTGAATAC | 105 | 588 |
| 954081 | N/A | N/A | 4200 | 4216 | GAACAGAACTGCCAAAA | 78 | 589 |
| 954089 | N/A | N/A | 4294 | 4310 | CCAAAGAGCAAGAGCCA | 110 | 590 |
| 954097 | N/A | N/A | 4384 | 4400 | CCATAAGGAACTCCTGC | 96 | 591 |
| 954105 | N/A | N/A | 4457 | 4473 | AAGTATTAATTGCCTCC | 94 | 592 |
| 954113 | N/A | N/A | 4481 | 4497 | GGAGAATAAGAAACTGC | 86 | 593 |
| 954121 | N/A | N/A | 4520 | 4536 | AATGAATAAAGTCAGGG | 85 | 594 |
| 954129 | N/A | N/A | 4570 | 4586 | CTATAACAGTGCCAGGC | 49 | 595 |
| 954137 | N/A | N/A | 4742 | 4758 | TAACACTCGATTAACCC | 88 | 596 |
| 954145 | N/A | N/A | 5279 | 5295 | TCACAAGGTCGGCGGAT | 90 | 597 |
| 954153 | N/A | N/A | 5511 | 5527 | GAACACCGTATTTAAAA | 89 | 598 |
| 954161 | N/A | N/A | 5726 | 5742 | TCAAAAGCCATCATCTG | 84 | 599 |
| 954169 | N/A | N/A | 5816 | 5832 | CAGGAAGATTTTGGACC | 95 | 600 |
| 954177 | N/A | N/A | 5846 | 5862 | ACTAAAATGACATCCTT | 113 | 601 |
| 954185 | N/A | N/A | 5935 | 5951 | AGGCAAGTAGTATTGGT | 81 | 602 |
| 954193 | N/A | N/A | 5968 | 5984 | CACATAAATGCCAATAT | 90 | 603 |
| 954201 | N/A | N/A | 6093 | 6109 | CCTAACTGGTTTCTGCT | 89 | 604 |
| 954209 | N/A | N/A | 6228 | 6244 | TTATACTTAGCAGAGTT | 88 | 605 |
| 954217 | N/A | N/A | 6254 | 6270 | TGATAACGCAAGCTACT | 108 | 606 |
| 954225 | N/A | N/A | 6350 | 6366 | CCCCAGACTTATTTTTG | 85 | 607 |
| 954233 | N/A | N/A | 6712 | 6728 | AGGTAAGTATAAGAAAG | 89 | 608 |
| 954241 | N/A | N/A | 6833 | 6849 | AGGAAGGGAGAGTTTTT | 92 | 609 |
| 954249 | N/A | N/A | 6917 | 6933 | CAAAATAGATAAACTCG | 97 | 610 |
| 954257 | N/A | N/A | 7087 | 7103 | GAATAAAGTTGGACTTC | 104 | 611 |
| 954265 | N/A | N/A | 7184 | 7200 | CGATTAATTTTCAACAA | 109 | 612 |
| 954273 | N/A | N/A | 7197 | 7213 | ATACAATTAAGGACGAT | 74 | 613 |
| 954281 | N/A | N/A | 7241 | 7257 | ACATAAAGACCAACAGG | 87 | 614 |
| 954289 | N/A | N/A | 7299 | 7315 | ATTTAAACTGTGACACA | 109 | 615 |
| 954297 | N/A | N/A | 7479 | 7495 | TAAGATGCGAATACTCC | 87 | 616 |
| 954305 | N/A | N/A | 7546 | 7562 | CGAAATACTTACAATCT | 90 | 617 |
| 954313 | N/A | N/A | 7628 | 7644 | TCATACATATTGCTTAT | 64 | 618 |
| 954321 | N/A | N/A | 7735 | 7751 | TTTTAAATGGTTGACCA | 88 | 619 |
| 954329 | N/A | N/A | 7809 | 7825 | TTTTAACCTAGGAGTGG | 79 | 620 |
| 954337 | N/A | N/A | 8273 | 8289 | GCAAGAAGGGCTTTGTT | 102 | 621 |
| 954345 | N/A | N/A | 8320 | 8336 | CAAGAAACACTTTTGTT | 120 | 622 |
| 954353 | N/A | N/A | 8345 | 8361 | CATATTAAAATCTGGGA | 82 | 623 |
| 954361 | N/A | N/A | 8409 | 8425 | ATACATATGTAATGTGA | 97 | 624 |
| 954369 | N/A | N/A | 8462 | 8478 | AATTACAGGTACTGCTA | 101 | 625 |
| 954377 | N/A | N/A | 8474 | 8490 | GCAATTAATAAGAATTA | 93 | 626 |
| 954385 | N/A | N/A | 8546 | 8562 | CAAAAATGTGAGACCTT | 86 | 627 |
| 954393 | N/A | N/A | 8584 | 8600 | AGGTAGAGAGAATATGT | 109 | 628 |
| 954401 | N/A | N/A | 8641 | 8657 | TATCAAGTGGTTGACTT | 94 | 629 |
| 954409 | N/A | N/A | 8689 | 8705 | TCTGAATAAGACTAAAT | 111 | 630 |

TABLE 10
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 111 | 78 |
| 953802 | N/A | N/A | 1408 | 1424 | GGACAGGGAGCTGCAGC | 95 | 631 |
| 953810 | N/A | N/A | 1626 | 1642 | CCCAACAAGGCTCTGCC | 87 | 632 |
| 953818 | N/A | N/A | 1761 | 1777 | CACAACGCAGTTAAAAG | 103 | 124 |
| 953826 | N/A | N/A | 1886 | 1902 | CCCCACATCTCTCCAGC | 109 | 633 |
| 953834 | N/A | N/A | 1966 | 1982 | GGACGCGCCCTCACTCC | 88 | 634 |
| 953842 | N/A | N/A | 2016 | 2032 | CTCAAATCTCAGGCTGT | 111 | 635 |
| 953850 | N/A | N/A | 2112 | 2128 | GCTTAGATGAGCAGATC | 97 | 636 |
| 953858 | N/A | N/A | 2194 | 2210 | AGATAATTTCATTAATC | 95 | 238 |
| 953866 | N/A | N/A | 2234 | 2250 | ACATAGGCAAAAAACTT | 92 | 637 |
| 953874 | N/A | N/A | 2316 | 2332 | GAATACTGGTAAAATGT | 112 | 638 |
| 953882 | N/A | N/A | 2417 | 2433 | TAACAAGCTGTCAGCAA | 106 | 639 |
| 953890 | N/A | N/A | 2454 | 2470 | CAAGAACAAGCAACAGA | 100 | 640 |
| 953898 | N/A | N/A | 2525 | 2541 | TTCCAGGGAAAGTCCTG | 86 | 641 |
| 953906 | N/A | N/A | 2610 | 2626 | GGAAATAGAATACTGAG | 67 | 642 |
| 953914 | N/A | N/A | 2656 | 2672 | TAACAGAGATATTCTTA | 111 | 643 |
| 953922 | N/A | N/A | 2675 | 2691 | ACTAAAAATTAATCAGT | 91 | 644 |
| 953930 | N/A | N/A | 2732 | 2748 | AATTAAACTTTGAAGAC | 109 | 645 |
| 953938 | N/A | N/A | 2769 | 2785 | AACAAATAAGCTAACAC | 98 | 646 |
| 953946 | N/A | N/A | 2819 | 2835 | CCAAAAAGTTTTTCCAT | 86 | 647 |
| 953954 | N/A | N/A | 2872 | 2888 | GAAATTACTTACTGAAG | 63 | 648 |
| 953962 | N/A | N/A | 2903 | 2919 | TAGAAAAGCACTACAGT | 79 | 649 |
| 953970 | N/A | N/A | 2947 | 2963 | CACAAGATACATGCAAT | 107 | 650 |
| 953978 | N/A | N/A | 3014 | 3030 | GATCAAGTGTCTGAAGC | 66 | 651 |
| 953986 | N/A | N/A | 3044 | 3060 | CATAAGAAAGAGAAGGC | 72 | 652 |
| 953994 | N/A | N/A | 3078 | 3094 | TGAAGATAAGCAAATGC | 90 | 653 |
| 954002 | N/A | N/A | 3116 | 3132 | GTCAAGGCAGCAAAGGA | 79 | 654 |
| 954010 | N/A | N/A | 3150 | 3166 | ACCTTAAAGTAATGAAA | 101 | 655 |
| 954018 | N/A | N/A | 3173 | 3189 | TTTAAATAAGGTAATCA | 96 | 656 |
| 954026 | N/A | N/A | 3538 | 3554 | TGTAATTACAGAATTTG | 80 | 657 |
| 954034 | N/A | N/A | 3626 | 3642 | AGGAACTGAAATAGTGC | 89 | 658 |
| 954042 | N/A | N/A | 3661 | 3677 | ACAATATGAACAGGTTA | 45 | 659 |
| 954050 | N/A | N/A | 3708 | 3724 | TATTATTCTAAACTACT | 102 | 660 |
| 954058 | N/A | N/A | 4060 | 4076 | ACCTAATGCAGTTGATT | 67 | 661 |
| 954066 | N/A | N/A | 4106 | 4122 | TGACAAATATGCATTTT | 60 | 662 |
| 954074 | N/A | N/A | 4156 | 4172 | CTCAAAATTAGTGAATA | 103 | 663 |
| 954082 | N/A | N/A | 4229 | 4245 | GAGAAGAGGATTTTTTG | 89 | 664 |
| 954090 | N/A | N/A | 4296 | 4312 | CACCAAAGAGCAAGAGC | 101 | 665 |
| 954098 | N/A | N/A | 4386 | 4402 | CCCCATAAGGAACTCCT | 113 | 666 |
| 954106 | N/A | N/A | 4460 | 4476 | AGCAAGTATTAATTGCC | 83 | 667 |
| 954114 | N/A | N/A | 4487 | 4503 | TCTGAAGGAGAATAAGA | 109 | 668 |
| 954122 | N/A | N/A | 4521 | 4537 | GAATGAATAAAGTCAGG | 82 | 669 |
| 954130 | N/A | N/A | 4571 | 4587 | CCTATAACAGTGCCAGG | 94 | 670 |
| 954138 | N/A | N/A | 4749 | 4765 | AATAAGTTAACACTCGA | 92 | 671 |
| 954146 | N/A | N/A | 5281 | 5297 | GATCACAAGGTCGGCGG | 126 | 672 |
| 954154 | N/A | N/A | 5542 | 5558 | AAAAAAGCTGTCTTCTC | 83 | 673 |
| 954162 | N/A | N/A | 5727 | 5743 | CTCAAAAGCCATCATCT | 89 | 674 |
| 954170 | N/A | N/A | 5819 | 5835 | AAACAGGAAGATTTTGG | 87 | 675 |
| 954178 | N/A | N/A | 5847 | 5863 | AACTAAAATGACATCCT | 88 | 676 |
| 954186 | N/A | N/A | 5939 | 5955 | TACAAGGCAAGTAGTAT | 95 | 677 |
| 954194 | N/A | N/A | 5969 | 5985 | TCACATAAATGCCAATA | 67 | 678 |
| 954202 | N/A | N/A | 6094 | 6110 | TCCTAACTGGTTTCTGC | 82 | 679 |
| 954210 | N/A | N/A | 6230 | 6246 | TGTTATACTTAGCAGAG | 74 | 680 |
| 954218 | N/A | N/A | 6263 | 6279 | ATACAAACCTGATAACG | 95 | 681 |
| 954226 | N/A | N/A | 6370 | 6386 | CTTAACTTATTGTTGAA | 107 | 682 |
| 954234 | N/A | N/A | 6716 | 6732 | TGCAAGGTAAGTATAAG | 79 | 683 |
| 954242 | N/A | N/A | 6837 | 6853 | CCAAAGGAAGGGAGAGT | 87 | 684 |
| 954250 | N/A | N/A | 6930 | 6946 | CAGCAACCAAAGACAAA | 88 | 685 |
| 954258 | N/A | N/A | 7088 | 7104 | AGAATAAAGTTGGACTT | 86 | 686 |
| 954266 | N/A | N/A | 7185 | 7201 | ACGATTAATTTTCAACA | 64 | 687 |
| 954274 | N/A | N/A | 7198 | 7214 | TATACAATTAAGGACGA | 63 | 688 |
| 954282 | N/A | N/A | 7242 | 7258 | GACATAAAGACCAACAG | 83 | 689 |
| 954290 | N/A | N/A | 7302 | 7318 | TGAATTTAAACTGTGAC | 64 | 690 |
| 954298 | N/A | N/A | 7502 | 7518 | TCATAGGTGGAAGACAA | 79 | 691 |
| 954306 | N/A | N/A | 7547 | 7563 | CCGAAATACTTACAATC | 71 | 692 |
| 954314 | N/A | N/A | 7630 | 7646 | ACTCATACATATTGCTT | 82 | 693 |
| 954322 | N/A | N/A | 7761 | 7777 | TCACAAGCTAAGAAATG | 103 | 694 |
| 954330 | N/A | N/A | 7816 | 7832 | TATCATATTTTAACCTA | 116 | 695 |
| 954338 | N/A | N/A | 8274 | 8290 | TGCAAGAAGGGCTTTGT | 96 | 696 |
| 954346 | N/A | N/A | 8322 | 8338 | CACAAGAAACACTTTTG | 103 | 697 |
| 954354 | N/A | N/A | 8346 | 8362 | GCATATTAAAATCTGGG | 73 | 698 |
| 954362 | N/A | N/A | 8410 | 8426 | AATACATATGTAATGTG | 95 | 699 |
| 954370 | N/A | N/A | 8463 | 8479 | GAATTACAGGTACTGCT | 71 | 700 |
| 954378 | N/A | N/A | 8475 | 8491 | AGCAATTAATAAGAATT | 82 | 701 |
| 954386 | N/A | N/A | 8547 | 8563 | ACAAAAATGTGAGACCT | 72 | 702 |
| 954394 | N/A | N/A | 8589 | 8605 | TCTCAAGGTAGAGAGAA | 88 | 703 |
| 954402 | N/A | N/A | 8653 | 8669 | TCAAAGGTAGTTTATCA | 83 | 704 |
| 954410 | N/A | N/A | 8722 | 8738 | CTTTATAAGGTATTTTA | 106 | 705 |

TABLE 11
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 118 | 78 |
| 953803 | N/A | N/A | 1458 | 1474 | CCGCAGGCTGCAGGGTT | 97 | 706 |
| 953811 | N/A | N/A | 1627 | 1643 | CCCCAACAAGGCTCTGC | 136 | 707 |
| 953819 | N/A | N/A | 1762 | 1778 | TCACAACGCAGTTAAAA | 120 | 708 |
| 953827 | N/A | N/A | 1904 | 1920 | CATTATGTCATCCACTG | 76 | 709 |
| 953835 | N/A | N/A | 1973 | 1989 | TCCCATTGGACGCGCCC | 103 | 710 |
| 953843 | N/A | N/A | 2033 | 2049 | GACAATGTAGGAAGAGC | 88 | 711 |
| 953851 | N/A | N/A | 2117 | 2133 | CTTAGGCTTAGATGAGC | 135 | 712 |
| 953859 | N/A | N/A | 2205 | 2221 | CCTAATATTTAAGATAA | 92 | 240 |
| 953867 | N/A | N/A | 2235 | 2251 | CACATAGGCAAAAAACT | 94 | 250 |
| 953875 | N/A | N/A | 2318 | 2334 | TGGAATACTGGTAAAAT | 122 | 713 |
| 953883 | N/A | N/A | 2424 | 2440 | CAAAAAGTAACAAGCTG | 98 | 714 |
| 953891 | N/A | N/A | 2455 | 2471 | CCAAGAACAAGCAACAG | 141 | 715 |
| 953899 | N/A | N/A | 2544 | 2560 | GTACACTCGACACACAG | 86 | 716 |
| 953907 | N/A | N/A | 2611 | 2627 | CGGAAATAGAATACTGA | 109 | 717 |
| 953915 | N/A | N/A | 2658 | 2674 | CTTAACAGAGATATTCT | 123 | 718 |
| 953923 | N/A | N/A | 2689 | 2705 | GAACAAGAAATATTACT | 132 | 719 |
| 953931 | N/A | N/A | 2733 | 2749 | TAATTAAACTTTGAAGA | 92 | 720 |
| 953939 | N/A | N/A | 2770 | 2786 | CAACAAATAAGCTAACA | 105 | 721 |
| 953947 | N/A | N/A | 2820 | 2836 | TCCAAAAAGTTTTTCCA | 93 | 722 |
| 953955 | N/A | N/A | 2889 | 2905 | AGTAAATTTCTGGATGA | 92 | 723 |
| 953963 | N/A | N/A | 2904 | 2920 | CTAGAAAAGCACTACAG | 118 | 724 |
| 953971 | N/A | N/A | 2948 | 2964 | ACACAAGATACATGCAA | 123 | 725 |
| 953979 | N/A | N/A | 3021 | 3037 | TTAGAATGATCAAGTGT | 64 | 726 |
| 953987 | N/A | N/A | 3045 | 3061 | GCATAAGAAAGAGAAGG | 85 | 727 |
| 953995 | N/A | N/A | 3079 | 3095 | TTGAAGATAAGCAAATG | 103 | 728 |
| 954003 | N/A | N/A | 3117 | 3133 | TGTCAAGGCAGCAAAGG | 88 | 729 |
| 954011 | N/A | N/A | 3156 | 3172 | TATATTACCTTAAAGTA | 113 | 730 |
| 954019 | N/A | N/A | 3217 | 3233 | TCGGATTAAAGGTGTAA | 127 | 731 |
| 954027 | N/A | N/A | 3554 | 3570 | TTAATTATTGCCCTTTT | 55 | 732 |
| 954035 | N/A | N/A | 3633 | 3649 | ATACATGAGGAACTGAA | 75 | 733 |
| 954043 | N/A | N/A | 3662 | 3678 | TACAATATGAACAGGTT | 63 | 734 |
| 954051 | N/A | N/A | 3709 | 3725 | ATATTATTCTAAACTAC | 124 | 735 |
| 954059 | N/A | N/A | 4065 | 4081 | AATAAACCTAATGCAGT | 130 | 736 |
| 954067 | N/A | N/A | 4119 | 4135 | ATACAGAGAAATATGAC | 122 | 737 |
| 954075 | N/A | N/A | 4157 | 4173 | ACTCAAAATTAGTGAAT | 137 | 738 |
| 954083 | N/A | N/A | 4244 | 4260 | CATCAATCTTACTTTGA | 106 | 739 |
| 954091 | N/A | N/A | 4336 | 4352 | ATAAACTGAGGCCCATG | 133 | 740 |
| 954099 | N/A | N/A | 4413 | 4429 | TGCTAGATTTGCTGAGC | 120 | 741 |
| 954107 | N/A | N/A | 4461 | 4477 | AAGCAAGTATTAATTGC | 98 | 742 |
| 954115 | N/A | N/A | 4497 | 4513 | CAGATAGGAATCTGAAG | 116 | 743 |
| 954123 | N/A | N/A | 4524 | 4540 | GATGAATGAATAAAGTC | 60 | 744 |
| 954131 | N/A | N/A | 4572 | 4588 | ACCTATAACAGTGCCAG | 103 | 745 |
| 954139 | N/A | N/A | 4750 | 4766 | AAATAAGTTAACACTCG | 83 | 746 |
| 954147 | N/A | N/A | 5498 | 5514 | AAAATATCAACACTGGC | 159 | 747 |
| 954155 | N/A | N/A | 5585 | 5601 | TTAAAGAGCAAGACCTT | 114 | 748 |
| 954163 | N/A | N/A | 5750 | 5766 | TCTTAACTCGCTTGAGG | 118 | 749 |
| 954171 | N/A | N/A | 5820 | 5836 | GAAACAGGAAGATTTTG | 104 | 750 |
| 954179 | N/A | N/A | 5850 | 5866 | CCCAACTAAAATGACAT | 109 | 751 |
| 954187 | N/A | N/A | 5940 | 5956 | CTACAAGGCAAGTAGTA | 92 | 752 |
| 954195 | N/A | N/A | 5971 | 5987 | ACTCACATAAATGCCAA | 110 | 753 |
| 954203 | N/A | N/A | 6132 | 6148 | ACAGAACCCACCATGCT | 103 | 754 |
| 954211 | N/A | N/A | 6236 | 6252 | CTAAATTGTTATACTTA | 102 | 755 |
| 954219 | N/A | N/A | 6268 | 6284 | GCTGAATACAAACCTGA | 79 | 756 |
| 954227 | N/A | N/A | 6389 | 6405 | AAAGAAAGAGCCTGGGT | 85 | 757 |
| 954235 | N/A | N/A | 6717 | 6733 | TTGCAAGGTAAGTATAA | 108 | 758 |
| 954243 | N/A | N/A | 6838 | 6854 | TCCAAAGGAAGGGAGAG | 130 | 759 |
| 954251 | N/A | N/A | 6951 | 6967 | TTAGATATGACACTTGA | 103 | 760 |
| 954259 | N/A | N/A | 7161 | 7177 | TAAGATAATTCCATGGG | 112 | 761 |
| 954267 | N/A | N/A | 7186 | 7202 | GACGATTAATTTTCAAC | 100 | 762 |
| 954275 | N/A | N/A | 7199 | 7215 | TTATACAATTAAGGACG | 101 | 763 |
| 954283 | N/A | N/A | 7243 | 7259 | CGACATAAAGACCAACA | 96 | 764 |
| 954291 | N/A | N/A | 7304 | 7320 | CATGAATTTAAACTGTG | 87 | 765 |
| 954299 | N/A | N/A | 7520 | 7536 | CACAAAGTTTGCCCATG | 75 | 766 |
| 954307 | N/A | N/A | 7548 | 7564 | CCCGAAATACTTACAAT | 106 | 767 |
| 954315 | N/A | N/A | 7640 | 7656 | GAACACAGACACTCATA | 119 | 768 |
| 954323 | N/A | N/A | 7780 | 7796 | CTGCATATTTTTGGCTA | 152 | 769 |
| 954331 | N/A | N/A | 7830 | 7846 | TCCAAATGTGGTCCTAT | 103 | 770 |
| 954339 | N/A | N/A | 8290 | 8306 | ACAAAGATGACAGACTT | 68 | 771 |
| 954347 | N/A | N/A | 8323 | 8339 | ACACAAGAAACACTTTT | 83 | 772 |
| 954355 | N/A | N/A | 8347 | 8363 | TGCATATTAAAATCTGG | 94 | 773 |
| 954363 | N/A | N/A | 8433 | 8449 | TATGACACATTGTTAAG | 147 | 774 |
| 954371 | N/A | N/A | 8464 | 8480 | AGAATTACAGGTACTGC | 116 | 775 |
| 954379 | N/A | N/A | 8476 | 8492 | TAGCAATTAATAAGAAT | 104 | 776 |
| 954387 | N/A | N/A | 8548 | 8564 | CACAAAAATGTGAGACC | 111 | 777 |
| 954395 | N/A | N/A | 8618 | 8634 | AGGAAATGGAGGTGATG | 76 | 778 |
| 954403 | N/A | N/A | 8654 | 8670 | TTCAAAGGTAGTTTATC | 149 | 779 |
| 954411 | N/A | N/A | 8723 | 8739 | GCTTTATAAGGTATTTT | 82 | 780 |

TABLE 12
Reduction of HTT RNA by modified oligonucleotides with a mixed MOE/cEt sugar motif
and mixed PO/PS internucleoside linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 646253 | N/A | N/A | 1447 | 1463 | AGGGTTACCGCCATCCC | 97 | 78 |
| 953804 | N/A | N/A | 1583 | 1599 | CCCAAGTGAGGGAGCGG | 98 | 781 |
| 953812 | N/A | N/A | 1674 | 1690 | GGCGAGAGGGCTGAGGC | 114 | 782 |
| 953820 | N/A | N/A | 1798 | 1814 | TGGCACTGAGCAAATCT | 89 | 783 |
| 953828 | N/A | N/A | 1905 | 1921 | GCATTATGTCATCCACT | 101 | 784 |
| 953836 | N/A | N/A | 1978 | 1994 | AAATCTCCCATTGGACG | 92 | 785 |
| 953844 | N/A | N/A | 2034 | 2050 | TGACAATGTAGGAAGAG | 99 | 786 |
| 953852 | N/A | N/A | 2123 | 2139 | GACCAACTTAGGCTTAG | 97 | 787 |
| 953860 | N/A | N/A | 2206 | 2222 | TCCTAATATTTAAGATA | 133 | 788 |
| 953868 | N/A | N/A | 2236 | 2252 | ACACATAGGCAAAAAAC | 87 | 789 |
| 953876 | N/A | N/A | 2361 | 2377 | GCCCACGGAGTCTGCGG | 100 | 790 |
| 953884 | N/A | N/A | 2425 | 2441 | CCAAAAAGTAACAAGCT | 100 | 791 |
| 953892 | N/A | N/A | 2456 | 2472 | CCCAAGAACAAGCAACA | 87 | 792 |
| 953900 | N/A | N/A | 2551 | 2567 | TCCTACTGTACACTCGA | 91 | 793 |
| 953908 | N/A | N/A | 2612 | 2628 | TCGGAAATAGAATACTG | 145 | 794 |
| 953916 | N/A | N/A | 2659 | 2675 | TCTTAACAGAGATATTC | 89 | 795 |
| 953924 | N/A | N/A | 2707 | 2723 | GATCATAACAGAAACAA | 136 | 796 |
| 953932 | N/A | N/A | 2735 | 2751 | TCTAATTAAACTTTGAA | 86 | 797 |
| 953940 | N/A | N/A | 2783 | 2799 | CTAAATTTTATTCCAAC | 79 | 798 |
| 953948 | N/A | N/A | 2821 | 2837 | ATCCAAAAAGTTTTTCC | 97 | 799 |
| 953956 | N/A | N/A | 2890 | 2906 | CAGTAAATTTCTGGATG | 82 | 800 |
| 953964 | N/A | N/A | 2905 | 2921 | CCTAGAAAAGCACTACA | 109 | 801 |
| 953972 | N/A | N/A | 2950 | 2966 | TTACACAAGATACATGC | 93 | 802 |
| 953980 | N/A | N/A | 3023 | 3039 | TTTTAGAATGATCAAGT | 74 | 803 |
| 953988 | N/A | N/A | 3054 | 3070 | GTCAAATCAGCATAAGA | 118 | 804 |
| 953996 | N/A | N/A | 3084 | 3100 | ATATTTTGAAGATAAGC | 101 | 805 |
| 954004 | N/A | N/A | 3133 | 3149 | CAAAATCTATCTCCTTT | 120 | 806 |
| 954012 | N/A | N/A | 3159 | 3175 | TCATATATTACCTTAAA | 115 | 807 |
| 954020 | N/A | N/A | 3510 | 3526 | TGTATTACTTATGCAAA | 89 | 808 |
| 954028 | N/A | N/A | 3557 | 3573 | ATTTTAATTATTGCCCT | 70 | 809 |
| 954036 | N/A | N/A | 3634 | 3650 | GATACATGAGGAACTGA | 108 | 810 |
| 954044 | N/A | N/A | 3663 | 3679 | TTACAATATGAACAGGT | 46 | 811 |
| 954052 | N/A | N/A | 3747 | 3763 | GACAATGTGAGACTACA | 103 | 812 |
| 954060 | N/A | N/A | 4066 | 4082 | AAATAAACCTAATGCAG | 130 | 813 |
| 954068 | N/A | N/A | 4124 | 4140 | GCAAAATACAGAGAAAT | 63 | 814 |
| 954076 | N/A | N/A | 4184 | 4200 | ATCCAAACTTTGTGAGC | 140 | 815 |
| 954084 | N/A | N/A | 4269 | 4285 | GAACAGATACTTTCTAA | 111 | 816 |
| 954092 | N/A | N/A | 4337 | 4353 | CATAAACTGAGGCCCAT | 118 | 817 |
| 954100 | N/A | N/A | 4422 | 4438 | TCCCAAGCATGCTAGAT | 122 | 818 |
| 954108 | N/A | N/A | 4465 | 4481 | CCAGAAGCAAGTATTAA | 88 | 819 |
| 954116 | N/A | N/A | 4500 | 4516 | CACCAGATAGGAATCTG | 110 | 820 |
| 954124 | N/A | N/A | 4533 | 4549 | ATATTTACTGATGAATG | 95 | 821 |
| 954132 | N/A | N/A | 4612 | 4628 | CACGAGGGCAGAGCTTT | 79 | 822 |
| 954140 | N/A | N/A | 4751 | 4767 | AAAATAAGTTAACACTC | 126 | 823 |
| 954148 | N/A | N/A | 5499 | 5515 | TAAAATATCAACACTGG | 104 | 824 |
| 954156 | N/A | N/A | 5614 | 5630 | ACGATAGTGATACTGCA | 79 | 825 |
| 954164 | N/A | N/A | 5761 | 5777 | TTACACTAGACTCTTAA | 85 | 826 |
| 954172 | N/A | N/A | 5821 | 5837 | TGAAACAGGAAGATTTT | 138 | 827 |
| 954180 | N/A | N/A | 5851 | 5867 | ACCCAACTAAAATGACA | 102 | 828 |
| 954188 | N/A | N/A | 5942 | 5958 | ATCTACAAGGCAAGTAG | 105 | 829 |
| 954196 | N/A | N/A | 6027 | 6043 | TTGAAATTCAGATCATG | 117 | 830 |
| 954204 | N/A | N/A | 6186 | 6202 | CAAAATCCAGTCAGTCA | 77 | 831 |
| 954212 | N/A | N/A | 6237 | 6253 | GCTAAATTGTTATACTT | 82 | 832 |
| 954220 | N/A | N/A | 6269 | 6285 | AGCTGAATACAAACCTG | 80 | 833 |
| 954228 | N/A | N/A | 6703 | 6719 | TAAGAAAGAGCCTGGGT | 148 | 834 |
| 954236 | N/A | N/A | 6726 | 6742 | AACAAGGGTTTGCAAGG | 116 | 835 |
| 954244 | N/A | N/A | 6839 | 6855 | ATCCAAAGGAAGGGAGA | 99 | 836 |
| 954252 | N/A | N/A | 6988 | 7004 | AGTAAACCTTTTTGACT | 105 | 837 |
| 954260 | N/A | N/A | 7162 | 7178 | CTAAGATAATTCCATGG | 109 | 838 |
| 954268 | N/A | N/A | 7190 | 7206 | TAAGGACGATTAATTTT | 126 | 839 |
| 954276 | N/A | N/A | 7201 | 7217 | ATTTATACAATTAAGGA | 126 | 840 |
| 954284 | N/A | N/A | 7244 | 7260 | TCGACATAAAGACCAAC | 125 | 841 |
| 954292 | N/A | N/A | 7350 | 7366 | GGCCAAATCAATCTTGA | 91 | 842 |
| 954300 | N/A | N/A | 7521 | 7537 | ACACAAAGTTTGCCCAT | 80 | 843 |
| 954308 | N/A | N/A | 7549 | 7565 | GCCCGAAATACTTACAA | 125 | 844 |
| 954316 | N/A | N/A | 7656 | 7672 | TAATAAAATTCTATTGG | 128 | 845 |
| 954324 | N/A | N/A | 7787 | 7803 | CTATAACCTGCATATTT | 98 | 846 |
| 954332 | N/A | N/A | 7976 | 7992 | TACTAGGGAGGCCGTGC | 143 | 847 |
| 954340 | N/A | N/A | 8291 | 8307 | GACAAAGATGACAGACT | 100 | 848 |
| 954348 | N/A | N/A | 8327 | 8343 | GAAGACACAAGAAACAC | 129 | 849 |
| 954356 | N/A | N/A | 8348 | 8364 | ATGCATATTAAAATCTG | 102 | 850 |
| 954364 | N/A | N/A | 8440 | 8456 | CAATATCTATGACACAT | 48 | 851 |
| 954372 | N/A | N/A | 8465 | 8481 | AAGAATTACAGGTACTG | 74 | 852 |
| 954380 | N/A | N/A | 8489 | 8505 | CTAAAATATTACATAGC | 96 | 853 |
| 954388 | N/A | N/A | 8549 | 8565 | CCACAAAAATGTGAGAC | 87 | 854 |
| 954396 | N/A | N/A | 8622 | 8638 | TTACAGGAAATGGAGGT | 97 | 855 |
| 954404 | N/A | N/A | 8655 | 8671 | ATTCAAAGGTAGTTTAT | 106 | 856 |
| 954412 | N/A | N/A | 8724 | 8740 | TGCTTTATAAGGTATTT | 78 | 857 |

### Example 3: Effect of 5-10-5 MOE Mixed Backbone Modified Oligonucleotides on Human HTT RNA In Vitro, Single Dose

Modified oligonucleotides complementary to human HTT nucleic acid were designed and tested for their single dose effects on HTT RNA in vitro. The modified oligonucleotides were tested in a series of experiments that had the same culture conditions.

The modified oligonucleotides in the tables below are 5-10-5 MOE gapmers with mixed PO/PS internucleoside linkages. The gapmers are 20 nucleosides in length, wherein the sugar motif for the gapmers is (from 5′ to 3′): eeeeeddddddddddeeeee; wherein each “d” represents a 2′-β-D-deoxyribosyl sugar, and each “e” represents a 2′-MOE sugar moiety. The internucleoside linkage motif for the gapmers is (from 5′ to 3′): sooosssssssssssooss; wherein each “o” represents a phosphodiester internucleoside linkage, and each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

Cultured A431 cells were treated with modified oligonucleotide at a concentration of 2000 nM by free uptake at a density of 10,000 cells per well. After a treatment period of approximately 48 hours, total RNA was isolated from the cells and HTT RNA levels were measured by quantitative real-time RTPCR. HTT RNA levels were measured by human primer probe set RTS36478 (forward sequence ACCCTTGCAGAGATTGACTT, designated herein as SEQ ID NO: 17; reverse sequence AGCACTCGTTCTTGCAGTT, designated herein as SEQ ID NO: 18; probe sequence TTTGCCTCCAAAAAGCTCACCAGC, designated herein as SEQ ID NO: 19). HTT RNA levels were normalized to total RNA content, as measured by RIBOGREEN®. Reduction of HTT RNA is presented in the tables below as percent HTT RNA relative to the amount of the HTT RNA in untreated control cells (% UTC). Each table represents results from an individual assay plate. The values marked with a “t” indicate that the modified oligonucleotide is complementary to the amplicon region of the primer probe set. Additional assays may be used to measure the potency and efficacy of the modified oligonucleotides complementary to the amplicon region.

In the tables below, Compound 388241 was used as a comparator compound. Compound 388241 is 20 nucleosides in length, wherein the sugar motif is (from 5′ to 3′): eeeeeddddddddddeeeee; wherein each “d” represents a 2′-β-D-deoxyribosyl sugar, and each “e” represents a 2′-MOE sugar moiety. The internucleoside linkage motif for Compound 388241 is (from 5′ to 3′): sssssssssssssssssss; wherein each “s” represents a phosphorothioate internucleoside linkage. Each cytosine residue is a 5-methylcytosine.

TABLE 13
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 19 | 858 |
| 1314495 | N/A | N/A | 3136 | 3155 | ATGAAACAAAATCTATCTCC | 82 | 859 |
| 1314500 | N/A | N/A | 129817 | 129836 | AAAACACTTCTTCCCCACTA | 54 | 860 |
| 1314528 | N/A | N/A | 46677 | 46696 | TGAATTTATTCTTTTTCCCT | 78 | 861 |
| 1314530 | N/A | N/A | 117826 | 117845 | CTGAAACCATAATTAAGTCA | 46 | 862 |
| 1314541 | N/A | N/A | 70544 | 70563 | ACAAAAGTTATACTTGCTCA | 12 | 863 |
| 1314544 | N/A | N/A | 60439 | 60458 | GTCATGGTTTCAACTCTCAA | 17 | 864 |
| 1314545 | N/A | N/A | 17085 | 17104 | TATGTATTTATTCTTGCAGT | 45 | 865 |
| 1314560 | N/A | N/A | 81880 | 81899 | TACACATATTTCAAACATCA | 55 | 866 |
| 1314580 | N/A | N/A | 68687 | 68706 | CATGTCAATTTCTATCCCCC | 25 | 867 |
| 1314618 | N/A | N/A | 17819 | 17838 | GTCAATACTTTCCCCAAGTT | 32 | 868 |
| 1314639 | N/A | N/A | 45022 | 45041 | CCCTTTTTTCCCCTTCACCC | 73 | 869 |
| 1314667 | N/A | N/A | 59084 | 59103 | AGCAACAAACATCATCATCT | 89 | 870 |
| 1314669 | N/A | N/A | 100946 | 100965 | GCACTCAGATATACTCCTGC | 79 | 871 |
| 1314691 | N/A | N/A | 34537 | 34556 | TTATTCCAAATCATTCAGCT | 68 | 872 |
| 1314751 | N/A | N/A | 10437 | 10456 | ACCACATTCACCTCCCTCTC | 66 | 873 |
| 1314769 | N/A | N/A | 56771 | 56790 | GCGATTTTAAATCCTCACAC | 54 | 874 |
| 1314774 | N/A | N/A | 111471 | 111490 | GCCAACTTAACCCATTGCCG | 40 | 875 |
| 1314793 | N/A | N/A | 48882 | 48901 | CAGTCAAGTATAACTTTTCA | 29 | 876 |
| 1314851 | N/A | N/A | 53293 | 53312 | AGTTCAAGTTCTTAACCGAC | 25 | 877 |
| 1314870 | N/A | N/A | 130450 | 130469 | AGGCATTTCAAACTTACGAC | 25 | 878 |
| 1314943 | N/A | N/A | 65893 | 65912 | ACTATTTTATCATATTGGTC | 19 | 879 |
| 1314955 | 2872 | 2891 | 62554 | 62573 | GGATGACAACATTATTGAGC | 3† | 880 |
| 1314959 | N/A | N/A | 25031 | 25050 | GTATAACCATATTTCACCCT | 46 | 881 |
| 1314969 | N/A | N/A | 142119 | 142138 | GCCACCATTTTCCTAGTAAA | 43 | 882 |
| 1315018 | N/A | N/A | 56029 | 56048 | CACTCATTCCCACTACATTA | 53 | 883 |
| 1315039 | N/A | N/A | 136561 | 136580 | CCAGATAGTTCTCATCCCAA | 10 | 884 |
| 1315041 | N/A | N/A | 114520 | 114539 | AGACTCACTTTTACTGGCTT | 21 | 885 |
| 1315070 | N/A | N/A | 12554 | 12573 | CTGTATTTCACCAATACTCA | 68 | 886 |
| 1315074 | N/A | N/A | 108167 | 108186 | GCACAAGTTTATTAGTGATC | 9 | 887 |
| 1315076 | N/A | N/A | 7428 | 7447 | TCTCAGAATCCTACTTGGCT | 62 | 888 |
| 1315087 | N/A | N/A | 5891 | 5910 | TTGGACCTTACTTCTTACTA | 49 | 889 |
| 1315110 | N/A | N/A | 104193 | 104212 | GGGAACACTTTCCTTCTGCA | 64 | 890 |
| 1315144 | 3320 | 3339 | 73126 | 73145 | GTACAGCTCTTCCTAGACTC | 27 | 891 |
| 1315202 | N/A | N/A | 94641 | 94660 | TCACCTCTCCAGCTACACTT | 90 | 892 |
| 1315204 | N/A | N/A | 133712 | 133731 | CCGTACAAATCTATATCCTT | 31 | 893 |
| 1315444 | N/A | N/A | 45574 | 45593 | TGGATGGTTACTAATTGCCT | 49 | 894 |
| 1315451 | N/A | N/A | 19656 | 19675 | TCTCTTTTTAATATTAGGTA | 77 | 895 |
| 1315474 | N/A | N/A | 41859 | 41878 | TTCACATTCAATCAACCAGT | 65 | 896 |
| 1315506 | N/A | N/A | 20046 | 20065 | TTCATCACTCCAATTTTCAA | 88 | 897 |
| 1315527 | N/A | N/A | 106954 | 106973 | AACATACATTCCTCTTACCT | 84 | 898 |
| 1315544 | N/A | N/A | 134658 | 134677 | CTGATAGGAATTCATGGTCA | 25 | 899 |
| 1315615 | N/A | N/A | 110081 | 110100 | AGTCAGGTATTTACTATGTA | 23 | 900 |
| 1315620 | N/A | N/A | 105918 | 105937 | ACTCTGACCCCTCAGTGACA | 94 | 901 |
| | | | 105969 | 105988 | | | |
| | | | 106024 | 106043 | | | |
| 1315688 | N/A | N/A | 18989 | 19008 | GTCACAAATATAAATATCTA | 78 | 902 |
| 1315692 | N/A | N/A | 69357 | 69376 | GTCATTTACATTCCTGGAGT | 16 | 903 |
| 1315714 | N/A | N/A | 125875 | 125894 | TTCTGATTTTTCTAAGCAGA | 91 | 904 |
| 1315804 | N/A | N/A | 75430 | 75449 | AGGACAGTAGCCATTAACCA | 33 | 905 |
| 1315847 | N/A | N/A | 51146 | 51165 | TGGGAATTTCTAATTTCTGC | 23 | 906 |
| 1315992 | N/A | N/A | 160801 | 160820 | CAGCCCAACACACCAGCACA | 92 | 907 |
| 1316076 | N/A | N/A | 33828 | 33847 | CGACATACAACATACCCCAA | 84 | 908 |
| 1316148 | N/A | N/A | 43578 | 43597 | GCTACACCCTTTTTACTCAA | 34 | 909 |
| 1316158 | N/A | N/A | 26702 | 26721 | AAGCAAAGATTCCCTGGGCA | 86 | 910 |
| 1316180 | N/A | N/A | 149496 | 149515 | GCAAAAGCCCATTTTTCAAA | 93 | 911 |
| 1316335 | N/A | N/A | 9945 | 9964 | TTCTAAATCCTCACCAGCAT | 54 | 912 |
| 1316376 | N/A | N/A | 118118 | 118137 | ACAACTTTAGACATCACCCA | 54 | 913 |
| 1316405 | N/A | N/A | 138747 | 138766 | GACATTGTTCCTCAGTGGCA | 53 | 914 |
| 1316523 | N/A | N/A | 116031 | 116050 | GGTCCTCTTAACTATTTTTA | 76 | 915 |
| 1316576 | N/A | N/A | 40111 | 40130 | ACTTCTCTCCTTGCCAGCCA | 55 | 916 |
| 1316597 | N/A | N/A | 22579 | 22598 | GGCTACTGAAATCTCTATCA | 28 | 917 |
| 1316688 | N/A | N/A | 81002 | 81021 | CCATCACCTGCAACCAGGGA | 99 | 918 |
| 1316689 | N/A | N/A | 119649 | 119668 | AAGCCATATTTTCTCAACTT | 36 | 919 |
| 1316695 | N/A | N/A | 35427 | 35446 | AGGAAGGGAACTACTATCTT | 50 | 920 |
| 1316719 | N/A | N/A | 23097 | 23116 | GCTTGTCATTTTCAAGTTTC | 73 | 921 |
| 1316747 | N/A | N/A | 47423 | 47442 | GACAAAGTTTATACCTTAAC | 38 | 922 |
| 1316813 | N/A | N/A | 52749 | 52768 | GGATTATACAACCAAAACCA | 78 | 923 |
| 1316816 | N/A | N/A | 32332 | 32351 | GGGTTTAGACCACATTCCCA | 86 | 924 |
| 1316869 | N/A | N/A | 83780 | 83799 | ACACGGATCCCTCCAGACTC | 97 | 925 |
| 1316872 | 12480 | 12499 | 169229 | 169248 | CTTCACATTTTCCCCTTGAA | 68 | 926 |
| 1316933 | N/A | N/A | 72510 | 72529 | GTAAGTCAAACTACCAAGCA | 32 | 927 |
| 1316988 | N/A | N/A | 127367 | 127386 | AACTAAATTTACCTTTGGTT | 85 | 928 |
| 1316999 | N/A | N/A | 22171 | 22190 | GAAATCTACCATCAAGCTCT | 61 | 929 |
| 1317036 | N/A | N/A | 132358 | 132377 | ACTCTTTTTCCAATTTTGTG | 32 | 930 |
| 1317065 | N/A | N/A | 79756 | 79775 | ATCGAGACTCCCAATGCTCT | 79 | 931 |
| 1317087 | N/A | N/A | 14033 | 14052 | AACATGAATTATTTATTCCA | 63 | 932 |
| 1317119 | 7435 | 7454 | 146527 | 146546 | TCTCAGGGAATGCTGTGCCA | 47 | 933 |
| 1317177 | N/A | N/A | 15545 | 15564 | GTAAAGATAACATATAGCTT | 51 | 934 |
| 1317181 | N/A | N/A | 98526 | 98545 | ACAAGATAACTTCCATTTAA | 72 | 935 |

TABLE 14
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314513 | N/A | N/A | 7351 | 7370 | GCATGGCCAAATCAATCTTG | 57 | 936 |
| 1314581 | N/A | N/A | 70467 | 70486 | CCCAAAATTTATCTTGGTGA | 49 | 937 |
| 1314632 | N/A | N/A | 81349 | 81368 | GCAGCAGTCACTTCATGCTT | 89 | 938 |
| 1314701 | 12479 | 12498 | 169228 | 169247 | TTCACATTTTCCCCTTGAAA | 70 | 939 |
| 1314723 | N/A | N/A | 119647 | 119666 | GCCATATTTTCTCAACTTTA | 19 | 940 |
| 1314754 | N/A | N/A | 52744 | 52763 | ATACAACCAAAACCACCCTC | 84 | 941 |
| 1314780 | N/A | N/A | 60433 | 60452 | GTTTCAACTCTCAAACAGCA | 80 | 942 |
| 1314831 | N/A | N/A | 20042 | 20061 | TCACTCCAATTTTCAAGGCC | 64 | 943 |
| 1314877 | N/A | N/A | 46668 | 46687 | TCTTTTTCCCTITAATTACA | 73 | 944 |
| 1314879 | N/A | N/A | 59083 | 59102 | GCAACAAACATCATCATCTA | 98 | 945 |
| 1314917 | N/A | N/A | 133711 | 133730 | CGTACAAATCTATATCCTTT | 43 | 946 |
| 1314938 | N/A | N/A | 48879 | 48898 | TCAAGTATAACTTTTCATTA | 61 | 947 |
| 1314995 | N/A | N/A | 138746 | 138765 | ACATTGTTCCTCAGTGGCAC | 88 | 948 |
| 1315026 | N/A | N/A | 18304 | 18323 | ATCTTAAATTTTCAATCCCC | 62 | 949 |
| 1315189 | N/A | N/A | 9909 | 9928 | GATCTTTTCATCTAAATGCA | 42 | 950 |
| 1315212 | N/A | N/A | 130449 | 130468 | GGCATTTCAAACTTACGACA | 42 | 951 |
| 1315238 | 3310 | 3329 | 73116 | 73135 | TCCTAGACTCATCTGAGGCA | 71 | 952 |
| 1315263 | N/A | N/A | 34527 | 34546 | TCATTCAGCTAATAAGAGCA | 74 | 953 |
| 1315281 | N/A | N/A | 19563 | 19582 | GCTGAGGTTTTCCACTCCAA | 70 | 954 |
| 1315293 | N/A | N/A | 26588 | 26607 | AAGAACACTTCTTACCACTT | 82 | 955 |
| 1315328 | N/A | N/A | 35423 | 35442 | AGGGAACTACTATCTTTGTT | 46 | 956 |
| 1315371 | N/A | N/A | 15450 | 15469 | ACAGTGTTTCACATGTACCA | 55 | 957 |
| 1315479 | N/A | N/A | 80880 | 80899 | TCTGTGTACATCAGTCATTA | 67 | 958 |
| 1315481 | N/A | N/A | 106953 | 106972 | ACATACATTCCTCTTACCTG | 93 | 959 |
| 1315513 | N/A | N/A | 114499 | 114518 | TCTTGGGTTTCAACTTCCCT | 77 | 960 |
| 1315549 | N/A | N/A | 14032 | 14051 | ACATGAATTATTTATTCCAA | 81 | 961 |
| 1315658 | N/A | N/A | 24894 | 24913 | GTTTAGGCCATCATGCTCCA | 57 | 962 |
| 1315673 | N/A | N/A | 55910 | 55929 | CACAAACAAATTTTACAGCA | 49 | 963 |
| 1315726 | N/A | N/A | 65817 | 65836 | GTAGAATTTTTTCTAGAATA | 88 | 964 |
| 1315736 | N/A | N/A | 62528 | 62547 | TCTTGCAGTTTTAAAAGCTT | 61† | 965 |
| 1315853 | N/A | N/A | 45021 | 45040 | CCTTTTTTCCCCTTCACCCT | 80 | 966 |
| 1315866 | N/A | N/A | 94540 | 94559 | AGCAAGGTCACAGATAAGGC | 62 | 967 |
| 1315872 | N/A | N/A | 129797 | 129816 | ACACAGGTTATTTCACTCTA | 36 | 968 |
| 1315977 | N/A | N/A | 79673 | 79692 | GCTCTGCTACTTACTCACTC | 89 | 969 |
| 1315983 | N/A | N/A | 125507 | 125526 | GCATTCCTCCTCTCTCACTG | 60 | 970 |
| 1316020 | N/A | N/A | 40095 | 40114 | GCCACAGGACTCCCTGACAA | 78 | 971 |
| 1316034 | N/A | N/A | 10436 | 10455 | CCACATTCACCTCCCTCTCT | 64 | 972 |
| 1316035 | N/A | N/A | 100944 | 100963 | ACTCAGATATACTCCTGCTC | 54 | 973 |
| 1316094 | N/A | N/A | 115960 | 115979 | CCACCATTCATTTCTAGAAT | 59 | 974 |
| 1316095 | N/A | N/A | 5890 | 5909 | TGGACCTTACTTCTTACTAC | 44 | 975 |
| 1316114 | N/A | N/A | 69334 | 69353 | CTGAGATTCTTTTCACAGAA | 66 | 976 |
| 1316190 | N/A | N/A | 41800 | 41819 | TTTATTTATTTCAAAGGTCT | 48 | 977 |
| 1316195 | N/A | N/A | 17064 | 17083 | AACAAGTTCAAAAATACCTC | 72 | 978 |
| 1316207 | N/A | N/A | 23051 | 23070 | TTGAAATCTTTCTTTCCTAA | 81 | 979 |
| 1316234 | N/A | N/A | 68685 | 68704 | TGTCAATTTCTATCCCCCAT | 29 | 980 |
| 1316381 | N/A | N/A | 12553 | 12572 | TGTATTTCACCAATACTCAA | 86 | 981 |
| 1316387 | N/A | N/A | 17818 | 17837 | TCAATACTTTCCCCAAGTTC | 64 | 982 |
| 1316442 | N/A | N/A | 83711 | 83730 | ACTGTAATCAATCTTAGCTG | 64 | 983 |
| 1316457 | N/A | N/A | 136515 | 136534 | TGGATTTCAAAACAACACCT | 93 | 984 |
| 1316466 | N/A | N/A | 105619 | 105638 | AGGAAACCAAACACCTATGC | 71 | 985 |
| 1316469 | N/A | N/A | 108151 | 108170 | GATCATTTCTAAACAGACAT | 47 | 986 |
| 1316476 | N/A | N/A | 3104 | 3123 | GCAAAGGAAATTTCTTGATA | 70 | 987 |
| 1316547 | N/A | N/A | 75380 | 75399 | GCCAGGAACATCAATTCTGA | 62 | 988 |
| 1316553 | N/A | N/A | 22518 | 22537 | TGCTTCATGTATTTTAGCTC | 78 | 989 |
| 1316583 | N/A | N/A | 43575 | 43594 | ACACCCTTTTTACTCAACAA | 71 | 990 |
| 1316626 | N/A | N/A | 160797 | 160816 | CCAACACACCAGCACAGGCA | 72 | 991 |
| 1316650 | N/A | N/A | 47422 | 47441 | ACAAAGTTTATACCTTAACT | 83 | 992 |
| 1316679 | N/A | N/A | 53292 | 53311 | GTTCAAGTTCTTAACCGACT | 32 | 993 |
| 1316681 | N/A | N/A | 72495 | 72514 | AAGCATTGAACTCCAACCAC | 69 | 994 |
| 1316687 | N/A | N/A | 132283 | 132302 | GTTGAATCCAAAATATGAAT | 90 | 995 |
| 1316707 | N/A | N/A | 111408 | 111427 | CTTCAAGTAAAACCTTTTCC | 73 | 996 |
| 1316726 | N/A | N/A | 45565 | 45584 | ACTAATTGCCTTTTTCTCCC | 83 | 997 |
| 1316738 | N/A | N/A | 149488 | 149507 | CCATTTTTCAAATCAAGGAC | 78 | 998 |
| 1316799 | N/A | N/A | 104192 | 104211 | GGAACACTTTCCTTCTGCAC | 80 | 999 |
| 1316856 | N/A | N/A | 98517 | 98536 | CTTCCATTTAAATTGTGTAA | 67 | 1000 |
| 1316873 | N/A | N/A | 127356 | 127375 | CCTTTGGTTTATTTCTTTTA | 57 | 1001 |
| 1316950 | N/A | N/A | 110053 | 110072 | TTCGATCTCCACTCTACTCA | 77 | 1002 |
| 1316956 | N/A | N/A | 134612 | 134631 | CACAAGATATTTATAATCTT | 80 | 1003 |
| 1316998 | 7419 | 7438 | 146511 | 146530 | GCCAAAATCCCCTCCCGGTT | 53 | 1004 |
| 1317021 | N/A | N/A | 33727 | 33746 | CTGAGGCAACAATCATACCT | 85 | 1005 |
| 1317056 | N/A | N/A | 56713 | 56732 | TCACCTTGTTTTCTTGATCA | 81 | 1006 |
| 1317108 | N/A | N/A | 118110 | 118129 | AGACATCACCCATTAACTCT | 54 | 1007 |
| 1317124 | N/A | N/A | 117789 | 117808 | CAGTCATACATTGATTTCAA | 33 | 1008 |
| 1317135 | N/A | N/A | 22161 | 22180 | ATCAAGCTCTTTCTTTCCTT | 60 | 1009 |
| 1317191 | N/A | N/A | 141855 | 141874 | CGAGCATTAATTTCTGAGAA | 44 | 1010 |
| 1317220 | N/A | N/A | 32324 | 32343 | ACCACATTCCCAGTGACTCA | 88 | 1011 |
| 1317231 | N/A | N/A | 51032 | 51051 | TACATGGAATTAACACAGCA | 51 | 1012 |

TABLE 15
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 29 | 858 |
| 1314519 | N/A | N/A | 130441 | 130460 | AAACTTACGACATAATCACA | 96 | 1013 |
| 1314577 | N/A | N/A | 108141 | 108160 | AAACAGACATATCCAGAGCT | 64 | 1014 |
| 1314599 | N/A | N/A | 17807 | 17826 | CCCAAGTTCAATTTACTTTC | 68 | 1015 |
| 1314623 | N/A | N/A | 106952 | 106971 | CATACATTCCTCTTACCTGA | 97 | 1016 |
| 1314738 | N/A | N/A | 55839 | 55858 | GGTTATCTCCTAAATTGCAA | 45 | 1017 |
| 1314807 | N/A | N/A | 62310 | 62329 | GCCTCACTTTCACCTTCTTG | 78 | 1018 |
| 1314842 | N/A | N/A | 43413 | 43432 | CAGCTGTTTATCAACACGCA | 90 | 1019 |
| 1314924 | N/A | N/A | 125053 | 125072 | CGGCAAATTTCTCCACAGCA | 39 | 1020 |
| 1314945 | N/A | N/A | 132174 | 132193 | TCATCTTGATTCATAATCCT | 64 | 1021 |
| 1315095 | N/A | N/A | 118109 | 118128 | GACATCACCCATTAACTCTA | 49 | 1022 |
| 1315123 | N/A | N/A | 39968 | 39987 | GACAAACTTTTTTAGCTGCA | 22 | 1023 |
| 1315175 | N/A | N/A | 14028 | 14047 | GAATTATTTATTCCAAGGGA | 50 | 1024 |
| 1315198 | N/A | N/A | 75376 | 75395 | GGAACATCAATTCTGAGCTT | 43 | 1025 |
| 1315199 | N/A | N/A | 45542 | 45561 | TTGTTGATAATATATGCATA | 91 | 1026 |
| 1315217 | N/A | N/A | 15442 | 15461 | TCACATGTACCATCTGATCC | 68 | 1027 |
| 1315221 | N/A | N/A | 44976 | 44995 | GCAATCCCAATTTAACACAG | 76 | 1028 |
| 1315330 | N/A | N/A | 3100 | 3119 | AGGAAATTTCTTGATACCCA | 36 | 1029 |
| 1315344 | N/A | N/A | 20041 | 20060 | CACTCCAATTTTCAAGGCCA | 85 | 1030 |
| 1315443 | N/A | N/A | 98479 | 98498 | AGGAAAGTTTTCAATTTCTT | 88 | 1031 |
| 1315477 | N/A | N/A | 22491 | 22510 | CGGTGCATAAACATTTCAGC | 64 | 1032 |
| 1315504 | N/A | N/A | 18303 | 18322 | TCTTAAATTTTCAATCCCCT | 64 | 1033 |
| 1315510 | N/A | N/A | 83710 | 83729 | CTGTAATCAATCTTAGCTGT | 77 | 1034 |
| 1315545 | N/A | N/A | 10404 | 10423 | GCCACCACTAACCTACTCTA | 83 | 1035 |
| 1315580 | N/A | N/A | 117436 | 117455 | AAGCAGTATTTACATTATGA | 56 | 1036 |
| 1315609 | N/A | N/A | 47419 | 47438 | AAGTTTATACCTTAACTTCT | 101 | 1037 |
| 1315654 | N/A | N/A | 32219 | 32238 | GCTTTTGTTTCTCAATCCGT | 54 | 1038 |
| 1315698 | N/A | N/A | 48875 | 48894 | GTATAACTTTTCATTAGCGA | 16 | 1039 |
| 1315743 | 12302 | 12321 | 169051 | 169070 | GTTTCATTAAAATCTTTCCC | 63 | 1040 |
| 1315764 | N/A | N/A | 149411 | 149430 | CCCTCATACATTTTACAGGC | 93 | 1041 |
| 1315806 | N/A | N/A | 59082 | 59101 | CAACAAACATCATCATCTAA | 98 | 1042 |
| 1315818 | N/A | N/A | 160724 | 160743 | CACTGCACCAACATATGTGC | 98 | 1043 |
| 1315819 | N/A | N/A | 41762 | 41781 | AGACTCTTCCACTCATATGC | 93 | 1044 |
| 1315826 | N/A | N/A | 72494 | 72513 | AGCATTGAACTCCAACCACC | 70 | 1045 |
| 1315832 | N/A | N/A | 79671 | 79690 | TCTGCTACTTACTCACTCCC | 89 | 1046 |
| 1315862 | N/A | N/A | 23033 | 23052 | AAAACAGGCATTTAATGCTA | 92 | 1047 |
| 1315920 | N/A | N/A | 138734 | 138753 | AGTGGCACATCTACACATCC | 87 | 1048 |
| 1315942 | N/A | N/A | 22156 | 22175 | GCTCTTTCTTTCCTTTATTT | 70 | 1049 |
| 1315962 | N/A | N/A | 65571 | 65590 | GCAATAGTTGCAATGAGCCA | 67 | 1050 |
| 1315978 | N/A | N/A | 110048 | 110067 | TCTCCACTCTACTCAGATTA | 85 | 1051 |
| 1316000 | N/A | N/A | 9726 | 9745 | GCAATCCTCCAGCCTCCTCA | 62 | 1052 |
| 1316024 | 2075 | 2094 | 56666 | 56685 | ACAAATTTATCAACACTGCT | 62 | 1053 |
| 1316064 | N/A | N/A | 68649 | 68668 | CTCTAATTCCTTTAGAACAC | 68 | 1054 |
| 1316080 | N/A | N/A | 19454 | 19473 | GGTCATTTCCGCACAAGCAA | 34 | 1055 |
| 1316096 | N/A | N/A | 7316 | 7335 | GGAGAACTCACATTTCATGA | 31 | 1056 |
| 1316112 | N/A | N/A | 69329 | 69348 | ATTCTTTTCACAGAAGCACA | 64 | 1057 |
| 1316212 | N/A | N/A | 134511 | 134530 | GTGACAACTTCATAATGCAA | 50 | 1058 |
| 1316226 | N/A | N/A | 52743 | 52762 | TACAACCAAAACCACCCTCA | 97 | 1059 |
| 1316246 | N/A | N/A | 53287 | 53306 | AGTTCTTAACCGACTACTCC | 77 | 1060 |
| 1316248 | N/A | N/A | 141854 | 141873 | GAGCATTAATTTCTGAGAAC | 36 | 1061 |
| 1316263 | N/A | N/A | 114261 | 114280 | CCAGGTATTATTCCAGACTT | 41 | 1062 |
| 1316312 | N/A | N/A | 80876 | 80895 | TGTACATCAGTCATTAGTTT | 16 | 1063 |
| 1316327 | N/A | N/A | 46663 | 46682 | TTCCCTTTAATTACAGTTGC | 71 | 1064 |
| 1316356 | N/A | N/A | 119107 | 119126 | GCTATCTTTTACTCTAAGAT | 47 | 1065 |
| 1316383 | 6512 | 6531 | 136200 | 136219 | GGAATCCGATTCACCAGCTC | 63 | 1066 |
| 1316390 | N/A | N/A | 105259 | 105278 | CACTAGCTCCTTCCACAGGC | 77 | 1067 |
| 1316425 | N/A | N/A | 33726 | 33745 | TGAGGCAACAATCATACCTT | 100 | 1068 |
| 1316500 | N/A | N/A | 70457 | 70476 | ATCTTGGTGATTATCTGCCA | 31 | 1069 |
| 1316531 | N/A | N/A | 12544 | 12563 | CCAATACTCAATCAATTTCA | 53 | 1070 |
| 1316562 | N/A | N/A | 35411 | 35430 | TCTTTGTTTATAATCCTGTT | 65 | 1071 |
| 1316566 | N/A | N/A | 51031 | 51050 | ACATGGAATTAACACAGCAA | 39 | 1072 |
| 1316598 | N/A | N/A | 24886 | 24905 | CATCATGCTCCAACTGTCTA | 95 | 1073 |
| 1316649 | N/A | N/A | 129796 | 129815 | CACAGGTTATTTCACTCTAC | 40 | 1074 |
| 1316653 | N/A | N/A | 5889 | 5908 | GGACCTTACTTCTTACTACA | 40 | 1075 |
| 1316682 | N/A | N/A | 100927 | 100946 | CTCAAACCAATTTCACATTC | 80 | 1076 |
| 1316848 | N/A | N/A | 111402 | 111421 | GTAAAACCTTTTCCCAAGTC | 77 | 1077 |
| 1316853 | N/A | N/A | 94516 | 94535 | TAGTTTGTATATCTCAAGCC | 89 | 1078 |
| 1316944 | N/A | N/A | 115953 | 115972 | TCATTTCTAGAATAACTCTC | 80 | 1079 |
| 1316987 | N/A | N/A | 17063 | 17082 | ACAAGTTCAAAAATACCTCT | 83 | 1080 |
| 1316992 | N/A | N/A | 34507 | 34526 | GTAGCAGCTAACATCTAAGC | 77 | 1081 |
| 1317053 | N/A | N/A | 60418 | 60437 | CAGCAACTTCACAAACATTG | 94 | 1082 |
| 1317083 | N/A | N/A | 81283 | 81302 | TGGATGGGAATTCAATAAAC | 61 | 1083 |
| 1317088 | N/A | N/A | 127355 | 127374 | CTTTGGTTTATTTCTTTTAA | 71 | 1084 |
| 1317120 | N/A | N/A | 73081 | 73100 | CTGGAAGCAGACATTTGCAA | 94 | 1085 |
| 1317193 | N/A | N/A | 133708 | 133727 | ACAAATCTATATCCTTTATA | 91 | 1086 |
| 1317212 | N/A | N/A | 104191 | 104210 | GAACACTTTCCTTCTGCACT | 79 | 1087 |
| 1317226 | N/A | N/A | 26582 | 26601 | ACTTCTTACCACTTTGAGGA | 93 | 1088 |
| 1317234 | N/A | N/A | 145879 | 145898 | CCCACAGCCCATCTGCACAA | 93 | 1089 |

TABLE 16
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 29 | 858 |
| 1314488 | N/A | N/A | 73065 | 73084 | GCAAAGTCAAATTATAGACA | 92 | 1090 |
| 1314547 | N/A | N/A | 22153 | 22172 | CTTTCTTTCCTTTATTTGCC | 64 | 1091 |
| 1314566 | N/A | N/A | 127345 | 127364 | TTTCTTTTAAACACTCATCA | 63 | 1092 |
| 1314617 | N/A | N/A | 138731 | 138750 | GGCACATCTACACATCCGTC | 62 | 1093 |
| 1314660 | N/A | N/A | 56541 | 56560 | ACAATTATACATTTATGCTA | 72 | 1094 |
| 1314730 | N/A | N/A | 46661 | 46680 | CCCTTTAATTACAGTTGCCA | 69 | 1095 |
| 1314794 | N/A | N/A | 125052 | 125071 | GGCAAATTTCTCCACAGCAT | 44 | 1096 |
| 1314804 | N/A | N/A | 26552 | 26571 | GCAATTAGTCCTCACACATA | 91 | 1097 |
| 1314817 | N/A | N/A | 117434 | 117453 | GCAGTATTTACATTATGACT | 50 | 1098 |
| 1315014 | N/A | N/A | 80867 | 80886 | GTCATTAGTTTCTAATATCT | 28 | 1099 |
| 1315029 | N/A | N/A | 134371 | 134390 | ACAAGGTACTCTAAAGACTG | 53 | 1100 |
| | | | 134404 | 134423 | | | |
| 1315116 | N/A | N/A | 160723 | 160742 | ACTGCACCAACATATGTGCC | 106 | 1101 |
| 1315135 | N/A | N/A | 149410 | 149429 | CCTCATACATTTTACAGGCC | 81 | 1102 |
| 1315137 | N/A | N/A | 39967 | 39986 | ACAAACTTTTTTAGCTGCAA | 27 | 1103 |
| 1315153 | N/A | N/A | 108139 | 108158 | ACAGACATATCCAGAGCTCA | 58 | 1104 |
| 1315159 | 4850 | 4869 | 106905 | 106924 | GACACCACCACCTCTTTTTG | 41 | 1105 |
| 1315185 | N/A | N/A | 20022 | 20041 | AGCACCTTCAAATCTTTCTG | 54 | 1106 |
| 1315244 | N/A | N/A | 141372 | 141391 | AGAAGAATTTCTACACACTT | 104 | 1107 |
| 1315391 | N/A | N/A | 45527 | 45546 | GCATATGCCTTCCCTCTGCC | 86 | 1108 |
| 1315454 | N/A | N/A | 17061 | 17080 | AAGTTCAAAAATACCTCTAC | 88 | 1109 |
| 1315518 | N/A | N/A | 98478 | 98497 | GGAAAGTTTTCAATTTCTTA | 70 | 1110 |
| 1315523 | N/A | N/A | 24836 | 24855 | GCCATGTACATGAAACTGAC | 90 | 1111 |
| 1315533 | N/A | N/A | 130439 | 130458 | ACTTACGACATAATCACAGA | 92 | 1112 |
| 1315538 | N/A | N/A | 23029 | 23048 | CAGGCATTTAATGCTAGAAA | 85 | 1113 |
| 1315566 | N/A | N/A | 55796 | 55815 | GTATGGTATTTCATATGTTT | 31 | 1114 |
| 1315588 | N/A | N/A | 132173 | 132192 | CATCTTGATTCATAATCCTA | 74 | 1115 |
| 1315679 | N/A | N/A | 105211 | 105230 | GGGTTCCTTATTTAAGCTCC | 87 | 1116 |
| 1315689 | N/A | N/A | 52733 | 52752 | ACCACCCTCATATAATCCAG | 59 | 1117 |
| 1315713 | N/A | N/A | 135638 | 135657 | CCAAGGCTCTACCAAGGCTC | 86 | 1118 |
| | | | 135649 | 135668 | | | |
| 1315745 | N/A | N/A | 53280 | 53299 | AACCGACTACTCCAAGCCTT | 76 | 1119 |
| 1315824 | 5164 | 5183 | 113978 | 113997 | CCAGAATTCCCGATATCCAC | 39 | 1120 |
| 1315840 | N/A | N/A | 70365 | 70384 | TTCTGCCTTTTTTCACTCAA | 56 | 1121 |
| 1315846 | N/A | N/A | 9588 | 9607 | GCAATCCTCCCACCACACCG | 68 | 1122 |
| 1315882 | N/A | N/A | 133704 | 133723 | ATCTATATCCTTTATATCCT | 86 | 1123 |
| 1315918 | N/A | N/A | 12510 | 12529 | GTTAACATCACTAAAGCCAA | 63 | 1124 |
| 1315941 | N/A | N/A | 109996 | 110015 | GCACCATTCTAAAACCATCT | 68 | 1125 |
| 1315943 | N/A | N/A | 44972 | 44991 | TCCCAATTTAACACAGTTTC | 98 | 1126 |
| 1316054 | 11875 | 11894 | 168624 | 168643 | GGTGAAGTCATTTTTACTAA | 57 | 1127 |
| 1316079 | N/A | N/A | 15363 | 15382 | GTTACAGCTTATCTAAAGTC | 67 | 1128 |
| 1316102 | N/A | N/A | 100926 | 100945 | TCAAACCAATTTCACATTCA | 89 | 1129 |
| 1316111 | N/A | N/A | 35389 | 35408 | TACTACTGAATTTCATTGAT | 66 | 1130 |
| 1316151 | N/A | N/A | 60414 | 60433 | AACTTCACAAACATTGATCA | 100 | 1131 |
| 1316173 | N/A | N/A | 94345 | 94364 | GCTTCTACCCTACATATCCC | 84 | 1132 |
| 1316220 | N/A | N/A | 32218 | 32237 | CTTTTGTTTCTCAATCCGTT | 67 | 1133 |
| 1316237 | N/A | N/A | 111363 | 111382 | AACACATTCCCATTAGGGTT | 102 | 1134 |
| 1316238 | N/A | N/A | 81279 | 81298 | TGGGAATTCAATAAACTGCA | 37 | 1135 |
| 1316273 | N/A | N/A | 83587 | 83606 | TCAAAGGGCTTATACATCCT | 78 | 1136 |
| 1316289 | N/A | N/A | 19345 | 19364 | GGTATTCTTCCATATGGGCT | 44 | 1137 |
| 1316294 | N/A | N/A | 3092 | 3111 | TCTTGATACCCATATTTTGA | 50 | 1138 |
| 1316304 | N/A | N/A | 72425 | 72444 | GAACTCATAAATATCAACCC | 70 | 1139 |
| 1316309 | N/A | N/A | 104179 | 104198 | TCTGCACTAAATTTTCCTAA | 79 | 1140 |
| 1316350 | N/A | N/A | 18302 | 18321 | CTTAAATTTTCAATCCCCTT | 77 | 1141 |
| 1316363 | N/A | N/A | 68579 | 68598 | CAGTGGTTTGAATAAAGCCA | 84 | 1142 |
| 1316444 | N/A | N/A | 65481 | 65500 | GAAGTTGCTATCATTTGCAG | 56 | 1143 |
| 1316451 | N/A | N/A | 69321 | 69340 | CACAGAAGCACAATTTCTTA | 75 | 1144 |
| 1316463 | N/A | N/A | 34506 | 34525 | TAGCAGCTAACATCTAAGCC | 85 | 1145 |
| 1316480 | N/A | N/A | 119106 | 119125 | CTATCTTTTACTCTAAGATA | 111 | 1146 |
| 1316498 | N/A | N/A | 13999 | 14018 | AAGGCAACTATTTCACGCAT | 64 | 1147 |
| 1316511 | N/A | N/A | 22489 | 22508 | GTGCATAAACATTTCAGCTT | 87 | 1148 |
| 1316512 | N/A | N/A | 33725 | 33744 | GAGGCAACAATCATACCTTA | 103 | 1149 |
| 1316556 | N/A | N/A | 115930 | 115949 | ATCTTGTAAAAATAACTCCC | 93 | 1150 |
| 1316560 | N/A | N/A | 47418 | 47437 | AGTTTATACCTTAACTTCTC | 92 | 1151 |
| 1316589 | N/A | N/A | 6824 | 6843 | GGAGAGTTTTTACAAATCAT | 27 | 1152 |
| 1316594 | N/A | N/A | 79607 | 79626 | CCCAAATTTATACACTGAAA | 83 | 1153 |
| 1316616 | N/A | N/A | 51029 | 51048 | ATGGAATTAACACAGCAACT | 33 | 1154 |
| 1316620 | N/A | N/A | 129793 | 129812 | AGGTTATTTCACTCTACACT | 31 | 1155 |
| 1316665 | N/A | N/A | 10402 | 10421 | CACCACTAACCTACTCTACT | 67 | 1156 |
| 1316676 | N/A | N/A | 17804 | 17823 | AAGTTCAATTTACTTTCAAT | 84 | 1157 |
| 1316705 | N/A | N/A | 75326 | 75345 | AGCTAGGTATTTCCCTCTTA | 46 | 1158 |
| 1316723 | N/A | N/A | 145835 | 145854 | GACCAGCTAATCTCTTACCT | 57 | 1159 |
| 1316731 | N/A | N/A | 59077 | 59096 | AACATCATCATCTAAACTTA | 93 | 1160 |
| 1316852 | N/A | N/A | 118108 | 118127 | ACATCACCCATTAACTCTAC | 67 | 1161 |
| 1316913 | N/A | N/A | 61811 | 61830 | CCAGATTTATAAACATTCTA | 87 | 1162 |
| 1317020 | N/A | N/A | 43354 | 43373 | AGCATTTTACAAACACTACC | 85 | 1163 |
| 1317072 | N/A | N/A | 41742 | 41761 | CTCCACCTTACCTAAGAGCA | 108 | 1164 |
| 1317143 | N/A | N/A | 5888 | 5907 | GACCTTACTTCTTACTACAA | 76 | 1165 |
| 1317178 | N/A | N/A | 48854 | 48873 | GGGTATTTCCACTCATTATT | 33 | 1166 |

TABLE 17
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314521 | N/A | N/A | 22150 | 22169 | TCTTTCCTTTATTTGCCTCT | 47 | 1167 |
| 1314537 | N/A | N/A | 23018 | 23037 | TGCTAGAAATTTCCATTTAC | 69 | 1168 |
| 1314571 | N/A | N/A | 117433 | 117452 | CAGTATTTACATTATGACTA | 62 | 1169 |
| 1314628 | N/A | N/A | 141361 | 141380 | TACACACTTCCATCAGTGCT | 97 | 1170 |
| 1314757 | N/A | N/A | 129792 | 129811 | GGTTATTTCACTCTACACTG | 36 | 1171 |
| 1314789 | N/A | N/A | 65447 | 65466 | GCAAAATCTCTGAATAGGCA | 18 | 1172 |
| 1314798 | N/A | N/A | 13996 | 14015 | GCAACTATTTCACGCATGCC | 66 | 1173 |
| 1314848 | N/A | N/A | 134292 | 134311 | GCATGATAATTATATTCTCC | 25 | 1174 |
| 1314865 | N/A | N/A | 39369 | 39388 | AGTGCCACTTTTCAACACAT | 103 | 1175 |
| 1314946 | N/A | N/A | 69318 | 69337 | AGAAGCACAATTTCTTAGCA | 70 | 1176 |
| 1314950 | N/A | N/A | 104176 | 104195 | GCACTAAATTTTCCTAATTA | 88 | 1177 |
| 1314961 | N/A | N/A | 73051 | 73070 | TAGACAATTTTTAAGCAAAC | 91 | 1178 |
| 1315072 | N/A | N/A | 19344 | 19363 | GTATTCTTCCATATGGGCTC | 56 | 1179 |
| 1315099 | N/A | N/A | 127335 | 127354 | ACACTCATCATCTAAGCATT | 67 | 1180 |
| 1315143 | N/A | N/A | 50850 | 50869 | GGGTAATTTCCTCAAAGGCA | 10 | 1181 |
| 1315207 | 5039 | 5058 | 112922 | 112941 | GTATTTAACACTCCAAGGGC | 29 | 1182 |
| 1315253 | N/A | N/A | 135640 | 135659 | TACCAAGGCTCTACCAAGGC | 64 | 1183 |
| | | | 135651 | 135670 | | | |
| 1315294 | N/A | N/A | 56481 | 56500 | TGATTATTCCTCAATTGGTC | 18 | 1184 |
| 1315295 | N/A | N/A | 74501 | 74520 | GGACATTACCTTTATTGCGG | 81 | 1185 |
| 1315307 | N/A | N/A | 109993 | 110012 | CCATTCTAAAACCATCTGTA | 77 | 1186 |
| 1315311 | 11870 | 11889 | 168619 | 168638 | AGTCATTTTTACTAATGAGC | 50 | 1187 |
| 1315368 | N/A | N/A | 72423 | 72442 | ACTCATAAATATCAACCCCT | 96 | 1188 |
| 1315386 | N/A | N/A | 79596 | 79615 | ACACTGAAACTTAATTACCA | 80 | 1189 |
| 1315449 | N/A | N/A | 80866 | 80885 | TCATTAGTTTCTAATATCTT | 76 | 1190 |
| 1315457 | N/A | N/A | 43329 | 43348 | CAGTGCTTAAATTATTAGCA | 63 | 1191 |
| 1315468 | N/A | N/A | 17759 | 17778 | TTGAACTATATTCCACTCAA | 77 | 1192 |
| 1315547 | N/A | N/A | 55763 | 55782 | GGGCATGATTCCACATCACA | 77 | 1193 |
| 1315560 | N/A | N/A | 22485 | 22504 | ATAAACATTTCAGCTTGCTA | 99 | 1194 |
| 1315645 | N/A | N/A | 145833 | 145852 | CCAGCTAATCTCTTACCTCA | 72 | 1195 |
| 1315720 | N/A | N/A | 130438 | 130457 | CTTACGACATAATCACAGAA | 98 | 1196 |
| 1315731 | N/A | N/A | 70353 | 70372 | TCACTCAATATTACAATCCA | 75 | 1197 |
| 1315752 | N/A | N/A | 48853 | 48872 | GGTATTTCCACTCATTATTT | 25 | 1198 |
| 1315759 | N/A | N/A | 59076 | 59095 | ACATCATCATCTAAACTTAA | 80 | 1199 |
| 1315761 | N/A | N/A | 133703 | 133722 | TCTATATCCTTTATATCCTG | 62 | 1200 |
| 1315780 | N/A | N/A | 46628 | 46647 | GCACTCAAATTTAAACTGTA | 78 | 1201 |
| 1315953 | N/A | N/A | 45506 | 45525 | AGCTTCTTAAACATACTATT | 87 | 1202 |
| 1315970 | N/A | N/A | 68426 | 68445 | TCATTGTATCTATCACGCAA | 38 | 1203 |
| 1316018 | N/A | N/A | 105194 | 105213 | TCCAGCAAAATCATCAGGCA | 69 | 1204 |
| 1316025 | N/A | N/A | 15323 | 15342 | AGAGATAGAATTCAAACCTT | 57 | 1205 |
| 1316048 | N/A | N/A | 3056 | 3075 | GTTCTGTCAAATCAGCATAA | 66 | 1206 |
| 1316110 | N/A | N/A | 115715 | 115734 | TCCACTGTAAATATACACCA | 57 | 1207 |
| 1316218 | N/A | N/A | 35333 | 35352 | GCATCTTGTTTTACCACCCA | 28 | 1208 |
| 1316299 | N/A | N/A | 20021 | 20040 | GCACCTTCAAATCTTTCTGT | 68 | 1209 |
| 1316314 | N/A | N/A | 12495 | 12514 | GCCAAACTGCATCTTACAAC | 39 | 1210 |
| 1316354 | N/A | N/A | 32156 | 32175 | ACAACCATATTTTTAACTCA | 84 | 1211 |
| 1316366 | N/A | N/A | 6821 | 6840 | GAGTTTTTACAAATCATCTA | 77 | 1212 |
| 1316398 | N/A | N/A | 53256 | 53275 | GGAGCAATATATAAAGACAT | 93 | 1213 |
| 1316403 | N/A | N/A | 52732 | 52751 | CCACCCTCATATAATCCAGT | 75 | 1214 |
| 1316422 | N/A | N/A | 9019 | 9038 | AACACAGGTCTTCCTCCCAA | 55 | 1215 |
| 1316428 | N/A | N/A | 83581 | 83600 | GGCTTATACATCCTACAAAT | 60 | 1216 |
| 1316578 | N/A | N/A | 26541 | 26560 | TCACACATACACAATTTAAT | 98 | 1217 |
| 1316581 | N/A | N/A | 160500 | 160519 | CAGCATGTCCCAAATCCTGC | 81 | 1218 |
| 1316586 | N/A | N/A | 118105 | 118124 | TCACCCATTAACTCTACCGT | 66 | 1219 |
| 1316600 | N/A | N/A | 10393 | 10412 | CCTACTCTACTACCCCATCT | 128 | 1220 |
| 1316632 | N/A | N/A | 18259 | 18278 | TTTTTAATTTTCTAATGGGA | 92 | 1221 |
| 1316659 | N/A | N/A | 61440 | 61459 | GCTACATTTTTTTAAAAGTT | 85 | 1222 |
| 1316678 | N/A | N/A | 24772 | 24791 | CTTGAAGTAATCCCAAGAAT | 109 | 1223 |
| 1316697 | N/A | N/A | 125051 | 125070 | GCAAATTTCTCCACAGCATC | 76 | 1224 |
| 1316702 | N/A | N/A | 47417 | 47436 | GTTTATACCTTAACTTCTCC | 86 | 1225 |
| 1316711 | N/A | N/A | 94182 | 94201 | GGTGAGTAAATCTACCTTTA | 101 | 1226 |
| 1316748 | N/A | N/A | 111323 | 111342 | AAAGCACTCATCAGTGCCCT | 100 | 1227 |
| 1316765 | N/A | N/A | 5886 | 5905 | CCTTACTTCTTACTACAAAT | 84 | 1228 |
| 1316832 | N/A | N/A | 17059 | 17078 | GTTCAAAAATACCTCTACTA | 76 | 1229 |
| 1316843 | N/A | N/A | 81259 | 81278 | GTCACACCTAAATCCTAGCC | 46 | 1230 |
| 1316954 | N/A | N/A | 119046 | 119065 | GCTTTGATCACAAAAGCACC | 95 | 1231 |
| 1316972 | 4826 | 4845 | 106881 | 106900 | TCAAGCTCTTTTCCTGCATC | 26 | 1232 |
| 1316977 | N/A | N/A | 138729 | 138748 | CACATCTACACATCCGTCCA | 80 | 1233 |
| 1316986 | N/A | N/A | 44949 | 44968 | CTGTTCACAAATGTTAGAAA | 51 | 1234 |
| 1317018 | 1019 | 1038 | 41729 | 41748 | AAGAGCACATTTAGTAGCCA | 60 | 1235 |
| 1317042 | N/A | N/A | 98440 | 98459 | AAGTCTTGAAATCCAAGTTA | 77 | 1236 |
| 1317110 | N/A | N/A | 131678 | 131697 | GGATAACACCTCTTTCAGTT | 46 | 1237 |
| 1317122 | N/A | N/A | 33722 | 33741 | GCAACAATCATACCTTAATT | 88 | 1238 |
| 1317146 | N/A | N/A | 100923 | 100942 | AACCAATTTCACATTCACAC | 76 | 1239 |
| 1317153 | N/A | N/A | 108109 | 108128 | GGCTGATGAACTCCTCTCCA | 34 | 1240 |
| 1317189 | N/A | N/A | 60362 | 60381 | CCACTTCTAAATAAATCTCA | 95 | 1241 |
| 1317201 | N/A | N/A | 34505 | 34524 | AGCAGCTAACATCTAAGCCC | 83 | 1242 |
| 1317208 | N/A | N/A | 149408 | 149427 | TCATACATTTTACAGGCCTT | 86 | 1243 |

TABLE 18
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 25 | 858 |
| 1314572 | N/A | N/A | 109959 | 109978 | GACCACACTAAAACTGCTGT | 89 | 1244 |
| 1314592 | N/A | N/A | 35332 | 35351 | CATCTTGTTTTACCACCCAT | 67 | 1245 |
| 1314619 | N/A | N/A | 130436 | 130455 | TACGACATAATCACAGAAGA | 81 | 1246 |
| 1314648 | N/A | N/A | 69317 | 69336 | GAAGCACAATTTCTTAGCAA | 41 | 1247 |
| 1314651 | N/A | N/A | 45496 | 45515 | ACATACTATTTATTCAAGAA | 88 | 1248 |
| 1314696 | 4825 | 4844 | 106880 | 106899 | CAAGCTCTTTTCCTGCATCA | 25 | 1249 |
| 1314706 | N/A | N/A | 138628 | 138647 | GACCATGTCAATGAATGGCA | 96 | 1250 |
| 1314753 | N/A | N/A | 98429 | 98448 | TCCAAGTTATATACATTACA | 68 | 1251 |
| 1314776 | N/A | N/A | 79592 | 79611 | TGAAACTTAATTACCAGGGT | 72 | 1252 |
| 1314790 | N/A | N/A | 94124 | 94143 | ATCCAGTTATTCTTATGCCA | 35 | 1253 |
| 1314802 | N/A | N/A | 80862 | 80881 | TAGTTTCTAATATCTTGAAC | 84 | 1254 |
| 1314825 | N/A | N/A | 131471 | 131490 | ACAGGAGGTTTTATTTTCCT | 97 | 1255 |
| 1314846 | N/A | N/A | 2987 | 3006 | CAGAAAGGTCTTCCTTCCCA | 78 | 1256 |
| 1314881 | N/A | N/A | 15312 | 15331 | TCAAACCTTTTTTTTTCCTA | 75 | 1257 |
| 1314922 | N/A | N/A | 129017 | 129036 | GCCTATAGATTCTATTTCTA | 81 | 1258 |
| 1314930 | N/A | N/A | 52720 | 52739 | AATCCAGTTATTAGTACCTT | 27 | 1259 |
| 1315051 | N/A | N/A | 26537 | 26556 | ACATACACAATTTAATCCTC | 95 | 1260 |
| 1315113 | N/A | N/A | 53161 | 53180 | TGGTCAGAAACAACTGTCAA | 76 | 1261 |
| 1315174 | N/A | N/A | 17673 | 17692 | TCATTTTTTCACATTTAGGT | 26 | 1262 |
| 1315200 | N/A | N/A | 68258 | 68277 | GCAACAATTTATTCTAAGCT | 34 | 1263 |
| 1315319 | N/A | N/A | 117425 | 117444 | ACATTATGACTATCTGAATC | 70 | 1264 |
| 1315335 | N/A | N/A | 24752 | 24771 | AGCAAATCCCAGTAATACGG | 91 | 1265 |
| 1315361 | N/A | N/A | 22454 | 22473 | TGAACGAATCCCCTTCATCA | 77 | 1266 |
| 1315383 | N/A | N/A | 73038 | 73057 | AGCAAACAACAGTCTTCCTT | 94 | 1267 |
| 1315390 | N/A | N/A | 135639 | 135658 | ACCAAGGCTCTACCAAGGCT | 69 | 1268 |
| | | | 135650 | 135669 | | | |
| 1315430 | N/A | N/A | 13912 | 13931 | GCCTAAATATATTTACCATC | 84 | 1269 |
| 1315431 | N/A | N/A | 18236 | 18255 | AGGGCACTGCACCAATATCA | 89 | 1270 |
| 1315639 | N/A | N/A | 22145 | 22164 | CCTTTATTTGCCTCTTCCCC | 75 | 1271 |
| 1315640 | N/A | N/A | 108108 | 108127 | GCTGATGAACTCCTCTCCAT | 53 | 1272 |
| 1315669 | N/A | N/A | 141360 | 141379 | ACACACTTCCATCAGTGCTT | 114 | 1273 |
| 1315702 | N/A | N/A | 56467 | 56486 | TTGGTCATTTTCATCAGCTA | 16 | 1274 |
| 1315757 | N/A | N/A | 59073 | 59092 | TCATCATCTAAACTTAAACC | 105 | 1275 |
| 1315775 | N/A | N/A | 33719 | 33738 | ACAATCATACCTTAATTTCA | 100 | 1276 |
| 1315790 | N/A | N/A | 112870 | 112889 | GACACCACAAACATTTCTAC | 101 | 1277 |
| 1315874 | N/A | N/A | 70351 | 70370 | ACTCAATATTACAATCCAGA | 25 | 1278 |
| 1315880 | N/A | N/A | 125012 | 125031 | TCATGACTTCATTCTATCCT | 73 | 1279 |
| 1315899 | 11869 | 11888 | 168618 | 168637 | GTCATTTTTACTAATGAGCT | 68 | 1280 |
| 1315948 | N/A | N/A | 119021 | 119040 | CCCATCCCTTTTTAAGGCAA | 91 | 1281 |
| 1315955 | N/A | N/A | 149261 | 149280 | CTGCATTCCACAACTTCCCA | 81 | 1282 |
| 1315972 | N/A | N/A | 60292 | 60311 | CACTATGTACTCCACAGCAC | 80 | 1283 |
| 1315979 | N/A | N/A | 105152 | 105171 | GTCAAACTATATCTCCAATA | 52 | 1284 |
| 1316010 | N/A | N/A | 74279 | 74298 | GGCACAGACATTACCTCTCC | 93 | 1285 |
| 1316030 | N/A | N/A | 44880 | 44899 | TTCAGGACCCACCCTTGCTC | 90 | 1286 |
| 1316069 | N/A | N/A | 61169 | 61188 | ACGGCACACACCTATTCTCC | 74 | 1287 |
| 1316166 | N/A | N/A | 46562 | 46581 | TGGAATATACTTACACCATA | 93 | 1288 |
| 1316169 | N/A | N/A | 115658 | 115677 | AGTTGGTTCCACATTTTAGC | 24 | 1289 |
| 1316202 | N/A | N/A | 100921 | 100940 | CCAATTTCACATTCACACGA | 81 | 1290 |
| 1316208 | N/A | N/A | 12494 | 12513 | CCAAACTGCATCTTACAACC | 78 | 1291 |
| 1316269 | N/A | N/A | 47397 | 47416 | CCCTAAATATTTCCCTATCA | 101 | 1292 |
| 1316290 | N/A | N/A | 48829 | 48848 | GGCACGCATTCCATACAGAA | 48 | 1293 |
| 1316328 | N/A | N/A | 19321 | 19340 | CCACCTTGCATCTCTGCACT | 86 | 1294 |
| 1316353 | N/A | N/A | 127297 | 127316 | GGGACACAATTCCCAGATTC | 84 | 1295 |
| 1316375 | N/A | N/A | 17029 | 17048 | CAAGATTATTTTAAATTTCT | 92 | 1296 |
| 1316397 | N/A | N/A | 118104 | 118123 | CACCCATTAACTCTACCGTA | 70 | 1297 |
| 1316423 | N/A | N/A | 158914 | 158933 | TTGCTAATTTTCTTTGCTGA | 71 | 1298 |
| 1316426 | N/A | N/A | 20009 | 20028 | CTTTCTGTTCCATCTTCACA | 72 | 1299 |
| 1316450 | N/A | N/A | 145700 | 145719 | AGGGAACTCCTCAATGCCAG | 68 | 1300 |
| 1316468 | N/A | N/A | 133699 | 133718 | TATCCTTTATATCCTGGGTT | 76 | 1301 |
| 1316484 | N/A | N/A | 10390 | 10409 | ACTCTACTACCCCATCTCTA | 87 | 1302 |
| 1316522 | N/A | N/A | 72375 | 72394 | GGTGTCTACCCATATTTTCT | 63 | 1303 |
| 1316524 | N/A | N/A | 6819 | 6838 | GTTTTTACAAATCATCTATC | 80 | 1304 |
| 1316559 | N/A | N/A | 83580 | 83599 | GCTTATACATCCTACAAATC | 81 | 1305 |
| 1316605 | N/A | N/A | 34504 | 34523 | GCAGCTAACATCTAAGCCCT | 86 | 1306 |
| 1316645 | N/A | N/A | 111084 | 111103 | TGAGTGGACATCTCAGATGC | 90 | 1307 |
| 1316759 | N/A | N/A | 43326 | 43345 | TGCTTAAATTATTAGCATTA | 91 | 1308 |
| 1316862 | N/A | N/A | 55762 | 55781 | GGCATGATTCCACATCACAA | 52 | 1309 |
| 1316923 | N/A | N/A | 5882 | 5901 | ACTTCTTACTACAAATGTCC | 91 | 1310 |
| 1316940 | N/A | N/A | 50762 | 50781 | CCAACACAACTCTACCTTCA | 60 | 1311 |
| 1316993 | N/A | N/A | 22977 | 22996 | AAGTTTAGAAATCTATTGTT | 91 | 1312 |
| 1317045 | N/A | N/A | 41509 | 41528 | TTAGAAGTTTCCAATTCTTG | 88 | 1313 |
| 1317046 | N/A | N/A | 8997 | 9016 | GCTTGCATTGTTATTAGCAA | 81 | 1314 |
| 1317089 | N/A | N/A | 134291 | 134310 | CATGATAATTATATTCTCCT | 52 | 1315 |
| 1317105 | N/A | N/A | 103959 | 103978 | ACCACAAGATTTCATTCCAC | 70 | 1316 |
| 1317138 | N/A | N/A | 65432 | 65451 | AGGCAAGTCATATAAACCTA | 32 | 1317 |
| 1317157 | N/A | N/A | 39367 | 39386 | TGCCACTTTTCAACACATGC | 61 | 1318 |
| 1317168 | N/A | N/A | 81258 | 81277 | TCACACCTAAATCCTAGCCC | 85 | 1319 |
| 1317235 | N/A | N/A | 32081 | 32100 | TGGTACCTCAACCTTCAACA | 96 | 1320 |

TABLE 19
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314554 | N/A | N/A | 65324 | 65343 | ACTTTGGTTTTCTTTTCAGT | 36 | 1321 |
| 1314586 | N/A | N/A | 34471 | 34490 | TATGCTACACTCTAACACAT | 96 | 1322 |
| 1314631 | N/A | N/A | 117377 | 117396 | AAGGATGGCATCTCTTTCTA | 94 | 1323 |
| 1314646 | N/A | N/A | 22966 | 22985 | TCTATTGTTTTTATATCAGT | 89 | 1324 |
| 1314668 | N/A | N/A | 68257 | 68276 | CAACAATTTATTCTAAGCTT | 79 | 1325 |
| 1314684 | N/A | N/A | 6218 | 6237 | TAGCAGAGTTCAACAAGTGC | 67 | 1326 |
| 1314688 | N/A | N/A | 32057 | 32076 | GGCAAGTATCTTTTAAGCAA | 50 | 1327 |
| 1314764 | 4801 | 4820 | 106856 | 106875 | TATTTGTTCCTCTTAATACA | 78 | 1328 |
| 1314894 | N/A | N/A | 72302 | 72321 | GTATGTTTACTTGAACGGAT | 35 | 1329 |
| 1314936 | N/A | N/A | 39365 | 39384 | CCACTTTTCAACACATGCAC | 83 | 1330 |
| 1314999 | N/A | N/A | 56466 | 56485 | TGGTCATTTTCATCAGCTAT | 12 | 1331 |
| 1315010 | N/A | N/A | 73034 | 73053 | AACAACAGTCTTCCTTTCTC | 89 | 1332 |
| 1315166 | N/A | N/A | 52714 | 52733 | GTTATTAGTACCTTCAGTTA | 53 | 1333 |
| 1315169 | N/A | N/A | 12487 | 12506 | GCATCTTACAACCAGTGGTA | 36 | 1334 |
| 1315284 | N/A | N/A | 41474 | 41493 | TTGAGGCTTTTCCTACAGCT | 51 | 1335 |
| 1315291 | N/A | N/A | 103559 | 103578 | GCTTCTTTTCCTCCTGCTTA | 53 | 1336 |
| 1315355 | N/A | N/A | 47372 | 47391 | ACCAATGTGATCATTAGCTT | 60 | 1337 |
| 1315393 | N/A | N/A | 134977 | 134996 | GATTCTAGAAATATACACTC | 67 | 1338 |
| 1315395 | N/A | N/A | 17672 | 17691 | CATTTTTTCACATTTAGGTA | 81 | 1339 |
| 1315416 | N/A | N/A | 26536 | 26555 | CATACACAATTTAATCCTCA | 99 | 1340 |
| 1315452 | N/A | N/A | 83551 | 83570 | ACATCAACTATCCATTAGAC | 72 | 1341 |
| 1315528 | N/A | N/A | 131470 | 131489 | CAGGAGGTTTTATTTTCCTC | 96 | 1342 |
| 1315556 | 5816 | 5835 | 130399 | 130418 | CGTACTATTTCTCTATTGCA | 24 | 1343 |
| 1315559 | N/A | N/A | 43314 | 43333 | TAGCATTATCTTCAATTCAT | 47 | 1344 |
| 1315570 | N/A | N/A | 33718 | 33737 | CAATCATACCTTAATTTCAT | 106 | 1345 |
| 1315605 | 11868 | 11887 | 168617 | 168636 | TCATTTTTACTAATGAGCTC | 87 | 1346 |
| 1315608 | N/A | N/A | 18233 | 18252 | GCACTGCACCAATATCATTA | 81 | 1347 |
| 1315613 | N/A | N/A | 53123 | 53142 | GCTTGCCTTCTTCCAAGCCA | 95 | 1348 |
| 1315687 | N/A | N/A | 119020 | 119039 | CCATCCCTTTTTAAGGCAAC | 82 | 1349 |
| 1315706 | N/A | N/A | 100854 | 100873 | GCAGCATCGCCCTCTTCAGA | 90 | 1350 |
| 1315710 | N/A | N/A | 109957 | 109976 | CCACACTAAAACTGCTGTCA | 99 | 1351 |
| 1315719 | N/A | N/A | 19250 | 19269 | TCTCAGTTTTTCCTCTTGCC | 67 | 1352 |
| 1315750 | N/A | N/A | 22432 | 22451 | ATGAAATGACCATCTCATCC | 82 | 1353 |
| 1315756 | N/A | N/A | 69256 | 69275 | GTAAGATGACCATACAACTA | 72 | 1354 |
| 1315811 | N/A | N/A | 145667 | 145686 | GGAGGAATTTTACCTACAGC | 85 | 1355 |
| 1315837 | N/A | N/A | 149255 | 149274 | TCCACAACTTCCCATTCTGC | 70 | 1356 |
| 1315838 | N/A | N/A | 78919 | 78938 | GTATGAGCCCATCCACAGTA | 82 | 1357 |
| 1315875 | N/A | N/A | 60153 | 60172 | AGCATGCTTCCTTCTGCTCA | 95 | 1358 |
| 1315890 | N/A | N/A | 61167 | 61186 | GGCACACACCTATTCTCCTA | 73 | 1359 |
| 1315914 | N/A | N/A | 45493 | 45512 | TACTATTTATTCAAGAAGAA | 100 | 1360 |
| 1316028 | N/A | N/A | 5783 | 5802 | TGGAATACCACTACTTCATA | 77 | 1361 |
| 1316081 | N/A | N/A | 55682 | 55701 | CTACCAGCAAATTATTACCA | 63 | 1362 |
| 1316136 | N/A | N/A | 44826 | 44845 | TGTCTCACCCTTTCACTCCT | 96 | 1363 |
| 1316242 | N/A | N/A | 133683 | 133702 | GGTTGTCCTAACACAAGGAC | 82 | 1364 |
| 1316295 | N/A | N/A | 15309 | 15328 | AACCTTTTTTTTTCCTACTT | 91 | 1365 |
| 1316369 | N/A | N/A | 22138 | 22157 | TTGCCTCTTCCCCTATTTCT | 75 | 1366 |
| 1316371 | N/A | N/A | 112846 | 112865 | GCCAAAAGAAACGACAGCTT | 89 | 1367 |
| 1316388 | N/A | N/A | 50760 | 50779 | AACACAACTCTACCTTCATC | 79 | 1368 |
| 1316393 | N/A | N/A | 20005 | 20024 | CTGTTCCATCTTCACAGGGC | 68 | 1369 |
| 1316427 | N/A | N/A | 10379 | 10398 | CCATCTCTAACCCCAGGCAA | 86 | 1370 |
| 1316445 | N/A | N/A | 134289 | 134308 | TGATAATTATATTCTCCTTC | 63 | 1371 |
| 1316497 | N/A | N/A | 115610 | 115629 | GGCATGCAAATATCTTTTCA | 80 | 1372 |
| 1316509 | N/A | N/A | 125011 | 125030 | CATGACTTCATTCTATCCTC | 78 | 1373 |
| 1316530 | N/A | N/A | 118070 | 118089 | TAGCTCATTTATACTACCTC | 48 | 1374 |
| 1316535 | N/A | N/A | 158886 | 158905 | TGTGTGCTTTTACCATAACA | 29 | 1375 |
| 1316587 | N/A | N/A | 46550 | 46569 | ACACCATAAAATTACTCATA | 102 | 1376 |
| 1316603 | N/A | N/A | 98424 | 98443 | GTTATATACATTACAACCAT | 83 | 1377 |
| 1316701 | N/A | N/A | 128896 | 128915 | GACAGCACTCCTCTATATCT | 53 | 1378 |
| 1316725 | N/A | N/A | 108043 | 108062 | CAGTTGCTCATTTTCAGACC | 19 | 1379 |
| 1316732 | N/A | N/A | 17028 | 17047 | AAGATTATTTTAAATTTCTG | 108 | 1380 |
| 1316744 | N/A | N/A | 59072 | 59091 | CATCATCTAAACTTAAACCT | 99 | 1381 |
| 1316749 | N/A | N/A | 70341 | 70360 | ACAATCCAGATTATACATCT | 62 | 1382 |
| 1316750 | N/A | N/A | 141349 | 141368 | TCAGTGCTTTTTATTCAACA | 103 | 1383 |
| 1316787 | N/A | N/A | 94007 | 94026 | GATCAACTAACATCAATCTT | 81 | 1384 |
| 1316808 | N/A | N/A | 80861 | 80880 | AGTTTCTAATATCTTGAACC | 63 | 1385 |
| 1316811 | N/A | N/A | 35329 | 35348 | CTTGTTTTACCACCCATGCT | 81 | 1386 |
| 1316890 | N/A | N/A | 138491 | 138510 | TGCAAAGTACTATACTGGCC | 78 | 1387 |
| 1316893 | N/A | N/A | 13897 | 13916 | CCATCTACAGCTACTAGGAA | 45 | 1388 |
| 1316898 | N/A | N/A | 24730 | 24749 | GATGTGCTATTTTTGAAGCA | 101 | 1389 |
| 1316911 | N/A | N/A | 8779 | 8798 | TGTCTCTAACATACATTCTA | 70 | 1390 |
| 1316925 | N/A | N/A | 110976 | 110995 | TGCTACATCCAGACAGGCTC | 89 | 1391 |
| 1316970 | N/A | N/A | 81232 | 81251 | TTCTGACCTTTTCATGGGTA | 103 | 1392 |
| 1317063 | N/A | N/A | 127113 | 127132 | AAGACATCCACCAAGCATTT | 75 | 1393 |
| 1317125 | N/A | N/A | 2979 | 2998 | TCTTCCTTCCCATCAGGAGC | 86 | 1394 |
| 1317130 | N/A | N/A | 105117 | 105136 | TCAAAATTTAATTCACGGCA | 32 | 1395 |
| 1317161 | N/A | N/A | 74278 | 74297 | GCACAGACATTACCTCTCCA | 89 | 1396 |
| 1317225 | N/A | N/A | 48828 | 48847 | GCACGCATTCCATACAGAAA | 45 | 1397 |

TABLE 20
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 22 | 858 |
| 1314492 | N/A | N/A | 106788 | 106807 | AAAGGGTACCCTTATCAGGC | 103 | 1398 |
| 1314497 | N/A | N/A | 52707 | 52726 | GTACCTTCAGTTAATCCTCA | 46 | 1399 |
| 1314501 | N/A | N/A | 43312 | 43331 | GCATTATCTTCAATTCATGA | 55 | 1400 |
| 1314522 | N/A | N/A | 73033 | 73052 | ACAACAGTCTTCCTTTCTCT | 91 | 1401 |
| 1314556 | N/A | N/A | 18231 | 18250 | ACTGCACCAATATCATTACA | 86 | 1402 |
| 1314565 | N/A | N/A | 22425 | 22444 | GACCATCTCATCCTGGGCAA | 83 | 1403 |
| 1314695 | N/A | N/A | 17012 | 17031 | TCTGTGTTAATTTAGAGAAA | 77 | 1404 |
| 1314731 | N/A | N/A | 46048 | 46067 | GCACCCACATTTCCTTCACC | 83 | 1405 |
| 1314810 | 5815 | 5834 | 130398 | 130417 | GTACTATTTCTCTATTGCAC | 10 | 1406 |
| 1314838 | N/A | N/A | 109946 | 109965 | CTGCTGTCAATCTCACCAGA | 76 | 1407 |
| 1314896 | N/A | N/A | 48815 | 48834 | ACAGAAAAAAATTCTTCCGT | 56 | 1408 |
| 1315009 | N/A | N/A | 117370 | 117389 | GCATCTCTTTCTATAGGTCT | 51 | 1409 |
| 1315027 | N/A | N/A | 107757 | 107776 | TGTCTTTGAATTCTTATGCC | 22 | 1410 |
| 1315069 | N/A | N/A | 112815 | 112834 | GCTATGTAACTCAAGGGCAA | 79 | 1411 |
| 1315094 | N/A | N/A | 81169 | 81188 | CTCAGATGACTTTATCCCCA | 48 | 1412 |
| 1315155 | N/A | N/A | 145069 | 145088 | ATCATCTTATTTCAGCGGGC | 21 | 1413 |
| 1315170 | N/A | N/A | 34469 | 34488 | TGCTACACTCTAACACATAT | 103 | 1414 |
| 1315261 | N/A | N/A | 100760 | 100779 | TCTGAGCTTTTTTCTGCTAC | 76 | 1415 |
| 1315363 | N/A | N/A | 118069 | 118088 | AGCTCATTTATACTACCTCC | 51 | 1416 |
| 1315404 | N/A | N/A | 45477 | 45496 | AGAATGGTTTCTAATACGAA | 78 | 1417 |
| 1315496 | N/A | N/A | 13547 | 13566 | GTTCAATTACCATAATGGTT | 38 | 1418 |
| 1315550 | N/A | N/A | 141344 | 141363 | GCTTTTTATTCAACATTTTA | 81 | 1419 |
| 1315552 | N/A | N/A | 65282 | 65301 | CGTTTTTTTATATTTTCCTT | 28 | 1420 |
| 1315589 | N/A | N/A | 80835 | 80854 | GGAGAAACTTTCTCTCACTT | 78 | 1421 |
| 1315611 | N/A | N/A | 26533 | 26552 | ACACAATTTAATCCTCACAC | 105 | 1422 |
| 1315655 | N/A | N/A | 53111 | 53130 | CCAAGCCACATTCAAGATCT | 80 | 1423 |
| 1315685 | N/A | N/A | 69250 | 69269 | TGACCATACAACTAACTGGC | 64 | 1424 |
| 1315767 | N/A | N/A | 39207 | 39226 | GCATCAGCAATTCCAAGTGC | 79 | 1425 |
| 1315858 | N/A | N/A | 68246 | 68265 | TCTAAGCTTATCAAATTCCT | 58 | 1426 |
| 1315871 | N/A | N/A | 33714 | 33733 | CATACCTTAATTTCATTGTC | 62 | 1427 |
| 1315910 | N/A | N/A | 128895 | 128914 | ACAGCACTCCTCTATATCTT | 45 | 1428 |
| 1315926 | N/A | N/A | 134916 | 134935 | CTTGTTTAAACCTTTTTCTT | 77 | 1429 |
| 1315980 | N/A | N/A | 60915 | 60934 | TACTTGATAACATCATTCCT | 86 | 1430 |
| 1316008 | N/A | N/A | 15308 | 15327 | ACCTTTTTTTTTCCTACTTT | 62 | 1431 |
| 1316011 | N/A | N/A | 60152 | 60171 | GCATGCTTCCTTCTGCTCAA | 97 | 1432 |
| 1316044 | N/A | N/A | 158859 | 158878 | GCTGAGAGTCTTTCTAGAAA | 31 | 1433 |
| 1316063 | N/A | N/A | 22965 | 22984 | CTATTGTTTTTATATCAGTC | 62 | 1434 |
| 1316084 | 3864 | 3883 | 83463 | 83482 | TTTCAGGACATCATGCAGTT | 35 | 1435 |
| 1316149 | N/A | N/A | 115609 | 115628 | GCATGCAAATATCTTTTCAC | 81 | 1436 |
| 1316164 | N/A | N/A | 44799 | 44818 | AGCAAAGACCACATTTTCTG | 75 | 1437 |
| 1316170 | N/A | N/A | 72290 | 72309 | GAACGGATTTTTAACTTTCT | 22 | 1438 |
| 1316177 | N/A | N/A | 133660 | 133679 | CTGTGTTTCCAGCATTACCT | 72 | 1439 |
| 1316182 | N/A | N/A | 55646 | 55665 | AGTTTGTTAACTACTATACT | 72 | 1440 |
| 1316187 | N/A | N/A | 94006 | 94025 | ATCAACTAACATCAATCTTC | 105 | 1441 |
| 1316217 | N/A | N/A | 74103 | 74122 | CCATGACTGTCACCACCACA | 58 | 1442 |
| 1316228 | N/A | N/A | 70339 | 70358 | AATCCAGATTATACATCTCC | 39 | 1443 |
| 1316251 | N/A | N/A | 105074 | 105093 | GTTGCATCGCTTCTTGGCCT | 95 | 1444 |
| 1316261 | N/A | N/A | 41328 | 41347 | CCTCAATTCTAGATACACTC | 71 | 1445 |
| 1316296 | N/A | N/A | 10358 | 10377 | CACTAATCTATTCTCCATCC | 91 | 1446 |
| 1316305 | 11851 | 11870 | 168600 | 168619 | CTCATATTCATCTCCGGAGC | 57 | 1447 |
| 1316315 | N/A | N/A | 24691 | 24710 | TCATGATCCCAATCTAACTA | 78 | 1448 |
| 1316348 | N/A | N/A | 2891 | 2910 | ACTACAGTAAATTTCTGGAT | 64 | 1449 |
| 1316378 | N/A | N/A | 59071 | 59090 | ATCATCTAAACTTAAACCTT | 88 | 1450 |
| 1316391 | N/A | N/A | 110920 | 110939 | TCCACAGCCACCCTGTCCTC | 93 | 1451 |
| 1316429 | N/A | N/A | 19249 | 19268 | CTCAGTTTTTCCTCTTGCCA | 47 | 1452 |
| 1316494 | N/A | N/A | 103431 | 103450 | TCAACAACTTTACTATACCC | 102 | 1453 |
| 1316534 | N/A | N/A | 12465 | 12484 | GTTTGATTAGAAACTTTCTT | 55 | 1454 |
| 1316563 | N/A | N/A | 56464 | 56483 | GTCATTTTCATCAGCTATTC | 16 | 1455 |
| 1316590 | N/A | N/A | 50757 | 50776 | ACAACTCTACCTTCATCTGT | 75 | 1456 |
| 1316612 | N/A | N/A | 78813 | 78832 | ACAGAGTTCCTTTATGGGTT | 73 | 1457 |
| 1316789 | N/A | N/A | 47351 | 47370 | CATGTTGTTTTCTAAATTCC | 88 | 1458 |
| 1316810 | N/A | N/A | 131469 | 131488 | AGGAGGTTTTATTTTCCTCC | 81 | 1459 |
| 1316824 | 6757 | 6776 | 138424 | 138443 | ACAGATCATTCAACTTGCTC | 86 | 1460 |
| 1316834 | N/A | N/A | 22137 | 22156 | TGCCTCTTCCCCTATTTCTG | 61 | 1461 |
| 1316861 | N/A | N/A | 127078 | 127097 | GTCTGTCTCCTCCAACAGAA | 87 | 1462 |
| 1316920 | N/A | N/A | 149164 | 149183 | GCCTGATAAAATCTCTGCAA | 70 | 1463 |
| 1316929 | N/A | N/A | 6190 | 6209 | CCGCATTCAAAATCCAGTCA | 34 | 1464 |
| 1316962 | N/A | N/A | 5768 | 5787 | TCATACACCCTTACACTAGA | 74 | 1465 |
| 1316963 | N/A | N/A | 98417 | 98436 | ACATTACAACCATTCTTTCA | 79 | 1466 |
| 1317027 | N/A | N/A | 35179 | 35198 | TTTCAGTTTTTTTAGAGATA | 81 | 1467 |
| 1317032 | N/A | N/A | 20004 | 20023 | TGTTCCATCTTCACAGGGCC | 65 | 1468 |
| 1317044 | N/A | N/A | 124824 | 124843 | CTGATTTAAATTTCAGGCCG | 81 | 1469 |
| 1317057 | N/A | N/A | 17658 | 17677 | TAGGTAATTAATCTGAACTT | 71 | 1470 |
| 1317145 | N/A | N/A | 134250 | 134269 | TCTCACACACACAATCCCTG | 95 | 1471 |
| 1317173 | N/A | N/A | 119001 | 119020 | CTACTTGTTATCATTTCTGT | 71 | 1472 |
| 1317200 | N/A | N/A | 8777 | 8796 | TCTCTAACATACATTCTAGC | 82 | 1473 |
| 1317213 | N/A | N/A | 31963 | 31982 | GGCACACATCCCATAAGTCT | 76 | 1474 |

TABLE 21
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314477 | N/A | N/A | 83308 | 83327 | GCCAAGTTTTAAAGAGATTA | 65 | 1475 |
| 1314525 | N/A | N/A | 65281 | 65300 | GTTTTTTTATATTTTCCTTA | 40 | 1476 |
| 1314550 | N/A | N/A | 34465 | 34484 | ACACTCTAACACATATTCCT | 94 | 1477 |
| 1314573 | N/A | N/A | 19248 | 19267 | TCAGTTTTTCCTCTTGCCAT | 79 | 1478 |
| 1314587 | N/A | N/A | 46045 | 46064 | CCCACATTTCCTTCACCTCA | 86 | 1479 |
| 1314624 | N/A | N/A | 112800 | 112819 | GGCAAATCATCATAATCATC | 49 | 1480 |
| 1314697 | N/A | N/A | 56457 | 56476 | TCATCAGCTATTCAGAACTC | 48 | 1481 |
| 1314709 | 11144 | 11163 | 167893 | 167912 | ACAGAGTTCCAACATTTCCT | 54 | 1482 |
| 1314728 | N/A | N/A | 53106 | 53125 | CCACATTCAAGATCTCGACT | 71 | 1483 |
| 1314755 | N/A | N/A | 72289 | 72308 | AACGGATTTTTAACTTTCTT | 32 | 1484 |
| 1314758 | N/A | N/A | 60914 | 60933 | ACTTGATAACATCATTCCTT | 88 | 1485 |
| 1314777 | N/A | N/A | 158715 | 158734 | TTCTTCACTTTCAATCTGCT | 54 | 1486 |
| 1314869 | N/A | N/A | 81167 | 81186 | CAGATGACTTTATCCCCACT | 40 | 1487 |
| 1314947 | N/A | N/A | 16983 | 17002 | AGTAATATAAAATCTTCCTA | 100 | 1488 |
| 1314991 | N/A | N/A | 52704 | 52723 | CCTTCAGTTAATCCTCATCT | 53 | 1489 |
| 1315007 | N/A | N/A | 31962 | 31981 | GCACACATCCCATAAGTCTC | 73 | 1490 |
| 1315032 | N/A | N/A | 98412 | 98431 | ACAACCATTCTTTCATGCAT | 86 | 1491 |
| 1315042 | N/A | N/A | 33713 | 33732 | ATACCTTAATTTCATTGTCA | 44 | 1492 |
| 1315047 | N/A | N/A | 17589 | 17608 | GAGAAATGTATCATTTCCCT | 101 | 1493 |
| 1315215 | N/A | N/A | 133652 | 133671 | CCAGCATTACCTCTGAGCGA | 94 | 1494 |
| 1315315 | N/A | N/A | 115526 | 115545 | AGGTGTACTCTTACAGCAAC | 43 | 1495 |
| 1315359 | N/A | N/A | 22397 | 22416 | GCTTTGAAATCCACTTTGTC | 91 | 1496 |
| 1315384 | N/A | N/A | 106787 | 106806 | AAGGGTACCCTTATCAGGCA | 90 | 1497 |
| 1315448 | 6755 | 6774 | 138422 | 138441 | AGATCATTCAACTTGCTCCA | 94 | 1498 |
| 1315472 | N/A | N/A | 77967 | 77986 | TTCCACCTCAAATCATCATC | 98 | 1499 |
| 1315517 | N/A | N/A | 141335 | 141354 | TCAACATTTTACATTTGTGC | 75 | 1500 |
| 1315577 | N/A | N/A | 131466 | 131485 | AGGTTTTATTTTCCTCCGTT | 82 | 1501 |
| 1315578 | N/A | N/A | 60025 | 60044 | GCTTTGATTTTTACCAGTTA | 13 | 1502 |
| 1315585 | N/A | N/A | 128815 | 128834 | GTATTGTAAATTATGTGTCA | 28 | 1503 |
| 1315587 | N/A | N/A | 118068 | 118087 | GCTCATTTATACTACCTCCT | 43 | 1504 |
| 1315616 | N/A | N/A | 144457 | 144476 | CCTCAGTTCATTTTAGAAAA | 91 | 1505 |
| 1315625 | N/A | N/A | 35002 | 35021 | ATAGATTTTTTTTAATGCCA | 44 | 1506 |
| 1315630 | N/A | N/A | 6176 | 6195 | CAGTCAGTCATTCCTCACAC | 44 | 1507 |
| 1315631 | N/A | N/A | 41169 | 41188 | GGACTGGAACATCTTCGATC | 74 | 1508 |
| 1315643 | N/A | N/A | 15307 | 15326 | CCTTTTTTTTTCCTACTTTT | 72 | 1509 |
| 1315690 | N/A | N/A | 118972 | 118991 | TGGTAGTTACCTCCAAAATT | 87 | 1510 |
| 1315715 | N/A | N/A | 18230 | 18249 | CTGCACCAATATCATTACAC | 71 | 1511 |
| 1315732 | N/A | N/A | 13500 | 13519 | AGGGAACATTATTCATTTCA | 48 | 1512 |
| 1315802 | N/A | N/A | 94005 | 94024 | TCAACTAACATCAATCTTCA | 97 | 1513 |
| 1315808 | N/A | N/A | 12451 | 12470 | TTTCTTTCTTTCTCAGCAGT | 23 | 1514 |
| 1315813 | N/A | N/A | 70338 | 70357 | ATCCAGATTATACATCTCCA | 29 | 1515 |
| 1315836 | N/A | N/A | 74004 | 74023 | TTCTGCCTCCTTCATTCTCT | 101 | 1516 |
| 1315873 | N/A | N/A | 48780 | 48799 | CAAGGCAGATTAACATTTCC | 88 | 1517 |
| 1315929 | N/A | N/A | 39108 | 39127 | TTGGCAGTTTCTACTGCAGC | 114 | 1518 |
| 1315995 | N/A | N/A | 69249 | 69268 | GACCATACAACTAACTGGCC | 88 | 1519 |
| 1315998 | N/A | N/A | 149127 | 149146 | ACCAGGTACTACTCATGGCC | 77 | 1520 |
| 1316038 | N/A | N/A | 8774 | 8793 | CTAACATACATTCTAGCCCC | 75 | 1521 |
| 1316065 | N/A | N/A | 126944 | 126963 | GTCCAGTATCCATACTGAGC | 77 | 1522 |
| 1316075 | N/A | N/A | 22133 | 22152 | TCTTCCCCTATTTCTGGCTT | 87 | 1523 |
| 1316133 | N/A | N/A | 134194 | 134213 | AAAGTTTAACTTATTCTGCA | 53 | 1524 |
| 1316225 | N/A | N/A | 44798 | 44817 | GCAAAGACCACATTTTCTGT | 83 | 1525 |
| 1316267 | N/A | N/A | 22963 | 22982 | ATTGTTTTTATATCAGTCAA | 97 | 1526 |
| 1316298 | N/A | N/A | 10357 | 10376 | ACTAATCTATTCTCCATCCC | 92 | 1527 |
| 1316329 | N/A | N/A | 134915 | 134934 | TTGTTTAAACCTTTTTCTTC | 81 | 1528 |
| 1316345 | N/A | N/A | 26532 | 26551 | CACAATTTAATCCTCACACA | 94 | 1529 |
| 1316399 | N/A | N/A | 43286 | 43305 | TAAGAATCATTTTAAGCCAC | 99 | 1530 |
| 1316456 | N/A | N/A | 45474 | 45493 | ATGGTTTCTAATACGAACTA | 90 | 1531 |
| 1316460 | N/A | N/A | 24652 | 24671 | TTTCTCTTTATCTCTAGGTA | 75 | 1532 |
| 1316467 | N/A | N/A | 68241 | 68260 | GCTTATCAAATTCCTGATAA | 67 | 1533 |
| 1316505 | N/A | N/A | 2748 | 2767 | GCTCTCCGAATCATTTTCTA | 92 | 1534 |
| 1316540 | N/A | N/A | 103281 | 103300 | GTTTAGTAATAAGATACCCA | 68 | 1535 |
| 1316622 | N/A | N/A | 107598 | 107617 | ACCAACACTGTCAATTGATT | 42 | 1536 |
| 1316640 | N/A | N/A | 55645 | 55664 | GTTTGTTAACTACTATACTA | 64 | 1537 |
| 1316768 | N/A | N/A | 117323 | 117342 | GGAGAACCTTTTTAATATCA | 28 | 1538 |
| 1316803 | 5814 | 5833 | 130397 | 130416 | TACTATTTCTCTATTGCACA | 25 | 1539 |
| 1316817 | 2526 | 2545 | 59005 | 59024 | TGTGAGGGTTCTAATGGTGC | 48 | 1540 |
| 1316865 | N/A | N/A | 110865 | 110884 | ACATGCCACATCTAAGCCAC | 97 | 1541 |
| 1316868 | N/A | N/A | 73030 | 73049 | ACAGTCTTCCTTTCTCTTGC | 85 | 1542 |
| 1316875 | N/A | N/A | 105008 | 105027 | ACAGGAACTTTATATTCAGA | 49 | 1543 |
| 1316897 | N/A | N/A | 109894 | 109913 | CTCAAGATAACATTCCATTA | 51 | 1544 |
| 1316966 | N/A | N/A | 80834 | 80853 | GAGAAACTTTCTCTCACTTT | 79 | 1545 |
| 1317028 | N/A | N/A | 50599 | 50618 | GGATAAACTCATCTGAGCAA | 52 | 1546 |
| 1317038 | N/A | N/A | 123151 | 123170 | TTGTTTTTCATTTAAGGCTG | 76 | 1547 |
| 1317176 | N/A | N/A | 47264 | 47283 | AATGAAGATCTCTATTTCCC | 81 | 1548 |
| 1317182 | N/A | N/A | 100740 | 100759 | AGGAAACTGACTACTCAGCC | 79 | 1549 |
| 1317187 | N/A | N/A | 19977 | 19996 | TGTGTGTAAAATCTCCCTCT | 87 | 1550 |
| 1317188 | N/A | N/A | 4723 | 4742 | CTGACATTTCCCTATCCCCT | 82 | 1551 |

TABLE 22
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 31 | 858 |
| 1314482 | N/A | N/A | 16953 | 16972 | GGTCACCTATATCTTTCAGA | 50 | 1552 |
| 1314539 | N/A | N/A | 65270 | 65289 | TTTTCCTTAAATTCTGTTCA | 43 | 1553 |
| 1314627 | N/A | N/A | 18229 | 18248 | TGCACCAATATCATTACACA | 86 | 1554 |
| 1314635 | N/A | N/A | 15280 | 15299 | CGTTGTATTCTACAATAGCA | 36 | 1555 |
| 1314677 | N/A | N/A | 118968 | 118987 | AGTTACCTCCAAAATTCTAA | 82 | 1556 |
| 1314683 | N/A | N/A | 53090 | 53109 | GACTCCAGTCCTATTCTCCT | 84 | 1557 |
| 1314692 | N/A | N/A | 107168 | 107187 | AAGGATACACACCAACAACA | 81 | 1558 |
| 1314812 | N/A | N/A | 52703 | 52722 | CTTCAGTTAATCCTCATCTT | 74 | 1559 |
| 1314815 | N/A | N/A | 104960 | 104979 | GTTTCTTGAATTTTTTTCCC | 36 | 1560 |
| 1314833 | N/A | N/A | 68216 | 68235 | TCCTCTTTCACTGCTTCTAC | 32 | 1561 |
| 1314867 | N/A | N/A | 56453 | 56472 | CAGCTATTCAGAACTCAGAA | 23 | 1562 |
| 1314874 | N/A | N/A | 2566 | 2585 | ACCAGAGTACTTCCTAACTC | 65 | 1563 |
| 1314886 | 883 | 902 | N/A | N/A | AAACCTTAATTTCATTGTCA | 39 | 1564 |
| 1314910 | N/A | N/A | 81162 | 81181 | GACTTTATCCCCACTTTCTC | 66 | 1565 |
| 1314911 | N/A | N/A | 58843 | 58862 | GGAGATAAATTGCTCAGCCA | 88 | 1566 |
| 1314966 | N/A | N/A | 128708 | 128727 | GCACTTTGCAATAATACAGC | 28 | 1567 |
| 1314994 | N/A | N/A | 117322 | 117341 | GAGAACCTTTTTAATATCAA | 56 | 1568 |
| 1315021 | N/A | N/A | 19954 | 19973 | TCCTTTTTATATATATGTGA | 76 | 1569 |
| 1315035 | N/A | N/A | 122708 | 122727 | GCAAAATTCAATTCCAGAGA | 49 | 1570 |
| 1315138 | N/A | N/A | 10356 | 10375 | CTAATCTATTCTCCATCCCT | 100 | 1571 |
| 1315141 | 5800 | 5819 | 130383 | 130402 | TGCACATTCCAAGTTTGGCT | 27 | 1572 |
| 1315152 | N/A | N/A | 35001 | 35020 | TAGATTTTTTTTAATGCCAA | 78 | 1573 |
| 1315161 | N/A | N/A | 72261 | 72280 | TACATTTTACTCCAATGCCA | 52 | 1574 |
| 1315178 | N/A | N/A | 70337 | 70356 | TCCAGATTATACATCTCCAT | 36 | 1575 |
| 1315214 | N/A | N/A | 126801 | 126820 | GGTCACAGCTTTTATTTCCA | 13 | 1576 |
| 1315228 | N/A | N/A | 22105 | 22124 | TTCACCATTTTTATTATTCT | 95 | 1577 |
| 1315259 | N/A | N/A | 106777 | 106796 | TTATCAGGCATTTCATGGAA | 96 | 1578 |
| 1315339 | N/A | N/A | 43220 | 43239 | GCACAATAAAAACCCTGACA | 101 | 1579 |
| 1315345 | N/A | N/A | 19198 | 19217 | GCTCTCCACATTCATTTTCC | 75 | 1580 |
| 1315405 | 6243 | 6262 | 133601 | 133620 | GTTGAGTTCTTCCATTGGCA | 26 | 1581 |
| 1315507 | N/A | N/A | 34464 | 34483 | CACTCTAACACATATTCCTT | 98 | 1582 |
| 1315525 | N/A | N/A | 77966 | 77985 | TCCACCTCAAATCATCATCA | 79 | 1583 |
| 1315543 | N/A | N/A | 12378 | 12397 | ACAGTCTAATACCTACTCTA | 77 | 1584 |
| 1315604 | N/A | N/A | 134191 | 134210 | GTTTAACTTATTCTGCAGTC | 31 | 1585 |
| 1315618 | N/A | N/A | 17582 | 17601 | GTATCATTTCCCTCATTTCC | 52 | 1586 |
| 1315659 | N/A | N/A | 31577 | 31596 | ACGAAGCATTTTCTTTCCTT | 73 | 1587 |
| 1315755 | N/A | N/A | 141297 | 141316 | AGGGATAGACAGACTAGCCT | 52 | 1588 |
| 1315772 | N/A | N/A | 109846 | 109865 | AGGCAGTAACATCAATATCA | 82 | 1589 |
| 1315787 | N/A | N/A | 47190 | 47209 | TCAGCTTGACACACATTTAA | 86 | 1590 |
| 1315789 | N/A | N/A | 60910 | 60929 | GATAACATCATTCCTTGCAT | 67 | 1591 |
| 1315798 | N/A | N/A | 103114 | 103133 | GCCTGGTTTTCTCAGAGCCT | 92 | 1592 |
| 1315800 | N/A | N/A | 80809 | 80828 | TGTGTATTATATTTATTTCT | 35 | 1593 |
| 1315968 | N/A | N/A | 48778 | 48797 | AGGCAGATTAACATTTCCTG | 41 | 1594 |
| 1315991 | N/A | N/A | 44708 | 44727 | GACTGAACTTCTCCAGGCCC | 92 | 1595 |
| 1315993 | N/A | N/A | 73027 | 73046 | GTCTTCCTTTCTCTTGCAAT | 82 | 1596 |
| 1315997 | N/A | N/A | 149126 | 149145 | CCAGGTACTACTCATGGCCT | 99 | 1597 |
| 1316003 | N/A | N/A | 94002 | 94021 | ACTAACATCAATCTTCATTA | 80 | 1598 |
| 1316017 | N/A | N/A | 55642 | 55661 | TGTTAACTACTATACTATTA | 65 | 1599 |
| 1316068 | N/A | N/A | 24525 | 24544 | TTAGCAGTATTTCAGACCTT | 70 | 1600 |
| 1316071 | N/A | N/A | 73997 | 74016 | TCCTTCATTCTCTTACAATC | 73 | 1601 |
| 1316074 | 6653 | 6672 | 138320 | 138339 | GCCAGAGTCACCTCACGGGC | 89 | 1602 |
| 1316156 | N/A | N/A | 22960 | 22979 | GTTTTTATATCAGTCAATCC | 36 | 1603 |
| 1316172 | N/A | N/A | 6175 | 6194 | AGTCAGTCATTCCTCACACT | 49 | 1604 |
| 1316193 | N/A | N/A | 83248 | 83267 | ATTCAAGTATTTCCTCTGAC | 35 | 1605 |
| 1316201 | N/A | N/A | 45473 | 45492 | TGGTTTCTAATACGAACTAC | 83 | 1606 |
| 1316271 | N/A | N/A | 118067 | 118086 | CTCATTTATACTACCTCCTA | 80 | 1607 |
| 1316276 | N/A | N/A | 98411 | 98430 | CAACCATTCTTTCATGCATT | 86 | 1608 |
| 1316370 | N/A | N/A | 13495 | 13514 | ACATTATTCATTTCAGTGTC | 55 | 1609 |
| 1316373 | N/A | N/A | 115514 | 115533 | ACAGCAACAAATATTTTCAA | 68 | 1610 |
| 1316402 | N/A | N/A | 8712 | 8731 | AGGTATTTTATTTTTCCTCC | 48 | 1611 |
| 1316462 | N/A | N/A | 50465 | 50484 | ACATTAATTTTTCCTAGAAT | 96 | 1612 |
| 1316474 | N/A | N/A | 26487 | 26506 | GCCACAAAAACATTGATCCC | 79 | 1613 |
| 1316481 | N/A | N/A | 59983 | 60002 | CAGTTGTGACTTATGAGCCA | 66 | 1614 |
| 1316506 | 3138 | 3157 | 69111 | 69130 | AAGGTTATTTTCCATAGTGA | 22 | 1615 |
| 1316514 | N/A | N/A | 112799 | 112818 | GCAAATCATCATAATCATCT | 68 | 1616 |
| 1316567 | N/A | N/A | 22381 | 22400 | TGTCTGATATTAACAGAACC | 83 | 1617 |
| 1316610 | N/A | N/A | 110832 | 110851 | TGGTTTTGCTTTCATGCTGC | 88 | 1618 |
| 1316647 | N/A | N/A | 46044 | 46063 | CCACATTTCCTTCACCTCAT | 87 | 1619 |
| 1316690 | N/A | N/A | 134914 | 134933 | TGTTTAAACCTTTTTCTTCA | 76 | 1620 |
| 1316724 | N/A | N/A | 144454 | 144473 | CAGTTCATTTTAGAAAACCC | 47 | 1621 |
| 1316739 | N/A | N/A | 131464 | 131483 | GTTTTATTTTCCTCCGTTTA | 82 | 1622 |
| 1316878 | N/A | N/A | 100616 | 100635 | CATCAATGATATCCCTTCTA | 78 | 1623 |
| 1316883 | N/A | N/A | 4638 | 4657 | GTCTATGTCCTTCATTAGAA | 49 | 1624 |
| 1316955 | N/A | N/A | 158714 | 158733 | TCTTCACTTTCAATCTGCTC | 43 | 1625 |
| 1317071 | 11139 | 11158 | 167888 | 167907 | GTTCCAACATTTCCTCCACT | 58 | 1626 |
| 1317098 | N/A | N/A | 41155 | 41174 | TCGATCGGAAATATCTGCAA | 87 | 1627 |
| 1317158 | N/A | N/A | 38809 | 38828 | AGAACACTTAATCCATGGTC | 75 | 1628 |

TABLE 23
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 36 | 858 |
| 1314607 | N/A | N/A | 19197 | 19216 | CTCTCCACATTCATTTTCCT | 70 | 1629 |
| 1314611 | N/A | N/A | 65256 | 65275 | TGTTCATTAAACATGTATTA | 64 | 1630 |
| 1314614 | N/A | N/A | 44660 | 44679 | GGTTTAGTAATTATTTGCTT | 61 | 1631 |
| 1314615 | N/A | N/A | 77963 | 77982 | ACCTCAAATCATCATCAGGC | 95 | 1632 |
| 1314719 | N/A | N/A | 34457 | 34476 | ACACATATTCCTTTATTTTA | 91 | 1633 |
| 1314746 | N/A | N/A | 22104 | 22123 | TCACCATTTTTATTATTCTA | 114 | 1634 |
| 1314772 | N/A | N/A | 48777 | 48796 | GGCAGATTAACATTTCCTGC | 74 | 1635 |
| 1314800 | N/A | N/A | 134912 | 134931 | TTTAAACCTTTTTCTTCACA | 89 | 1636 |
| 1314803 | N/A | N/A | 109841 | 109860 | GTAACATCAATATCACATGC | 64 | 1637 |
| 1314837 | N/A | N/A | 141281 | 141300 | GCCTAGATTCCTTAGAACCA | 66 | 1638 |
| 1314859 | N/A | N/A | 98375 | 98394 | CACTCATTAATTCAATAAAC | 107 | 1639 |
| 1314900 | N/A | N/A | 15242 | 15261 | ATATTTTTATTTTCAGGACA | 80 | 1640 |
| 1314903 | 5778 | 5797 | 130361 | 130380 | CAAGTCAGAATCCTCCTCTT | 76 | 1641 |
| 1314905 | N/A | N/A | 8630 | 8649 | GGTTGACTTATTTACAGGAA | 9 | 1642 |
| 1314909 | N/A | N/A | 19939 | 19958 | TGTGATTGCATTTAGTGCCT | 64 | 1643 |
| 1315012 | N/A | N/A | 122518 | 122537 | GAGAAGGATTCATCACTCTC | 72 | 1644 |
| 1315016 | N/A | N/A | 118961 | 118980 | TCCAAAATTCTAACTGCTGT | 65 | 1645 |
| 1315020 | N/A | N/A | 118006 | 118025 | AAGCCTCTATATTTTGGTCT | 69 | 1646 |
| 1315034 | N/A | N/A | 18228 | 18247 | GCACCAATATCATTACACAC | 69 | 1647 |
| 1315064 | N/A | N/A | 149098 | 149117 | TGCCTTTTCATTTTAATTCA | 89 | 1648 |
| 1315106 | N/A | N/A | 133576 | 133595 | GCCATGCTGCTCTAATAGAC | 66 | 1649 |
| 1315282 | N/A | N/A | 17581 | 17600 | TATCATTTCCCTCATTTCCC | 42 | 1650 |
| 1315323 | N/A | N/A | 67762 | 67781 | CATGTGTTTTTTAAAGCAGT | 61 | 1651 |
| 1315343 | N/A | N/A | 126800 | 126819 | GTCACAGCTTTTATTTCCAT | 19 | 1652 |
| 1315351 | N/A | N/A | 22941 | 22960 | CAAAAGACTTTCAAATTTCC | 87 | 1653 |
| 1315374 | N/A | N/A | 83247 | 83266 | TTCAAGTATTTCCTCTGACC | 44 | 1654 |
| 1315433 | N/A | N/A | 24461 | 24480 | GGTCTTTGAAATGATAGATC | 86 | 1655 |
| 1315462 | N/A | N/A | 117318 | 117337 | ACCTTTTTAATATCAAGGGC | 94 | 1656 |
| 1315495 | N/A | N/A | 22378 | 22397 | CTGATATTAACAGAACCAGT | 79 | 1657 |
| 1315503 | N/A | N/A | 13351 | 13370 | TACAAAGTTCTTATTTTCTC | 89 | 1658 |
| 1315515 | 868 | 887 | 33698 | 33717 | TGTCATTTGCAAAATTGCCA | 64 | 1659 |
| 1315629 | N/A | N/A | 69010 | 69029 | ACATATATTTTTAAAGATCA | 98 | 1660 |
| 1315704 | N/A | N/A | 59956 | 59975 | TGGAAAGGTTCAACATGCTA | 92 | 1661 |
| 1315724 | N/A | N/A | 81127 | 81146 | CCAAATGTAAATATTCCATT | 77 | 1662 |
| 1315765 | N/A | N/A | 100615 | 100634 | ATCAATGATATCCCTTCTAA | 80 | 1663 |
| 1315782 | N/A | N/A | 41143 | 41162 | ATCTGCAATTTAACTGGACA | 84 | 1664 |
| 1315863 | N/A | N/A | 128650 | 128669 | TTGCAATTCAAATATGAGCA | 59 | 1665 |
| 1315916 | N/A | N/A | 26475 | 26494 | TTGATCCCATTTTATGGTTA | 86 | 1666 |
| 1315954 | N/A | N/A | 158626 | 158645 | GGACACAGAATTCCCATGGA | 74 | 1667 |
| 1315982 | N/A | N/A | 45456 | 45475 | TACGCGCCAAACAGCAATCA | 99 | 1668 |
| 1315985 | N/A | N/A | 52693 | 52712 | TCCTCATCTTTTATAAAGTA | 79 | 1669 |
| 1315994 | N/A | N/A | 10344 | 10363 | CCATCCCTAAAACTGTGCCA | 71 | 1670 |
| 1316045 | N/A | N/A | 34972 | 34991 | AAGGAAGTAATTAACGGATT | 79 | 1671 |
| 1316056 | N/A | N/A | 38581 | 38600 | GGTATGTACATTTCCTGTCA | 59 | 1672 |
| 1316057 | N/A | N/A | 107166 | 107185 | GGATACACACCAACAACACC | 69 | 1673 |
| 1316116 | N/A | N/A | 106764 | 106783 | CATGGAAATTACATTTGGAA | 101 | 1674 |
| 1316130 | N/A | N/A | 60909 | 60928 | ATAACATCATTCCTTGCATC | 87 | 1675 |
| 1316229 | N/A | N/A | 50454 | 50473 | TCCTAGAATCCTTTTGGCTT | 41 | 1676 |
| 1316257 | N/A | N/A | 143913 | 143932 | CTGCAGATTCTACCTTCATA | 75 | 1677 |
| 1316278 | N/A | N/A | 58828 | 58847 | AGCCATGGAACAATTCCCTA | 99 | 1678 |
| 1316288 | N/A | N/A | 31572 | 31591 | GCATTTTCTTTCCTTGTTAC | 75 | 1679 |
| 1316331 | N/A | N/A | 12365 | 12384 | TACTCTATATATACCAGGCT | 74 | 1680 |
| 1316389 | N/A | N/A | 72164 | 72183 | GGGAAAGCAATCATTCTAGA | 56 | 1681 |
| 1316609 | N/A | N/A | 72997 | 73016 | AGGCATGTCACAATACATTT | 93 | 1682 |
| 1316628 | N/A | N/A | 55577 | 55596 | GTAAAAGCAACTCCATTTCT | 69 | 1683 |
| 1316677 | N/A | N/A | 56443 | 56462 | GAACTCAGAAAACAGTATCA | 62 | 1684 |
| 1316683 | N/A | N/A | 16942 | 16961 | TCTTTCAGAAACATTAACAG | 83 | 1685 |
| 1316700 | N/A | N/A | 4262 | 4281 | AGATACTTTCTAAATTGCTT | 53 | 1686 |
| 1316708 | N/A | N/A | 70319 | 70338 | ATATTGTTAAAAATTCTCCA | 79 | 1687 |
| 1316727 | N/A | N/A | 53086 | 53105 | CCAGTCCTATTCTCCTGGAA | 91 | 1688 |
| 1316743 | 6603 | 6622 | 138270 | 138289 | AGAAATTTCACTCATCCCTA | 59 | 1689 |
| 1316764 | N/A | N/A | 103067 | 103086 | AGCTCAATAAATCTTTATCA | 84 | 1690 |
| 1316855 | N/A | N/A | 112730 | 112749 | AAGCAAACATTTTTACAACA | 86 | 1691 |
| 1316871 | N/A | N/A | 46040 | 46059 | ATTTCCTTCACCTCATCTGC | 91 | 1692 |
| 1316879 | N/A | N/A | 115513 | 115532 | CAGCAACAAATATTTTCAAC | 72 | 1693 |
| 1316903 | 11138 | 11157 | 167887 | 167906 | TTCCAACATTTCCTCCACTT | 69 | 1694 |
| 1316949 | N/A | N/A | 110735 | 110754 | CTCAAGGCTATTCTCAACTC | 91 | 1695 |
| 1316976 | N/A | N/A | 104955 | 104974 | TTGAATTTTTTTCCCTGAGA | 88 | 1696 |
| 1316984 | N/A | N/A | 134190 | 134209 | TTTAACTTATTCTGCAGTCA | 32 | 1697 |
| 1317000 | N/A | N/A | 43184 | 43203 | GTTCAAAAAATCCAATTGCT | 79 | 1698 |
| 1317054 | N/A | N/A | 93976 | 93995 | GACTTATTAATAATTTACTT | 98 | 1699 |
| 1317055 | N/A | N/A | 131436 | 131455 | TTTGTAAATTCTCCATGGTA | 87 | 1700 |
| 1317069 | N/A | N/A | 73990 | 74009 | TTCTCTTACAATCAGTGTCT | 65 | 1701 |
| 1317082 | N/A | N/A | 2565 | 2584 | CCAGAGTACTTCCTAACTCC | 88 | 1702 |
| 1317086 | N/A | N/A | 47189 | 47208 | CAGCTTGACACACATTTAAA | 96 | 1703 |
| 1317218 | N/A | N/A | 80807 | 80826 | TGTATTATATTTATTTCTCT | 84 | 1704 |
| 1317224 | N/A | N/A | 6173 | 6192 | TCAGTCATTCCTCACACTGC | 90 | 1705 |

TABLE 24
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 29 | 858 |
| 1314516 | N/A | N/A | 77962 | 77981 | CCTCAAATCATCATCAGGCA | 78 | 1706 |
| 1314578 | N/A | N/A | 107165 | 107184 | GATACACACCAACAACACCT | 81 | 1707 |
| 1314604 | N/A | N/A | 128216 | 128235 | GCAGCAGGAATTTCTGCAAC | 83 | 1708 |
| 1314629 | N/A | N/A | 48775 | 48794 | CAGATTAACATTTCCTGCTC | 82 | 1709 |
| 1314665 | N/A | N/A | 17573 | 17592 | CCCTCATTTCCCTAATACAT | 83 | 1710 |
| 1314742 | N/A | N/A | 58822 | 58841 | GGAACAATTCCCTACTATCA | 91 | 1711 |
| 1314748 | N/A | N/A | 12326 | 12345 | GTTAACTGACTACAATCAAC | 78 | 1712 |
| 1314775 | N/A | N/A | 22358 | 22377 | CTAGCTTGCATCTAATTCTT | 81 | 1713 |
| 1314813 | N/A | N/A | 8629 | 8648 | GTTGACTTATTTACAGGAAA | 20 | 1714 |
| 1314835 | N/A | N/A | 112729 | 112748 | AGCAAACATTTTTACAACAA | 58 | 1715 |
| 1314862 | N/A | N/A | 131319 | 131338 | ACAAAGAACATCTAACACCT | 73 | 1716 |
| 1314893 | N/A | N/A | 133545 | 133564 | AGTGTGTTTGTAACACCTAA | 90 | 1717 |
| 1314978 | N/A | N/A | 117976 | 117995 | GATGATGTAACCATATAATC | 66 | 1718 |
| 1314992 | N/A | N/A | 31509 | 31528 | TGGTAACTACTTTAAGGTCT | 71 | 1719 |
| 1315056 | N/A | N/A | 4246 | 4265 | GCTTTCATCAATCTTACTTT | 19 | 1720 |
| 1315058 | N/A | N/A | 72161 | 72180 | AAAGCAATCATTCTAGAGCA | 86 | 1721 |
| 1315062 | N/A | N/A | 117317 | 117336 | CCTTTTTAATATCAAGGGCT | 98 | 1722 |
| 1315086 | N/A | N/A | 34455 | 34474 | ACATATTCCTTTATTTTACT | 90 | 1723 |
| 1315092 | N/A | N/A | 100569 | 100588 | ATGCACACACTCACATGCTT | 91 | 1724 |
| 1315112 | N/A | N/A | 19191 | 19210 | ACATTCATTTTCCTGGGATA | 74 | 1725 |
| 1315119 | N/A | N/A | 115509 | 115528 | AACAAATATTTTCAACTGGC | 17 | 1726 |
| 1315173 | N/A | N/A | 72995 | 73014 | GCATGTCACAATACATTTTA | 77 | 1727 |
| 1315258 | N/A | N/A | 18226 | 18245 | ACCAATATCATTACACACTT | 64 | 1728 |
| 1315266 | N/A | N/A | 83246 | 83265 | TCAAGTATTTCCTCTGACCT | 45 | 1729 |
| 1315303 | N/A | N/A | 80806 | 80825 | GTATTATATTTATTTCTCTA | 56 | 1730 |
| 1315360 | N/A | N/A | 134909 | 134928 | AAACCTTTTTCTTCACAGCT | 66 | 1731 |
| 1315381 | N/A | N/A | 26400 | 26419 | AGTGACATTTTCCCAGGAGC | 43 | 1732 |
| 1315418 | N/A | N/A | 110734 | 110753 | TCAAGGCTATTCTCAACTCA | 84 | 1733 |
| 1315460 | N/A | N/A | 55378 | 55397 | TAGGAAGTTCTAACATAGGC | 42 | 1734 |
| 1315535 | N/A | N/A | 149097 | 149116 | GCCTTTTCATTTTAATTCAA | 102 | 1735 |
| 1315540 | N/A | N/A | 6172 | 6191 | CAGTCATTCCTCACACTGCC | 89 | 1736 |
| 1315573 | N/A | N/A | 45998 | 46017 | ACAGCACTGCTTTCATACTA | 97 | 1737 |
| 1315597 | N/A | N/A | 70299 | 70318 | AAGCATCACACTATATACTA | 55 | 1738 |
| 1315602 | 5774 | 5793 | 130357 | 130376 | TCAGAATCCTCCTCTTCTCC | 74 | 1739 |
| 1315622 | N/A | N/A | 22103 | 22122 | CACCATTTTTATTATTCTAA | 108 | 1740 |
| 1315763 | N/A | N/A | 22939 | 22958 | AAAGACTTTCAAATTTCCCT | 71 | 1741 |
| 1315779 | N/A | N/A | 93885 | 93904 | ACAGTCATAATAATAAGGTT | 79 | 1742 |
| 1315783 | N/A | N/A | 104939 | 104958 | GAGAAGATTACAAATTGCTG | 63 | 1743 |
| 1315805 | N/A | N/A | 109839 | 109858 | AACATCAATATCACATGCCT | 70 | 1744 |
| 1315822 | N/A | N/A | 143890 | 143909 | GCCTATACCATACAATTCTC | 82 | 1745 |
| 1315827 | N/A | N/A | 103066 | 103085 | GCTCAATAAATCTTTATCAG | 59 | 1746 |
| 1315849 | N/A | N/A | 67687 | 67706 | AGCTGAGTTCCTAATTCAGA | 77 | 1747 |
| 1315894 | 11137 | 11156 | 167886 | 167905 | TCCAACATTTCCTCCACTTA | 72 | 1748 |
| 1315896 | N/A | N/A | 73959 | 73978 | CCAAAGCCATTATCTTGCCT | 64 | 1749 |
| 1315924 | N/A | N/A | 118942 | 118961 | TGCTGCTATCCCTTGCAGCA | 113 | 1750 |
| 1315936 | N/A | N/A | 98237 | 98256 | ACACAACTATGATACAGCAA | 88 | 1751 |
| 1316033 | N/A | N/A | 42656 | 42675 | AGTCCACACCTCTAACTCTC | 85 | 1752 |
| 1316066 | N/A | N/A | 44659 | 44678 | GTTTAGTAATTATTTGCTTT | 90 | 1753 |
| 1316142 | N/A | N/A | 45447 | 45466 | AACAGCAATCATCCTAGAAT | 82 | 1754 |
| 1316184 | N/A | N/A | 59893 | 59912 | GGTTGAGTAAATAATCCCTA | 46 | 1755 |
| 1316223 | N/A | N/A | 52691 | 52710 | CTCATCTTTTATAAAGTACA | 79 | 1756 |
| 1316240 | N/A | N/A | 34941 | 34960 | AAAGTGTTAGCATCATCATA | 63 | 1757 |
| 1316274 | N/A | N/A | 158314 | 158333 | GCACGGATTTCACAACGGTG | 34 | 1758 |
| 1316319 | N/A | N/A | 50425 | 50444 | GTTTCACTAAAATCTCAAAC | 55 | 1759 |
| 1316340 | 763 | 782 | 33593 | 33612 | GCAGAAGGTTCACCAGGTAA | 54 | 1760 |
| 1316347 | N/A | N/A | 122516 | 122535 | GAAGGATTCATCACTCTCTC | 89 | 1761 |
| 1316361 | N/A | N/A | 10316 | 10335 | AAACATAGAATCATACAGTA | 91 | 1762 |
| 1316520 | N/A | N/A | 15227 | 15246 | GGACATATGCTCATTTGTTA | 38 | 1763 |
| 1316528 | N/A | N/A | 134175 | 134194 | AGTCATGGAAATACACTCAC | 51 | 1764 |
| 1316558 | N/A | N/A | 56406 | 56425 | AGCAAAGATATAATTAAGAT | 92 | 1765 |
| 1316564 | N/A | N/A | 106748 | 106767 | GGAAAACTGCTCTCAACATC | 83 | 1766 |
| 1316571 | N/A | N/A | 68944 | 68963 | GTCTCAACTTTTAAGTGAAA | 56 | 1767 |
| 1316737 | N/A | N/A | 41126 | 41145 | ACATGACCGCATACTACCCA | 75 | 1768 |
| 1316755 | N/A | N/A | 38404 | 38423 | AAGCATATATTTACACGAAC | 85 | 1769 |
| 1316770 | N/A | N/A | 24392 | 24411 | AGCTACACTTTTATATTTCC | 67 | 1770 |
| 1316802 | N/A | N/A | 126799 | 126818 | TCACAGCTTTTATTTCCATA | 17 | 1771 |
| 1316830 | N/A | N/A | 19905 | 19924 | AGATAGCCCCCTCATCTCAA | 79 | 1772 |
| 1316851 | N/A | N/A | 53071 | 53090 | TGGAAGATAAACCTGTAACA | 73 | 1773 |
| 1316931 | N/A | N/A | 138040 | 138059 | TTTGAACAATTTCATTCAAC | 77 | 1774 |
| 1316942 | N/A | N/A | 16926 | 16945 | ACAGAAGTTTTTATAAAGTT | 84 | 1775 |
| 1316953 | N/A | N/A | 141280 | 141299 | CCTAGATTCCTTAGAACCAA | 58 | 1776 |
| 1317081 | N/A | N/A | 47187 | 47206 | GCTTGACACACATTTAAAAA | 86 | 1777 |
| 1317091 | N/A | N/A | 2564 | 2583 | CAGAGTACTTCCTAACTCCT | 92 | 1778 |
| 1317104 | N/A | N/A | 81122 | 81141 | TGTAAATATTCCATTTCCAA | 56 | 1779 |
| 1317117 | N/A | N/A | 13346 | 13365 | AGTTCTTATTTTCTCTAGAC | 52 | 1780 |
| 1317205 | N/A | N/A | 64817 | 64836 | GGCACAATCACATTCTGGTA | 19 | 1781 |
| 1317230 | N/A | N/A | 60882 | 60901 | CCCAGAACTTTCAATTGAGC | 76 | 1782 |

TABLE 25
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 31 | 858 |
| 1314562 | N/A | N/A | 15211 | 15230 | GTTATTTCATTTTCTTCCCA | 36 | 1783 |
| 1314576 | N/A | N/A | 48774 | 48793 | AGATTAACATTTCCTGCTCA | 76 | 1784 |
| 1314601 | N/A | N/A | 31458 | 31477 | GTTACCATTTTTAATTCTCA | 72 | 1785 |
| 1314642 | N/A | N/A | 109838 | 109857 | ACATCAATATCACATGCCTC | 72 | 1786 |
| 1314644 | N/A | N/A | 110729 | 110748 | GCTATTCTCAACTCAGGAAT | 91 | 1787 |
| 1314661 | N/A | N/A | 77961 | 77980 | CTCAAATCATCATCAGGCAT | 81 | 1788 |
| 1314724 | N/A | N/A | 134908 | 134927 | AACCTTTTTCTTCACAGCTA | 72 | 1789 |
| 1314779 | N/A | N/A | 33554 | 33573 | ACGCAAGTCATTCTTCCAGC | 91 | 1790 |
| 1314823 | N/A | N/A | 98187 | 98206 | TTGTCTTAAAATAATCTGCA | 81 | 1791 |
| 1314834 | N/A | N/A | 59871 | 59890 | GCTCACTTTAACTCTTACCA | 57 | 1792 |
| 1314855 | N/A | N/A | 112728 | 112747 | GCAAACATTTTTACAACAAC | 52 | 1793 |
| 1314940 | N/A | N/A | 115508 | 115527 | ACAAATATTTTCAACTGGCA | 37 | 1794 |
| 1314954 | N/A | N/A | 133541 | 133560 | TGTTTGTAACACCTAACAAC | 98 | 1795 |
| 1314970 | N/A | N/A | 149096 | 149115 | CCTTTTCATTTTAATTCAAC | 90 | 1796 |
| 1314981 | N/A | N/A | 128194 | 128213 | GATACAGCAATTTTATGGCT | 69 | 1797 |
| 1315063 | N/A | N/A | 13345 | 13364 | GTTCTTATTTTCTCTAGACA | 68 | 1798 |
| 1315091 | N/A | N/A | 80782 | 80801 | ACCAAGTTACTTAAATCCTA | 42 | 1799 |
| 1315097 | N/A | N/A | 58808 | 58827 | CTATCAACTTTCTAACTCGA | 80 | 1800 |
| 1315131 | N/A | N/A | 24391 | 24410 | GCTACACTTTTATATTTCCT | 77 | 1801 |
| 1315171 | N/A | N/A | 19174 | 19193 | ATAACTATTCTTCTTTGCCT | 80 | 1802 |
| 1315379 | N/A | N/A | 67686 | 67705 | GCTGAGTTCCTAATTCAGAA | 61 | 1803 |
| 1315413 | N/A | N/A | 6130 | 6149 | GACAGAACCCACCATGCTTA | 76 | 1804 |
| 1315414 | N/A | N/A | 16906 | 16925 | TTTGCATATTTATCGATTCC | 38 | 1805 |
| 1315466 | N/A | N/A | 41082 | 41101 | GGAATACTTTCTCATGGGCC | 72 | 1806 |
| 1315473 | N/A | N/A | 68920 | 68939 | CTGAAAATAAATTTTGATCA | 92 | 1807 |
| 1315482 | N/A | N/A | 70297 | 70316 | GCATCACACTATATACTAGC | 25 | 1808 |
| 1315505 | N/A | N/A | 19883 | 19902 | TCCTGAATTTAATCACATCA | 82 | 1809 |
| 1315534 | N/A | N/A | 44646 | 44665 | TTGCTTTTCCCATCAGACCA | 76 | 1810 |
| 1315546 | N/A | N/A | 117975 | 117994 | ATGATGTAACCATATAATCC | 75 | 1811 |
| 1315644 | N/A | N/A | 107164 | 107183 | ATACACACCAACAACACCTC | 95 | 1812 |
| 1315670 | N/A | N/A | 126796 | 126815 | CAGCTTTTATTTCCATACAT | 40 | 1813 |
| 1315771 | N/A | N/A | 47162 | 47181 | AAACACTATTATGAGTGCCA | 71 | 1814 |
| 1315829 | N/A | N/A | 22931 | 22950 | TCAAATTTCCCTTTTGACTT | 76 | 1815 |
| 1315841 | N/A | N/A | 81121 | 81140 | GTAAATATTCCATTTCCAAT | 66 | 1816 |
| 1315848 | N/A | N/A | 133958 | 133977 | ACCTGATAACCTATTTTCCC | 69 | 1817 |
| 1315901 | N/A | N/A | 143889 | 143908 | CCTATACCATACAATTCTCC | 97 | 1818 |
| 1315944 | N/A | N/A | 26339 | 26358 | CGTGCAGGCACAATTGTCAA | 70 | 1819 |
| 1315952 | N/A | N/A | 100565 | 100584 | ACACACTCACATGCTTATTA | 59 | 1820 |
| 1315960 | 11136 | 11155 | 167885 | 167904 | CCAACATTTCCTCCACTTAC | 67 | 1821 |
| 1315996 | N/A | N/A | 22354 | 22373 | CTTGCATCTAATTCTTGTAA | 82 | 1822 |
| 1316019 | N/A | N/A | 53062 | 53081 | AACCTGTAACACATTTTCCT | 78 | 1823 |
| 1316077 | N/A | N/A | 12322 | 12341 | ACTGACTACAATCAACATAC | 89 | 1824 |
| 1316083 | N/A | N/A | 42627 | 42646 | GCTTCTCTTCCAAATTAATC | 91 | 1825 |
| 1316099 | N/A | N/A | 130251 | 130270 | GCAGTGAGCTTATTTGCAAA | 79 | 1826 |
| 1316115 | N/A | N/A | 122399 | 122418 | GCCACCACTAATCCCATGGT | 93 | 1827 |
| 1316118 | N/A | N/A | 93812 | 93831 | TAGATGATTTTTATTTCAGT | 69 | 1828 |
| 1316191 | N/A | N/A | 34937 | 34956 | TGTTAGCATCATCATACTCA | 61 | 1829 |
| 1316205 | N/A | N/A | 10299 | 10318 | GTATGTATAACCTTTTGGGA | 51 | 1830 |
| 1316214 | N/A | N/A | 141270 | 141289 | TTAGAACCAATTTTAGACAA | 72 | 1831 |
| 1316243 | N/A | N/A | 83245 | 83264 | CAAGTATTTCCTCTGACCTT | 40 | 1832 |
| 1316253 | N/A | N/A | 60881 | 60900 | CCAGAACTTTCAATTGAGCT | 57 | 1833 |
| 1316307 | N/A | N/A | 52690 | 52709 | TCATCTTTTATAAAGTACAC | 85 | 1834 |
| 1316318 | N/A | N/A | 118929 | 118948 | TGCAGCAGACCATCAGTTAC | 101 | 1835 |
| 1316413 | N/A | N/A | 8564 | 8583 | CACCAGCACATTCTACAGCC | 78 | 1836 |
| 1316418 | N/A | N/A | 45977 | 45996 | GGAGATTTCCATTTACATGC | 46 | 1837 |
| 1316464 | N/A | N/A | 45446 | 45465 | ACAGCAATCATCCTAGAATT | 98 | 1838 |
| 1316489 | N/A | N/A | 72052 | 72071 | AGTCACCAAAATCATTCTCA | 64 | 1839 |
| 1316519 | N/A | N/A | 38350 | 38369 | GGTGTCAACAAAACTTCGGA | 63 | 1840 |
| 1316541 | N/A | N/A | 138039 | 138058 | TTGAACAATTTCATTCAACA | 68 | 1841 |
| 1316546 | N/A | N/A | 64257 | 64276 | GTCTACACAAAATCTTGCAG | 50 | 1842 |
| 1316570 | N/A | N/A | 106743 | 106762 | ACTGCTCTCAACATCTACAA | 92 | 1843 |
| 1316613 | N/A | N/A | 17572 | 17591 | CCTCATTTCCCTAATACATA | 77 | 1844 |
| 1316629 | N/A | N/A | 2558 | 2577 | ACTTCCTAACTCCTACTGTA | 80 | 1845 |
| 1316657 | N/A | N/A | 157506 | 157525 | CCACCATGCCATTCTCACCA | 85 | 1846 |
| 1316780 | N/A | N/A | 56350 | 56369 | ACCTGTAGTCCACATACCCA | 77 | 1847 |
| 1316885 | N/A | N/A | 103064 | 103083 | TCAATAAATCTTTATCAGCA | 55 | 1848 |
| 1316887 | N/A | N/A | 72991 | 73010 | GTCACAATACATTTTATACC | 48 | 1849 |
| 1316909 | N/A | N/A | 131314 | 131333 | GAACATCTAACACCTTTTCT | 77 | 1850 |
| 1316922 | N/A | N/A | 4145 | 4164 | TAGTGAATACCTCCTTTCTC | 49 | 1851 |
| 1316937 | N/A | N/A | 34452 | 34471 | TATTCCTTTATTTTACTCTC | 68 | 1852 |
| 1316964 | N/A | N/A | 18225 | 18244 | CCAATATCATTACACACTTT | 54 | 1853 |
| 1316968 | N/A | N/A | 117297 | 117316 | TCCAACAGATCAACTGCTGA | 93 | 1854 |
| 1316979 | N/A | N/A | 104875 | 104894 | AGTACCTTCACATACACAGA | 81 | 1855 |
| 1317074 | N/A | N/A | 73939 | 73958 | TAGCATCAACATTAATCTCC | 70 | 1856 |
| 1317115 | N/A | N/A | 50380 | 50399 | GACGCAAATATTATAGATTC | 29 | 1857 |
| 1317128 | N/A | N/A | 55377 | 55396 | AGGAAGTTCTAACATAGGCT | 27 | 1858 |
| 1317174 | N/A | N/A | 22102 | 22121 | ACCATTTTTATTATTCTAAT | 95 | 1859 |

TABLE 26
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 32 | 858 |
| 1314490 | N/A | N/A | 22921 | 22940 | CTTTTGACTTCACTTCTGAC | 83 | 1860 |
| 1314518 | N/A | N/A | 107163 | 107182 | TACACACCAACAACACCTCC | 82 | 1861 |
| 1314531 | N/A | N/A | 112727 | 112746 | CAAACATTTTTACAACAACA | 96 | 1862 |
| 1314552 | N/A | N/A | 18222 | 18241 | ATATCATTACACACTTTCCT | 69 | 1863 |
| 1314555 | N/A | N/A | 33552 | 33571 | GCAAGTCATTCTTCCAGCTC | 113 | 1864 |
| 1314640 | N/A | N/A | 6122 | 6141 | CCACCATGCTTATCTGAGCA | 95 | 1865 |
| 1314662 | N/A | N/A | 19134 | 19153 | CCACTGTTACATATATTTCT | 71 | 1866 |
| 1314693 | N/A | N/A | 133957 | 133976 | CCTGATAACCTATTTTCCCA | 69 | 1867 |
| 1314710 | N/A | N/A | 110701 | 110720 | TCCTGCTAAAACCTACACCA | 94 | 1868 |
| 1314752 | N/A | N/A | 72990 | 73009 | TCACAATACATTTTATACCA | 75 | 1869 |
| 1314821 | N/A | N/A | 71905 | 71924 | CAGTAGTTCCTAAATCATCA | 65 | 1870 |
| 1314863 | N/A | N/A | 157438 | 157457 | GAAATGAGTTTCCATTGCTA | 44 | 1871 |
| 1314888 | N/A | N/A | 50377 | 50396 | GCAAATATTATAGATTCCAA | 26 | 1872 |
| 1314902 | N/A | N/A | 19882 | 19901 | CCTGAATTTAATCACATCAT | 72 | 1873 |
| 1315055 | N/A | N/A | 48749 | 48768 | CTGGCAAACATTTATGTTCA | 49 | 1874 |
| 1315057 | N/A | N/A | 44565 | 44584 | GCATATAGTTTCAATACTTT | 46 | 1875 |
| 1315157 | N/A | N/A | 52689 | 52708 | CATCTTTTATAAAGTACACA | 51 | 1876 |
| 1315194 | N/A | N/A | 47130 | 47149 | TACCAGACATTTTAAAGGCA | 85 | 1877 |
| 1315251 | N/A | N/A | 131313 | 131332 | AACATCTAACACCTTTTCTA | 90 | 1878 |
| 1315272 | N/A | N/A | 102989 | 103008 | CTGTACTGAATCTTAAGAAC | 89 | 1879 |
| 1315285 | N/A | N/A | 68907 | 68926 | TTGATCATTATTCCATAACA | 81 | 1880 |
| 1315380 | N/A | N/A | 8563 | 8582 | ACCAGCACATTCTACAGCCA | 68 | 1881 |
| 1315401 | N/A | N/A | 149095 | 149114 | CTTTTCATTTTAATTCAACC | 98 | 1882 |
| 1315476 | N/A | N/A | 24386 | 24405 | ACTTTTATATTTCCTAGCTG | 94 | 1883 |
| 1315486 | N/A | N/A | 70296 | 70315 | CATCACACTATATACTAGCA | 57 | 1884 |
| 1315539 | N/A | N/A | 31454 | 31473 | CCATTTTTAATTCTCATCTC | 102 | 1885 |
| 1315558 | N/A | N/A | 98130 | 98149 | ACAAGCATTTTTATCAACAT | 54 | 1886 |
| 1315569 | N/A | N/A | 45442 | 45461 | CAATCATCCTAGAATTGCTT | 107 | 1887 |
| 1315624 | N/A | N/A | 133540 | 133559 | GTTTGTAACACCTAACAACC | 86 | 1888 |
| 1315675 | N/A | N/A | 4144 | 4163 | AGTGAATACCTCCTTTCTCA | 49 | 1889 |
| 1315697 | N/A | N/A | 93811 | 93830 | AGATGATTTTTATTTCAGTA | 68 | 1890 |
| 1315735 | N/A | N/A | 134904 | 134923 | TTTTTCTTCACAGCTATCTT | 94 | 1891 |
| 1315781 | N/A | N/A | 55126 | 55145 | AATGTATTTAATACCAGCGA | 24 | 1892 |
| 1315788 | N/A | N/A | 26266 | 26285 | GCATCTCCAAACCATACAAT | 77 | 1893 |
| 1315803 | N/A | N/A | 83107 | 83126 | GCTTTGTGAGAAATCAGTTA | 34 | 1894 |
| 1315821 | N/A | N/A | 2555 | 2574 | TCCTAACTCCTACTGTACAC | 92 | 1895 |
| 1315852 | N/A | N/A | 128125 | 128144 | TCTAGTAATTCATTTTTCTC | 66 | 1896 |
| 1315959 | N/A | N/A | 22070 | 22089 | TTCATTAGCTATAATTCCTT | 93 | 1897 |
| 1315966 | N/A | N/A | 58806 | 58825 | ATCAACTTTCTAACTCGAAA | 85 | 1898 |
| 1316001 | N/A | N/A | 118881 | 118900 | AGACCAGCCCCACCTCCTCA | 86 | 1899 |
| 1316067 | N/A | N/A | 16841 | 16860 | GTTATATTTTCTAGCTGATT | 53 | 1900 |
| 1316107 | N/A | N/A | 117974 | 117993 | TGATGTAACCATATAATCCC | 83 | 1901 |
| 1316123 | N/A | N/A | 53061 | 53080 | ACCTGTAACACATTTTCCTA | 88 | 1902 |
| 1316224 | N/A | N/A | 80778 | 80797 | AGTTACTTAAATCCTATGTT | 74 | 1903 |
| 1316241 | N/A | N/A | 15207 | 15226 | TTTCATTTTCTTCCCACAAC | 99 | 1904 |
| 1316258 | N/A | N/A | 100559 | 100578 | TCACATGCTTATTAATACCC | 93 | 1905 |
| 1316264 | N/A | N/A | 56349 | 56368 | CCTGTAGTCCACATACCCAT | 90 | 1906 |
| 1316323 | N/A | N/A | 130244 | 130263 | GCTTATTTGCAAAATACTGA | 57 | 1907 |
| 1316324 | N/A | N/A | 117258 | 117277 | GTTTGCCTCATATTTGGACC | 32 | 1908 |
| 1316326 | N/A | N/A | 106741 | 106760 | TGCTCTCAACATCTACAAAA | 86 | 1909 |
| 1316344 | N/A | N/A | 34449 | 34468 | TCCTTTATTTTACTCTCAGA | 58 | 1910 |
| 1316396 | N/A | N/A | 64229 | 64248 | TCACAGTACCATAATTCATA | 36 | 1911 |
| 1316415 | N/A | N/A | 22333 | 22352 | CACAGTGAACTTCTTTCCGT | 77 | 1912 |
| 1316417 | N/A | N/A | 12320 | 12339 | TGACTACAATCAACATACCA | 72 | 1913 |
| 1316565 | N/A | N/A | 34936 | 34955 | GTTAGCATCATCATACTCAA | 48 | 1914 |
| 1316568 | N/A | N/A | 81114 | 81133 | TTCCATTTCCAATCAGAATT | 77 | 1915 |
| 1316585 | 11132 | 11151 | 167881 | 167900 | CATTTCCTCCACTTACCACA | 92 | 1916 |
| 1316601 | N/A | N/A | 115507 | 115526 | CAAATATTTTCAACTGGCAC | 54 | 1917 |
| 1316634 | N/A | N/A | 17547 | 17566 | TACACCCATCATAAATTCAT | 93 | 1918 |
| 1316654 | N/A | N/A | 59860 | 59879 | CTCTTACCATACTATGGGCC | 93 | 1919 |
| 1316713 | N/A | N/A | 41024 | 41043 | TGATGGCCATTTAAATCTTG | 95 | 1920 |
| 1316718 | N/A | N/A | 73938 | 73957 | AGCATCAACATTAATCTCCT | 67 | 1921 |
| 1316729 | N/A | N/A | 10278 | 10297 | TGGCTTTTTTCATTAGCATA | 54 | 1922 |
| 1316774 | N/A | N/A | 122319 | 122338 | CTGTTTTTATCCTCACAGAA | 105 | 1923 |
| 1316800 | N/A | N/A | 77713 | 77732 | GGCACTCTCCTTTCTCCCTC | 69 | 1924 |
| 1316809 | N/A | N/A | 143883 | 143902 | CCATACAATTCTCCCCATTT | 68 | 1925 |
| 1316854 | N/A | N/A | 45884 | 45903 | AGTATGTATCTTACACATCA | 75 | 1926 |
| 1316938 | 442 | 461 | 13271 | 13290 | GACAATGATTCACACGGTCT | 46 | 1927 |
| 1316973 | N/A | N/A | 42624 | 42643 | TCTCTTCCAAATTAATCGGT | 104 | 1928 |
| 1317001 | N/A | N/A | 104874 | 104893 | GTACCTTCACATACACAGAC | 74 | 1929 |
| 1317058 | N/A | N/A | 109830 | 109849 | ATCACATGCCTCTAAGTTTA | 79 | 1930 |
| 1317061 | N/A | N/A | 67659 | 67678 | ATCAAGTGATCATTTAAGCT | 27 | 1931 |
| 1317111 | N/A | N/A | 60880 | 60899 | CAGAACTTTCAATTGAGCTT | 73 | 1932 |
| 1317163 | N/A | N/A | 126795 | 126814 | AGCTTTTATTTCCATACATA | 32 | 1933 |
| 1317190 | N/A | N/A | 141233 | 141252 | GAGGTACCAATCTCTTGGTT | 55 | 1934 |
| 1317211 | N/A | N/A | 37845 | 37864 | GAGTAATTTCCTTCCAGGGA | 53 | 1935 |
| 1317219 | N/A | N/A | 138016 | 138035 | GCTTAGTAAAATATATATCT | 76 | 1936 |

TABLE 27
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 35 | 858 |
| 1314503 | N/A | N/A | 45374 | 45393 | CCTCTATTTTTCAATTTATT | 97 | 1937 |
| 1314508 | N/A | N/A | 10272 | 10291 | TTTTCATTAGCATAACTTCA | 69 | 1938 |
| 1314514 | N/A | N/A | 15206 | 15225 | TTCATTTTCTTCCCACAACA | 95 | 1939 |
| 1314523 | N/A | N/A | 83030 | 83049 | GCCTAAGATTCCTTAGTGGC | 114 | 1940 |
| 1314574 | N/A | N/A | 12319 | 12338 | GACTACAATCAACATACCAC | 88 | 1941 |
| 1314620 | N/A | N/A | 117243 | 117262 | GGACCATGACTTAATGCTGT | 39 | 1942 |
| 1314633 | N/A | N/A | 45816 | 45835 | ACAAATGTTATTCACTGCAA | 53 | 1943 |
| 1314636 | N/A | N/A | 22018 | 22037 | ACTATGTATCTTCATCACAA | 64 | 1944 |
| 1314650 | 433 | 452 | 13262 | 13281 | TCACACGGTCTTTCTTGGTA | 31 | 1945 |
| 1314672 | N/A | N/A | 107160 | 107179 | ACACCAACAACACCTCCTGC | 98 | 1946 |
| 1314685 | N/A | N/A | 130188 | 130207 | AGTTGTTTTAAATATTGCCA | 25 | 1947 |
| 1314694 | N/A | N/A | 133953 | 133972 | ATAACCTATTTTCCCACTCA | 77 | 1948 |
| 1314735 | N/A | N/A | 2298 | 2317 | GTACACCTCATTCATCCCTC | 46 | 1949 |
| 1314744 | N/A | N/A | 137926 | 137945 | GGTATGACAATTTTAATTCA | 33 | 1950 |
| 1314826 | N/A | N/A | 34447 | 34466 | CTTTATTTTACTCTCAGAGT | 94 | 1951 |
| 1314850 | N/A | N/A | 106740 | 106759 | GCTCTCAACATCTACAAAAT | 88 | 1952 |
| 1314901 | N/A | N/A | 70295 | 70314 | ATCACACTATATACTAGCAT | 53 | 1953 |
| 1314933 | N/A | N/A | 52672 | 52691 | ACAGAGAATTTCATTAGATA | 75 | 1954 |
| 1314937 | N/A | N/A | 80767 | 80786 | TCCTATGTTTCTACATGACA | 90 | 1955 |
| 1314939 | N/A | N/A | 104859 | 104878 | CAGACACACATGCAGAGCCT | 89 | 1956 |
| 1314951 | N/A | N/A | 6043 | 6062 | AGCCAGAGTTCACTTAAGAT | 71 | 1957 |
| 1314987 | N/A | N/A | 22919 | 22938 | TTTGACTTCACTTCTGACCC | 92 | 1958 |
| 1315068 | N/A | N/A | 72988 | 73007 | ACAATACATTTTATACCAGC | 54 | 1959 |
| 1315105 | N/A | N/A | 112716 | 112735 | ACAACAACACTGTATAGCTA | 96 | 1960 |
| 1315142 | N/A | N/A | 8562 | 8581 | CCAGCACATTCTACAGCCAC | 77 | 1961 |
| 1315160 | N/A | N/A | 19133 | 19152 | CACTGTTACATATATTTCTT | 72 | 1962 |
| 1315193 | N/A | N/A | 117973 | 117992 | GATGTAACCATATAATCCCA | 54 | 1963 |
| 1315241 | N/A | N/A | 33547 | 33566 | TCATTCTTCCAGCTCTCCTT | 106 | 1964 |
| 1315269 | N/A | N/A | 44497 | 44516 | CAGATATAAAATATACACTA | 103 | 1965 |
| 1315304 | N/A | N/A | 131312 | 131331 | ACATCTAACACCTTTTCTAC | 92 | 1966 |
| 1315399 | N/A | N/A | 47129 | 47148 | ACCAGACATTTTAAAGGCAG | 95 | 1967 |
| 1315442 | N/A | N/A | 53057 | 53076 | GTAACACATTTTCCTAGTTG | 54 | 1968 |
| 1315445 | N/A | N/A | 128119 | 128138 | AATTCATTTTTCTCATCTCA | 42 | 1969 |
| 1315446 | N/A | N/A | 58739 | 58758 | TCCAAATTCAGAAATGACTT | 84 | 1970 |
| 1315447 | N/A | N/A | 115478 | 115497 | GGCAGAGTTTCTACCGGGCA | 36 | 1971 |
| 1315617 | N/A | N/A | 19881 | 19900 | CTGAATTTAATCACATCATA | 94 | 1972 |
| 1315642 | N/A | N/A | 59579 | 59598 | GCCCATAGTCTTGAACTCCT | 67 | 1973 |
| 1315686 | N/A | N/A | 81081 | 81100 | AATCAGTTTTTTCACTCACT | 46 | 1974 |
| 1315742 | N/A | N/A | 37844 | 37863 | AGTAATTTCCTTCCAGGGAT | 90 | 1975 |
| 1315886 | N/A | N/A | 60864 | 60883 | GCTTAGTTCATGAAATCTAA | 34 | 1976 |
| 1315923 | N/A | N/A | 143387 | 143406 | AGAAGGACTTCTCATTGGAC | 47 | 1977 |
| 1315933 | 1974 | 1993 | 56308 | 56327 | CGAGGCTTCATCAGGAAGAA | 77 | 1978 |
| 1315946 | N/A | N/A | 141189 | 141208 | ACTTCAGATTTCATTCAGAT | 80 | 1979 |
| 1315957 | N/A | N/A | 118805 | 118824 | GTTTATGGTCACCCATATTA | 106 | 1980 |
| 1315986 | N/A | N/A | 17546 | 17565 | ACACCCATCATAAATTCATA | 115 | 1981 |
| 1316002 | N/A | N/A | 110696 | 110715 | CTAAAACCTACACCACATCA | 108 | 1982 |
| 1316090 | N/A | N/A | 22332 | 22351 | ACAGTGAACTTCTTTCCGTT | 74 | 1983 |
| 1316196 | N/A | N/A | 73934 | 73953 | TCAACATTAATCTCCTGAGT | 97 | 1984 |
| 1316270 | N/A | N/A | 41021 | 41040 | TGGCCATTTAAATCTTGATA | 97 | 1985 |
| 1316281 | N/A | N/A | 93765 | 93784 | ATAGTATTTTCCATTTTGCT | 64 | 1986 |
| 1316310 | N/A | N/A | 126794 | 126813 | GCTTTTATTTCCATACATAT | 34 | 1987 |
| 1316357 | N/A | N/A | 156630 | 156649 | GTAGCATTTCCACCATCTAC | 112 | 1988 |
| 1316431 | N/A | N/A | 149093 | 149112 | TTTCATTTTAATTCAACCTA | 119 | 1989 |
| 1316433 | N/A | N/A | 109708 | 109727 | GGCAACCTCAAGCTGTGCCA | 96 | 1990 |
| 1316515 | N/A | N/A | 42623 | 42642 | CTCTTCCAAATTAATCGGTT | 91 | 1991 |
| 1316579 | N/A | N/A | 18221 | 18240 | TATCATTACACACTTTCCTA | 84 | 1992 |
| 1316625 | N/A | N/A | 98129 | 98148 | CAAGCATTTTTATCAACATC | 95 | 1993 |
| 1316758 | N/A | N/A | 4138 | 4157 | TACCTCCTTTCTCAACAGCA | 55 | 1994 |
| 1316771 | 11128 | 11147 | 167877 | 167896 | TCCTCCACTTACCACATTCA | 95 | 1995 |
| 1316785 | N/A | N/A | 64198 | 64217 | ACATGAACTCTATAATATCC | 79 | 1996 |
| 1316796 | N/A | N/A | 133538 | 133557 | TTGTAACACCTAACAACCCA | 109 | 1997 |
| 1316833 | N/A | N/A | 34871 | 34890 | ATACCATCTATCATCTGAGC | 95 | 1998 |
| 1316874 | N/A | N/A | 16607 | 16626 | TTAACAAATTTTCCAGATGA | 102 | 1999 |
| 1316880 | N/A | N/A | 134899 | 134918 | CTTCACAGCTATCTTCTCAT | 76 | 2000 |
| 1316881 | N/A | N/A | 100557 | 100576 | ACATGCTTATTAATACCCCA | 47 | 2001 |
| 1316886 | N/A | N/A | 121393 | 121412 | GCTACACTCACCACTATCAA | 100 | 2002 |
| 1316889 | N/A | N/A | 50338 | 50357 | ACTTAATTCAATGAAGGCTA | 46 | 2003 |
| 1316905 | N/A | N/A | 67657 | 67676 | CAAGTGATCATTTAAGCTTA | 73 | 2004 |
| 1316943 | N/A | N/A | 24238 | 24257 | GTATATCTCAATTCAGACAC | 73 | 2005 |
| 1316946 | N/A | N/A | 71903 | 71922 | GTAGTTCCTAAATCATCACC | 68 | 2006 |
| 1316952 | N/A | N/A | 102912 | 102931 | GTCTTTGTTCTTACTAGAAT | 73 | 2007 |
| 1316959 | N/A | N/A | 48722 | 48741 | CAGCAGTAAGACTTCAGTCA | 56 | 2008 |
| 1317005 | N/A | N/A | 77686 | 77705 | CCTCTCAATCTTTCTTTTTC | 106 | 2009 |
| 1317010 | N/A | N/A | 31384 | 31403 | ACAGCATTTCCTTCTAACAT | 87 | 2010 |
| 1317112 | N/A | N/A | 68888 | 68907 | AATGGCTGAATTTATTCCCT | 49 | 2011 |
| 1317167 | N/A | N/A | 26106 | 26125 | TGCAAACCATACAATAGAGC | 95 | 2012 |
| 1317227 | N/A | N/A | 55125 | 55144 | ATGTATTTAATACCAGCGAA | 29 | 2013 |

TABLE 28
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 24 | 858 |
| 1314502 | N/A | N/A | 110691 | 110710 | ACCTACACCACATCAGCGCA | 100 | 2014 |
| 1314542 | 1963 | 1982 | 56297 | 56316 | CAGGAAGAATACCTGTGGCT | 46 | 2015 |
| 1314548 | N/A | N/A | 55067 | 55086 | GTTACACATATTTAACCACA | 19 | 2016 |
| 1314553 | N/A | N/A | 52296 | 52315 | TTCAAGTTAAACTATCCATA | 82 | 2017 |
| 1314605 | N/A | N/A | 13241 | 13260 | CTGAAAGTTCTTTCTTTCTA | 94 | 2018 |
| 1314621 | N/A | N/A | 2294 | 2313 | ACCTCATTCATCCCTCGGCA | 49 | 2019 |
| 1314663 | N/A | N/A | 22882 | 22901 | GGGTTTTTCCACATATCTGA | 60 | 2020 |
| 1314698 | N/A | N/A | 25930 | 25949 | TCCAAACCATACAAGAGCAC | 84 | 2021 |
| | | | 25951 | 25970 | | | |
| | | | 26062 | 26081 | | | |
| | | | 26083 | 26102 | | | |
| | | | 26150 | 26169 | | | |
| | | | 26171 | 26190 | | | |
| | | | 26238 | 26257 | | | |
| 1314704 | N/A | N/A | 106736 | 106755 | TCAACATCTACAAAATGCTT | 92 | 2022 |
| 1314795 | N/A | N/A | 149092 | 149111 | TTCATTTTAATTCAACCTAC | 85 | 2023 |
| 1314801 | N/A | N/A | 134897 | 134916 | TCACAGCTATCTTCTCATCA | 63 | 2024 |
| 1314964 | N/A | N/A | 72986 | 73005 | AATACATTTTATACCAGCTC | 30 | 2025 |
| 1314980 | N/A | N/A | 15205 | 15224 | TCATTTTCTTCCCACAACAT | 93 | 2026 |
| 1314986 | N/A | N/A | 22014 | 22033 | TGTATCTTCATCACAATCTA | 69 | 2027 |
| 1315003 | N/A | N/A | 127983 | 128002 | GATTTATTTTTTCCTTCCAC | 46 | 2028 |
| 1315090 | N/A | N/A | 53042 | 53061 | AGTTGACTTTCTAAATTACC | 76 | 2029 |
| 1315107 | N/A | N/A | 117970 | 117989 | GTAACCATATAATCCCAACA | 36 | 2030 |
| 1315177 | N/A | N/A | 117197 | 117216 | CTTTCTATTTTCTTGAGAGA | 85 | 2031 |
| 1315277 | N/A | N/A | 98128 | 98147 | AAGCATTTTTATCAACATCT | 52 | 2032 |
| 1315283 | N/A | N/A | 68886 | 68905 | TGGCTGAATTTATTCCCTCC | 61 | 2033 |
| 1315305 | N/A | N/A | 58723 | 58742 | ACTTCACTATAATAACCCTA | 66 | 2034 |
| 1315321 | N/A | N/A | 130187 | 130206 | GTTGTTTTAAATATTGCCAT | 11 | 2035 |
| 1315385 | N/A | N/A | 6029 | 6048 | TAAGATTGAAATTCAGATCA | 77 | 2036 |
| 1315392 | N/A | N/A | 34421 | 34440 | ATATGGGTCCCATTAAGGCT | 53 | 2037 |
| 1315420 | N/A | N/A | 100455 | 100474 | CATGGGTATTTTCCTTGACT | 63 | 2038 |
| 1315424 | N/A | N/A | 12318 | 12337 | ACTACAATCAACATACCACA | 101 | 2039 |
| 1315529 | N/A | N/A | 17542 | 17561 | CCATCATAAATTCATACAGA | 76 | 2040 |
| 1315575 | N/A | N/A | 19132 | 19151 | ACTGTTACATATATTTCTTC | 49 | 2041 |
| 1315636 | N/A | N/A | 48604 | 48623 | GCAGAAGTTTATCCATAGCC | 29 | 2042 |
| 1315661 | N/A | N/A | 30256 | 30275 | ATATACATTTACATGTACCT | 112 | 2043 |
| 1315691 | N/A | N/A | 50287 | 50306 | ACTGCAAATAATATTAGGCA | 34 | 2044 |
| 1315727 | N/A | N/A | 19870 | 19889 | CACATCATAAAACAATTTTC | 79 | 2045 |
| 1315814 | N/A | N/A | 81079 | 81098 | TCAGTTTTTTCACTCACTAC | 41 | 2046 |
| 1315869 | N/A | N/A | 156305 | 156324 | CCTGTCACTTACCATCCCAA | 78 | 2047 |
| 1315906 | N/A | N/A | 37202 | 37221 | CCAACATCCAACTAACCAAT | 82 | 2048 |
| 1315909 | N/A | N/A | 112707 | 112726 | CTGTATAGCTACATTTTTCC | 68 | 2049 |
| 1315921 | N/A | N/A | 67642 | 67661 | GCTTAGAAGTCATTTAGTCC | 6 | 2050 |
| 1316004 | N/A | N/A | 16585 | 16604 | TTGATGCTCATCCATACTTA | 73 | 2051 |
| 1316021 | N/A | N/A | 93764 | 93783 | TAGTATTTTCCATTTTGCTA | 77 | 2052 |
| 1316047 | N/A | N/A | 22306 | 22325 | CCTGTAGGTTATTTTTGCCT | 56 | 2053 |
| 1316078 | N/A | N/A | 33539 | 33558 | CCAGCTCTCCTTCAGGAGAA | 93 | 2054 |
| 1316121 | N/A | N/A | 64192 | 64211 | ACTCTATAATATCCATCTGA | 63 | 2055 |
| 1316145 | N/A | N/A | 70279 | 70298 | GCATATGTACATAATTTAAT | 72 | 2056 |
| 1316171 | N/A | N/A | 131308 | 131327 | CTAACACCTTTTCTACAAAT | 79 | 2057 |
| 1316186 | N/A | N/A | 47128 | 47147 | CCAGACATTTTAAAGGCAGT | 61 | 2058 |
| 1316372 | N/A | N/A | 77684 | 77703 | TCTCAATCTTTCTTTTTCTC | 86 | 2059 |
| 1316461 | N/A | N/A | 107158 | 107177 | ACCAACAACACCTCCTGCCA | 91 | 2060 |
| 1316584 | N/A | N/A | 18171 | 18190 | GGTCAGACATATAATAATCT | 45 | 2061 |
| 1316606 | N/A | N/A | 83029 | 83048 | CCTAAGATTCCTTAGTGGCC | 72 | 2062 |
| 1316607 | N/A | N/A | 44472 | 44491 | GACAAGGCTTTTCCTCCAAT | 38 | 2063 |
| 1316637 | N/A | N/A | 141147 | 141166 | CTGTTGGCCATCTCTCACCC | 89 | 2064 |
| 1316662 | N/A | N/A | 115458 | 115477 | GCACAGCTCCTACACTCTGA | 60 | 2065 |
| 1316666 | N/A | N/A | 34870 | 34889 | TACCATCTATCATCTGAGCT | 81 | 2066 |
| 1316686 | N/A | N/A | 24225 | 24244 | CAGACACTAAATTTTCATCA | 57 | 2067 |
| 1316694 | N/A | N/A | 133537 | 133556 | TGTAACACCTAACAACCCAA | 87 | 2068 |
| 1316720 | N/A | N/A | 143372 | 143391 | TGGACTTAAGCAATTAAGCC | 84 | 2069 |
| 1316721 | N/A | N/A | 109548 | 109567 | GCCAGGCCATTTCCTTCCAC | 81 | 2070 |
| 1316778 | N/A | N/A | 4051 | 4070 | TGCAGTTGATTTCAGAGCAT | 64 | 2071 |
| 1316791 | N/A | N/A | 121302 | 121321 | GGCACAGGATCTTTTAGAAC | 33 | 2072 |
| 1316823 | N/A | N/A | 42593 | 42612 | CAGTCATTCACATGCAGGGC | 33 | 2073 |
| 1316836 | N/A | N/A | 80718 | 80737 | ACGCTGTTTCCTACCTGCAC | 95 | 2074 |
| 1316919 | N/A | N/A | 45371 | 45390 | CTATTTTTCAATTTATTCCT | 92 | 2075 |
| 1316935 | N/A | N/A | 45777 | 45796 | GAGTCCAGTCACAATAACCT | 97 | 2076 |
| 1316978 | N/A | N/A | 118800 | 118819 | TGGTCACCCATATTACTCAT | 37 | 2077 |
| 1316996 | N/A | N/A | 126780 | 126799 | ACATATACAAATAATGGAGT | 40 | 2078 |
| 1317023 | N/A | N/A | 137925 | 137944 | GTATGACAATTTTAATTCAT | 56 | 2079 |
| 1317037 | N/A | N/A | 73882 | 73901 | TTGGATGAATGATCTTATTA | 71 | 2080 |
| 1317100 | 11122 | 11141 | 167871 | 167890 | ACTTACCACATTCAATACCC | 84 | 2081 |
| 1317106 | N/A | N/A | 133952 | 133971 | TAACCTATTTTCCCACTCAT | 97 | 2082 |
| 1317121 | N/A | N/A | 102713 | 102732 | TAGACGGATTTTCTTCGGAC | 39 | 2083 |
| 1317140 | N/A | N/A | 60844 | 60863 | TGGATGTGACTCACTTACCT | 111† | 2084 |
| 1317141 | N/A | N/A | 10243 | 10262 | TTGCATGCTATCAGTAGCTC | 44 | 2085 |
| 1317148 | N/A | N/A | 59495 | 59514 | GCCCAGCATATTCAACAGAC | 89 | 2086 |
| 1317172 | N/A | N/A | 104782 | 104801 | GCCAAGCACATCACGACACC | 63 | 2087 |
| 1317180 | N/A | N/A | 71902 | 71921 | TAGTTCCTAAATCATCACCA | 58 | 2088 |
| 1317198 | N/A | N/A | 41019 | 41038 | GCCATTTAAATCTTGATAAA | 99 | 2089 |
| 1317206 | N/A | N/A | 8488 | 8507 | TTCTAAAATATTACATAGCA | 100 | 2090 |

TABLE 29
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314527 | N/A | N/A | 34404 | 34423 | GCTTATTTTTCAGAATACGA | 41 | 2091 |
| 1314570 | N/A | N/A | 19128 | 19147 | TTACATATATTTCTTCCGTT | 32 | 2092 |
| 1314797 | N/A | N/A | 26052 | 26071 | ACAAGAGCACTTCCAAACCA | 85 | 2093 |
| | | | 26140 | 26159 | | | |
| 1314806 | N/A | N/A | 142925 | 142944 | AAGCATAATCATCTTCACCC | 41 | 2094 |
| 1314824 | N/A | N/A | 48603 | 48622 | CAGAAGTTTATCCATAGCCT | 26 | 2095 |
| 1314841 | N/A | N/A | 10209 | 10228 | CTGAGTAGTTTAACCATTCA | 59 | 2096 |
| 1314872 | 2750 | 2769 | 60811 | 60830 | GCAAGGGTTTCCAGAAGCTC | 731 | 2097 |
| 1314919 | N/A | N/A | 45776 | 45795 | AGTCCAGTCACAATAACCTA | 104 | 2098 |
| 1314982 | N/A | N/A | 126685 | 126704 | GTAAGCCTTTTTGAATGGTA | 30 | 2099 |
| 1314983 | N/A | N/A | 77634 | 77653 | TCTCTCTTTTTCCTGTGGGC | 55 | 2100 |
| 1315011 | N/A | N/A | 72985 | 73004 | ATACATTTTATACCAGCTCA | 41 | 2101 |
| 1315025 | N/A | N/A | 137869 | 137888 | TCAGTGATACACCAACCACC | 74 | 2102 |
| 1315075 | N/A | N/A | 33519 | 33538 | ACAGAAGCAATTCATTCCCT | 84 | 2103 |
| 1315088 | N/A | N/A | 81077 | 81096 | AGTTTTTTCACTCACTACAT | 62 | 2104 |
| 1315176 | 4740 | 4759 | 104727 | 104746 | TGTCACAGCCTTCCTTCCAC | 69 | 2105 |
| 1315179 | N/A | N/A | 6024 | 6043 | TTGAAATTCAGATCATGCTA | 60 | 2106 |
| 1315188 | N/A | N/A | 83013 | 83032 | GGCCAACAACTTCATGAGCA | 86 | 2107 |
| 1315190 | 1947 | 1966 | 56281 | 56300 | GGCTTCCTCATCTTCATCCT | 49 | 2108 |
| 1315234 | 666 | 685 | N/A | N/A | ACCATTCTTTTTAATTTCCT | 27 | 2109 |
| 1315278 | N/A | N/A | 19869 | 19888 | ACATCATAAAACAATTTTCC | 74 | 2110 |
| 1315301 | N/A | N/A | 68884 | 68903 | GCTGAATTTATTCCCTCCCT | 55 | 2111 |
| 1315320 | N/A | N/A | 112705 | 112724 | GTATAGCTACATTTTTCCCA | 69 | 2112 |
| 1315324 | N/A | N/A | 133950 | 133969 | ACCTATTTTCCCACTCATCA | 83 | 2113 |
| 1315325 | 3703 | 3722 | 80650 | 80669 | CTGGTTCTTTCTCCTTCCCC | 51 | 2114 |
| 1315403 | N/A | N/A | 58666 | 58685 | TAGAGATTCATCATATTGCC | 43 | 2115 |
| 1315429 | N/A | N/A | 107157 | 107176 | CCAACAACACCTCCTGCCAC | 68 | 2116 |
| 1315493 | 1212 | 1231 | N/A | N/A | CAGTTCATAAACCTGGACAA | 63 | 2117 |
| 1315583 | N/A | N/A | 34869 | 34888 | ACCATCTATCATCTGAGCTC | 107 | 2118 |
| 1315660 | N/A | N/A | 22261 | 22280 | GGCAGCAGCATATACCGAGT | 53 | 2119 |
| 1315693 | N/A | N/A | 15200 | 15219 | TTCTTCCCACAACATTTCCA | 68 | 2120 |
| 1315711 | N/A | N/A | 24156 | 24175 | CGTATACCATTTCCAACCAC | 60 | 2121 |
| 1315725 | N/A | N/A | 47110 | 47129 | GTATGCCATATTTATTAGCA | 80 | 2122 |
| 1315741 | N/A | N/A | 59493 | 59512 | CCAGCATATTCAACAGACTT | 67 | 2123 |
| 1315831 | N/A | N/A | 13194 | 13213 | TGTGTTGAATTACCCACTCC | 92 | 2124 |
| 1315864 | N/A | N/A | 121270 | 121289 | CGGGAAATACTTCACAGAGC | 48 | 2125 |
| 1315870 | N/A | N/A | 45314 | 45333 | TGCATATCTAAAATTATCAA | 88 | 2126 |
| 1315877 | N/A | N/A | 54999 | 55018 | GTAGCAAACATTATTAGACT | 17 | 2127 |
| 1315927 | N/A | N/A | 52293 | 52312 | AAGTTAAACTATCCATAGGC | 68 | 2128 |
| 1315989 | N/A | N/A | 127982 | 128001 | ATTTATTTTTTCCTTCCACC | 43 | 2129 |
| 1316005 | N/A | N/A | 18169 | 18188 | TCAGACATATAATAATCTAA | 76 | 2130 |
| 1316031 | N/A | N/A | 149091 | 149110 | TCATTTTAATTCAACCTACA | 111 | 2131 |
| 1316060 | N/A | N/A | 87154 | 87173 | GAGACTTTTAACTCAGGCTC | 68 | 2132 |
| 1316087 | N/A | N/A | 71901 | 71920 | AGTTCCTAAATCATCACCAT | 69 | 2133 |
| 1316093 | N/A | N/A | 134895 | 134914 | ACAGCTATCTTCTCATCAAT | 72 | 2134 |
| 1316098 | N/A | N/A | 16582 | 16601 | ATGCTCATCCATACTTAGGA | 33 | 2135 |
| 1316120 | N/A | N/A | 22879 | 22898 | TTTTTCCACATATCTGAGGC | 76 | 2136 |
| 1316155 | N/A | N/A | 102145 | 102164 | GTAATATTTCTAACTCACAA | 68 | 2137 |
| 1316176 | N/A | N/A | 64128 | 64147 | GCCAATAAAAATGAACAAAC | 78 | 2138 |
| 1316216 | N/A | N/A | 44471 | 44490 | ACAAGGCTTTTCCTCCAATT | 64 | 2139 |
| 1316219 | N/A | N/A | 98127 | 98146 | AGCATTTTTATCAACATCTA | 67 | 2140 |
| 1316292 | 11121 | 11140 | 167870 | 167889 | CTTACCACATTCAATACCCA | 109 | 2141 |
| 1316333 | N/A | N/A | 22010 | 22029 | TCTTCATCACAATCTACCTT | 74 | 2142 |
| 1316336 | N/A | N/A | 50286 | 50305 | CTGCAAATAATATTAGGCAT | 50 | 2143 |
| 1316377 | N/A | N/A | 41009 | 41028 | TCTTGATAAAAATATCCTAA | 93 | 2144 |
| 1316401 | 8300 | 8319 | 156196 | 156215 | GTCACATACATCAGCTCAAA | 48 | 2145 |
| 1316404 | N/A | N/A | 73872 | 73891 | GATCTTATTAAAATACTCAT | 66 | 2146 |
| 1316483 | N/A | N/A | 133536 | 133555 | GTAACACCTAACAACCCAAA | 86 | 2147 |
| 1316510 | N/A | N/A | 110690 | 110709 | CCTACACCACATCAGCGCAA | 89 | 2148 |
| 1316516 | N/A | N/A | 117149 | 117168 | GGCTTCTAGCTATTTAGTCA | 76 | 2149 |
| 1316518 | N/A | N/A | 70274 | 70293 | TGTACATAATTTAATTATTC | 100 | 2150 |
| 1316551 | N/A | N/A | 67530 | 67549 | ACAAGTCTTATCACTTCTGC | 20 | 2151 |
| 1316658 | N/A | N/A | 17539 | 17558 | TCATAAATTCATACAGATAC | 79 | 2152 |
| 1316691 | N/A | N/A | 141140 | 141159 | CCATCTCTCACCCTGAGGTT | 80 | 2153 |
| 1316709 | N/A | N/A | 12295 | 12314 | ACCTCAGTAAAATATAAGCA | 85 | 2154 |
| 1316710 | N/A | N/A | 117968 | 117987 | AACCATATAATCCCAACAAT | 77 | 2155 |
| 1316769 | N/A | N/A | 100453 | 100472 | TGGGTATTTTCCTTGACTCC | 82 | 2156 |
| 1316772 | N/A | N/A | 130155 | 130174 | TATGTGTAATATATACTCTC | 29 | 2157 |
| 1316857 | N/A | N/A | 131302 | 131321 | CCTTTTCTACAAATTAACTT | 96 | 2158 |
| 1316901 | N/A | N/A | 53041 | 53060 | GTTGACTTTCTAAATTACCA | 69 | 2159 |
| 1316904 | N/A | N/A | 108878 | 108897 | GGAGAACTGCTATCAAGGCA | 65 | 2160 |
| 1317006 | N/A | N/A | 118798 | 118817 | GTCACCCATATTACTCATGC | 47 | 2161 |
| 1317015 | N/A | N/A | 37074 | 37093 | GTACCTAGCACTTCACCTCC | 82 | 2162 |
| 1317041 | N/A | N/A | 2278 | 2297 | GGCAATTAACCTCCCCATCA | 51 | 2163 |
| 1317078 | 5486 | 5505 | 115364 | 115383 | TGGATCAGACACATTAGCAG | 54 | 2164 |
| 1317099 | N/A | N/A | 106700 | 106719 | ACCCTTACACATACTTGGTT | 88 | 2165 |
| 1317204 | N/A | N/A | 8344 | 8363 | TGCATATTAAAATCTGGGAA | 22 | 2166 |
| 1317210 | N/A | N/A | 4014 | 4033 | GTTATTATTCTAGCCAGGCA | 68 | 2167 |

TABLE 30
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 24 | 858 |
| 1314479 | N/A | N/A | 22878 | 22897 | TTTTCCACATATCTGAGGCT | 78 | 2168 |
| 1314486 | 2697 | 2716 | 60758 | 60777 | AGTCAGCACATCGATGATCA | 75 | 2169 |
| 1314487 | N/A | N/A | 10160 | 10179 | GTTCTTAGATATTATGGGTA | 17 | 2170 |
| 1314582 | N/A | N/A | 48405 | 48424 | CAGACGGATTTTCTCAAGTC | 84 | 2171 |
| 1314670 | N/A | N/A | 26046 | 26065 | GCACTTCCAAACCATACAAT | 65 | 2172 |
| | | | 26134 | 26153 | | | |
| 1314674 | N/A | N/A | 45772 | 45791 | CAGTCACAATAACCTAGACT | 90 | 2173 |
| 1314703 | 4739 | 4758 | 104726 | 104745 | GTCACAGCCTTCCTTCCACT | 52 | 2174 |
| 1314711 | N/A | N/A | 142923 | 142942 | GCATAATCATCTTCACCCAA | 42 | 2175 |
| 1314784 | N/A | N/A | 24155 | 24174 | GTATACCATTTCCAACCACA | 66 | 2176 |
| 1314840 | N/A | N/A | 47107 | 47126 | TGCCATATTTATTAGCACTT | 73 | 2177 |
| 1314868 | N/A | N/A | 118797 | 118816 | TCACCCATATTACTCATGCA | 63 | 2178 |
| 1314904 | N/A | N/A | 8261 | 8280 | GCTTTGTTTTTCATCTGTGC | 21 | 2179 |
| 1314929 | N/A | N/A | 52292 | 52311 | AGTTAAACTATCCATAGGCA | 65 | 2180 |
| 1314956 | N/A | N/A | 2254 | 2273 | CGTGTTGGTTTTATTCCCAA | 67 | 2181 |
| 1314962 | N/A | N/A | 106660 | 106679 | GGTTATCAATCATTATACCA | 85 | 2182 |
| 1314993 | N/A | N/A | 67526 | 67545 | GTCTTATCACTTCTGCAATC | 37 | 2183 |
| 1315080 | N/A | N/A | 130141 | 130160 | ACTCTCAGTTATCTATTCCT | 42 | 2184 |
| 1315208 | N/A | N/A | 13193 | 13212 | GTGTTGAATTACCCACTCCA | 88 | 2185 |
| 1315255 | N/A | N/A | 153568 | 153587 | GTGGCACTGTCCTTCAGCCC | 73 | 2186 |
| | | | 153636 | 153655 | | | |
| | | | 153670 | 153689 | | | |
| | | | 153704 | 153723 | | | |
| | | | 153738 | 153757 | | | |
| | | | 153772 | 153791 | | | |
| | | | 153806 | 153825 | | | |
| | | | 153840 | 153859 | | | |
| | | | 153874 | 153893 | | | |
| | | | 153908 | 153927 | | | |
| | | | 153942 | 153961 | | | |
| | | | 153976 | 153995 | | | |
| | | | 154010 | 154029 | | | |
| | | | 154044 | 154063 | | | |
| | | | 154078 | 154097 | | | |
| 1315280 | N/A | N/A | 22260 | 22279 | GCAGCAGCATATACCGAGTC | 54 | 2187 |
| 1315336 | N/A | N/A | 19114 | 19133 | TCCGTTTTCAACTATTTCAA | 54 | 2188 |
| 1315342 | N/A | N/A | 117135 | 117154 | TAGTCATTTATTTAAAGCAA | 77 | 2189 |
| 1315400 | N/A | N/A | 100416 | 100435 | TTGTAGCTATTTAATCGCTA | 94 | 2190 |
| 1315428 | N/A | N/A | 107008 | 107027 | CACATGGCACTTCCTTCCAT | 70 | 2191 |
| 1315439 | N/A | N/A | 45313 | 45332 | GCATATCTAAAATTATCAAT | 93 | 2192 |
| 1315500 | N/A | N/A | 72947 | 72966 | ATCAACTAACATCTTTCCTA | 98 | 2193 |
| 1315674 | N/A | N/A | 126670 | 126689 | TGGTAAAATAACATTTCCAA | 81 | 2194 |
| 1315681 | N/A | N/A | 34748 | 34767 | GAGCACCTACTCCCATTCCA | 69 | 2195 |
| 1315696 | N/A | N/A | 133527 | 133546 | AACAACCCAAAATAACCTCA | 80 | 2196 |
| 1315718 | N/A | N/A | 19868 | 19887 | CATCATAAAACAATTTTCCA | 105 | 2197 |
| 1315733 | N/A | N/A | 16581 | 16600 | TGCTCATCCATACTTAGGAA | 28 | 2198 |
| 1315737 | N/A | N/A | 133948 | 133967 | CTATTTTCCCACTCATCACA | 91 | 2199 |
| 1315740 | N/A | N/A | 15199 | 15218 | TCTTCCCACAACATTTCCAC | 84 | 2200 |
| 1315760 | N/A | N/A | 33515 | 33534 | AAGCAATTCATTCCCTGTTA | 82 | 2201 |
| 1315778 | 10934 | 10953 | 167683 | 167702 | CACTTCCATTTTAATGACTT | 82 | 2202 |
| 1315876 | N/A | N/A | 17526 | 17545 | CAGATACACACATACACTCT | 85 | 2203 |
| 1315922 | N/A | N/A | 121269 | 121288 | GGGAAATACTTCACAGAGCA | 32 | 2204 |
| 1315937 | N/A | N/A | 137723 | 137742 | GTTTAGCCATTTAAAGATTG | 82 | 2205 |
| 1315949 | N/A | N/A | 81076 | 81095 | GTTTTTTCACTCACTACATC | 72 | 2206 |
| 1315988 | N/A | N/A | 37032 | 37051 | GAAAAGTACATTCATAGGTT | 89 | 2207 |
| 1316061 | 5436 | 5455 | 115314 | 115333 | TTGCTGCTCACTCATTTCCA | 26 | 2208 |
| 1316132 | N/A | N/A | 3714 | 3733 | ATAGATATATATTATTCTAA | 116 | 2209 |
| 1316153 | N/A | N/A | 130780 | 130799 | TACTTTGTTTTTTAAATCAT | 95 | 2210 |
| 1316154 | N/A | N/A | 58665 | 58684 | AGAGATTCATCATATTGCCA | 25 | 2211 |
| 1316255 | N/A | N/A | 6012 | 6031 | TCATGCTACTCCTCTGCTCT | 64 | 2212 |
| 1316256 | N/A | N/A | 34403 | 34422 | CTTATTTTTCAGAATACGAA | 75 | 2213 |
| 1316262 | N/A | N/A | 68832 | 68851 | AGCCATACTCCCCTTACAGC | 36 | 2214 |
| 1316285 | N/A | N/A | 108824 | 108843 | ACCCACCTTTCACAAGGACC | 87 | 2215 |
| 1316286 | N/A | N/A | 44419 | 44438 | AGGTGTTAATTTACACAGTG | 84 | 2216 |
| 1316320 | N/A | N/A | 64097 | 64116 | GCTACAACAAGAACCAATCT | 75 | 2217 |
| 1316334 | N/A | N/A | 30175 | 30194 | GCCCACCTTTTTAATTTCCT | 90 | 2218 |
| 1316367 | N/A | N/A | 149088 | 149107 | TTTTAATTCAACCTACACTC | 98 | 2219 |
| 1316453 | N/A | N/A | 50263 | 50282 | GCTCTCAATTAAATACTCAA | 36 | 2220 |
| 1316459 | N/A | N/A | 134891 | 134910 | CTATCTTCTCATCAATACTC | 87 | 2221 |
| 1316492 | N/A | N/A | 97718 | 97737 | CCATTCTGAATGCCTAGATA | 107 | 2222 |
| 1316521 | N/A | N/A | 110505 | 110524 | CCCAGGTATCTCCTAGGCTT | 89 | 2223 |
| 1316543 | N/A | N/A | 53038 | 53057 | GACTTTCTAAATTACCATTT | 45 | 2224 |
| 1316582 | N/A | N/A | 77632 | 77651 | TCTCTTTTTCCTGTGGGCCA | 49 | 2225 |
| 1316604 | 1175 | 1194 | 42524 | 42543 | GAGACTTCCATTTCTTTCCT | 19 | 2226 |
| 1316611 | 3702 | 3721 | 80649 | 80668 | TGGTTCTTTCTCCTTCCCCT | 41 | 2227 |
| 1316646 | 1912 | 1931 | 56246 | 56265 | GCAGGCCCAAATACTGGTTG | 49 | 2228 |
| 1316651 | N/A | N/A | 73838 | 73857 | CACGAAACTCTCCTATAGCC | 85 | 2229 |
| 1316699 | N/A | N/A | 87068 | 87087 | GCTCTGTTACTTCCTCAGGC | 71 | 2230 |
| 1316760 | N/A | N/A | 71900 | 71919 | GTTCCTAAATCATCACCATT | 80 | 2231 |
| 1316818 | N/A | N/A | 70273 | 70292 | GTACATAATTTAATTATTCT | 87 | 2232 |
| 1316840 | N/A | N/A | 18154 | 18173 | TCTAAAGCTTTATATTTTCC | 79 | 2233 |
| 1316842 | N/A | N/A | 59479 | 59498 | AGACTTTACACTTAATGATA | 72 | 2234 |
| 1316896 | N/A | N/A | 40948 | 40967 | ACTATGTTACTTAAGAACAT | 85 | 2235 |
| 1316900 | N/A | N/A | 83012 | 83031 | GCCAACAACTTCATGAGCAC | 98 | 2236 |
| 1316947 | N/A | N/A | 117966 | 117985 | CCATATAATCCCAACAATGC | 93 | 2237 |
| 1317003 | N/A | N/A | 22009 | 22028 | CTTCATCACAATCTACCTTA | 75 | 2238 |
| 1317137 | N/A | N/A | 127953 | 127972 | CCTTAGACACTCACATGCTT | 49 | 2239 |
| 1317139 | N/A | N/A | 54878 | 54897 | AGTTCTTAAAATAATGCACA | 43 | 2240 |
| 1317171 | N/A | N/A | 141101 | 141120 | TACTATCAAAATCATCACTA | 82 | 2241 |
| 1317184 | N/A | N/A | 12168 | 12187 | AGATGCCTCCTTCCCATCCA | 84 | 2242 |
| 1317209 | N/A | N/A | 112701 | 112720 | AGCTACATTTTTCCCATAAC | 60 | 2243 |
| 1317222 | N/A | N/A | 101905 | 101924 | GCAGAGCCACACAGCTCCCA | 90 | 2244 |

TABLE 31
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 31 | 858 |
| 1314483 | N/A | N/A | 130779 | 130798 | ACTTTGTTTTTTAAATCATT | 89 | 2245 |
| 1314484 | N/A | N/A | 45770 | 45789 | GTCACAATAACCTAGACTCA | 95 | 2246 |
| 1314622 | 665 | 684 | N/A | N/A | CCATTCTTTTTAATTTCCTT | 36 | 2247 |
| 1314625 | N/A | N/A | 118780 | 118799 | GCAAGGCCAATCACATAGGC | 93 | 2248 |
| 1314666 | N/A | N/A | 8212 | 8231 | TGTAACTTTTATAATTTTCA | 47 | 2249 |
| 1314808 | N/A | N/A | 19110 | 19129 | TTTTCAACTATTTCAAGGCA | 85 | 2250 |
| 1314830 | N/A | N/A | 106565 | 106584 | GCTCCTATAATTTAACACCA | 69 | 2251 |
| 1314878 | N/A | N/A | 16580 | 16599 | GCTCATCCATACTTAGGAAC | 54 | 2252 |
| 1314884 | N/A | N/A | 26045 | 26064 | CACTTCCAAACCATACAATA | 92 | 2253 |
| | | | 26133 | 26152 | | | |
| 1314915 | N/A | N/A | 52281 | 52300 | CCATAGGCAATTTTTCATCC | 40 | 2254 |
| 1314944 | N/A | N/A | 36952 | 36971 | GGAAAATGATTTCTACCCCA | 83 | 2255 |
| 1314963 | N/A | N/A | 110471 | 110490 | TGCAAATTCACTCAAATGTC | 78 | 2256 |
| 1314990 | N/A | N/A | 126632 | 126651 | ACACTGTTACTTCATTATCT | 62 | 2257 |
| 1315013 | N/A | N/A | 24152 | 24171 | TACCATTTCCAACCACACTT | 94 | 2258 |
| 1315023 | N/A | N/A | 130140 | 130159 | CTCTCAGTTATCTATTCCTA | 46 | 2259 |
| 1315066 | N/A | N/A | 71875 | 71894 | TGCTAGGTCACAATTAAGTG | 49 | 2260 |
| 1315108 | N/A | N/A | 13191 | 13210 | GTTGAATTACCCACTCCACT | 90 | 2261 |
| 1315132 | N/A | N/A | 73728 | 73747 | GGCCACATCCTCCATTATCA | 96 | 2262 |
| 1315210 | N/A | N/A | 60655 | 60674 | CAGGTTAGTCCCACTCAGTA | 91 | 2263 |
| 1315220 | N/A | N/A | 53037 | 53056 | ACTTTCTAAATTACCATTTA | 74 | 2264 |
| 1315225 | N/A | N/A | 2250 | 2269 | TTGGTTTTATTCCCAACACA | 90 | 2265 |
| 1315229 | N/A | N/A | 112700 | 112719 | GCTACATTTTTCCCATAACA | 52 | 2266 |
| 1315265 | N/A | N/A | 68831 | 68850 | GCCATACTCCCCTTACAGCG | 51 | 2267 |
| 1315267 | N/A | N/A | 22867 | 22886 | TCTGAGGCTTTTCCAGGTTA | 48 | 2268 |
| 1315292 | N/A | N/A | 137675 | 137694 | TCTTAGTTTAATACTCAGAA | 84 | 2269 |
| 1315296 | N/A | N/A | 44393 | 44412 | GAAGCAGTATATCAGACCAC | 66 | 2270 |
| 1315302 | N/A | N/A | 22250 | 22269 | ATACCGAGTCTTACTTTTCC | 49 | 2271 |
| 1315326 | N/A | N/A | 17520 | 17539 | CACACATACACTCTTCTTTC | 77 | 2272 |
| 1315394 | N/A | N/A | 141093 | 141112 | AAATCATCACTATAACCTCC | 84 | 2273 |
| 1315417 | N/A | N/A | 42357 | 42376 | AGCAAGACATCTTAACATCT | 98 | 2274 |
| 1315511 | N/A | N/A | 134883 | 134902 | TCATCAATACTCAAACAGGT | 51 | 2275 |
| 1315541 | N/A | N/A | 107007 | 107026 | ACATGGCACTTCCTTCCATC | 78 | 2276 |
| 1315562 | N/A | N/A | 108445 | 108464 | AACACAAGTTCCTCTAGGGC | 28 | 2277 |
| 1315671 | N/A | N/A | 40940 | 40959 | ACTTAAGAACATCCATTTCC | 80 | 2278 |
| 1315747 | N/A | N/A | 10100 | 10119 | ATTAAGGTTTCATTTCTCTA | 18 | 2279 |
| 1315754 | N/A | N/A | 54865 | 54884 | ATGCACAGTATTAATCACAT | 25 | 2280 |
| 1315766 | N/A | N/A | 56227 | 56246 | GTCGGTACCGTCTAACACCT | 76 | 2281 |
| 1315843 | N/A | N/A | 100379 | 100398 | ACAAGGGATTCTATTATTGC | 46 | 2282 |
| 1315861 | N/A | N/A | 142801 | 142820 | TGAGTATTCATAACAAGACT | 71 | 2283 |
| 1315868 | N/A | N/A | 19867 | 19886 | ATCATAAAACAATTTTCCAT | 97 | 2284 |
| 1315902 | N/A | N/A | 45281 | 45300 | CTTGCTTAATAGTTTGGCCA | 86 | 2285 |
| 1315907 | N/A | N/A | 117964 | 117983 | ATATAATCCCAACAATGCTA | 89 | 2286 |
| 1315947 | N/A | N/A | 15198 | 15217 | CTTCCCACAACATTTCCACA | 91 | 2287 |
| 1315956 | N/A | N/A | 80421 | 80440 | ACACTGTACACATTTTATTT | 84 | 2288 |
| 1315999 | N/A | N/A | 82942 | 82961 | GCACAAACATTATCATTAAA | 58 | 2289 |
| 1316016 | N/A | N/A | 127949 | 127968 | AGACACTCACATGCTTGTTA | 41 | 2290 |
| 1316104 | N/A | N/A | 97693 | 97712 | GCATTCAGAATGCACAGCCT | 111 | 2291 |
| 1316134 | N/A | N/A | 58633 | 58652 | TACCACAGACTTTCTGGATT | 84 | 2292 |
| 1316141 | N/A | N/A | 86309 | 86328 | TTCCTAACTTTCTTTCATAA | 114 | 2293 |
| 1316179 | N/A | N/A | 47093 | 47112 | GCACTTTTAATACTATATCA | 79 | 2294 |
| 1316197 | N/A | N/A | 18151 | 18170 | AAAGCTTTATATTTTCCCAA | 45 | 2295 |
| 1316215 | N/A | N/A | 153260 | 153279 | TGGTCATTTTCACTTATCTT | 48 | 2296 |
| 1316303 | N/A | N/A | 81070 | 81089 | TCACTCACTACATCTATTCC | 84 | 2297 |
| 1316317 | N/A | N/A | 22003 | 22022 | CACAATCTACCTTATTACAC | 87 | 2298 |
| 1316439 | N/A | N/A | 133943 | 133962 | TTCCCACTCATCACAGGACA | 103 | 2299 |
| 1316536 | N/A | N/A | 72939 | 72958 | ACATCTTTCCTATAAATCAG | 85 | 2300 |
| 1316555 | N/A | N/A | 148916 | 148935 | GTCAAGACACCATTCAGCTA | 83 | 2301 |
| 1316561 | N/A | N/A | 133522 | 133541 | CCCAAAATAACCTCATGCTC | 61 | 2302 |
| 1316573 | N/A | N/A | 70222 | 70241 | TGTCAGCAACATAAATTCTT | 44 | 2303 |
| 1316631 | N/A | N/A | 59475 | 59494 | TTTACACTTAATGATAGGTT | 65 | 2304 |
| 1316633 | N/A | N/A | 121128 | 121147 | CAATGTCAACTTTATTACTA | 89 | 2305 |
| 1316636 | N/A | N/A | 11756 | 11775 | GTAGTCACTTTCACTTCACC | 35 | 2306 |
| 1316685 | N/A | N/A | 3678 | 3697 | GAGCATTTTAAATCTATCTT | 37 | 2307 |
| 1316742 | N/A | N/A | 77458 | 77477 | GAACGAACTCATACACGCTC | 76 | 2308 |
| 1316757 | N/A | N/A | 64094 | 64113 | ACAACAAGAACCAATCTCAC | 100 | 2309 |
| 1316776 | 5432 | 5451 | 115310 | 115329 | TGCTCACTCATTTCCACCTT | 35 | 2310 |
| 1316841 | N/A | N/A | 117134 | 117153 | AGTCATTTATTTAAAGCAAA | 85 | 2311 |
| 1316882 | N/A | N/A | 33511 | 33530 | AATTCATTCCCTGTTAGGGA | 98 | 2312 |
| 1316891 | 10930 | 10949 | 167679 | 167698 | TCCATTTTAATGACTTGGCT | 66 | 2313 |
| 1316965 | N/A | N/A | 50196 | 50215 | GTGACAGCAATTCTTTTCCT | 29 | 2314 |
| 1316994 | 4736 | 4755 | 104723 | 104742 | ACAGCCTTCCTTCCACTGGC | 98 | 2315 |
| 1317035 | N/A | N/A | 48404 | 48423 | AGACGGATTTTCTCAAGTCA | 98 | 2316 |
| 1317079 | N/A | N/A | 101851 | 101870 | CCTGCTAAATTCCATTCCTT | 66 | 2317 |
| 1317147 | N/A | N/A | 34330 | 34349 | GAGGACAGAATTCTAATCCA | 81 | 2318 |
| 1317149 | N/A | N/A | 67048 | 67067 | CTGAAGCTTTTTTCTAGAAA | 51 | 2319 |
| 1317166 | N/A | N/A | 6011 | 6030 | CATGCTACTCCTCTGCTCTA | 85 | 2320 |
| 1317228 | N/A | N/A | 34747 | 34766 | AGCACCTACTCCCATTCCAA | 80 | 2321 |

TABLE 32
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 24 | 858 |
| 1314533 | N/A | N/A | 33490 | 33509 | AAGCCAGTTTTATATTCCAA | 47 | 2322 |
| 1314567 | N/A | N/A | 3677 | 3696 | AGCATTTTAAATCTATCTTA | 80 | 2323 |
| 1314590 | N/A | N/A | 53006 | 53025 | GGCTATATAAAAATTATGGC | 74 | 2324 |
| 1314596 | N/A | N/A | 130139 | 130158 | TCTCAGTTATCTATTCCTAA | 26 | 2325 |
| 1314657 | N/A | N/A | 133521 | 133540 | CCAAAATAACCTCATGCTCA | 57 | 2326 |
| 1314681 | N/A | N/A | 15195 | 15214 | CCCACAACATTTCCACAAAT | 90 | 2327 |
| 1314687 | N/A | N/A | 2249 | 2268 | TGGTTTTATTCCCAACACAC | 77 | 2328 |
| 1314712 | 3090 | 3109 | N/A | N/A | GCCTCTATATATTCTGGTTA | 66 | 2329 |
| 1314739 | N/A | N/A | 126630 | 126649 | ACTGTTACTTCATTATCTAA | 54 | 2330 |
| 1314763 | N/A | N/A | 68813 | 68832 | CGTTGCCTACTATCTGTTTA | 36 | 2331 |
| 1314819 | N/A | N/A | 80417 | 80436 | TGTACACATTTTATTTACTT | 94 | 2332 |
| 1314873 | N/A | N/A | 22244 | 22263 | AGTCTTACTTTTCCATTCAA | 44 | 2333 |
| 1314899 | N/A | N/A | 108444 | 108463 | ACACAAGTTCCTCTAGGGCA | 24 | 2334 |
| 1315073 | 5428 | 5447 | 115306 | 115325 | CACTCATTTCCACCTTCAGC | 29 | 2335 |
| 1315115 | N/A | N/A | 19109 | 19128 | TTTCAACTATTICAAGGCAA | 57 | 2336 |
| 1315121 | N/A | N/A | 30084 | 30103 | GGAAGACACATCCCATCACT | 93 | 2337 |
| 1315148 | N/A | N/A | 70218 | 70237 | AGCAACATAAATTCTTATTA | 62 | 2338 |
| 1315211 | N/A | N/A | 47028 | 47047 | GACAATTTTTCCACAGGGCC | 65 | 2339 |
| 1315213 | N/A | N/A | 34329 | 34348 | AGGACAGAATTCTAATCCAA | 80 | 2340 |
| 1315239 | N/A | N/A | 148850 | 148869 | CCTTATTTTATTCCCCACCA | 77 | 2341 |
| 1315249 | N/A | N/A | 22856 | 22875 | TCCAGGTTATCTTCCTATAA | 58 | 2342 |
| 1315289 | N/A | N/A | 16531 | 16550 | AAGCAGACTACATTTTCACA | 63 | 2343 |
| 1315434 | N/A | N/A | 130775 | 130794 | TGTTTTTTAAATCATTAGTT | 84 | 2344 |
| 1315441 | N/A | N/A | 77383 | 77402 | TGCTTGCTCACTTCTTCCTG | 70 | 2345 |
| 1315485 | N/A | N/A | 142746 | 142765 | ACTGATTGATTTCAATCCAC | 39 | 2346 |
| 1315571 | N/A | N/A | 26043 | 26062 | CTTCCAAACCATACAATAGA | 95 | 2347 |
| | | | 26131 | 26150 | | | |
| 1315581 | N/A | N/A | 17517 | 17536 | ACATACACTCTTCTTTCTGT | 59 | 2348 |
| 1315590 | N/A | N/A | 44344 | 44363 | TTCTCAATAAATGAACTCTA | 70 | 2349 |
| 1315619 | N/A | N/A | 60653 | 60672 | GGTTAGTCCCACTCAGTACC | 97 | 2350 |
| 1315641 | N/A | N/A | 106414 | 106433 | CCACATAATACATTTAGTCA | 45 | 2351 |
| 1315664 | N/A | N/A | 18148 | 18167 | GCTTTATATTTTCCCAAATT | 46 | 2352 |
| 1315672 | N/A | N/A | 127938 | 127957 | TGCTTGTTACATATTATCTT | 18 | 2353 |
| 1315707 | N/A | N/A | 140849 | 140868 | GCCACTTTTATTTTACAGTA | 75 | 2354 |
| 1315708 | N/A | N/A | 107004 | 107023 | TGGCACTTCCTTCCATCTGC | 51 | 2355 |
| 1315730 | N/A | N/A | 86303 | 86322 | ACTTTCTTTCATAATGAGAA | 93 | 2356 |
| 1315809 | N/A | N/A | 45741 | 45760 | AAAGATTTCAGTACTACTCC | 53 | 2357 |
| 1315815 | N/A | N/A | 117936 | 117955 | TTTTAGTTCCCCTTCCACTC | 89 | 2358 |
| 1315839 | N/A | N/A | 6005 | 6024 | ACTCCTCTGCTCTAATCCTC | 82 | 2359 |
| 1315930 | N/A | N/A | 73678 | 73697 | TGCTCTTTCCTACCAGCCCA | 76 | 2360 |
| 1315935 | N/A | N/A | 82902 | 82921 | GCCAAGCACTATATACAACT | 58 | 2361 |
| 1315964 | N/A | N/A | 10094 | 10113 | GTTTCATTTCTCTAGGATTA | 14 | 2362 |
| 1315965 | N/A | N/A | 34746 | 34765 | GCACCTACTCCCATTCCAAA | 79 | 2363 |
| 1315990 | N/A | N/A | 81069 | 81088 | CACTCACTACATCTATTCCT | 87 | 2364 |
| 1316052 | 10190 | 10209 | 166939 | 166958 | TCACATACTTTACACGGGCA | 34 | 2365 |
| 1316073 | N/A | N/A | 59459 | 59478 | GGTTATTTCTAAATATATCC | 76 | 2366 |
| 1316088 | N/A | N/A | 50195 | 50214 | TGACAGCAATTCTTTTCCTA | 29 | 2367 |
| 1316119 | N/A | N/A | 101840 | 101859 | CCATTCCTTTTTAAAGAGGT | 60 | 2368 |
| 1316126 | N/A | N/A | 133918 | 133937 | TACAGGCATTCTAAATGCTA | 86 | 2369 |
| 1316140 | N/A | N/A | 52279 | 52298 | ATAGGCAATTTTTCATCCCA | 43 | 2370 |
| 1316157 | N/A | N/A | 40939 | 40958 | CTTAAGAACATCCATTTCCC | 74 | 2371 |
| 1316250 | N/A | N/A | 117119 | 117138 | GCAAAGCATTCTCATTACAA | 29 | 2372 |
| 1316254 | N/A | N/A | 54861 | 54880 | ACAGTATTAATCACATTTCA | 24 | 2373 |
| 1316316 | N/A | N/A | 56127 | 56146 | AAAGAGCACATTTTACACAC | 80 | 2374 |
| 1316358 | N/A | N/A | 19859 | 19878 | ACAATTTTCCATATAAAGTA | 110 | 2375 |
| 1316365 | N/A | N/A | 152925 | 152944 | GCTTTCACTAAATCAGGAAC | 63 | 2376 |
| 1316416 | N/A | N/A | 13184 | 13203 | TACCCACTCCACTTAGTTCT | 86 | 2377 |
| 1316465 | N/A | N/A | 45260 | 45279 | GCCTAGGATTATATTAAATT | 91 | 2378 |
| 1316542 | N/A | N/A | 72933 | 72952 | TTCCTATAAATCAGAGCTCC | 76 | 2379 |
| 1316572 | N/A | N/A | 24064 | 24083 | AGCTTATCTATATCTTCTTG | 67 | 2380 |
| 1316638 | 4717 | 4736 | 104704 | 104723 | CCATGATGCCATCACAGAGC | 30 | 2381 |
| 1316652 | N/A | N/A | 11415 | 11434 | GCCAGCCTCCTCTAATTGTG | 86 | 2382 |
| 1316669 | N/A | N/A | 137634 | 137653 | TTGAAGCTTTCTTTTACAGT | 45 | 2383 |
| 1316692 | N/A | N/A | 7818 | 7837 | GGTCCTATCATATTTTAACC | 60 | 2384 |
| 1316698 | N/A | N/A | 36896 | 36915 | TGGCATTTTTATGAATGCAA | 85 | 2385 |
| 1316714 | N/A | N/A | 118749 | 118768 | GCTGAGAACACAGCCTGCTA | 95 | 2386 |
| 1316716 | N/A | N/A | 121124 | 121143 | GTCAACTTTATTACTATAGA | 24 | 2387 |
| 1316752 | N/A | N/A | 71846 | 71865 | TCCTAAACTTCTAATTCACA | 103 | 2388 |
| 1316761 | N/A | N/A | 100345 | 100364 | GCAACTCAAATACCTGTTCA | 49 | 2389 |
| 1316838 | N/A | N/A | 64085 | 64104 | ACCAATCTCACAATTAAGTA | 47 | 2390 |
| 1316860 | N/A | N/A | 22002 | 22021 | ACAATCTACCTTATTACACC | 76 | 2391 |
| 1316884 | N/A | N/A | 58620 | 58639 | CTGGATTATAATTCTAGGTT | 59 | 2392 |
| 1316941 | N/A | N/A | 110470 | 110489 | GCAAATTCACTCAAATGTCA | 74 | 2393 |
| 1317052 | N/A | N/A | 48299 | 48318 | CCTACTGTTTTCATTTCTCA | 61 | 2394 |
| 1317113 | N/A | N/A | 134881 | 134900 | ATCAATACTCAAACAGGTCA | 56 | 2395 |
| 1317129 | N/A | N/A | 42356 | 42375 | GCAAGACATCTTAACATCTG | 97 | 2396 |
| 1317195 | N/A | N/A | 97647 | 97666 | AACTGGTTTCCATCAGCTGC | 91 | 2397 |
| 1317197 | N/A | N/A | 112697 | 112716 | ACATTTTTCCCATAACAGAA | 82 | 2398 |

TABLE 33
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 42 | 858 |
| 1314526 | N/A | N/A | 59378 | 59397 | GAAGCATTAACTTTACTGGC | 45 | 2399 |
| 1314536 | N/A | N/A | 25828 | 25847 | AACAGAGCACCTCCAAACCA | 96 | 2400 |
| | | | 25851 | 25870 | | | |
| | | | 25897 | 25916 | | | |
| | | | 25962 | 25981 | | | |
| | | | 26182 | 26201 | | | |
| | | | 26205 | 26224 | | | |
| 1314549 | N/A | N/A | 22243 | 22262 | GTCTTACTTTTCCATTCAAT | 42 | 2401 |
| 1314551 | N/A | N/A | 50190 | 50209 | GCAATTCTTTTCCTATGCAA | 74 | 2402 |
| 1314594 | N/A | N/A | 133914 | 133933 | GGCATTCTAAATGCTACCTC | 72 | 2403 |
| 1314653 | N/A | N/A | 30083 | 30102 | GAAGACACATCCCATCACTA | 105 | 2404 |
| 1314713 | N/A | N/A | 18117 | 18136 | AGGTTGTATCACCTCTGACA | 83 | 2405 |
| 1314734 | N/A | N/A | 56125 | 56144 | AGAGCACATTTTACACACAG | 70 | 2406 |
| 1314796 | N/A | N/A | 53005 | 53024 | GCTATATAAAAATTATGGCA | 100 | 2407 |
| 1314822 | N/A | N/A | 16529 | 16548 | GCAGACTACATTTTCACAAT | 33 | 2408 |
| 1314854 | N/A | N/A | 22846 | 22865 | CTTCCTATAATATATTCCAA | 95 | 2409 |
| 1314898 | N/A | N/A | 24063 | 24082 | GCTTATCTATATCTTCTTGA | 83 | 2410 |
| 1314960 | N/A | N/A | 34681 | 34700 | GCATGAAGAAATATTACACT | 59 | 2411 |
| 1314979 | N/A | N/A | 118732 | 118751 | CTAGAGGTCATCTTCGGTCA | 44 | 2412 |
| 1314989 | N/A | N/A | 19076 | 19095 | GTCCTTGTTATTCTATCTTA | 56 | 2413 |
| 1315030 | N/A | N/A | 3676 | 3695 | GCATTTTAAATCTATCTTAC | 45 | 2414 |
| 1315044 | N/A | N/A | 33475 | 33494 | TCCAACAGTCCCTAATGGGA | 90 | 2415 |
| 1315050 | N/A | N/A | 48296 | 48315 | ACTGTTTTCATTTCTCAGCA | 56 | 2416 |
| 1315089 | N/A | N/A | 52278 | 52297 | TAGGCAATTTTTCATCCCAT | 40 | 2417 |
| 1315100 | N/A | N/A | 106068 | 106087 | CCTCAGTGACACATACGCTC | 93 | 2418 |
| | | | 106124 | 106143 | | | |
| | | | 106180 | 106199 | | | |
| 1315126 | N/A | N/A | 13176 | 13195 | CCACTTAGTTCTACACCTCA | 77 | 2419 |
| 1315180 | N/A | N/A | 82901 | 82920 | CCAAGCACTATATACAACTA | 83 | 2420 |
| 1315288 | N/A | N/A | 15165 | 15184 | CAGAACAGAATCATACACAG | 69 | 2421 |
| 1315318 | N/A | N/A | 42352 | 42371 | GACATCTTAACATCTGACTC | 94 | 2422 |
| 1315332 | N/A | N/A | 2247 | 2266 | GTTTTATTCCCAACACACAT | 82 | 2423 |
| 1315358 | N/A | N/A | 112576 | 112595 | CTGACACAATCATTACCAAC | 156 | 2424 |
| 1315407 | N/A | N/A | 142745 | 142764 | CTGATTGATTTCAATCCACA | 55 | 2425 |
| 1315453 | N/A | N/A | 86216 | 86235 | GTCCTATTCCTTATACACTA | 86 | 2426 |
| 1315455 | N/A | N/A | 45735 | 45754 | TTCAGTACTACTCCATGTCA | 83 | 2427 |
| 1315456 | N/A | N/A | 19845 | 19864 | AAAGTAATATTTACAGGCTC | 78 | 2428 |
| 1315469 | N/A | N/A | 5998 | 6017 | TGCTCTAATCCTCCAGTGAC | 89 | 2429 |
| 1315516 | N/A | N/A | 117915 | 117934 | CCACCTCTATCTCTACCATC | 91 | 2430 |
| 1315607 | N/A | N/A | 133480 | 133499 | TGTACAGTTCATCCTAGTAA | 92 | 2431 |
| 1315663 | N/A | N/A | 106980 | 106999 | GTCAATAAAGTATCCACGAC | 88 | 2432 |
| 1315667 | N/A | N/A | 81068 | 81087 | ACTCACTACATCTATTCCTA | 75 | 2433 |
| 1315717 | N/A | N/A | 45257 | 45276 | TAGGATTATATTAAATTCTA | 101 | 2434 |
| 1315723 | N/A | N/A | 117026 | 117045 | ATCTTATGACTTTCTTGGGT | 29 | 2435 |
| 1315796 | N/A | N/A | 130136 | 130155 | CAGTTATCTATTCCTAAGCT | 79 | 2436 |
| 1315797 | N/A | N/A | 140553 | 140572 | GATGCATATTTTATCTACCT | 47 | 2437 |
| 1315835 | N/A | N/A | 104554 | 104573 | TTCTTTTAAAATTAAGATAC | 95 | 2438 |
| 1315867 | 3009 | 3028 | 66873 | 66892 | ACTGCTTTGATCTCTTGCCA | 20 | 2439 |
| 1315883 | N/A | N/A | 58326 | 58345 | GCTTTAGGTTTAAATCCGAT | 32 | 2440 |
| 1315911 | N/A | N/A | 54852 | 54871 | ATCACATTTCATTAAGACCA | 36 | 2441 |
| 1315987 | N/A | N/A | 64053 | 64072 | TGCAAAAGTCCATCTATACT | 76 | 2442 |
| 1316106 | N/A | N/A | 108443 | 108462 | CACAAGTTCCTCTAGGGCAC | 34 | 2443 |
| 1316144 | N/A | N/A | 40937 | 40956 | TAAGAACATCCATTTCCCAT | 85 | 2444 |
| 1316159 | N/A | N/A | 126625 | 126644 | TACTTCATTATCTAAGGATA | 84 | 2445 |
| 1316183 | N/A | N/A | 77035 | 77054 | GTCCGGTTTTTAACAGAGGT | 80 | 2446 |
| 1316185 | N/A | N/A | 11357 | 11376 | GGGCATGTAAACTCAAGGAC | 52 | 2447 |
| 1316200 | N/A | N/A | 72918 | 72937 | GCTCCATAAAAATCATTTGA | 84 | 2448 |
| 1316209 | N/A | N/A | 36874 | 36893 | CCTCATACAATTTCCCTCCC | 97 | 2449 |
| 1316227 | N/A | N/A | 17494 | 17513 | ATACCTGTTTTAACATCCAA | 78 | 2450 |
| 1316230 | N/A | N/A | 60616 | 60635 | AGAACCGTCACTCTTCAACA | 117 | 2451 |
| 1316287 | N/A | N/A | 21999 | 22018 | ATCTACCTTATTACACCATT | 73 | 2452 |
| 1316338 | N/A | N/A | 110398 | 110417 | GCAGACACCACCTAACTCTC | 83 | 2453 |
| 1316374 | N/A | N/A | 134846 | 134865 | CCCTAGTTTACTTTTAAGTA | 88 | 2454 |
| 1316395 | N/A | N/A | 121123 | 121142 | TCAACTTTATTACTATAGAA | 92 | 2455 |
| 1316436 | N/A | N/A | 97545 | 97564 | CTTGTTGGTTTCAAATACTC | 68 | 2456 |
| 1316479 | N/A | N/A | 34313 | 34332 | CCAAGTCCATCTGATTCCAC | 67 | 2457 |
| 1316538 | N/A | N/A | 10051 | 10070 | TGGGCTGTATACTAACGGTA | 71 | 2458 |
| 1316550 | N/A | N/A | 70217 | 70236 | GCAACATAAATTCTTATTAT | 78 | 2459 |
| 1316591 | N/A | N/A | 80405 | 80424 | ATTTACTTTTACCCACACAA | 71 | 2460 |
| 1316614 | N/A | N/A | 99836 | 99855 | CTGTTCTTTCATCCTTGTTA | 50 | 2461 |
| 1316664 | N/A | N/A | 137618 | 137637 | CAGTCAGAACTTTTACAGCT | 55 | 2462 |
| 1316675 | N/A | N/A | 152835 | 152854 | CCAGCATACACCTGTAGGGC | 68 | 2463 |
| 1316795 | N/A | N/A | 44299 | 44318 | AACCAGTTCATCAAAGAGAT | 95 | 2464 |
| 1316804 | N/A | N/A | 47027 | 47046 | ACAATTTTTCCACAGGGCCA | 89 | 2465 |
| 1316814 | N/A | N/A | 148560 | 148579 | GCAGTGAGTCCCTTAAACCT | 77 | 2466 |
| 1316858 | N/A | N/A | 165381 | 165400 | TGTCAGCATTTTACTTCCCT | 47 | 2467 |
| 1316877 | N/A | N/A | 7813 | 7832 | TATCATATTTTAACCTAGGA | 94 | 2468 |
| 1316980 | N/A | N/A | 71708 | 71727 | TGGCATTCATTTCCCACAGC | 62 | 2469 |
| 1317024 | 4486 | 4505 | 101339 | 101358 | GCAAATCTAAAACCTGCTTC | 62 | 2470 |
| 1317084 | 5427 | 5446 | 115305 | 115324 | ACTCATTTCCACCTTCAGCT | 33 | 2471 |
| 1317090 | N/A | N/A | 127937 | 127956 | GCTTGTTACATATTATCTTA | 16 | 2472 |
| 1317094 | N/A | N/A | 130764 | 130783 | TCATTAGTTATTATTATTCC | 53 | 2473 |
| 1317216 | N/A | N/A | 68794 | 68813 | AAGTTGAAATACATGCTTCA | 73 | 2474 |
| 1317236 | N/A | N/A | 73559 | 73578 | GTCCATACATTATACTTTCT | 46 | 2475 |

TABLE 34
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314506 | N/A | N/A | 133876 | 133895 | TGCAAACTGCCTCCACTCAC | 85 | 2476 |
| 1314509 | N/A | N/A | 44288 | 44307 | CAAAGAGATTTACAATCCTT | 90 | 2477 |
| 1314532 | N/A | N/A | 21995 | 22014 | ACCTTATTACACCATTTCAC | 69 | 2478 |
| 1314557 | N/A | N/A | 22242 | 22261 | TCTTACTTTTCCATTCAATC | 41 | 2479 |
| 1314584 | N/A | N/A | 19073 | 19092 | CTTGTTATTCTATCTTAGCC | 80 | 2480 |
| 1314598 | N/A | N/A | 121103 | 121122 | AGGACCACTTTCTCTCTCTT | 70 | 2481 |
| 1314613 | N/A | N/A | 2226 | 2245 | GGCAAAAAACTTCAATCAAC | 41 | 2482 |
| 1314641 | N/A | N/A | 56124 | 56143 | GAGCACATTTTACACACAGA | 37 | 2483 |
| 1314690 | N/A | N/A | 97432 | 97451 | TTTTTGTTTTTACTAGCTCC | 70 | 2484 |
| 1314761 | N/A | N/A | 7724 | 7743 | GGTTGACCAAAATAATCAAA | 55 | 2485 |
| 1314778 | N/A | N/A | 164361 | 164380 | GTAGGGACCAGAACTCCTAA | 92 | 2486 |
| | | | 164423 | 164442 | | | |
| 1314791 | N/A | N/A | 59377 | 59396 | AAGCATTAACTTTACTGGCA | 34 | 2487 |
| 1314849 | N/A | N/A | 58282 | 58301 | GCTGAGGTTTCTACTTTTCT | 13 | 2488 |
| 1314880 | N/A | N/A | 40935 | 40954 | AGAACATCCATTTCCCATTA | 67 | 2489 |
| 1314882 | N/A | N/A | 130135 | 130154 | AGTTATCTATTCCTAAGCTA | 81 | 2490 |
| 1314908 | N/A | N/A | 73558 | 73577 | TCCATACATTATACTTTCTT | 63 | 2491 |
| 1314948 | N/A | N/A | 72840 | 72859 | GGAATGCTTTTACTAGGGCA | 45 | 2492 |
| 1314968 | N/A | N/A | 60613 | 60632 | ACCGTCACTCTTCAACACAC | 75 | 2493 |
| 1315201 | N/A | N/A | 13171 | 13190 | TAGTTCTACACCTCATTCAT | 73 | 2494 |
| 1315256 | N/A | N/A | 140551 | 140570 | TGCATATTTTATCTACCTCT | 46 | 2495 |
| 1315260 | N/A | N/A | 19844 | 19863 | AAGTAATATTTACAGGCTCC | 51 | 2496 |
| 1315270 | N/A | N/A | 68776 | 68795 | CATGAAGTTATTCCTGCTTC | 78 | 2497 |
| 1315322 | N/A | N/A | 5997 | 6016 | GCTCTAATCCTCCAGTGACA | 77 | 2498 |
| 1315346 | N/A | N/A | 106966 | 106985 | CACGACAGTTCCAACATACA | 120 | 2499 |
| 1315370 | N/A | N/A | 137616 | 137635 | GTCAGAACTTTTACAGCTGT | 63 | 2500 |
| 1315438 | N/A | N/A | 17491 | 17510 | CCTGTTTTAACATCCAATCA | 89 | 2501 |
| 1315461 | N/A | N/A | 18001 | 18020 | AGCTTGAGTTTCTATAACAA | 54 | 2502 |
| 1315464 | N/A | N/A | 81064 | 81083 | ACTACATCTATTCCTAAGCA | 84 | 2503 |
| 1315470 | N/A | N/A | 110318 | 110337 | CACTTGCTTTTCTATTGTCT | 71 | 2504 |
| 1315522 | N/A | N/A | 117908 | 117927 | TATCTCTACCATCTACTTGC | 85 | 2505 |
| 1315557 | N/A | N/A | 104538 | 104557 | ATACATATCAATATAAGCAA | 97 | 2506 |
| 1315621 | N/A | N/A | 115130 | 115149 | GTAGAGTACATTAACCGCTA | 25 | 2507 |
| 1315705 | N/A | N/A | 106113 | 106132 | CATACGCTCACACATGAGCA | 104 | 2508 |
| | | | 106169 | 106188 | | | |
| 1315807 | N/A | N/A | 30080 | 30099 | GACACATCCCATCACTAGTT | 91 | 2509 |
| 1315881 | N/A | N/A | 117024 | 117043 | CTTATGACTTTCTTGGGTTC | 31 | 2510 |
| 1315940 | N/A | N/A | 48295 | 48314 | CTGTTTTCATTTCTCAGCAC | 48 | 2511 |
| 1315969 | N/A | N/A | 22845 | 22864 | TTCCTATAATATATTCCAAC | 91 | 2512 |
| 1315973 | N/A | N/A | 112574 | 112593 | GACACAATCATTACCAACAT | 79 | 2513 |
| 1316181 | N/A | N/A | 45684 | 45703 | AAATGGGTTCTCTTACTCTA | 86 | 2514 |
| 1316221 | N/A | N/A | 46788 | 46807 | GGCCATGGACCAATACCGCT | 87 | 2515 |
| 1316252 | N/A | N/A | 127923 | 127942 | ATCTTAATTTTACCTTTACA | 52 | 2516 |
| 1316300 | N/A | N/A | 142742 | 142761 | ATTGATTTCAATCCACACGG | 41 | 2517 |
| 1316308 | N/A | N/A | 86175 | 86194 | GTCAAATTCACATAGGGTTG | 38 | 2518 |
| 1316321 | N/A | N/A | 33474 | 33493 | CCAACAGTCCCTAATGGGAT | 97 | 2519 |
| 1316346 | N/A | N/A | 25822 | 25841 | GCACCTCCAAACCATACATC | 29 | 2520 |
| 1316360 | N/A | N/A | 147227 | 147246 | GTAATTTCTAAATCTGCAAC | 59 | 2521 |
| 1316384 | N/A | N/A | 15163 | 15182 | GAACAGAATCATACACAGTA | 104 | 2522 |
| 1316392 | N/A | N/A | 52277 | 52296 | AGGCAATTTTTCATCCCATA | 13 | 2523 |
| 1316414 | N/A | N/A | 69840 | 69859 | TAGTATATATTACCAGGTCA | 18 | 2524 |
| 1316554 | N/A | N/A | 118649 | 118668 | GGGTGACACCTATAACCTCA | 97 | 2525 |
| 1316557 | N/A | N/A | 82897 | 82916 | GCACTATATACAACTATTCT | 65 | 2526 |
| 1316577 | N/A | N/A | 130761 | 130780 | TTAGTTATTATTATTCCATT | 60 | 2527 |
| 1316602 | N/A | N/A | 64047 | 64066 | AGTCCATCTATACTATGACC | 57 | 2528 |
| 1316615 | N/A | N/A | 36873 | 36892 | CTCATACAATTTCCCTCCCA | 89 | 2529 |
| 1316617 | N/A | N/A | 108421 | 108440 | ACAAGGTGCACACTTTGCTC | 67 | 2530 |
| 1316663 | N/A | N/A | 3661 | 3680 | CTTACAATATGAACAGGTTA | 39 | 2531 |
| 1316671 | N/A | N/A | 11011 | 11030 | TGCTGTATTTTAATTGATCT | 48 | 2532 |
| 1316680 | N/A | N/A | 133368 | 133387 | TGTCAAGACCATACTTACGG | 53 | 2533 |
| 1316704 | N/A | N/A | 126604 | 126623 | CCAGAACCACATTTACTTTA | 29 | 2534 |
| 1316773 | N/A | N/A | 42351 | 42370 | ACATCTTAACATCTGACTCA | 98 | 2535 |
| 1316835 | N/A | N/A | 76958 | 76977 | GTACACACTTTCTTCTCGCA | 32 | 2536 |
| 1316859 | N/A | N/A | 10025 | 10044 | GTTTTATGACAAACTGCCAA | 69 | 2537 |
| 1316927 | N/A | N/A | 134843 | 134862 | TAGTTTACTTTTAAGTATCA | 79 | 2538 |
| 1316957 | N/A | N/A | 16526 | 16545 | GACTACATTTTCACAATATA | 57 | 2539 |
| 1316971 | N/A | N/A | 99789 | 99808 | GCCACACCACTCTACAGGTC | 59 | 2540 |
| 1316991 | N/A | N/A | 34182 | 34201 | AGGGTCACTATTTCTGGACA | 77 | 2541 |
| 1316997 | N/A | N/A | 50117 | 50136 | ACTAGCAGATTATATCACAA | 51 | 2542 |
| 1317066 | N/A | N/A | 52927 | 52946 | CATGTGAGCTTTATAGTATC | 73 | 2543 |
| 1317070 | N/A | N/A | 24015 | 24034 | AAGGAACTCATCCATTTCAT | 76 | 2544 |
| 1317076 | N/A | N/A | 80036 | 80055 | GGATCTCACACTATGATGCC | 81 | 2545 |
| 1317085 | N/A | N/A | 45194 | 45213 | TCATGGTAATTTAATTAAGA | 110 | 2546 |
| 1317123 | N/A | N/A | 54830 | 54849 | GATCTGTTTATAATAGGCTC | 34 | 2547 |
| 1317136 | 2962 | 2981 | 66826 | 66845 | GGTCACATTTATAAAACAGC | 13 | 2548 |
| 1317144 | N/A | N/A | 34680 | 34699 | CATGAAGAAATATTACACTA | 87 | 2549 |
| 1317152 | N/A | N/A | 101146 | 101165 | CTCCAGTTTCCTCTCTAGCA | 66 | 2550 |
| 1317214 | N/A | N/A | 152615 | 152634 | ACCCAGACCCTTGAATCCCT | 77 | 2551 |
| 1317237 | N/A | N/A | 71707 | 71726 | GGCATTCATTTCCCACAGCT | 48 | 2552 |

TABLE 35
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 33 | 858 |
| 1314512 | N/A | N/A | 164356 | 164375 | GACCAGAACTCCTAAGAGGA | 86 | 2553 |
| | | | 164418 | 164437 | | | |
| 1314534 | N/A | N/A | 13170 | 13189 | AGTTCTACACCTCATTCATT | 64 | 2554 |
| 1314597 | N/A | N/A | 71706 | 71725 | GCATTCATTTCCCACAGCTC | 75 | 2555 |
| 1314606 | 2961 | 2980 | 66825 | 66844 | GTCACATTTATAAAACAGCT | 35 | 2556 |
| 1314741 | N/A | N/A | 69821 | 69840 | ACTGAGTTAATTATAATCTA | 53 | 2557 |
| 1314743 | N/A | N/A | 42223 | 42242 | GCTACACACCCACATCCCTA | 84 | 2558 |
| 1314765 | N/A | N/A | 10656 | 10675 | TTTGGATGAACATTACCTCT | 60 | 2559 |
| 1314828 | N/A | N/A | 133367 | 133386 | GTCAAGACCATACTTACGGA | 27 | 2560 |
| 1314866 | N/A | N/A | 127922 | 127941 | TCTTAATTTTACCTTTACAT | 71 | 2561 |
| 1314914 | N/A | N/A | 17490 | 17509 | CTGTTTTAACATCCAATCAC | 89 | 2562 |
| 1314971 | N/A | N/A | 19765 | 19784 | ATACATGTCAATTTCTGCTC | 58 | 2563 |
| 1314998 | N/A | N/A | 110283 | 110302 | TCCCAACAAAATCTTCACTC | 104 | 2564 |
| 1315000 | N/A | N/A | 59375 | 59394 | GCATTAACTTTACTGGCAAC | 67 | 2565 |
| 1315028 | N/A | N/A | 45679 | 45698 | GGTTCTCTTACTCTAATCTA | 68 | 2566 |
| 1315043 | N/A | N/A | 60590 | 60609 | GTTCTGGGTTTAACTGCAAC | 53 | 2567 |
| 1315054 | N/A | N/A | 40932 | 40951 | ACATCCATTTCCCATTATTC | 55 | 2568 |
| 1315122 | N/A | N/A | 106965 | 106984 | ACGACAGTTCCAACATACAT | 90 | 2569 |
| 1315125 | N/A | N/A | 68748 | 68767 | AAGAAATTCCTTTCTCCACT | 68 | 2570 |
| 1315134 | N/A | N/A | 22843 | 22862 | CCTATAATATATTCCAACTT | 86 | 2571 |
| 1315139 | N/A | N/A | 56123 | 56142 | AGCACATTTTACACACAGAA | 48 | 2572 |
| 1315149 | N/A | N/A | 46787 | 46806 | GCCATGGACCAATACCGCTC | 87 | 2573 |
| 1315195 | N/A | N/A | 16525 | 16544 | ACTACATTTTCACAATATAC | 68 | 2574 |
| 1315276 | N/A | N/A | 130134 | 130153 | GTTATCTATTCCTAAGCTAC | 74 | 2575 |
| 1315286 | N/A | N/A | 52887 | 52906 | TTGTTGTTTAATTTGTGTAA | 53 | 2576 |
| 1315387 | N/A | N/A | 58079 | 58098 | TGTGAACTTTTAACATACCA | 94 | 2577 |
| 1315519 | N/A | N/A | 5984 | 6003 | AGTGACATCCCATCTCACTC | 93 | 2578 |
| 1315565 | N/A | N/A | 133841 | 133860 | CTGCCGGTCATCCTTTACTC | 106 | 2579 |
| 1315572 | N/A | N/A | 3562 | 3581 | AGGAAGATATTTTAATTATT | 100 | 2580 |
| 1315586 | N/A | N/A | 9989 | 10008 | GCCGTACTATATTATATTCC | 48 | 2581 |
| 1315638 | N/A | N/A | 22240 | 22259 | TTACTTTTCCATTCAATCTG | 86 | 2582 |
| 1315656 | N/A | N/A | 106112 | 106131 | ATACGCTCACACATGAGCAC | 98 | 2583 |
| | | | 106168 | 106187 | | | |
| 1315694 | N/A | N/A | 130757 | 130776 | TTATTATTATTCCATTGCTT | 36 | 2584 |
| 1315703 | N/A | N/A | 73557 | 73576 | CCATACATTATACTTTCTTA | 39 | 2585 |
| 1315746 | N/A | N/A | 54736 | 54755 | TAGCAGAGTTCATATAATCA | 27 | 2586 |
| 1315748 | N/A | N/A | 52266 | 52285 | CATCCCATAATTTTTCATCC | 82 | 2587 |
| 1315801 | N/A | N/A | 81063 | 81082 | CTACATCTATTCCTAAGCAT | 95 | 2588 |
| 1315820 | N/A | N/A | 7719 | 7738 | ACCAAAATAATCAAATACTA | 100 | 2589 |
| 1315828 | N/A | N/A | 15152 | 15171 | TACACAGTATTTCACAAACA | 90 | 2590 |
| 1315830 | N/A | N/A | 126602 | 126621 | AGAACCACATTTACTTTATA | 85 | 2591 |
| 1315919 | N/A | N/A | 120724 | 120743 | TCATCTGTTTCTAAACCAGT | 87 | 2592 |
| 1316007 | N/A | N/A | 44280 | 44299 | TTTACAATCCTTATCGACCA | 73 | 2593 |
| 1316026 | N/A | N/A | 25817 | 25836 | TCCAAACCATACATCAGAAA | 92 | 2594 |
| 1316037 | N/A | N/A | 76957 | 76976 | TACACACTTTCTTCTCGCAA | 38 | 2595 |
| 1316092 | N/A | N/A | 147001 | 147020 | CTGCTTGTTCCCATATGAGA | 59 | 2596 |
| 1316109 | N/A | N/A | 72786 | 72805 | AGCTACCCAGACATTTGGGC | 73 | 2597 |
| 1316352 | N/A | N/A | 99751 | 99770 | GGGACATTATGAACTGCATT | 49 | 2598 |
| 1316380 | N/A | N/A | 134816 | 134835 | TACTCCATTAATATTTTCCA | 56 | 2599 |
| 1316435 | N/A | N/A | 108398 | 108417 | AGGATTTGCCATCATTGTTA | 13 | 2600 |
| 1316455 | N/A | N/A | 116999 | 117018 | CCTTGTCATTTTCTTTCTCT | 54 | 2601 |
| 1316487 | N/A | N/A | 64027 | 64046 | CCAGTACTATAAAATGTCCA | 69 | 2602 |
| 1316539 | N/A | N/A | 45193 | 45212 | CATGGTAATTTAATTAAGAA | 86 | 2603 |
| 1316548 | N/A | N/A | 117906 | 117925 | TCTCTACCATCTACTTGCCA | 80 | 2604 |
| 1316580 | N/A | N/A | 50115 | 50134 | TAGCAGATTATATCACAAAC | 41 | 2605 |
| 1316593 | N/A | N/A | 34666 | 34685 | ACACTAAATTCTTTAGATTC | 91 | 2606 |
| 1316644 | N/A | N/A | 137553 | 137572 | ACTAAGCAAATTTATCACTC | 59 | 2607 |
| 1316693 | N/A | N/A | 112526 | 112545 | TTGTATACACATACATGGGA | 87 | 2608 |
| 1316741 | N/A | N/A | 142723 | 142742 | GTTGGCACTATTCTTATCCT | 42 | 2609 |
| 1316746 | N/A | N/A | 82891 | 82910 | TATACAACTATTCTACTTCA | 77 | 2610 |
| 1316753 | N/A | N/A | 118564 | 118583 | ACAGCAAGACTTCCATTTCT | 75 | 2611 |
| 1316788 | N/A | N/A | 18000 | 18019 | GCTTGAGTTTCTATAACAAA | 33 | 2612 |
| 1316797 | N/A | N/A | 97366 | 97385 | CTGTGAATCACTACTTGTCT | 93 | 2613 |
| 1316815 | N/A | N/A | 101118 | 101137 | CCTGAGTTCACATGCCACAA | 64 | 2614 |
| 1316837 | N/A | N/A | 24006 | 24025 | ATCCATTTCATCTATAAGTT | 57 | 2615 |
| 1316876 | N/A | N/A | 29886 | 29905 | TTTAACTTTCATTATCATCA | 67 | 2616 |
| 1316914 | N/A | N/A | 21994 | 22013 | CCTTATTACACCATTTCACA | 79 | 2617 |
| 1316915 | N/A | N/A | 33397 | 33416 | GGAGACATCCTTCATTTCTC | 100 | 2618 |
| 1316982 | N/A | N/A | 79921 | 79940 | GCAGCATTTTAAACTCCTGT | 63 | 2619 |
| 1316983 | N/A | N/A | 36872 | 36891 | TCATACAATTTCCCTCCCAT | 98 | 2620 |
| 1316989 | N/A | N/A | 34028 | 34047 | TGGCACAGTCATCTTCCCGC | 75 | 2621 |
| 1317009 | N/A | N/A | 2225 | 2244 | GCAAAAAACTTCAATCAACT | 69 | 2622 |
| 1317043 | N/A | N/A | 104537 | 104556 | TACATATCAATATAAGCAAC | 97 | 2623 |
| 1317060 | N/A | N/A | 152563 | 152582 | CCAATGTGACCTGCTAGGAA | 49 | 2624 |
| 1317127 | N/A | N/A | 86072 | 86091 | GCAATTCTATTTTACAGACT | 65 | 2625 |
| 1317151 | N/A | N/A | 19054 | 19073 | CAGAAGCAAAAATCTTTCAT | 79 | 2626 |
| 1317164 | N/A | N/A | 48293 | 48312 | GTTTTCATTTCTCAGCACCT | 32 | 2627 |
| 1317185 | N/A | N/A | 140549 | 140568 | CATATTTTATCTACCTCTCC | 81 | 2628 |
| 1317215 | N/A | N/A | 115085 | 115104 | GTGGACACAGTCACTTGGTC | 72 | 2629 |

TABLE 36
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 27 | 858 |
| 1314498 | N/A | N/A | 36856 | 36875 | CCATTCATCTTCTCTCAGGA | 80 | 2630 |
| 1314520 | 2960 | 2979 | 66824 | 66843 | TCACATTTATAAAACAGCTT | 74 | 2631 |
| 1314588 | N/A | N/A | 73550 | 73569 | TTATACTTTCTTAACATCCC | 101 | 2632 |
| 1314654 | N/A | N/A | 19764 | 19783 | TACATGTCAATTTCTGCTCT | 52 | 2633 |
| 1314679 | N/A | N/A | 22239 | 22258 | TACTTTTCCATTCAATCTGA | 87 | 2634 |
| 1314686 | N/A | N/A | 2218 | 2237 | ACTTCAATCAACTCTTCCTA | 72 | 2635 |
| 1314702 | N/A | N/A | 96631 | 96650 | GCATGGTACCTAATTATTCC | 89 | 2636 |
| 1314715 | N/A | N/A | 126551 | 126570 | ATGGAACCAATTTAAGGACT | 42 | 2637 |
| 1314749 | N/A | N/A | 116998 | 117017 | CTTGTCATTTTCTTTCTCTA | 34 | 2638 |
| 1314782 | N/A | N/A | 13166 | 13185 | CTACACCTCATTCATTCATT | 48 | 2639 |
| 1314829 | N/A | N/A | 9988 | 10007 | CCGTACTATATTATATTCCC | 16 | 2640 |
| 1314843 | N/A | N/A | 45191 | 45210 | TGGTAATTTAATTAAGAACA | 72 | 2641 |
| 1314883 | N/A | N/A | 52263 | 52282 | CCCATAATTTTTCATCCCAT | 65 | 2642 |
| 1315082 | N/A | N/A | 106077 | 106096 | CTCTGACCCCCTCAGTGACA | 93 | 2643 |
| | | | 106133 | 106152 | | | |
| 1315083 | N/A | N/A | 117878 | 117897 | TGGTTAGTTTTCCTTTTATA | 16 | 2644 |
| 1315186 | N/A | N/A | 151463 | 151482 | CCACTTAACCTTTCACACCC | 98 | 2645 |
| 1315246 | N/A | N/A | 106964 | 106983 | CGACAGTTCCAACATACATT | 82 | 2646 |
| 1315297 | N/A | N/A | 130028 | 130047 | AAGCTGCACATTCAATCAGC | 86 | 2647 |
| 1315299 | N/A | N/A | 134815 | 134834 | ACTCCATTAATATTTTCCAG | 43 | 2648 |
| 1315306 | N/A | N/A | 34658 | 34677 | TTCTTTAGATTCATCATCTC | 93 | 2649 |
| 1315398 | N/A | N/A | 72717 | 72736 | GGGTGAACTCTTTATTACTA | 54 | 2650 |
| 1315426 | N/A | N/A | 45673 | 45692 | CTTACTCTAATCTATTACTT | 94 | 2651 |
| 1315512 | N/A | N/A | 137549 | 137568 | AGCAAATTTATCACTCAGTA | 30 | 2652 |
| 1315531 | N/A | N/A | 147000 | 147019 | TGCTTGTTCCCATATGAGAA | 86 | 2653 |
| 1315632 | N/A | N/A | 120649 | 120668 | GACAGCTATCTTTCTTGGCT | 80 | 2654 |
| 1315634 | N/A | N/A | 56122 | 56141 | GCACATTTTACACACAGAAC | 39 | 2655 |
| 1315652 | N/A | N/A | 54734 | 54753 | GCAGAGTTCATATAATCAAC | 15 | 2656 |
| 1315728 | N/A | N/A | 33389 | 33408 | CCTTCATTTCTCTTATGATC | 96 | 2657 |
| 1315729 | N/A | N/A | 23967 | 23986 | AGTTGTTCATAATATTACCA | 44 | 2658 |
| 1315770 | N/A | N/A | 130720 | 130739 | ATACCCAGCATCATGTAGGC | 82 | 2659 |
| 1315774 | N/A | N/A | 115025 | 115044 | GTAGCATTTTTTTAAGCTTT | 98 | 2660 |
| 1315776 | N/A | N/A | 22832 | 22851 | TTCCAACTTAATGATACTGA | 52 | 2661 |
| 1315851 | N/A | N/A | 118316 | 118335 | GCATAAATATACAATGCAGA | 81 | 2662 |
| 1315856 | N/A | N/A | 104536 | 104555 | ACATATCAATATAAGCAACA | 94 | 2663 |
| 1315913 | N/A | N/A | 15151 | 15170 | ACACAGTATTTCACAAACAA | 83 | 2664 |
| 1315928 | N/A | N/A | 140531 | 140550 | CCATCCCCAATTCCTAGCTT | 90 | 2665 |
| 1315975 | N/A | N/A | 99585 | 99604 | GCCTTAAATCATCTTTGCAT | 77 | 2666 |
| 1316032 | N/A | N/A | 59295 | 59314 | GTGCATGGAAATATTTCCTA | 32 | 2667 |
| 1316036 | N/A | N/A | 34027 | 34046 | GGCACAGTCATCTTCCCGCA | 76 | 2668 |
| 1316041 | N/A | N/A | 69817 | 69836 | AGTTAATTATAATCTAGACA | 84 | 2669 |
| 1316089 | N/A | N/A | 44272 | 44291 | CCTTATCGACCAATTTTATT | 95 | 2670 |
| 1316103 | N/A | N/A | 17489 | 17508 | TGTTTTAACATCCAATCACT | 89 | 2671 |
| 1316122 | N/A | N/A | 57883 | 57902 | CAGATTGTTTTCAAATGAAC | 99 | 2672 |
| 1316128 | N/A | N/A | 133317 | 133336 | CTGCTAAATTTTCAATATAA | 81 | 2673 |
| 1316139 | N/A | N/A | 110282 | 110301 | CCCAACAAAATCTTCACTCC | 90 | 2674 |
| 1316204 | N/A | N/A | 48191 | 48210 | CCTGCTAAACTTTAATTCTC | 67 | 2675 |
| 1316233 | N/A | N/A | 46779 | 46798 | CCAATACCGCTCTGCAGCCT | 94 | 2676 |
| 1316236 | N/A | N/A | 79862 | 79881 | TTCAAGGTCACAGCTGGTTA | 62 | 2677 |
| 1316244 | N/A | N/A | 81062 | 81081 | TACATCTATTCCTAAGCATC | 103 | 2678 |
| 1316272 | N/A | N/A | 60568 | 60587 | GGATGTTGCATTTTTCCCAC | 91 | 2679 |
| 1316280 | N/A | N/A | 108373 | 108392 | ATAGAATTTGTAATATTTCA | 87 | 2680 |
| 1316351 | N/A | N/A | 68740 | 68759 | CCTTTCTCCACTTCTCATCT | 66 | 2681 |
| 1316394 | N/A | N/A | 17999 | 18018 | CTTGAGTTTCTATAACAAAA | 59 | 2682 |
| 1316443 | N/A | N/A | 40931 | 40950 | CATCCATTTCCCATTATTCA | 81 | 2683 |
| 1316452 | N/A | N/A | 101112 | 101131 | TTCACATGCCACAAACGGCA | 96 | 2684 |
| 1316499 | N/A | N/A | 64012 | 64031 | GTCCAGAAAATACAAATCTT | 33 | 2685 |
| 1316527 | N/A | N/A | 19042 | 19061 | TCTTTCATTATTTTTTAACT | 96 | 2686 |
| 1316532 | N/A | N/A | 142722 | 142741 | TTGGCACTATTCTTATCCTC | 48 | 2687 |
| 1316588 | N/A | N/A | 71644 | 71663 | TCCATCAGATATTTTTCCAA | 61 | 2688 |
| 1316599 | N/A | N/A | 7709 | 7728 | TCAAATACTATCTCATGACA | 84 | 2689 |
| 1316661 | N/A | N/A | 76568 | 76587 | TCTTCAGTCACACTAGCTGC | 99 | 2690 |
| 1316684 | N/A | N/A | 42098 | 42117 | CCACAGTTACCCAAACAACA | 74 | 2691 |
| 1316696 | N/A | N/A | 16507 | 16526 | ACATGAAGTTTTAAATGACT | 86 | 2692 |
| 1316784 | N/A | N/A | 10652 | 10671 | GATGAACATTACCTCTGGTT | 70 | 2693 |
| 1316846 | N/A | N/A | 133837 | 133856 | CGGTCATCCTTTACTCTTTA | 29 | 2694 |
| 1316910 | N/A | N/A | 5983 | 6002 | GTGACATCCCATCTCACTCA | 82 | 2695 |
| 1316916 | N/A | N/A | 50114 | 50133 | AGCAGATTATATCACAAACA | 37 | 2696 |
| 1316990 | N/A | N/A | 85103 | 85122 | CGGCATTTTTCAACATGCCG | 88 | 2697 |
| 1317013 | N/A | N/A | 162972 | 162991 | GTGTGGGTTCCACCTTCATC | 93 | 2698 |
| 1317049 | N/A | N/A | 21990 | 22009 | ATTACACCATTICACATGCA | 68 | 2699 |
| 1317051 | N/A | N/A | 3561 | 3580 | GGAAGATATTTTAATTATTG | 106 | 2700 |
| 1317093 | N/A | N/A | 127916 | 127935 | TTTTACCTTTACATTACCAT | 57 | 2701 |
| 1317114 | N/A | N/A | 25790 | 25809 | AGTGCAGACATCCTCAGGGA | 70 | 2702 |
| 1317126 | N/A | N/A | 112329 | 112348 | TCTGCTCTTCTCACTTAACC | 78 | 2703 |
| 1317183 | N/A | N/A | 82889 | 82908 | TACAACTATTCTACTTCAGT | 71 | 2704 |
| 1317199 | N/A | N/A | 52844 | 52863 | TTGTAAATAAATAATTACTA | 99 | 2705 |
| 1317202 | N/A | N/A | 28970 | 28989 | GTCGATGATCTCTTTAACCG | 59 | 2706 |

TABLE 37
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 34 | 858 |
| 1314494 | N/A | N/A | 5982 | 6001 | TGACATCCCATCTCACTCAC | 75 | 2707 |
| 1314510 | N/A | N/A | 46703 | 46722 | TGCTATATCACATTATATGA | 111 | 2708 |
| 1314524 | N/A | N/A | 105990 | 106009 | TGAGCACACACACAGAGGCA | 97 | 2709 |
| | | | 106099 | 106118 | | | |
| | | | 106155 | 106174 | | | |
| 1314546 | N/A | N/A | 126550 | 126569 | TGGAACCAATTTAAGGACTA | 59 | 2710 |
| 1314568 | N/A | N/A | 10484 | 10503 | GACAAAGCATTTCCATTACA | 52 | 2711 |
| 1314593 | N/A | N/A | 127890 | 127909 | ACGATATTTTTTGAAATTTA | 108 | 2712 |
| 1314656 | N/A | N/A | 110281 | 110300 | CCAACAAAATCTTCACTCCA | 102 | 2713 |
| 1314678 | N/A | N/A | 45120 | 45139 | CTAGAGTTTAGAATTTGCCA | 35 | 2714 |
| 1314716 | N/A | N/A | 63588 | 63607 | TTGGAGTTTACTATACACTC | 49 | 2715 |
| 1314717 | N/A | N/A | 120586 | 120605 | TGGGATGAATACCCTCCTCT | 108 | 2716 |
| 1314736 | N/A | N/A | 130027 | 130046 | AGCTGCACATTCAATCAGCA | 94 | 2717 |
| 1314858 | N/A | N/A | 60564 | 60583 | GTTGCATTTTTCCCACACCT | 85 | 2718 |
| 1314895 | N/A | N/A | 82847 | 82866 | CCTTAGTTTATTTAATTCAT | 79 | 2719 |
| 1314897 | N/A | N/A | 137088 | 137107 | AGGTCTCTTTTCATGAACAC | 127 | 2720 |
| 1314935 | N/A | N/A | 3558 | 3577 | AGATATTTTAATTATTGCCC | 18 | 2721 |
| 1314958 | N/A | N/A | 99569 | 99588 | GCATCAACCATATATATCTC | 50 | 2722 |
| 1314974 | N/A | N/A | 84856 | 84875 | GCTATGTGAAATAATCTTTA | 79 | 2723 |
| 1315001 | N/A | N/A | 52262 | 52281 | CCATAATTTTTCATCCCATT | 83 | 2724 |
| 1315037 | N/A | N/A | 19763 | 19782 | ACATGTCAATTTCTGCTCTT | 62 | 2725 |
| 1315084 | N/A | N/A | 104523 | 104542 | AGCAACATAAAATAATTCAT | 101 | 2726 |
| 1315109 | N/A | N/A | 48189 | 48208 | TGCTAAACTTTAATTCTCTT | 57 | 2727 |
| 1315129 | N/A | N/A | 22238 | 22257 | ACTTTTCCATTCAATCTGAA | 92 | 2728 |
| 1315165 | N/A | N/A | 116994 | 117013 | TCATTTTCTTTCTCTAGAGA | 84 | 2729 |
| 1315183 | N/A | N/A | 79817 | 79836 | CCAGATGTATTCATAAAGCC | 82 | 2730 |
| 1315247 | N/A | N/A | 101095 | 101114 | GCACCATTTACCTTATCTGC | 103 | 2731 |
| 1315271 | N/A | N/A | 44267 | 44286 | TCGACCAATTTTATTTAGAT | 84 | 2732 |
| 1315287 | N/A | N/A | 21988 | 22007 | TACACCATTTCACATGCAGC | 54 | 2733 |
| 1315309 | N/A | N/A | 142721 | 142740 | TGGCACTATTCTTATCCTCA | 33 | 2734 |
| 1315334 | N/A | N/A | 45669 | 45688 | CTCTAATCTATTACTTGCTT | 84 | 2735 |
| 1315347 | N/A | N/A | 9987 | 10006 | CGTACTATATTATATTCCCA | 17 | 2736 |
| 1315350 | N/A | N/A | 40929 | 40948 | TCCATTTCCCATTATTCAAA | 61 | 2737 |
| 1315354 | N/A | N/A | 134798 | 134817 | CAGAAATTTCATTTATACTC | 96 | 2738 |
| 1315372 | N/A | N/A | 34657 | 34676 | TCTTTAGATTCATCATCTCC | 106 | 2739 |
| 1315406 | 3283 | 3302 | N/A | N/A | GAGGCACTCCACAGTGCCAA | 118 | 2740 |
| 1315423 | N/A | N/A | 56102 | 56121 | ACAAATGTACTCATCTGTCA | 64 | 2741 |
| 1315471 | N/A | N/A | 81061 | 81080 | ACATCTATTCCTAAGCATCT | 99 | 2742 |
| 1315499 | N/A | N/A | 23965 | 23984 | TTGTTCATAATATTACCATA | 44 | 2743 |
| 1315501 | N/A | N/A | 133790 | 133809 | TAGGAAGGTTCTATTATGGC | 19 | 2744 |
| 1315584 | N/A | N/A | 72713 | 72732 | GAACTCTTTATTACTATGGA | 36 | 2745 |
| 1315627 | N/A | N/A | 133292 | 133311 | TATCAGGGTTTCTCTTCCCA | 64 | 2746 |
| 1315738 | N/A | N/A | 28780 | 28799 | CTGAAAGTCCTTAAATGAGC | 87 | 2747 |
| 1315816 | N/A | N/A | 68737 | 68756 | TTCTCCACTTCTCATCTCCC | 78 | 2748 |
| 1315855 | N/A | N/A | 19039 | 19058 | TTCATTATTTTTTAACTGAA | 102 | 2749 |
| 1315885 | N/A | N/A | 69805 | 69824 | TCTAGACATTTTAAACTTCT | 34 | 2750 |
| 1315932 | N/A | N/A | 75570 | 75589 | CTGGTCTATCTTAAATCCAT | 95 | 2751 |
| 1315951 | N/A | N/A | 96506 | 96525 | ATGTAACTCAACTAACCTCA | 111 | 2752 |
| 1316042 | N/A | N/A | 112322 | 112341 | TTCTCACTTAACCCAGTGCA | 102 | 2753 |
| 1316058 | N/A | N/A | 151461 | 151480 | ACTTAACCTTTCACACCCCC | 95 | 2754 |
| 1316101 | N/A | N/A | 146784 | 146803 | AACTGGGATCTATTTTCTCA | 88 | 2755 |
| 1316113 | N/A | N/A | 15149 | 15168 | ACAGTATTTCACAAACAACA | 90 | 2756 |
| 1316163 | N/A | N/A | 42077 | 42096 | GAGAAACTGTCAACTGACCT | 99 | 2757 |
| 1316198 | N/A | N/A | 16506 | 16525 | CATGAAGTTTTAAATGACTT | 97 | 2758 |
| 1316203 | N/A | N/A | 59273 | 59292 | AAGCTCCAATTTTAATTGTA | 85 | 2759 |
| 1316330 | N/A | N/A | 108367 | 108386 | TTTGTAATATTTCACAATCA | 82 | 2760 |
| 1316339 | N/A | N/A | 118298 | 118317 | GACCACAGAATTACAACACT | 72 | 2761 |
| 1316342 | N/A | N/A | 13139 | 13158 | TGTTTCTCGACTACTATGAA | 49 | 2762 |
| 1316368 | N/A | N/A | 17488 | 17507 | GTTTTAACATCCAATCACTA | 91 | 2763 |
| 1316409 | N/A | N/A | 7678 | 7697 | TATGAAATTCAAACTTCCTT | 69 | 2764 |
| 1316473 | N/A | N/A | 162971 | 162990 | TGTGGGTTCCACCTTCATCC | 101 | 2765 |
| 1316482 | N/A | N/A | 32882 | 32901 | GAATCTCTATCTAAACGGCA | 75 | 2766 |
| 1316507 | N/A | N/A | 115000 | 115019 | CTGTACACCATTACACAAAT | 64 | 2767 |
| 1316608 | N/A | N/A | 33981 | 34000 | GCCGAAAACAAATCTGCTCT | 99 | 2768 |
| 1316627 | N/A | N/A | 2206 | 2225 | TCTTCCTAATATTTAAGATA | 98 | 2769 |
| 1316639 | N/A | N/A | 117877 | 117896 | GGTTAGTTTTCCTTTTATAT | 14 | 2770 |
| 1316660 | N/A | N/A | 50083 | 50102 | GTTAAGTTTTATCAGAACAT | 63 | 2771 |
| 1316706 | N/A | N/A | 130685 | 130704 | GGCATTTGATAAATGAGTCA | 92 | 2772 |
| 1316715 | N/A | N/A | 22814 | 22833 | GAGGTCAGAAAACATACTCT | 97 | 2773 |
| 1316820 | N/A | N/A | 25694 | 25713 | CACACGGTTCTTACTTTGAT | 96 | 2774 |
| 1316828 | N/A | N/A | 106963 | 106982 | GACAGTTCCAACATACATTC | 106 | 2775 |
| 1316831 | N/A | N/A | 54715 | 54734 | CTATTCCAATTTTTTACCTC | 74 | 2776 |
| 1316870 | N/A | N/A | 17963 | 17982 | GGTGGTTTAAACAATAAGCA | 83 | 2777 |
| 1316888 | N/A | N/A | 36777 | 36796 | TGCAAAGGAAATTCCAGGCT | 85 | 2778 |
| 1316918 | N/A | N/A | 140495 | 140514 | GCAGCACAAAATAAAAACAT | 68 | 2779 |
| 1316924 | N/A | N/A | 66757 | 66776 | GATGAAACTCACCTGTGATA | 109 | 2780 |
| 1316958 | N/A | N/A | 73440 | 73459 | GAGGCTACTTTAAAATACTC | 93 | 2781 |
| 1317068 | N/A | N/A | 52828 | 52847 | ACTACTAGAATTATTCAGAT | 64 | 2782 |
| 1317169 | N/A | N/A | 57835 | 57854 | CCATCATTTCCTCCCTCCCC | 84 | 2783 |

TABLE 38
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 36 | 858 |
| 1314496 | N/A | N/A | 60563 | 60582 | TTGCATTTTTCCCACACCTC | 75 | 2784 |
| 1314511 | 4387 | 4406 | N/A | N/A | GAATAGCATTCTTATCTGCA | 27 | 2785 |
| 1314540 | N/A | N/A | 117872 | 117891 | GTTTTCCTTTTATATTCATC | 27 | 2786 |
| 1314655 | N/A | N/A | 17887 | 17906 | ACAGTGTTTTTCTTAACGAT | 105 | 2787 |
| 1314722 | N/A | N/A | 28751 | 28770 | TGGACAAACATTTATTTCAA | 52 | 2788 |
| 1314725 | N/A | N/A | 19732 | 19751 | GCTATTGTCATACAATTCAC | 51 | 2789 |
| 1314729 | N/A | N/A | 3557 | 3576 | GATATTTTAATTATTGCCCT | 33 | 2790 |
| 1314737 | N/A | N/A | 44266 | 44285 | CGACCAATTTTATTTAGATG | 67 | 2791 |
| 1314750 | N/A | N/A | 45089 | 45108 | TGCCACATCATTTATTTACT | 57 | 2792 |
| 1314771 | N/A | N/A | 21987 | 22006 | ACACCATTTCACATGCAGCA | 41 | 2793 |
| 1314787 | N/A | N/A | 52817 | 52836 | TATTCAGATTTTCCTCTCTA | 75 | 2794 |
| 1314792 | N/A | N/A | 33954 | 33973 | TGCCTTTTCAATTTGCATCT | 84 | 2795 |
| 1314814 | N/A | N/A | 34656 | 34675 | CTTTAGATTCATCATCTCCC | 78 | 2796 |
| 1314949 | N/A | N/A | 130026 | 130045 | GCTGCACATTCAATCAGCAC | 91 | 2797 |
| 1315045 | N/A | N/A | 134790 | 134809 | TCATTTATACTCAAACACTG | 91 | 2798 |
| 1315103 | N/A | N/A | 56092 | 56111 | TCATCTGTCATTTATTTCAA | 64 | 2799 |
| 1315118 | N/A | N/A | 106079 | 106098 | CACTCTGACCCCCTCAGTGA | 93 | 2800 |
| | | | 106135 | 106154 | | | |
| 1315136 | N/A | N/A | 72712 | 72731 | AACTCTTTATTACTATGGAA | 64 | 2801 |
| 1315187 | N/A | N/A | 57728 | 57747 | ACCTAGTTATTTCCTTCAAC | 64 | 2802 |
| 1315349 | N/A | N/A | 46702 | 46721 | GCTATATCACATTATATGAA | 63 | 2803 |
| 1315530 | N/A | N/A | 22813 | 22832 | AGGTCAGAAAACATACTCTG | 63 | 2804 |
| 1315537 | N/A | N/A | 126509 | 126528 | GCATGACAAGCATATCCCAC | 26 | 2805 |
| 1315683 | N/A | N/A | 23559 | 23578 | TCACTGTTTTTCCTATCAGC | 74 | 2806 |
| 1315699 | N/A | N/A | 120346 | 120365 | GATGAATTATTTCAACTCTT | 49 | 2807 |
| 1315716 | N/A | N/A | 19038 | 19057 | TCATTATTTTTTAACTGAAT | 100 | 2808 |
| 1315762 | N/A | N/A | 15059 | 15078 | GGCATTAAAAATAAGCAGAT | 76 | 2809 |
| 1315768 | N/A | N/A | 146737 | 146756 | AAAATGTATTCACATAGGTT | 109 | 2810 |
| 1315825 | N/A | N/A | 130654 | 130673 | TCTCACAGTCCAACTTAGAA | 74 | 2811 |
| 1315925 | N/A | N/A | 50053 | 50072 | ACAAAGTTAAATATGGCTGA | 101 | 2812 |
| 1315945 | N/A | N/A | 108325 | 108344 | TCAAATGTATATTAATCTTT | 117 | 2813 |
| 1315950 | N/A | N/A | 69804 | 69823 | CTAGACATTTTAAACTTCTG | 14 | 2814 |
| 1315961 | N/A | N/A | 66744 | 66763 | TGTGATAGACCACCCTCCTC | 96 | 2815 |
| 1315976 | N/A | N/A | 118297 | 118316 | ACCACAGAATTACAACACTA | 79 | 2816 |
| 1316013 | N/A | N/A | 63188 | 63207 | GGTAACTCAACTTCCAGGAA | 36 | 2817 |
| 1316053 | 3221 | 3240 | 71459 | 71478 | CACAAAGCTTCACAGCATCC | 63 | 2818 |
| 1316097 | N/A | N/A | 84837 | 84856 | AAGTTTTTTTTAAAATCCTT | 123 | 2819 |
| 1316146 | N/A | N/A | 114996 | 115015 | ACACCATTACACAAATACAA | 66 | 2820 |
| 1316160 | N/A | N/A | 79816 | 79835 | CAGATGTATTCATAAAGCCT | 61 | 2821 |
| 1316165 | N/A | N/A | 2191 | 2210 | AGATAATTTCATTAATCACC | 91 | 2822 |
| 1316192 | N/A | N/A | 99562 | 99581 | CCATATATATCTCCTAGAGT | 63 | 2823 |
| 1316249 | N/A | N/A | 68736 | 68755 | TCTCCACTTCTCATCTCCCA | 78 | 2824 |
| 1316322 | N/A | N/A | 160915 | 160934 | CCACGCACCATTTCTACCTG | 99 | 2825 |
| 1316359 | N/A | N/A | 127864 | 127883 | AGGTTAAAAATCCATTGATC | 39 | 2826 |
| 1316362 | N/A | N/A | 81060 | 81079 | CATCTATTCCTAAGCATCTA | 93 | 2827 |
| 1316400 | N/A | N/A | 82783 | 82802 | GCTGAGGCTTTTCAACAGGT | 23 | 2828 |
| 1316424 | N/A | N/A | 32823 | 32842 | AGACCCAGTTATATTCTTTA | 80 | 2829 |
| 1316440 | N/A | N/A | 42037 | 42056 | CGAGGCACAGTCTCAAGCCT | 71 | 2830 |
| 1316449 | N/A | N/A | 140095 | 140114 | GCTCCATTACATTCAGTCCT | 44 | 2831 |
| 1316485 | N/A | N/A | 75566 | 75585 | TCTATCTTAAATCCATTGTA | 69 | 2832 |
| 1316491 | N/A | N/A | 73436 | 73455 | CTACTTTAAAATACTCAGCA | 59 | 2833 |
| 1316517 | N/A | N/A | 96461 | 96480 | GAACACAAAAATATATAGCA | 96 | 2834 |
| 1316525 | N/A | N/A | 45655 | 45674 | TTGCTTAAAATTTATTCTGC | 94 | 2835 |
| 1316549 | N/A | N/A | 36069 | 36088 | GGTACAGTCACCAACTTCAT | 67 | 2836 |
| 1316592 | N/A | N/A | 133291 | 133310 | ATCAGGGTTTCTCTTCCCAA | 72 | 2837 |
| 1316619 | N/A | N/A | 25570 | 25589 | CTGGAGAATTTCTAAGCAAA | 104 | 2838 |
| 1316621 | N/A | N/A | 54712 | 54731 | TTCCAATTTTTTACCTCAGT | 20 | 2839 |
| 1316672 | N/A | N/A | 48188 | 48207 | GCTAAACTTTAATTCTCTTA | 25 | 2840 |
| 1316733 | N/A | N/A | 22226 | 22245 | AATCTGAAAAATCGCTGCCT | 75 | 2841 |
| 1316735 | N/A | N/A | 40928 | 40947 | CCATTTCCCATTATTCAAAA | 59 | 2842 |
| 1316751 | N/A | N/A | 104489 | 104508 | AGCAAGCACACATCAAGCTA | 107 | 2843 |
| 1316766 | N/A | N/A | 133788 | 133807 | GGAAGGTTCTATTATGGCAC | 25 | 2844 |
| 1316783 | N/A | N/A | 116993 | 117012 | CATTTTCTTTCTCTAGAGAC | 78 | 2845 |
| 1316825 | N/A | N/A | 7660 | 7679 | TTGTCATTAATAAAATTCTA | 104 | 2846 |
| 1316826 | N/A | N/A | 10483 | 10502 | ACAAAGCATTTCCATTACAA | 57 | 2847 |
| 1316829 | N/A | N/A | 16503 | 16522 | GAAGTTTTAAATGACTTCCT | 78 | 2848 |
| 1316847 | N/A | N/A | 106962 | 106981 | ACAGTTCCAACATACATTCC | 121 | 2849 |
| 1316867 | N/A | N/A | 142717 | 142736 | ACTATTCTTATCCTCACCAC | 83 | 2850 |
| 1316906 | N/A | N/A | 17485 | 17504 | TTAACATCCAATCACTATTT | 85 | 2851 |
| 1316936 | N/A | N/A | 13061 | 13080 | CACGAGTTCACCAATGGCTG | 42 | 2852 |
| 1316961 | N/A | N/A | 137081 | 137100 | TTTTCATGAACACCTGCCGT | 101 | 2853 |
| 1316969 | N/A | N/A | 110180 | 110199 | TCTGAATGCAACATGCACCT | 92 | 2854 |
| 1317014 | 1497 | 1516 | 51878 | 51897 | AGAGTCATCCTCCAAGGCTT | 73 | 2855 |
| 1317016 | N/A | N/A | 59269 | 59288 | TCCAATTTTAATTGTAGCAA | 78 | 2856 |
| 1317017 | N/A | N/A | 9986 | 10005 | GTACTATATTATATTCCCAC | 19 | 2857 |
| 1317150 | N/A | N/A | 151420 | 151439 | TCATGTCTTTCTGCTGGGCA | 86 | 2858 |
| 1317203 | N/A | N/A | 5976 | 5995 | CCCATCTCACTCACATAAAT | 85 | 2859 |
| 1317217 | N/A | N/A | 112229 | 112248 | TCCATTTATTTCCTTCCTTC | 63 | 2860 |

TABLE 39
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 31 | 858 |
| 1314480 | N/A | N/A | 57724 | 57743 | AGTTATTTCCTTCAACTGTG | 94 | 2861 |
| 1314505 | N/A | N/A | 23557 | 23576 | ACTGTTTTTCCTATCAGCTT | 91 | 2862 |
| 1314517 | N/A | N/A | 133767 | 133786 | TCTGCAGTAAACCCACCTAT | 74 | 2863 |
| 1314559 | N/A | N/A | 33940 | 33959 | GCATCTAGAAACCATCTCCC | 79 | 2864 |
| 1314603 | N/A | N/A | 114949 | 114968 | GTATCATCAATTTTAGCCAG | 26 | 2865 |
| 1314609 | N/A | N/A | 75525 | 75544 | GAAGTTGATTTTATTGCTTC | 88 | 2866 |
| 1314699 | N/A | N/A | 60561 | 60580 | GCATTTTTCCCACACCTCTC | 92 | 2867 |
| 1314721 | N/A | N/A | 22797 | 22816 | TCTGAATAATTTGAATGCTT | 57 | 2868 |
| 1314818 | N/A | N/A | 16464 | 16483 | CTGCCATTTATATAACCTCA | 48 | 2869 |
| 1314925 | N/A | N/A | 56088 | 56107 | CTGTCATTTATTTCAAGGCA | 76 | 2870 |
| 1314965 | N/A | N/A | 45088 | 45107 | GCCACATCATTTATTTACTT | 67 | 2871 |
| 1315005 | N/A | N/A | 116695 | 116714 | CCTCCAAATTTAACACAGGA | 85 | 2872 |
| 1315008 | N/A | N/A | 106078 | 106097 | ACTCTGACCCCCTCAGTGAC | 117 | 2873 |
| | | | 106134 | 106153 | | | |
| 1315049 | N/A | N/A | 106961 | 106980 | CAGTTCCAACATACATTCCT | 92 | 2874 |
| 1315053 | N/A | N/A | 46701 | 46720 | CTATATCACATTATATGAAC | 92 | 2875 |
| 1315071 | N/A | N/A | 111916 | 111935 | CAGAACATTTTTAATCATGC | 65 | 2876 |
| 1315102 | N/A | N/A | 104488 | 104507 | GCAAGCACACATCAAGCTAA | 115 | 2877 |
| 1315163 | N/A | N/A | 40921 | 40940 | CCATTATTCAAAACTATGCT | 94 | 2878 |
| 1315206 | N/A | N/A | 45654 | 45673 | TGCTTAAAATTTATTCTGCA | 73 | 2879 |
| 1315219 | N/A | N/A | 130024 | 130043 | TGCACATTCAATCAGCACCT | 63 | 2880 |
| 1315236 | N/A | N/A | 137043 | 137062 | GCATTTGTCCTCCCAGGTGC | 102 | 2881 |
| 1315275 | N/A | N/A | 52806 | 52825 | TCCTCTCTAAATATTTAAGT | 96 | 2882 |
| 1315317 | N/A | N/A | 126438 | 126457 | AACTGCTATTTTGAATGCCT | 13 | 2883 |
| 1315365 | N/A | N/A | 110117 | 110136 | TGTTGTATCACACAATTTCC | 90 | 2884 |
| 1315367 | 2633 | 2652 | N/A | N/A | ACACAGTTCCTCACAGCTGT | 100 | 2885 |
| 1315408 | N/A | N/A | 130589 | 130608 | GCAGAAGTAAACATTCATTC | 23 | 2886 |
| 1315412 | N/A | N/A | 69803 | 69822 | TAGACATTTTAAACTTCTGA | 29 | 2887 |
| 1315419 | 1487 | 1506 | 51868 | 51887 | TCCAAGGCTTCTTCTTCTCC | 60 | 2888 |
| 1315421 | N/A | N/A | 44183 | 44202 | GTTTAATATTTTCAGGTCTA | 37 | 2889 |
| 1315536 | N/A | N/A | 108320 | 108339 | TGTATATTAATCTTTGGCAA | 25 | 2890 |
| 1315563 | N/A | N/A | 101094 | 101113 | CACCATTTACCTTATCTGCA | 107 | 2891 |
| 1315592 | N/A | N/A | 96457 | 96476 | ACAAAAATATATAGCATCCA | 86 | 2892 |
| 1315600 | N/A | N/A | 21986 | 22005 | CACCATTTCACATGCAGCAT | 46 | 2893 |
| 1315606 | N/A | N/A | 22191 | 22210 | CTTTTTGACATAGTTAGGTT | 69 | 2894 |
| 1315647 | N/A | N/A | 14985 | 15004 | GCAGTTACACTCATACAGAC | 129 | 2895 |
| 1315653 | N/A | N/A | 19026 | 19045 | AACTGAATTTTTTAACACAT | 82 | 2896 |
| 1315777 | N/A | N/A | 28750 | 28769 | GGACAAACATTTATTTCAAC | 33 | 2897 |
| 1315792 | N/A | N/A | 32819 | 32838 | CCAGTTATATTCTTTAAGGT | 88 | 2898 |
| 1315794 | N/A | N/A | 3516 | 3535 | TCATGTCAATGTATTACTTA | 29 | 2899 |
| 1315887 | N/A | N/A | 42028 | 42047 | GTCTCAAGCCTACAAGACTC | 112 | 2900 |
| 1315903 | N/A | N/A | 36062 | 36081 | TCACCAACTTCATCTGTCCT | 94 | 2901 |
| 1315908 | N/A | N/A | 84823 | 84842 | ATCCTTTAAATTTCATGTGC | 50 | 2902 |
| 1315917 | N/A | N/A | 19731 | 19750 | CTATTGTCATACAATTCACT | 76 | 2903 |
| 1315958 | N/A | N/A | 160912 | 160931 | CGCACCATTTCTACCTGGAT | 104 | 2904 |
| 1315967 | N/A | N/A | 17482 | 17501 | ACATCCAATCACTATTTTAA | 84 | 2905 |
| 1316162 | N/A | N/A | 127448 | 127467 | ACAGTCACAGAATTTACCTA | 30 | 2906 |
| 1316283 | N/A | N/A | 120223 | 120242 | CACACACAACTTACTTGCAA | 65 | 2907 |
| 1316302 | N/A | N/A | 118229 | 118248 | CCAACACATTCTTTCTCTTA | 71 | 2908 |
| 1316432 | N/A | N/A | 17886 | 17905 | CAGTGTTTTTCTTAACGATA | 100 | 2909 |
| 1316434 | N/A | N/A | 66632 | 66651 | TTGTCTCCAATCTCAGGGTC | 70 | 2910 |
| 1316437 | N/A | N/A | 134780 | 134799 | TCAAACACTGCAACAGGCAA | 90 | 2911 |
| 1316472 | N/A | N/A | 139710 | 139729 | TATAAAGTATTTCATCCTGC | 101 | 2912 |
| 1316504 | N/A | N/A | 34632 | 34651 | TCATTCCAAAACCCCATGAC | 105 | 2913 |
| 1316575 | N/A | N/A | 10480 | 10499 | AAGCATTTCCATTACAAGTA | 41 | 2914 |
| 1316635 | N/A | N/A | 68733 | 68752 | CCACTTCTCATCTCCCACGG | 59 | 2915 |
| 1316648 | N/A | N/A | 117871 | 117890 | TTTTCCTTTTATATTCATCA | 48 | 2916 |
| 1316745 | N/A | N/A | 9984 | 10003 | ACTATATTATATTCCCACCA | 33 | 2917 |
| 1316775 | N/A | N/A | 49694 | 49713 | ACAACACATTCCCTAAGGCA | 30 | 2918 |
| 1316779 | N/A | N/A | 25218 | 25237 | GCAATTCTCATTTTTCTTTT | 38 | 2919 |
| 1316792 | N/A | N/A | 72711 | 72730 | ACTCTTTATTACTATGGAAA | 63 | 2920 |
| 1316807 | N/A | N/A | 2190 | 2209 | GATAATTTCATTAATCACCA | 51 | 2921 |
| 1316844 | N/A | N/A | 81053 | 81072 | TCCTAAGCATCTACACCACT | 88 | 2922 |
| 1316866 | N/A | N/A | 54638 | 54657 | CAAGCTCTCCATTAATGATC | 63 | 2923 |
| 1316908 | N/A | N/A | 150725 | 150744 | TGGTTGAGTTCTTAACATAA | 27 | 2924 |
| 1316932 | N/A | N/A | 79800 | 79819 | GCCTCAACTATCTCCAATAC | 103 | 2925 |
| 1316967 | N/A | N/A | 142716 | 142735 | CTATTCTTATCCTCACCACA | 82 | 2926 |
| 1317007 | N/A | N/A | 73356 | 73375 | GGGACAAGCCATCACTGGCA | 117 | 2927 |
| 1317025 | N/A | N/A | 71392 | 71411 | GTTTATTGAATTATATCTCC | 87 | 2928 |
| 1317031 | N/A | N/A | 82782 | 82801 | CTGAGGCTTTTCAACAGGTC | 20 | 2929 |
| 1317033 | N/A | N/A | 99492 | 99511 | TTATTCTTTTACAAAGTATT | 95 | 2930 |
| 1317034 | N/A | N/A | 63187 | 63206 | GTAACTCAACTTCCAGGAAT | 89 | 2931 |
| 1317050 | N/A | N/A | 13000 | 13019 | AAGCAGTTCATTTCCCCCAT | 29 | 2932 |
| 1317080 | N/A | N/A | 5973 | 5992 | ATCTCACTCACATAAATGCC | 92 | 2933 |
| 1317159 | 6194 | 6213 | 133255 | 133274 | GCCAGAAGCATTTCTACCCG | 85 | 2934 |
| 1317192 | N/A | N/A | 7636 | 7655 | AACACAGACACTCATACATA | 77 | 2935 |
| 1317194 | 1259 | 1278 | 47572 | 47591 | GCTCCGGTCACAACATTGTG | 64 | 2936 |
| 1317207 | N/A | N/A | 146723 | 146742 | TAGGTTTGTATTATTATCAT | 58 | 2937 |

TABLE 40
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 34 | 858 |
| 1314561 | N/A | N/A | 36060 | 36079 | ACCAACTTCATCTGTCCTCC | 77 | 2938 |
| 1314575 | N/A | N/A | 56087 | 56106 | TGTCATTTATTTCAAGGCAC | 67 | 2939 |
| 1314610 | N/A | N/A | 68731 | 68750 | ACTTCTCATCTCCCACGGCA | 61 | 2940 |
| 1314638 | N/A | N/A | 79798 | 79817 | CTCAACTATCTCCAATACAC | 85 | 2941 |
| 1314675 | N/A | N/A | 22178 | 22197 | TTAGGTTGAAATCTACCATC | 94 | 2942 |
| 1314718 | N/A | N/A | 44174 | 44193 | TTTCAGGTCTAACAATGGCA | 55 | 2943 |
| 1314762 | 2234 | 2253 | N/A | N/A | AGCACATTTTTTCCCCCTGT | 8 | 2944 |
| 1314781 | N/A | N/A | 130023 | 130042 | GCACATTCAATCAGCACCTT | 48 | 2945 |
| 1314847 | N/A | N/A | 71296 | 71315 | CACCATGGTACAACTTCCTA | 87 | 2946 |
| 1314860 | N/A | N/A | 134771 | 134790 | GCAACAGGCAATACACAGTA | 49 | 2947 |
| 1314871 | N/A | N/A | 136978 | 136997 | CAGGAGGTTTCTCCCGTCCT | 84 | 2948 |
| 1314875 | N/A | N/A | 19025 | 19044 | ACTGAATTTTTTAACACATT | 84 | 2949 |
| 1314885 | N/A | N/A | 45084 | 45103 | CATCATTTATTTACTTTGGA | 68 | 2950 |
| 1314890 | N/A | N/A | 75524 | 75543 | AAGTTGATTTTATTGCTTCA | 72 | 2951 |
| 1315033 | N/A | N/A | 9955 | 9974 | GACCTAGTTTTTCTAAATCC | 60 | 2952 |
| 1315061 | N/A | N/A | 45653 | 45672 | GCTTAAAATTTATTCTGCAC | 84 | 2953 |
| 1315067 | N/A | N/A | 22784 | 22803 | AATGCTTTTAATTTAGAGAC | 83 | 2954 |
| 1315164 | N/A | N/A | 40891 | 40910 | ACTTGATTCCAATCATTGTT | 88 | 2955 |
| 1315191 | N/A | N/A | 99480 | 99499 | AAAGTATTTCCTGCTTGCTC | 49 | 2956 |
| 1315196 | N/A | N/A | 12774 | 12793 | ACGATGTTGCCCAATTGCTC | 41 | 2957 |
| 1315203 | N/A | N/A | 146719 | 146738 | TTTGTATTATTATCATGCTT | 86 | 2958 |
| 1315233 | N/A | N/A | 106067 | 106086 | CTCAGTGACACATACGCTCA | 99 | 2959 |
| | | | 106123 | 106142 | | | |
| | | | 106179 | 106198 | | | |
| 1315300 | N/A | N/A | 73248 | 73267 | ACTCAGTACCAGTACCTGCA | 96 | 2960 |
| 1315329 | N/A | N/A | 130583 | 130602 | GTAAACATTCATTCTAGATT | 61 | 2961 |
| 1315338 | N/A | N/A | 7632 | 7651 | CAGACACTCATACATATTGC | 59 | 2962 |
| 1315369 | N/A | N/A | 52805 | 52824 | CCTCTCTAAATATTTAAGTC | 94 | 2963 |
| 1315373 | 4328 | 4347 | 101035 | 101054 | GTAGACACTTTCTGGAGGAC | 38 | 2964 |
| 1315422 | N/A | N/A | 46699 | 46718 | ATATCACATTATATGAACAT | 90 | 2965 |
| 1315427 | N/A | N/A | 17478 | 17497 | CCAATCACTATTTTAATGTC | 67 | 2966 |
| 1315463 | N/A | N/A | 117867 | 117886 | CCTTTTATATTCATCAGAAC | 81 | 2967 |
| 1315475 | N/A | N/A | 111915 | 111934 | AGAACATTTTTAATCATGCA | 89 | 2968 |
| 1315483 | 1486 | 1505 | 51867 | 51886 | CCAAGGCTTCTTCTTCTCCT | 46 | 2969 |
| 1315484 | N/A | N/A | 127445 | 127464 | GTCACAGAATTTACCTACAT | 42 | 2970 |
| 1315488 | N/A | N/A | 34613 | 34632 | CTAGTTTGAATATCTCCTTA | 65 | 2971 |
| 1315526 | N/A | N/A | 42018 | 42037 | TACAAGACTCACCATAGTCA | 75 | 2972 |
| 1315554 | N/A | N/A | 25180 | 25199 | CTTCAGATACTTCAATCCTA | 72 | 2973 |
| 1315598 | N/A | N/A | 19727 | 19746 | TGTCATACAATTCACTTCTA | 63 | 2974 |
| 1315612 | N/A | N/A | 106960 | 106979 | AGTTCCAACATACATTCCTC | 84 | 2975 |
| 1315712 | N/A | N/A | 108305 | 108324 | GGCAAACTTCAAACACAGCC | 64 | 2976 |
| 1315749 | N/A | N/A | 49605 | 49624 | TCATGTCTTTACAACTGCAT | 30 | 2977 |
| 1315758 | N/A | N/A | 104460 | 104479 | CGTGATCTACTCTAACCCCA | 92 | 2978 |
| 1315791 | N/A | N/A | 33929 | 33948 | CCATCTCCCAATTCAACTCT | 81 | 2979 |
| 1315793 | N/A | N/A | 120198 | 120217 | GGGCAGATTAAATAAGGTCA | 73 | 2980 |
| 1315860 | N/A | N/A | 116347 | 116366 | CCATCTTAACCATTTTTGGC | 72 | 2981 |
| 1315905 | N/A | N/A | 96357 | 96376 | GAGGAGTATCAATCAACCAA | 75 | 2982 |
| 1315939 | N/A | N/A | 150478 | 150497 | GCCAGTCTTCTCTCATTCTA | 76 | 2983 |
| 1316006 | N/A | N/A | 114843 | 114862 | TCCTTCGACAACACAGGGCA | 91 | 2984 |
| 1316086 | N/A | N/A | 82772 | 82791 | TCAACAGGTCAATCTTCCTC | 58 | 2985 |
| 1316108 | 1256 | 1275 | 47569 | 47588 | CCGGTCACAACATTGTGGTC | 87 | 2986 |
| 1316127 | N/A | N/A | 66581 | 66600 | TGCTGTACTTTCTTTTTTTC | 54 | 2987 |
| 1316174 | N/A | N/A | 17875 | 17894 | TTAACGATATTTATAACTGC | 101 | 2988 |
| 1316247 | 6190 | 6209 | 133251 | 133270 | GAAGCATTTCTACCCGGCGA | 71 | 2989 |
| 1316301 | N/A | N/A | 21885 | 21904 | TTTCACAGTTTTAGAGGCCA | 54 | 2990 |
| 1316385 | N/A | N/A | 69792 | 69811 | AACTTCTGATACATTACCAA | 46 | 2991 |
| 1316408 | N/A | N/A | 2170 | 2189 | CGTAAGTTCACCACTTTACT | 87 | 2992 |
| 1316419 | N/A | N/A | 14982 | 15001 | GTTACACTCATACAGACAGA | 93 | 2993 |
| 1316438 | N/A | N/A | 133762 | 133781 | AGTAAACCCACCTATAAGGC | 86 | 2994 |
| 1316471 | N/A | N/A | 5972 | 5991 | TCTCACTCACATAAATGCCA | 85 | 2995 |
| 1316477 | N/A | N/A | 81050 | 81069 | TAAGCATCTACACCACTTCC | 79 | 2996 |
| 1316537 | 1722 | 1741 | 53737 | 53756 | CAAGATATCCTCCTCATCCC | 73 | 2997 |
| 1316544 | N/A | N/A | 3513 | 3532 | TGTCAATGTATTACTTATGC | 18 | 2998 |
| 1316668 | N/A | N/A | 16461 | 16480 | CCATTTATATAACCTCATTA | 75 | 2999 |
| 1316673 | 2617 | 2636 | 59189 | 59208 | CTGTACAAGCTAACTTGCAA | 78 | 3000 |
| 1316674 | N/A | N/A | 72620 | 72639 | CCAATGGTATCCTTTAACAC | 86 | 3001 |
| 1316849 | N/A | N/A | 60528 | 60547 | AAGAATACTTTATCTCAGTA | 74 | 3002 |
| 1316850 | N/A | N/A | 142693 | 142712 | GCACAGGATCCACAGCCTGA | 97 | 3003 |
| 1316892 | N/A | N/A | 139709 | 139728 | ATAAAGTATTTCATCCTGCA | 77 | 3004 |
| 1316894 | N/A | N/A | 23556 | 23575 | CTGTTTTTCCTATCAGCTTT | 101 | 3005 |
| 1316945 | N/A | N/A | 63067 | 63086 | CCATAACTAAAATTACTGTT | 71 | 3006 |
| 1317047 | N/A | N/A | 126404 | 126423 | TGTTTGTCCCAATAATATGC | 61 | 3007 |
| 1317048 | N/A | N/A | 28748 | 28767 | ACAAACATTTATTTCAACAT | 110 | 3008 |
| 1317075 | N/A | N/A | 110116 | 110135 | GTTGTATCACACAATTTCCA | 60 | 3009 |
| 1317092 | N/A | N/A | 160873 | 160892 | CCAAACGCAACTCCTGGACC | 82 | 3010 |
| 1317116 | N/A | N/A | 84821 | 84840 | CCTTTAAATTTCATGTGCTT | 69 | 3011 |
| 1317156 | N/A | N/A | 32818 | 32837 | CAGTTATATTCTTTAAGGTC | 72 | 3012 |
| 1317186 | N/A | N/A | 118226 | 118245 | ACACATTCTTTCTCTTACTC | 64 | 3013 |
| 1317223 | N/A | N/A | 10454 | 10473 | TGTGTTGTCCTTTTATAACC | 46 | 3014 |

TABLE 41
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 42 | 858 |
| 1314543 | 2592 | 2611 | 59164 | 59183 | AGAAGACTCATCCTTCAGTG | 58 | 3015 |
| 1314558 | N/A | N/A | 16051 | 16070 | CCAACATTCCATGAATTCCA | 59 | 3016 |
| 1314563 | N/A | N/A | 142638 | 142657 | TGGGTGTTTTCATATTCCTT | 86 | 3017 |
| 1314602 | N/A | N/A | 126360 | 126379 | GCAGCAGAAATTTCACAAGC | 47 | 3018 |
| 1314645 | N/A | N/A | 57687 | 57706 | TCACCATTTTTTCCCCCTGT | 63 | 3019 |
| 1314705 | N/A | N/A | 19024 | 19043 | CTGAATTTTTTAACACATTG | 74 | 3020 |
| 1314727 | N/A | N/A | 19726 | 19745 | GTCATACAATTCACTTCTAC | 45 | 3021 |
| 1314788 | N/A | N/A | 127444 | 127463 | TCACAGAATTTACCTACATT | 78 | 3022 |
| 1314827 | N/A | N/A | 108304 | 108323 | GCAAACTTCAAACACAGCCT | 45 | 3023 |
| 1314861 | N/A | N/A | 106959 | 106978 | GTTCCAACATACATTCCTCT | 104 | 3024 |
| 1314891 | N/A | N/A | 130010 | 130029 | GCACCTTTATTTCAGAACTC | 40 | 3025 |
| 1314892 | N/A | N/A | 41867 | 41886 | CAGTGCAGTTCACATTCAAT | 66 | 3026 |
| 1314913 | N/A | N/A | 72618 | 72637 | AATGGTATCCTTTAACACGC | 71 | 3027 |
| 1314920 | N/A | N/A | 5957 | 5976 | TGCCAATATCTTTATTCCAT | 41 | 3028 |
| 1314967 | N/A | N/A | 62837 | 62856 | GCATTACTTTTTACAATGGA | 8 | 3029 |
| 1314984 | N/A | N/A | 9954 | 9973 | ACCTAGTTTTTCTAAATCCT | 51 | 3030 |
| 1315022 | N/A | N/A | 17873 | 17892 | AACGATATTTATAACTGCTC | 93 | 3031 |
| 1315158 | N/A | N/A | 133761 | 133780 | GTAAACCCACCTATAAGGCA | 94 | 3032 |
| 1315172 | N/A | N/A | 17477 | 17496 | CAATCACTATTTTAATGTCA | 81 | 3033 |
| 1315184 | N/A | N/A | 118224 | 118243 | ACATTCTTTCTCTTACTCTA | 85 | 3034 |
| 1315218 | N/A | N/A | 106065 | 106084 | CAGTGACACATACGCTCACA | 103 | 3035 |
| | | | 106121 | 106140 | | | |
| | | | 106177 | 106196 | | | |
| 1315231 | N/A | N/A | 111858 | 111877 | AGTGTGTTCAACCATTACTT | 88 | 3036 |
| 1315240 | N/A | N/A | 101020 | 101039 | AGGACATCAAACCATCTGCC | 87 | 3037 |
| 1315252 | N/A | N/A | 22177 | 22196 | TAGGTTGAAATCTACCATCA | 101 | 3038 |
| 1315268 | N/A | N/A | 104340 | 104359 | CAGCAGCTACGATCTTCCTA | 81 | 3039 |
| 1315331 | N/A | N/A | 20135 | 20154 | CACCACATTCCCTCTGGAGT | 95 | 3040 |
| 1315340 | N/A | N/A | 3512 | 3531 | GTCAATGTATTACTTATGCA | 13 | 3041 |
| 1315362 | 1557 | 1576 | 53572 | 53591 | ACTGATCTCATCCTTCACTG | 49 | 3042 |
| 1315366 | N/A | N/A | 56037 | 56056 | TTACTGATCACTCATTCCCA | 70 | 3043 |
| 1315375 | N/A | N/A | 51836 | 51855 | GCCTAGTAAATAATGTACCA | 94 | 3044 |
| 1315458 | N/A | N/A | 117849 | 117868 | ACCGAAGACATTTTCATTCT | 113 | 3045 |
| 1315459 | N/A | N/A | 84110 | 84129 | AGACACATACTTCATATCTA | 92 | 3046 |
| 1315532 | N/A | N/A | 46697 | 46716 | ATCACATTATATGAACATAA | 84 | 3047 |
| 1315579 | N/A | N/A | 32764 | 32783 | GCTTCAGAAATATAACACAT | 78 | 3048 |
| 1315626 | N/A | N/A | 75478 | 75497 | TGGCAGAACATTCTACAACC | 47 | 3049 |
| 1315633 | N/A | N/A | 110115 | 110134 | TTGTATCACACAATTTCCAT | 78 | 3050 |
| 1315676 | N/A | N/A | 68707 | 68726 | TTTGCATTCCCTTCATGGCA | 69 | 3051 |
| 1315786 | N/A | N/A | 40890 | 40909 | CTTGATTCCAATCATTGTTC | 86 | 3052 |
| 1315844 | N/A | N/A | 7631 | 7650 | AGACACTCATACATATTGCT | 83 | 3053 |
| 1315854 | N/A | N/A | 114786 | 114805 | GTCACAGCCACTTAGCAGAA | 95 | 3054 |
| 1315859 | N/A | N/A | 116260 | 116279 | GGCATGAACACATCTCACTA | 71 | 3055 |
| 1315884 | N/A | N/A | 45617 | 45636 | CTTGGATCACAAATTTCCTT | 99 | 3056 |
| 1315900 | N/A | N/A | 96356 | 96375 | AGGAGTATCAATCAACCAAT | 87 | 3057 |
| 1316022 | N/A | N/A | 130507 | 130526 | CAAGTGTCATCTACACAGGA | 81 | 3058 |
| 1316040 | N/A | N/A | 79794 | 79813 | ACTATCTCCAATACACAACA | 84 | 3059 |
| 1316062 | N/A | N/A | 65968 | 65987 | GGGTAACATTTTCCTACAAA | 17 | 3060 |
| 1316085 | 3350 | 3369 | 73156 | 73175 | AGGGTCAGAATCATTGTGGC | 53 | 3061 |
| 1316131 | N/A | N/A | 14860 | 14879 | GGCTGTACAACATTACGAAT | 63 | 3062 |
| 1316167 | N/A | N/A | 12677 | 12696 | GCCTCACTTCCCCCATTATA | 95 | 3063 |
| 1316168 | N/A | N/A | 45038 | 45057 | AAGTCTCACATCCATACCCT | 103 | 3064 |
| 1316178 | N/A | N/A | 71283 | 71302 | CTTCCTACTTTAGACACCTC | 76 | 3065 |
| 1316189 | N/A | N/A | 34599 | 34618 | TCCTTAGTATTAACAAGAAA | 111 | 3066 |
| 1316211 | N/A | N/A | 132766 | 132785 | GATGAGAGATTCATTTTCAA | 102 | 3067 |
| 1316213 | N/A | N/A | 36019 | 36038 | TCCAGTTTCAACCCAGAGCA | 103 | 3068 |
| 1316291 | N/A | N/A | 60517 | 60536 | ATCTCAGTAAATACAAGCTT | 105 | 3069 |
| 1316332 | N/A | N/A | 49604 | 49623 | CATGTCTTTACAACTGCATT | 60 | 3070 |
| 1316349 | N/A | N/A | 28160 | 28179 | CAGGAAGCCTTTCCTGCCTC | 96 | 3071 |
| 1316382 | N/A | N/A | 22783 | 22802 | ATGCTTTTAATTTAGAGACT | 92 | 3072 |
| 1316410 | N/A | N/A | 23474 | 23493 | GTTTTCTATTTTACTGACTT | 65 | 3073 |
| 1316454 | N/A | N/A | 25176 | 25195 | AGATACTTCAATCCTAGACA | 63 | 3074 |
| 1316470 | N/A | N/A | 139683 | 139702 | AGGCACATAATATAATCAGC | 75 | 3075 |
| 1316475 | N/A | N/A | 52804 | 52823 | CTCTCTAAATATTTAAGTCT | 100 | 3076 |
| 1316486 | N/A | N/A | 33924 | 33943 | TCCCAATTCAACTCTTCACC | 88 | 3077 |
| 1316495 | N/A | N/A | 44172 | 44191 | TCAGGTCTAACAATGGCACT | 103 | 3078 |
| 1316529 | N/A | N/A | 81049 | 81068 | AAGCATCTACACCACTTCCC | 88 | 3079 |
| 1316641 | N/A | N/A | 99445 | 99464 | AACGACAGCATTCTTGCCAC | 88 | 3080 |
| 1316643 | N/A | N/A | 134700 | 134719 | TCAAATGCACAAATTCCACT | 96 | 3081 |
| 1316722 | N/A | N/A | 10448 | 10467 | GTCCTTTTATAACCACATTC | 44 | 3082 |
| 1316740 | N/A | N/A | 120197 | 120216 | GGCAGATTAAATAAGGTCAA | 86 | 3083 |
| 1316786 | N/A | N/A | 160857 | 160876 | GACCACCTCAACTCTCCTTC | 80 | 3084 |
| 1316895 | N/A | N/A | 136929 | 136948 | CCATGGCTGCACCATACCCA | 94 | 3085 |
| 1316907 | N/A | N/A | 146718 | 146737 | TTGTATTATTATCATGCTTA | 51 | 3086 |
| 1316948 | 7827 | 7846 | 150346 | 150365 | TCTCTTTGAAACCATTGCTT | 68 | 3087 |
| 1317011 | N/A | N/A | 2164 | 2183 | TTCACCACTTTACTTGGCAA | 63 | 3088 |
| 1317073 | N/A | N/A | 47525 | 47544 | CAGTTCATAAACCTAGAAAC | 101 | 3089 |
| 1317102 | N/A | N/A | 69788 | 69807 | TCTGATACATTACCAATGCA | 48 | 3090 |
| 1317134 | N/A | N/A | 82771 | 82790 | CAACAGGTCAATCTTCCTCC | 72 | 3091 |

TABLE 42
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | Sequence | (% | ID |
| Number | Site | Site | Site | Site | (5′ to 3′) | UTC) | NO |
| 388241 | 4862 | 4881 | 106917 | 106936 | CTCAGTAACATTGACACCAC | 44 | 858 |
| 1314481 | N/A | N/A | 46683 | 46702 | ACATAATGAATTTATTCTTT | 85 | 3092 |
| 1314515 | N/A | N/A | 79789 | 79808 | CTCCAATACACAACACATGA | 94 | 3093 |
| 1314634 | N/A | N/A | 23197 | 23216 | GCCAGTCTCAAACAATCCAC | 78 | 3094 |
| 1314647 | N/A | N/A | 110110 | 110129 | TCACACAATTTCCATTGCGG | 47 | 3095 |
| 1314708 | N/A | N/A | 142633 | 142652 | GTTTTCATATTCCTTAGGTA | 16 | 3096 |
| 1314714 | N/A | N/A | 136758 | 136777 | GTAGAGGAAATCTCGCTGCA | 64 | 3097 |
| 1314760 | N/A | N/A | 52755 | 52774 | TAGATAGGATTATACAACCA | 63 | 3098 |
| 1314799 | N/A | N/A | 14805 | 14824 | ACAGTCAATTTTATGTGCAT | 61 | 3099 |
| 1314839 | N/A | N/A | 17476 | 17495 | AATCACTATTTTAATGTCAC | 69 | 3100 |
| 1314853 | N/A | N/A | 34542 | 34561 | ACAGTTTATTCCAAATCATT | 65 | 3101 |
| 1314857 | N/A | N/A | 117847 | 117866 | CGAAGACATTTTCATTCTGT | 83 | 3102 |
| 1314864 | N/A | N/A | 19010 | 19029 | ACATTGTTAATATATTTTCC | 52 | 3103 |
| 1314907 | N/A | N/A | 62836 | 62855 | CATTACTTTTTACAATGGAC | 17 | 3104 |
| 1314918 | 2233 | 2252 | N/A | N/A | GCACATTTTTTCCCCCTGTT | 8 | 3105 |
| 1314926 | N/A | N/A | 22176 | 22195 | AGGTTGAAATCTACCATCAA | 77 | 3106 |
| 1314977 | N/A | N/A | 32695 | 32714 | TTAGTGATTATACCTCTGCA | 67 | 3107 |
| 1314996 | N/A | N/A | 15972 | 15991 | GAGATCTTATTTCCTGGGTC | 43 | 3108 |
| 1315093 | N/A | N/A | 45031 | 45050 | ACATCCATACCCTTTTTTCC | 98 | 3109 |
| 1315146 | N/A | N/A | 27915 | 27934 | ACCTAGTTTTCACCATACAC | 70 | 3110 |
| 1315156 | N/A | N/A | 116253 | 116272 | ACACATCTCACTAGAGACTT | 50 | 3111 |
| 1315162 | N/A | N/A | 106957 | 106976 | TCCAACATACATTCCTCTTA | 69 | 3112 |
| 1315242 | N/A | N/A | 146707 | 146726 | TCATGCTTAAATCTTTTCTG | 79 | 3113 |
| 1315290 | N/A | N/A | 105978 | 105997 | CAGAGGCACACTCTGACCCC | 97 | 3114 |
| | | | 106033 | 106052 | | | |
| | | | 106087 | 106106 | | | |
| | | | 106143 | 106162 | | | |
| 1315308 | N/A | N/A | 20134 | 20153 | ACCACATTCCCTCTGGAGTA | 81 | 3115 |
| 1315389 | N/A | N/A | 7629 | 7648 | ACACTCATACATATTGCTTA | 62 | 3116 |
| 1315410 | N/A | N/A | 133729 | 133748 | GTCCTGTGAATTTATTCCCG | 60 | 3117 |
| 1315467 | N/A | N/A | 56033 | 56052 | TGATCACTCATTCCCACTAC | 86 | 3118 |
| 1315514 | N/A | N/A | 99153 | 99172 | ACAACACTTTTAAGAATCTC | 99 | 3119 |
| 1315521 | N/A | N/A | 111717 | 111736 | CCACATCTTACAACAAGGGA | 60 | 3120 |
| 1315551 | N/A | N/A | 130464 | 130483 | TCAACCCCGTTTACAGGCAT | 73 | 3121 |
| 1315564 | 3349 | 3368 | 73155 | 73174 | GGGTCAGAATCATTGTGGCC | 92 | 3122 |
| 1315568 | N/A | N/A | 75477 | 75496 | GGCAGAACATTCTACAACCA | 51 | 3123 |
| 1315678 | N/A | N/A | 65967 | 65986 | GGTAACATTTTCCTACAAAA | 22 | 3124 |
| 1315682 | N/A | N/A | 139682 | 139701 | GGCACATAATATAATCAGCA | 69 | 3125 |
| 1315684 | N/A | N/A | 108287 | 108306 | CCTTGACTAAACTATTCTGC | 83 | 3126 |
| 1315709 | N/A | N/A | 17872 | 17891 | ACGATATTTATAACTGCTCT | 56 | 3127 |
| 1315795 | N/A | N/A | 10442 | 10461 | TTATAACCACATTCACCTCC | 70 | 3128 |
| 1315799 | N/A | N/A | 114688 | 114707 | GCAAGCACTTACGAGTGCCT | 89 | 3129 |
| 1315812 | N/A | N/A | 49039 | 49058 | TCGCAGTTCAACATTTTAAT | 33 | 3130 |
| 1315850 | N/A | N/A | 100969 | 100988 | AATATCTTTAAATAATCTCC | 90 | 3131 |
| 1315865 | N/A | N/A | 126359 | 126378 | CAGCAGAAATTTCACAAGCC | 52 | 3132 |
| 1315879 | N/A | N/A | 19673 | 19692 | GCGAACAATTTAATTAATCT | 33 | 3133 |
| 1315912 | N/A | N/A | 25119 | 25138 | AACCTACAATATTCTTGGAA | 82 | 3134 |
| 1315981 | N/A | N/A | 82403 | 82422 | TCAAGGATACAAAATTTCAT | 68 | 3135 |
| 1316015 | N/A | N/A | 134696 | 134715 | ATGCACAAATTCCACTATCA | 63 | 3136 |
| 1316039 | N/A | N/A | 95286 | 95305 | GCCTAAAATTTTCCAAACTT | 79 | 3137 |
| 1316051 | N/A | N/A | 5955 | 5974 | CCAATATCTTTATTCCATCT | 42 | 3138 |
| 1316070 | N/A | N/A | 160850 | 160869 | TCAACTCTCCTTCCCTGAGT | 100 | 3139 |
| 1316147 | N/A | N/A | 150214 | 150233 | ACCAGAACTTTCCCTCCGCT | 88 | 3140 |
| 1316210 | N/A | N/A | 41865 | 41884 | GTGCAGTTCACATTCAATCA | 55 | 3141 |
| 1316235 | N/A | N/A | 68706 | 68725 | TTGCATTCCCTTCATGGCAC | 47 | 3142 |
| 1316275 | N/A | N/A | 118171 | 118190 | GACTGTTGAATACAATCACC | 79 | 3143 |
| 1316407 | N/A | N/A | 127443 | 127462 | CACAGAATTTACCTACATTT | 62 | 3144 |
| 1316411 | N/A | N/A | 69445 | 69464 | CTGGCTTATTTTAATTGTTG | 87 | 3145 |
| 1316441 | N/A | N/A | 53405 | 53424 | CAGTGCATCCTTCAAGCATC | 74 | 3146 |
| 1316446 | N/A | N/A | 132618 | 132637 | GAAACTCGATTTACCAAGGC | 56 | 3147 |
| 1316501 | N/A | N/A | 72616 | 72635 | TGGTATCCTTTAACACGCCA | 74 | 3148 |
| 1316712 | N/A | N/A | 33845 | 33864 | GATGGTAGTCTAAATTACGA | 61 | 3149 |
| 1316730 | N/A | N/A | 3511 | 3530 | TCAATGTATTACTTATGCAA | 50 | 3150 |
| 1316762 | N/A | N/A | 12593 | 12612 | CCAGCATTTAAACCCAGGTC | 31 | 3151 |
| 1316790 | N/A | N/A | 59112 | 59131 | GAAAATGTATTTCCTAACCA | 83 | 3152 |
| 1316798 | N/A | N/A | 22749 | 22768 | GCATCTGCTCTATCTTGGCA | 73 | 3153 |
| 1316821 | N/A | N/A | 84108 | 84127 | ACACATACTTCATATCTAAC | 93 | 3154 |
| 1316822 | N/A | N/A | 60516 | 60535 | TCTCAGTAAATACAAGCTTA | 72 | 3155 |
| 1316902 | N/A | N/A | 43973 | 43992 | CTGATTTTTTTCTTTAAGAC | 78 | 3156 |
| 1316974 | N/A | N/A | 119915 | 119934 | TGGACAACACTTCAACAGTA | 74 | 3157 |
| 1316981 | N/A | N/A | 45615 | 45634 | TGGATCACAAATTTCCTTTA | 71 | 3158 |
| 1316985 | N/A | N/A | 2149 | 2168 | GGCAACCACAACTCATTCAA | 39 | 3159 |
| 1317004 | N/A | N/A | 51827 | 51846 | ATAATGTACCAACATTCCTT | 73 | 3160 |
| 1317012 | N/A | N/A | 47485 | 47504 | GTCAGAGTTTATAAAATGCA | 67 | 3161 |
| 1317022 | N/A | N/A | 40889 | 40908 | TTGATTCCAATCATTGTTCA | 68 | 3162 |
| 1317064 | N/A | N/A | 104336 | 104355 | AGCTACGATCTTCCTAGCTC | 80 | 3163 |
| 1317095 | N/A | N/A | 130006 | 130025 | CTTTATTTCAGAACTCACAC | 83 | 3164 |
| 1317155 | N/A | N/A | 9953 | 9972 | CCTAGTTTTTCTAAATCCTC | 65 | 3165 |
| 1317196 | N/A | N/A | 81048 | 81067 | AGCATCTACACCACTTCCCT | 81 | 3166 |
| 1317229 | N/A | N/A | 71084 | 71103 | ATCCTGTTTTTCCATCCCAA | 47 | 3167 |
| 1317232 | N/A | N/A | 35996 | 36015 | AACACAGCTTTCTCATCAAA | 85 | 3168 |

TABLE 43
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | Sequence | (% | ID |
| Number | Site | Site | Site | Site | (5′ to 3′) | UTC) | NO |
| 388241 | 4862 | 4881 | 106917 | 106936 | CTCAGTAACATTGACACCAC | 25 | 858 |
| 1314489 | N/A | N/A | 22601 | 22620 | TGTTCTACAAATCATTGAGA | 74 | 3169 |
| 1314538 | N/A | N/A | 119789 | 119808 | AGAACACACCACCCTTATCC | 90 | 3170 |
| 1314591 | N/A | N/A | 3163 | 3182 | AAGGTAATCATATATTACCT | 99 | 3171 |
| 1314600 | N/A | N/A | 23196 | 23215 | CCAGTCTCAAACAATCCACC | 89 | 3172 |
| 1314652 | N/A | N/A | 52754 | 52773 | AGATAGGATTATACAACCAA | 50 | 3173 |
| 1314720 | N/A | N/A | 118167 | 118186 | GTTGAATACAATCACCACAT | 52 | 3174 |
| 1314732 | N/A | N/A | 46679 | 46698 | AATGAATTTATTCTTTTTCC | 87 | 3175 |
| 1314766 | N/A | N/A | 160838 | 160857 | CCCTGAGTCCTCCATGCCCA | 67 | 3176 |
| 1314816 | N/A | N/A | 45613 | 45632 | GATCACAAATTTCCTTTATA | 74 | 3177 |
| 1314836 | N/A | N/A | 17860 | 17879 | ACTGCTCTTTACACAGGCTA | 77 | 3178 |
| 1314889 | N/A | N/A | 47484 | 47503 | TCAGAGTTTATAAAATGCAT | 95 | 3179 |
| 1314921 | N/A | N/A | 106956 | 106975 | CCAACATACATTCCTCTTAC | 91 | 3180 |
| 1314928 | N/A | N/A | 56032 | 56051 | GATCACTCATTCCCACTACA | 37 | 3181 |
| 1314932 | N/A | N/A | 79767 | 79786 | GCATATAGAACATCGAGACT | 74 | 3182 |
| 1314942 | N/A | N/A | 5948 | 5967 | CTTTATTCCATCTACAAGGC | 44 | 3183 |
| 1314957 | N/A | N/A | 75476 | 75495 | GCAGAACATTCTACAACCAT | 39 | 3184 |
| 1314973 | N/A | N/A | 15971 | 15990 | AGATCTTATTTCCTGGGTCA | 56 | 3185 |
| 1314985 | N/A | N/A | 32693 | 32712 | AGTGATTATACCTCTGCAGC | 63 | 3186 |
| 1314988 | N/A | N/A | 17461 | 17480 | GTCACATACATTAAGTCTCA | 43 | 3187 |
| 1315031 | N/A | N/A | 83974 | 83993 | GCTGACATAAACTTTCCCTA | 58 | 3188 |
| 1315060 | N/A | N/A | 108286 | 108305 | CTTGACTAAACTATTCTGCA | 80 | 3189 |
| 1315077 | N/A | N/A | 82236 | 82255 | GGTTAATTAGCCATTCCACA | 28 | 3190 |
| 1315117 | N/A | N/A | 117846 | 117865 | GAAGACATTTTCATTCTGTT | 63 | 3191 |
| 1315140 | N/A | N/A | 60469 | 60488 | TGGTTCTTTCTTCCTTCCAT | 58 | 3192 |
| 1315147 | N/A | N/A | 129960 | 129979 | GCTTTTGAAATTCTCCATCT | 23 | 3193 |
| 1315181 | N/A | N/A | 114647 | 114666 | TCCAAACAATTTATGAGGGA | 39 | 3194 |
| 1315226 | N/A | N/A | 19671 | 19690 | GAACAATTTAATTAATCTCT | 59 | 3195 |
| 1315235 | N/A | N/A | 139638 | 139657 | ACGCTGACTTTCCCACTTCC | 63 | 3196 |
| 1315279 | N/A | N/A | 20055 | 20074 | GCTTATAGCTTCATCACTCC | 66 | 3197 |
| 1315341 | N/A | N/A | 68690 | 68709 | GCACATGTCAATTTCTATCC | 14 | 3198 |
| 1315378 | N/A | N/A | 2148 | 2167 | GCAACCACAACTCATTCAAA | 68 | 3199 |
| 1315388 | N/A | N/A | 81047 | 81066 | GCATCTACACCACTTCCCTT | 65 | 3200 |
| 1315396 | 3348 | 3367 | 73154 | 73173 | GGTCAGAATCATTGTGGCCA | 71 | 3201 |
| 1315450 | N/A | N/A | 72563 | 72582 | GCAACCTCAAATAAGCAGGA | 44 | 3202 |
| 1315490 | N/A | N/A | 116182 | 116201 | ACCACATGCACACCACTATC | 101 | 3203 |
| 1315491 | N/A | N/A | 130455 | 130474 | TTTACAGGCATTTCAAACTT | 90 | 3204 |
| 1315502 | N/A | N/A | 41863 | 41882 | GCAGTTCACATTCAATCAAC | 52 | 3205 |
| 1315548 | N/A | N/A | 45025 | 45044 | ATACCCTTTTTTCCCCTTCA | 80 | 3206 |
| 1315595 | N/A | N/A | 9952 | 9971 | CTAGTTTTTCTAAATCCTCA | 53 | 3207 |
| 1315610 | N/A | N/A | 51816 | 51835 | ACATTCCTTTTAGTTTGCTA | 97 | 3208 |
| 1315662 | N/A | N/A | 49035 | 49054 | AGTTCAACATTTTAATTTCA | 53 | 3209 |
| 1315677 | N/A | N/A | 133727 | 133746 | CCTGTGAATTTATTCCCGTA | 66 | 3210 |
| 1315680 | N/A | N/A | 104335 | 104354 | GCTACGATCTTCCTAGCTCT | 88 | 3211 |
| 1315695 | N/A | N/A | 105919 | 105938 | CACTCTGACCCCTCAGTGAC | 96 | 3212 |
| | | | 105970 | 105989 | | | |
| | | | 106025 | 106044 | | | |
| 1315722 | N/A | N/A | 35939 | 35958 | CCATGACTATTTACTTAGGC | 30 | 3213 |
| 1315773 | N/A | N/A | 25118 | 25137 | ACCTACAATATTCTTGGAAC | 77 | 3214 |
| 1315784 | N/A | N/A | 142507 | 142526 | TGGGTCATTCTCTAATTACA | 37 | 3215 |
| 1315785 | N/A | N/A | 70585 | 70604 | AGCAAATTAACATTACTTTT | 56 | 3216 |
| 1315823 | N/A | N/A | 40806 | 40825 | AGTAAACTAGTAACTGGCAT | 43 | 3217 |
| 1315888 | N/A | N/A | 65920 | 65939 | ACTCAGTAATACTTTTAGAA | 78 | 3218 |
| 1315893 | N/A | N/A | 53390 | 53409 | GCATCTCTCACACTGCCTCC | 42 | 3219 |
| 1315938 | N/A | N/A | 127442 | 127461 | ACAGAATTTACCTACATTTA | 70 | 3220 |
| 1315974 | N/A | N/A | 69408 | 69427 | TGAATTTTTTTATTTTGGGC | 29 | 3221 |
| 1316014 | N/A | N/A | 12571 | 12590 | CTGGCTTTAAATCTCCTCTG | 54 | 3222 |
| 1316029 | N/A | N/A | 22173 | 22192 | TTGAAATCTACCATCAAGCT | 81 | 3223 |
| 1316049 | N/A | N/A | 14077 | 14096 | TCTGACTGACTATAAAGGCT | 66 | 3224 |
| 1316050 | N/A | N/A | 111602 | 111621 | TGCTCTGTCATCCATGCAGC | 98 | 3225 |
| 1316082 | N/A | N/A | 62709 | 62728 | CTGCTCTACATCCTTTGAAT | 65 | 3226 |
| 1316161 | N/A | N/A | 27667 | 27686 | CCAAACTTAAGCATAAGGGA | 77 | 3227 |
| 1316259 | N/A | N/A | 59086 | 59105 | CAAGCAACAAACATCATCAT | 106 | 3228 |
| 1316279 | N/A | N/A | 126357 | 126376 | GCAGAAATTTCACAAGCCCT | 39 | 3229 |
| 1316293 | N/A | N/A | 19009 | 19028 | CATTGTTAATATATTTTCCA | 67 | 3230 |
| 1316313 | N/A | N/A | 150170 | 150189 | ACCAAATGAAACTCACTTCC | 87 | 3231 |
| 1316490 | 4017 | 4036 | 98625 | 98644 | TCCTAGGATCTCTTCAACAC | 72 | 3232 |
| 1316503 | N/A | N/A | 110109 | 110128 | CACACAATTTCCATTGCGGC | 47 | 3233 |
| 1316569 | N/A | N/A | 34541 | 34560 | CAGTTTATTCCAAATCATTC | 69 | 3234 |
| 1316574 | N/A | N/A | 57535 | 57554 | TCAACAATACATTCATGCAT | 88 | 3235 |
| 1316656 | N/A | N/A | 33842 | 33861 | GGTAGTCTAAATTACGACAT | 58 | 3236 |
| 1316782 | N/A | N/A | 10440 | 10459 | ATAACCACATTCACCTCCCT | 76 | 3237 |
| 1316801 | N/A | N/A | 132598 | 132617 | CGTCACCTCCAACCACGCAC | 93 | 3238 |
| 1316805 | N/A | N/A | 100968 | 100987 | ATATCTTTAAATAATCTCCT | 91 | 3239 |
| 1316812 | N/A | N/A | 94658 | 94677 | CCGTCGCCACATCAAGCTCA | 68 | 3240 |
| 1316951 | N/A | N/A | 134695 | 134714 | TGCACAAATTCCACTATCAA | 83 | 3241 |
| 1316960 | N/A | N/A | 7528 | 7547 | CTGACCATTTACACAAAGTT | 60 | 3242 |
| 1317097 | N/A | N/A | 43595 | 43614 | AATGTATTATTACCATGGCT | 54 | 3243 |
| 1317132 | N/A | N/A | 136720 | 136739 | GACTAAAGATCAACTTCCAA | 90 | 3244 |
| 1317160 | 7482 | 7501 | 146574 | 146593 | GATGAACTCCTTAAAGACTT | 75 | 3245 |

TABLE 44
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 31 | 858 |
| 1314569 | N/A | N/A | 23113 | 23132 | ACTCTGGATTCCATTTGCTT | 68 | 3246 |
| 1314579 | N/A | N/A | 100949 | 100968 | TCTGCACTCAGATATACTCC | 97 | 3247 |
| 1314682 | N/A | N/A | 20048 | 20067 | GCTTCATCACTCCAATTTTC | 66 | 3248 |
| 1314700 | N/A | N/A | 52753 | 52772 | GATAGGATTATACAACCAAA | 39 | 3249 |
| 1314759 | N/A | N/A | 47448 | 47467 | TTGTTGTTTTTAATAGGACT | 64 | 3250 |
| 1314845 | N/A | N/A | 108248 | 108267 | TGGACATTCACTTCAATTCT | 21 | 3251 |
| 1314912 | N/A | N/A | 106955 | 106974 | CAACATACATTCCTCTTACC | 100 | 3252 |
| 1314916 | N/A | N/A | 70545 | 70564 | AACAAAAGTTATACTTGCTC | 25 | 3253 |
| 1315120 | N/A | N/A | 34540 | 34559 | AGTTTATTCCAAATCATTCA | 70 | 3254 |
| 1315128 | N/A | N/A | 5894 | 5913 | CTCTTGGACCTTACTTCTTA | 72 | 3255 |
| 1315197 | N/A | N/A | 59085 | 59104 | AAGCAACAAACATCATCATC | 76 | 3256 |
| 1315230 | N/A | N/A | 12555 | 12574 | TCTGTATTTCACCAATACTC | 84 | 3257 |
| 1315243 | N/A | N/A | 110108 | 110127 | ACACAATTTCCATTGCGGCA | 47 | 3258 |
| 1315274 | N/A | N/A | 68688 | 68707 | ACATGTCAATTTCTATCCCC | 50 | 3259 |
| 1315333 | N/A | N/A | 19670 | 19689 | AACAATTTAATTAATCTCTT | 91 | 3260 |
| 1315425 | N/A | N/A | 53331 | 53350 | GGTGATATTCTCAAATGCTG | 20 | 3261 |
| 1315478 | N/A | N/A | 133725 | 133744 | TGTGAATTTATTCCCGTACA | 58 | 3262 |
| 1315498 | 3321 | 3340 | 73127 | 73146 | GGTACAGCTCTTCCTAGACT | 28 | 3263 |
| 1315520 | N/A | N/A | 105929 | 105948 | CACACAGGCACACTCTGACC | 99 | 3264 |
| | | | 106232 | 106251 | | | |
| 1315596 | N/A | N/A | 130451 | 130470 | CAGGCATTTCAAACTTACGA | 57 | 3265 |
| 1315603 | N/A | N/A | 9949 | 9968 | GTTTTTCTAAATCCTCACCA | 87 | 3266 |
| 1315665 | N/A | N/A | 60440 | 60459 | TGTCATGGTTTCAACTCTCA | 47 | 3267 |
| 1315666 | N/A | N/A | 83970 | 83989 | ACATAAACTTTCCCTAGCTG | 97 | 3268 |
| 1315734 | N/A | N/A | 104194 | 104213 | TGGGAACACTTTCCTTCTGC | 73 | 3269 |
| 1315897 | N/A | N/A | 69362 | 69381 | ACATAGTCATTTACATTCCT | 32 | 3270 |
| 1315963 | N/A | N/A | 62700 | 62719 | ATCCTTTGAATTCTTCCCTC | 46 | 3271 |
| 1315984 | N/A | N/A | 81881 | 81900 | GTACACATATTTCAAACATC | 36 | 3272 |
| 1316009 | N/A | N/A | 119664 | 119683 | CTATCATTCATTAACAAGCC | 69 | 3273 |
| 1316072 | N/A | N/A | 142147 | 142166 | CCAAAGAATCTTCCTCCCAA | 70 | 3274 |
| 1316105 | N/A | N/A | 125991 | 126010 | TCTTTGCTTTCATCTCGGTC | 91 | 3275 |
| 1316135 | 7752 | 7771 | 149840 | 149859 | GTCGAGAGCTTTCAGAGGCT | 75 | 3276 |
| 1316137 | N/A | N/A | 10439 | 10458 | TAACCACATTCACCTCCCTC | 85 | 3277 |
| 1316188 | N/A | N/A | 116179 | 116198 | ACATGCACACCACTATCCCC | 93 | 3278 |
| 1316194 | N/A | N/A | 51147 | 51166 | TTGGGAATTTCTAATTTCTG | 62 | 3279 |
| 1316232 | N/A | N/A | 15877 | 15896 | GCCAAGTATTTAAAGTCATT | 30 | 3280 |
| 1316245 | N/A | N/A | 118156 | 118175 | TCACCACATCATAATTTGTC | 78 | 3281 |
| 1316260 | N/A | N/A | 14066 | 14085 | ATAAAGGCTTTTTCTAGCTT | 83 | 3282 |
| 1316268 | N/A | N/A | 22172 | 22191 | TGAAATCTACCATCAAGCTC | 70 | 3283 |
| 1316277 | N/A | N/A | 56030 | 56049 | TCACTCATTCCCACTACATT | 69 | 3284 |
| 1316284 | N/A | N/A | 46678 | 46697 | ATGAATTTATTCTTTTTCCC | 77 | 3285 |
| 1316343 | N/A | N/A | 33829 | 33848 | ACGACATACAACATACCCCA | 83 | 3286 |
| 1316355 | 7474 | 7493 | 146566 | 146585 | CCTTAAAGACTTCCTTTTCC | 81 | 3287 |
| 1316406 | N/A | N/A | 25082 | 25101 | GCAGTCTATCTCTCCTGCCC | 65 | 3288 |
| 1316412 | N/A | N/A | 139636 | 139655 | GCTGACTTTCCCACTTCCCC | 50 | 3289 |
| 1316420 | N/A | N/A | 111482 | 111501 | CCTTCATTTCTGCCAACTTA | 66 | 3290 |
| 1316421 | N/A | N/A | 7494 | 7513 | GGTGGAAGACAATATTGTTA | 28 | 3291 |
| 1316447 | N/A | N/A | 98619 | 98638 | GATCTCTTCAACACACTGTA | 65 | 3292 |
| 1316458 | N/A | N/A | 3162 | 3181 | AGGTAATCATATATTACCTT | 90 | 3293 |
| 1316493 | N/A | N/A | 17448 | 17467 | AGTCTCACCATATTAGTGTT | 59 | 3294 |
| 1316502 | N/A | N/A | 19005 | 19024 | GTTAATATATTTTCCAGTCA | 34 | 3295 |
| 1316508 | N/A | N/A | 35938 | 35957 | CATGACTATTTACTTAGGCA | 33 | 3296 |
| 1316526 | N/A | N/A | 129865 | 129884 | TCACCACTCTAAACATGTTG | 83 | 3297 |
| 1316642 | N/A | N/A | 114576 | 114595 | GAGAAGACTTTTATTACTGA | 58 | 3298 |
| 1316667 | N/A | N/A | 134685 | 134704 | CCACTATCAAATATCCCAGT | 69 | 3299 |
| 1316670 | N/A | N/A | 49033 | 49052 | TTCAACATTTTAATTTCAAT | 69 | 3300 |
| 1316717 | N/A | N/A | 94648 | 94667 | ATCAAGCTCACCTCTCCAGC | 75 | 3301 |
| 1316734 | N/A | N/A | 22599 | 22618 | TTCTACAAATCATTGAGAGC | 90 | 3302 |
| 1316736 | N/A | N/A | 127413 | 127432 | GTAGTTTGCCCTCAGAGGGC | 101 | 3303 |
| 1316763 | N/A | N/A | 32691 | 32710 | TGATTATACCTCTGCAGCCA | 83 | 3304 |
| 1316777 | N/A | N/A | 75473 | 75492 | GAACATTCTACAACCATGGA | 63 | 3305 |
| 1316781 | N/A | N/A | 45602 | 45621 | TCCTTTATAAATCTTGCTGA | 81 | 3306 |
| 1316793 | N/A | N/A | 41862 | 41881 | CAGTTCACATTCAATCAACC | 67 | 3307 |
| 1316794 | N/A | N/A | 40143 | 40162 | GCCACAGGACTCCCTCCCAA | 83 | 3308 |
| 1316921 | N/A | N/A | 17823 | 17842 | TGCTGTCAATACTTTCCCCA | 52 | 3309 |
| 1316930 | N/A | N/A | 111914 | 111933 | GAACATTTTTAATCATGCAA | 42 | 3310 |
| 1316934 | N/A | N/A | 57518 | 57537 | CATTAGTTCATTCAACAGAT | 74 | 3311 |
| 1316939 | N/A | N/A | 81041 | 81060 | ACACCACTTCCCTTACTGAA | 68 | 3312 |
| 1316995 | N/A | N/A | 132366 | 132385 | CTCCAATTACTCTTTTTCCA | 34 | 3313 |
| 1317029 | N/A | N/A | 79758 | 79777 | ACATCGAGACTCCCAATGCT | 76 | 3314 |
| 1317039 | N/A | N/A | 117845 | 117864 | AAGACATTTTCATTCTGTTC | 76 | 3315 |
| 1317077 | N/A | N/A | 160816 | 160835 | GCAAGAGCCATAGAACAGCC | 71 | 3316 |
| 1317096 | N/A | N/A | 43583 | 43602 | CCATGGCTACACCCTTTTTA | 74 | 3317 |
| 1317103 | N/A | N/A | 136631 | 136650 | CTTGACTTCCATTCTGTCTT | 88 | 3318 |
| 1317109 | N/A | N/A | 65894 | 65913 | AACTATTTTATCATATTGGT | 61 | 3319 |
| 1317165 | N/A | N/A | 26786 | 26805 | GGAGCTAGAATCCCATGGCT | 100 | 3320 |
| 1317170 | N/A | N/A | 72552 | 72571 | TAAGCAGGACTACCCAGGGC | 59 | 3321 |
| 1317221 | N/A | N/A | 45023 | 45042 | ACCCTTTTTTCCCCTTCACC | 79 | 3322 |

TABLE 45
Reduction of HTT RNA by 5-10-5 MOE gapmers with mixed PO/PS internucleoside
linkages in A431 cells

| | SEQ | SEQ | | | | | |
|---|---|---|---|---|---|---|---|
| | ID | ID | SEQ | SEQ | | | |
| | No: 1 | No: 1 | ID No: | ID No: | | HTT | SEQ |
| Compound | Start | Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 42 | 858 |
| 1231500 | 4275 | 4294 | 99393 | 99412 | CGCCTGCACCATGTTCCTCA | 89 | 3323 |
| 1231544 | 6011 | 6030 | 132941 | 132960 | CGAGACTGAATTGCCTGGAT | 37 | 3324 |
| 1315753 | 8661 | 8680 | 161607 | 161626 | ATAGTTCTCAATGAGGTAAA | 78 | 3325 |
| 1315817 | 2795 | 2814 | 62219 | 62238 | TCTGCTTTTGCCTCCAAAAA | 10† | 3326 |
| 1315833 | 4858 | 4877 | 106913 | 106932 | GTAACATTGACACCACCACC | 45 | 3327 |
| 1316023 | 4609 | 4628 | N/A | N/A | TTGCCTCTGATTCCCTGAAC | 35 | 3328 |
| 1316059 | 4733 | 4752 | 104720 | 104739 | GCCTTCCTTCCACTGGCCAT | 89 | 3329 |
| 1316117 | 4813 | 4832 | 106868 | 106887 | CTGCATCAGCTTTATTTGTT | 45 | 3330 |
| 1316152 | 4605 | 4624 | N/A | N/A | CTCTGATTCCCTGAACTGGC | 66 | 3331 |
| 1316206 | 171 | 190 | 1156 | 1175 | CTTGAGGGACTCGAAGGCCT | 64 | 3332 |
| 1316266 | N/A | N/A | 1446 | 1465 | GCAGGGTTACCGCCATCCCC | 87 | 3333 |
| 1316341 | 7724 | 7743 | 149812 | 149831 | GGCTGCTGCTCCAAGCAGCT | 94 | 3334 |
| 1316545 | 5808 | 5827 | 130391 | 130410 | TTCTCTATTGCACATTCCAA | 36 | 3335 |
| 1316899 | 4713 | 4732 | 104700 | 104719 | GATGCCATCACAGAGCTGAA | 56 | 3336 |
| 1316926 | 4384 | 4403 | N/A | N/A | TAGCATTCTTATCTGCACGG | 20 | 3337 |
| 1316928 | 127 | 146 | 1112 | 1131 | TGGCGGTCTCCCGCCCGGCA | 101 | 3338 |
| 1316972 | 4826 | 4845 | 106881 | 106900 | TCAAGCTCTTTTCCTGCATC | 26 | 1232 |
| 1317019 | 107 | 126 | 1092 | 1111 | CGGCAGTCCCCGGAGGCCTC | 92 | 3339 |
| 1317101 | N/A | N/A | 1437 | 1456 | CCGCCATCCCCGCCGTAGCC | 85 | 3340 |
| 1317179 | 389 | 408 | 1368 | 1387 | TGCAGCGGCTCCTCAGCCAC | 91 | 3341 |
| 1318565 | 4820 | 4839 | 106875 | 106894 | TCTTTTCCTGCATCAGCTTT | 57 | 3342 |
| 1318568 | 967 | 986 | 41677 | 41696 | GGCAGATGCTCACTGCTGAT | 84 | 3343 |
| 1318569 | 4810 | 4829 | 106865 | 106884 | CATCAGCTTTATTTGTTCCT | 26 | 3344 |
| 1318579 | 12 | 31 | 997 | 1016 | CGGCCGTCCATCTTGGACCC | 80 | 3345 |
| 1318584 | 3554 | 3573 | 74416 | 74435 | AGCTGCTCCACCATGGGCAC | 98 | 3346 |
| 1318585 | N/A | N/A | 115063 | 115082 | TCTGTGTATCACCTTCCTCA | 51 | 3347 |
| 1318588 | N/A | N/A | 1630 | 1649 | GACAGGCCCCAACAAGGCTC | 94 | 3348 |
| 1318592 | 2121 | 2140 | N/A | N/A | AGGCTTGTTTTCTTGATCAC | 32 | 3349 |
| 1318595 | N/A | N/A | 1498 | 1517 | GGGTCACTCTGTCTCTGCGG | 86 | 3350 |
| 1318596 | N/A | N/A | 1442 | 1461 | GGTTACCGCCATCCCCGCCG | 78 | 3351 |
| 1318600 | 3213 | 3232 | N/A | N/A | TTCACAGCATCCAAATGTGA | 85 | 3352 |
| 1318601 | 5230 | 5249 | 114044 | 114063 | AGAGCTCCTGAATACGAGAA | 47 | 3353 |
| 1318602 | 161 | 180 | 1146 | 1165 | TCGAAGGCCTTCATCAGCTT | 97 | 3354 |
| 1318609 | 702 | 721 | 31684 | 31703 | AAACCTCCACAGGGCAGCAC | 66 | 3355 |
| 1318610 | 3218 | 3237 | 71456 | 71475 | AAAGCTTCACAGCATCCAAA | 55 | 3356 |
| 1318614 | N/A | N/A | 1609 | 1628 | GCCTCCCCTCGCGAGAGGAC | 98 | 3357 |
| 1318615 | 4701 | 4720 | 104688 | 104707 | GAGCTGAATGATTTTAGGAA | 21 | 3358 |
| 1318621 | 4933 | 4952 | 108690 | 108709 | CTTCATTCTCCTTGTGGCAC | 40 | 3359 |
| 1318622 | 4593 | 4612 | 103670 | 103689 | GAACTGGCCCACTTCAATGT | 53 | 3360 |
| 1318627 | N/A | N/A | 101088 | 101107 | TTACCTTATCTGCACGGTTC | 72 | 3361 |
| 1318632 | 4827 | 4846 | 106882 | 106901 | TTCAAGCTCTTTTCCTGCAT | 33 | 3362 |
| 1318638 | 4043 | 4062 | 98651 | 98670 | TCTCGACTAAAGCAGGATTT | 34 | 3363 |
| 1318640 | 4562 | 4581 | 103639 | 103658 | TTCAATACAAAGCCAATAAA | 84 | 3364 |
| 1318642 | 4513 | 4532 | 101366 | 101385 | TAACCCGTAACTGAACCAGC | 52 | 3365 |
| 1318645 | 5529 | 5548 | 126045 | 126064 | GGCAGCTGCTGTGATTCTCC | 79 | 3366 |
| 1318648 | 4565 | 4584 | 103642 | 103661 | TGTTTCAATACAAAGCCAAT | 66 | 3367 |
| 1318653 | 5804 | 5823 | 130387 | 130406 | CTATTGCACATTCCAAGTTT | 80 | 3368 |
| 1318656 | 386 | 405 | 1365 | 1384 | AGCGGCTCCTCAGCCACAGC | 98 | 3369 |
| 1318660 | 7721 | 7740 | 149809 | 149828 | TGCTGCTCCAAGCAGCTTAC | 93 | 3370 |
| 1318663 | 6770 | 6789 | N/A | N/A | GCAGCATCCCCAAACAGATC | 76 | 3371 |
| 1318666 | 6767 | 6786 | N/A | N/A | GCATCCCCAAACAGATCATT | 85 | 3372 |
| 1318670 | 7392 | 7411 | N/A | N/A | CCATCCAAGCTTCCACACCA | 79 | 3373 |
| 1318672 | 7387 | 7406 | N/A | N/A | CAAGCTTCCACACCAGTGGG | 85 | 3374 |
| 1318674 | 6777 | 6796 | N/A | N/A | ATACAGTGCAGCATCCCCAA | 89 | 3375 |
| 1318677 | 6774 | 6793 | N/A | N/A | CAGTGCAGCATCCCCAAACA | 71 | 3376 |
| 1318678 | 7833 | 7852 | 150352 | 150371 | ATTCTCTCTCTTTGAAACCA | 35 | 3377 |
| 1318680 | 9767 | 9786 | 166516 | 166535 | CCAGGACTGCAGACACTCCC | 75 | 3378 |
| 1318681 | N/A | N/A | 141020 | 141039 | ACAAATGCACAGATGAAAAC | 80 | 3379 |
| 1318684 | 7829 | 7848 | 150348 | 150367 | TCTCTCTTTGAAACCATTGC | 41 | 3380 |
| 1318686 | 6153 | 6172 | 133214 | 133233 | CATGCGAGCCAGCACACGGA | 56 | 3381 |
| 1318691 | 4832 | 4851 | 106887 | 106906 | TGGGTTTCAAGCTCTTTTCC | 46 | 3382 |
| 1318696 | 4864 | 4883 | 106919 | 106938 | GTCTCAGTAACATTGACACC | 96 | 3383 |
| 1318697 | 7180 | 7199 | 141490 | 141509 | TGTTTGGATCTACTTCCTCC | 69 | 3384 |
| 1318703 | 4855 | 4874 | 106910 | 106929 | ACATTGACACCACCACCTCT | 73 | 3385 |
| 1318705 | 4952 | 4971 | 108709 | 108728 | GACAGTCGCTTCCACTTGTC | 70 | 3386 |
| 1318706 | N/A | N/A | 115060 | 115079 | GTGTATCACCTTCCTCACTG | 78 | 3387 |
| 1318708 | N/A | N/A | 104599 | 104618 | TGATTGCCTCTGATTCCCTA | 23 | 3388 |
| 1318710 | 157 | 176 | 1142 | 1161 | AGGCCTTCATCAGCTTTTCC | 92 | 3389 |
| 1318715 | 4622 | 4641 | 104609 | 104628 | ATGTTTGGAATGATTGCCTC | 28 | 3390 |
| 1318724 | 5507 | 5526 | N/A | N/A | AACATTCCAGACTTGAAGAT | 79 | 3391 |
| 1318725 | 706 | 725 | 31688 | 31707 | CAGCAAACCTCCACAGGGCA | 46 | 3392 |
| 1318727 | 6788 | 6807 | 138876 | 138895 | GGCAGGGACTGATACAGTGC | 66 | 3393 |
| 1318731 | 4569 | 4588 | 103646 | 103665 | AAACTGTTTCAATACAAAGC | 51 | 3394 |
| 1318733 | 4506 | 4525 | 101359 | 101378 | TAACTGAACCAGCTGCGCCA | 80 | 3395 |
| 1318734 | 4873 | 4892 | 106928 | 106947 | ACTGGATGAGTCTCAGTAAC | 74 | 3396 |
| 1318740 | 2790 | 2809 | 62214 | 62233 | TTTTGCCTCCAAAAAGCTCA | 27 | 3397 |
| 1318746 | 4738 | 4757 | 104725 | 104744 | TCACAGCCTTCCTTCCACTG | 76 | 3398 |

TABLE 46
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 43 | 858 |
| 1231501 | 4277 | 4296 | 99395 | 99414 | TCCGCCTGCACCATGTTCCT | 82 | 3399 |
| 1231516 | 4604 | 4623 | N/A | N/A | TCTGATTCCCTGAACTGGCC | 77 | 3400 |
| 1315878 | 5806 | 5825 | 130389 | 130408 | CTCTATTGCACATTCCAAGT | 29 | 3401 |
| 1315892 | N/A | N/A | 1441 | 1460 | GTTACCGCCATCCCCGCCGT | 91 | 3402 |
| 1315898 | 4608 | 4627 | N/A | N/A | TGCCTCTGATTCCCTGAACT | 55 | 3403 |
| 1316129 | N/A | N/A | 115065 | 115084 | TCTCTGTGTATCACCTTCCT | 67 | 3404 |
| 1316143 | 5801 | 5820 | 130384 | 130403 | TTGCACATTCCAAGTTTGGC | 34 | 3405 |
| 1316282 | 6769 | 6788 | N/A | N/A | CAGCATCCCCAAACAGATCA | 84 | 3406 |
| 1316297 | N/A | N/A | 1629 | 1648 | ACAGGCCCCAACAAGGCTCT | 84 | 3407 |
| 1316325 | 2123 | 2142 | N/A | N/A | CAAGGCTTGTTTTCTTGATC | 57 | 3408 |
| 1316430 | 6152 | 6171 | 133213 | 133232 | ATGCGAGCCAGCACACGGAA | 76 | 3409 |
| 1316552 | 2829 | 2848 | 62253 | 62272 | CCCTGTATAATGATGAGCCC | 17† | 3410 |
| 1317030 | 4822 | 4841 | 106877 | 106896 | GCTCTTTTCCTGCATCAGCT | 57 | 3411 |
| 1317131 | 4511 | 4530 | 101364 | 101383 | ACCCGTAACTGAACCAGCTG | 70 | 3412 |
| 1317175 | 4564 | 4583 | 103641 | 103660 | GTTTCAATACAAAGCCAATA | 36 | 3413 |
| 1318567 | N/A | N/A | 1444 | 1463 | AGGGTTACCGCCATCCCCGC | 84 | 3414 |
| 1318570 | 4735 | 4754 | 104722 | 104741 | CAGCCTTCCTTCCACTGGCC | 104 | 3415 |
| 1318574 | N/A | N/A | 1436 | 1455 | CGCCATCCCCGCCGTAGCCT | 83 | 3416 |
| 1318575 | N/A | N/A | 1466 | 1485 | GTGTCGCCGGCCCGCAGGCT | 80 | 3417 |
| 1318576 | 966 | 985 | 41676 | 41695 | GCAGATGCTCACTGCTGATC | 76 | 3418 |
| 1318577 | 4812 | 4831 | 106867 | 106886 | TGCATCAGCTTTATTTGTTC | 62 | 3419 |
| 1318578 | 11 | 30 | 996 | 1015 | GGCCGTCCATCTTGGACCCG | 93 | 3420 |
| 1318580 | N/A | N/A | 115062 | 115081 | CTGTGTATCACCTTCCTCAC | 62 | 3421 |
| 1318582 | 3556 | 3575 | 74418 | 74437 | AGAGCTGCTCCACCATGGGC | 83 | 3422 |
| 1318587 | N/A | N/A | 1503 | 1522 | TTGCTGGGTCACTCTGTCTC | 64 | 3423 |
| 1318593 | N/A | N/A | 1632 | 1651 | AGGACAGGCCCCAACAAGGC | 73 | 3424 |
| 1318598 | 160 | 179 | 1145 | 1164 | CGAAGGCCTTCATCAGCTTT | 82 | 3425 |
| 1318599 | 5233 | 5252 | 114047 | 114066 | AGGAGAGCTCCTGAATACGA | 45 | 3426 |
| 1318603 | 5229 | 5248 | 114043 | 114062 | GAGCTCCTGAATACGAGAAA | 64 | 3427 |
| 1318604 | 4615 | 4634 | 104602 | 104621 | GAATGATTGCCTCTGATTCC | 48 | 3428 |
| 1318607 | 165 | 184 | 1150 | 1169 | GGACTCGAAGGCCTTCATCA | 81 | 3429 |
| 1318613 | 704 | 723 | 31686 | 31705 | GCAAACCTCCACAGGGCAGC | 83 | 3430 |
| 1318617 | 3217 | 3236 | N/A | N/A | AAGCTTCACAGCATCCAAAT | 82 | 3431 |
| 1318623 | 4932 | 4951 | 108689 | 108708 | TTCATTCTCCTTGTGGCACT | 41 | 3432 |
| 1318624 | 4716 | 4735 | 104703 | 104722 | CATGATGCCATCACAGAGCT | 57 | 3433 |
| 1318625 | 4592 | 4611 | 103669 | 103688 | AACTGGCCCACTTCAATGTA | 73 | 3434 |
| 1318626 | 4712 | 4731 | 104699 | 104718 | ATGCCATCACAGAGCTGAAT | 71 | 3435 |
| 1318630 | 4867 | 4886 | 106922 | 106941 | TGAGTCTCAGTAACATTGAC | 62 | 3436 |
| 1318631 | 129 | 148 | 1114 | 1133 | CATGGCGGTCTCCCGCCCGG | 55 | 3437 |
| 1318633 | 388 | 407 | 1367 | 1386 | GCAGCGGCTCCTCAGCCACA | 86 | 3438 |
| 1318636 | 4819 | 4838 | 106874 | 106893 | CTTTTCCTGCATCAGCTTTA | 48 | 3439 |
| 1318637 | 126 | 145 | 1111 | 1130 | GGCGGTCTCCCGCCCGGCAC | 89 | 3440 |
| 1318641 | 4613 | 4632 | 104600 | 104619 | ATGATTGCCTCTGATTCCCT | 30 | 3441 |
| 1318643 | 5525 | 5544 | 126041 | 126060 | GCTGCTGTGATTCTCCGGAA | 46 | 3442 |
| 1318649 | 6786 | 6805 | 138874 | 138893 | CAGGGACTGATACAGTGCAG | 72 | 3443 |
| 1318654 | 8664 | 8683 | 161610 | 161629 | AGGATAGTTCTCAATGAGGT | 41 | 3444 |
| 1318655 | 4567 | 4586 | 103644 | 103663 | ACTGTTTCAATACAAAGCCA | 59 | 3445 |
| 1318661 | 6772 | 6791 | N/A | N/A | GTGCAGCATCCCCAAACAGA | 84 | 3446 |
| 1318665 | 6766 | 6785 | N/A | N/A | CATCCCCAAACAGATCATTC | 96 | 3447 |
| 1318667 | 9789 | 9808 | 166538 | 166557 | GAAGGCCTCAGGCTCAGCCC | 90 | 3448 |
| 1318668 | 7723 | 7742 | 149811 | 149830 | GCTGCTGCTCCAAGCAGCTT | 88 | 3449 |
| 1318671 | 7390 | 7409 | N/A | N/A | ATCCAAGCTTCCACACCAGT | 87 | 3450 |
| 1318673 | 6776 | 6795 | N/A | N/A | TACAGTGCAGCATCCCCAAA | 72 | 3451 |
| 1318675 | 4863 | 4882 | 106918 | 106937 | TCTCAGTAACATTGACACCA | 43 | 3452 |
| 1318682 | N/A | N/A | 141016 | 141035 | ATGCACAGATGAAAACAATT | 82 | 3453 |
| 1318685 | 7832 | 7851 | 150351 | 150370 | TTCTCTCTCTTTGAAACCAT | 37 | 3454 |
| 1318688 | 7184 | 7203 | 141494 | 141513 | TGTGTGTTTGGATCTACTTC | 59 | 3455 |
| 1318695 | 4829 | 4848 | 106884 | 106903 | GTTTCAAGCTCTTTTCCTGC | 26 | 3456 |
| 1318699 | 2793 | 2812 | 62217 | 62236 | TGCTTTTGCCTCCAAAAAGC | 43† | 3457 |
| 1318701 | 4857 | 4876 | 106912 | 106931 | TAACATTGACACCACCACCT | 38 | 3458 |
| 1318713 | 3552 | 3571 | 74414 | 74433 | CTGCTCCACCATGGGCACCA | 99 | 3459 |
| 1318717 | 102 | 121 | 1087 | 1106 | GTCCCCGGAGGCCTCGGGCC | 92 | 3460 |
| 1318718 | 988 | 1007 | 41698 | 41717 | ATTGTGTCCTTCTTGAGTGC | 57 | 3461 |
| 1318719 | 385 | 404 | 1364 | 1383 | GCGGCTCCTCAGCCACAGCC | 107 | 3462 |
| 1318720 | N/A | N/A | 101091 | 101110 | CATTTACCTTATCTGCACGG | 89 | 3463 |
| 1318721 | 4389 | 4408 | N/A | N/A | ATGAATAGCATTCTTATCTG | 54 | 3464 |
| 1318723 | 4698 | 4717 | 104685 | 104704 | CTGAATGATTTTAGGAATTC | 52 | 3465 |
| 1318726 | 4047 | 4066 | 98655 | 98674 | TGGTTCTCGACTAAAGCAGG | 27 | 3466 |
| 1318729 | 7624 | 7643 | 149712 | 149731 | TCTGGGTCCTCTCTGTGTCT | 62 | 3467 |
| 1318732 | 4559 | 4578 | 103636 | 103655 | AATACAAAGCCAATAAACAC | 79 | 3468 |
| 1318737 | 4950 | 4969 | 108707 | 108726 | CAGTCGCTTCCACTTGTCTT | 66 | 3469 |
| 1318738 | 4853 | 4872 | 106908 | 106927 | ATTGACACCACCACCTCTTT | 99 | 3470 |
| 1318739 | 7178 | 7197 | 141488 | 141507 | TTTGGATCTACTTCCTCCTC | 87 | 3471 |
| 1318742 | 5813 | 5832 | 130396 | 130415 | ACTATTTCTCTATTGCACAT | 34 | 3472 |
| 1318743 | 7836 | 7855 | 150355 | 150374 | AATATTCTCTCTCTTTGAAA | 98 | 3473 |
| 1318744 | 7826 | 7845 | 150345 | 150364 | CTCTTTGAAACCATTGCTTG | 63 | 3474 |
| 1318747 | 4809 | 4828 | 106864 | 106883 | ATCAGCTTTATTTGTTCCTC | 33 | 3475 |

TABLE 47
Reduction of HTT RNA by 5-10-5 MOE gapmers with mixed PO/PS internucleoside
linkages in A431 cells

| | SEQ | SEQ | SEQ | SEQ | | | |
|---|---|---|---|---|---|---|---|
| | ID No: | ID No: | ID No: | ID No: | | HTT | SEQ |
| Compound | 1 Start | 1 Stop | 2 Start | 2 Stop | | (% | ID |
| Number | Site | Site | Site | Site | Sequence (5′ to 3′) | UTC) | NO |
| 388241 | 4862 | 4881 | 106917 | 106936 | CTCAGTAACATTGACACCAC | 42 | 858 |
| 627246 | 4862 | 4881 | 106917 | 106936 | CTCAGTAACATTGACACCAC | 67 | 858 |
| 1231517 | 4607 | 4626 | N/A | N/A | GCCTCTGATTCCCTGAACTG | 57 | 3476 |
| 1231536 | 5812 | 5831 | 130395 | 130414 | CTATTTCTCTATTGCACATT | 45 | 3477 |
| 1315904 | 968 | 987 | 41678 | 41697 | TGGCAGATGCTCACTGCTGA | 89 | 3478 |
| 1316055 | N/A | N/A | 1443 | 1462 | GGGTTACCGCCATCCCCGCC | 94 | 3479 |
| 1316138 | N/A | N/A | 1502 | 1521 | TGCTGGGTCACTCTGTCTCT | 77 | 3480 |
| 1316364 | 6775 | 6794 | N/A | N/A | ACAGTGCAGCATCCCCAAAC | 71 | 3481 |
| 1316386 | 7389 | 7408 | N/A | N/A | TCCAAGCTTCCACACCAGTG | 80 | 3482 |
| 1316513 | 7831 | 7850 | 150350 | 150369 | TCTCTCTCTTTGAAACCATT | 39 | 3483 |
| 1316596 | 162 | 181 | 1147 | 1166 | CTCGAAGGCCTTCATCAGCT | 98 | 3484 |
| 1316624 | 5231 | 5250 | 114045 | 114064 | GAGAGCTCCTGAATACGAGA | 54 | 3485 |
| 1317002 | 390 | 409 | 1369 | 1388 | GTGCAGCGGCTCCTCAGCCA | 80 | 3486 |
| 1317142 | 4610 | 4629 | N/A | N/A | ATTGCCTCTGATTCCCTGAA | 25 | 3487 |
| 1318566 | 4821 | 4840 | 106876 | 106895 | CTCTTTTCCTGCATCAGCTT | 47 | 3488 |
| 1318571 | 4734 | 4753 | 104721 | 104740 | AGCCTTCCTTCCACTGGCCA | 89 | 3489 |
| 1318572 | 965 | 984 | 41675 | 41694 | CAGATGCTCACTGCTGATCC | 60 | 3490 |
| 1318573 | 4811 | 4830 | 106866 | 106885 | GCATCAGCTTTATTTGTTCC | 39 | 3491 |
| 1318581 | N/A | N/A | 1440 | 1459 | TTACCGCCATCCCCGCCGTA | 102 | 3492 |
| 1318583 | 3555 | 3574 | 74417 | 74436 | GAGCTGCTCCACCATGGGCA | 92 | 3493 |
| 1318586 | N/A | N/A | 115064 | 115083 | CTCTGTGTATCACCTTCCTC | 47 | 3494 |
| 1318589 | N/A | N/A | 1631 | 1650 | GGACAGGCCCCAACAAGGCT | 87 | 3495 |
| 1318590 | 159 | 178 | 1144 | 1163 | GAAGGCCTTCATCAGCTTTT | 91 | 3496 |
| 1318591 | 4276 | 4295 | 99394 | 99413 | CCGCCTGCACCATGTTCCTC | 78 | 3497 |
| 1318594 | 2122 | 2141 | N/A | N/A | AAGGCTTGTTTTCTTGATCA | 36 | 3498 |
| 1318597 | N/A | N/A | 1447 | 1466 | TGCAGGGTTACCGCCATCCC | 91 | 3499 |
| 1318605 | 4614 | 4633 | 104601 | 104620 | AATGATTGCCTCTGATTCCC | 26 | 3500 |
| 1318606 | 109 | 128 | 1094 | 1113 | CACGGCAGTCCCCGGAGGCC | 91 | 3501 |
| 1318608 | 3214 | 3233 | N/A | N/A | CTTCACAGCATCCAAATGTG | 91 | 3502 |
| 1318611 | 4603 | 4622 | N/A | N/A | CTGATTCCCTGAACTGGCCC | 58 | 3503 |
| 1318612 | 4706 | 4725 | 104693 | 104712 | TCACAGAGCTGAATGATTTT | 58 | 3504 |
| 1318616 | 703 | 722 | 31685 | 31704 | CAAACCTCCACAGGGCAGCA | 68 | 3505 |
| 1318618 | 4934 | 4953 | 108691 | 108710 | TCTTCATTCTCCTTGTGGCA | 52 | 3506 |
| 1318619 | 4715 | 4734 | 104702 | 104721 | ATGATGCCATCACAGAGCTG | 54 | 3507 |
| 1318620 | 4383 | 4402 | N/A | N/A | AGCATTCTTATCTGCACGGT | 24 | 3508 |
| 1318628 | 387 | 406 | 1366 | 1385 | CAGCGGCTCCTCAGCCACAG | 97 | 3509 |
| 1318629 | 4386 | 4405 | N/A | N/A | AATAGCATTCTTATCTGCAC | 43 | 3510 |
| 1318634 | 4818 | 4837 | 106873 | 106892 | TTTTCCTGCATCAGCTTTAT | 75 | 3511 |
| 1318635 | 128 | 147 | 1113 | 1132 | ATGGCGGTCTCCCGCCCGGC | 87 | 3512 |
| 1318639 | 4563 | 4582 | 103640 | 103659 | TTTCAATACAAAGCCAATAA | 75 | 3513 |
| 1318644 | 4045 | 4064 | 98653 | 98672 | GTTCTCGACTAAAGCAGGAT | 41 | 3514 |
| 1318646 | 4508 | 4527 | 101361 | 101380 | CGTAACTGAACCAGCTGCGC | 65 | 3515 |
| 1318647 | 5524 | 5543 | 126040 | 126059 | CTGCTGTGATTCTCCGGAAC | 46 | 3516 |
| 1318650 | 6785 | 6804 | 138873 | 138892 | AGGGACTGATACAGTGCAGC | 58 | 3517 |
| 1318651 | 8663 | 8682 | 161609 | 161628 | GGATAGTTCTCAATGAGGTA | 49 | 3518 |
| 1318652 | 5805 | 5824 | 130388 | 130407 | TCTATTGCACATTCCAAGTT | 48 | 3519 |
| 1318657 | 4566 | 4585 | 103643 | 103662 | CTGTTTCAATACAAAGCCAA | 43 | 3520 |
| 1318658 | 7722 | 7741 | 149810 | 149829 | CTGCTGCTCCAAGCAGCTTA | 97 | 3521 |
| 1318659 | 9788 | 9807 | 166537 | 166556 | AAGGCCTCAGGCTCAGCCCC | 97 | 3522 |
| 1318662 | 6771 | 6790 | N/A | N/A | TGCAGCATCCCCAAACAGAT | 74 | 3523 |
| 1318664 | 6768 | 6787 | N/A | N/A | AGCATCCCCAAACAGATCAT | 110 | 3524 |
| 1318669 | 6151 | 6170 | 133212 | 133231 | TGCGAGCCAGCACACGGAAA | 73 | 3525 |
| 1318676 | 7725 | 7744 | 149813 | 149832 | GGGCTGCTGCTCCAAGCAGC | 70 | 3526 |
| 1318679 | N/A | N/A | 141021 | 141040 | TACAAATGCACAGATGAAAA | 78 | 3527 |
| 1318683 | N/A | N/A | 141015 | 141034 | TGCACAGATGAAAACAATTA | 91 | 3528 |
| 1318687 | 7834 | 7853 | 150353 | 150372 | TATTCTCTCTCTTTGAAACC | 69 | 3529 |
| 1318689 | 7181 | 7200 | 141491 | 141510 | GTGTTTGGATCTACTTCCTC | 53 | 3530 |
| 1318690 | 4828 | 4847 | 106883 | 106902 | TTTCAAGCTCTTTTCCTGCA | 42 | 3531 |
| 1318692 | 2792 | 2811 | 62216 | 62235 | GCTTTTGCCTCCAAAAAGCT | 61† | 3532 |
| 1318693 | 4865 | 4884 | 106920 | 106939 | AGTCTCAGTAACATTGACAC | 76 | 3533 |
| 1318694 | 4922 | 4941 | 108679 | 108698 | TTGTGGCACTGCTGCAGGAC | 69 | 3534 |
| 1318698 | 4676 | 4695 | 104663 | 104682 | ATGATCTGTTTTGAATGATA | 26 | 3535 |
| 1318700 | 4953 | 4972 | 108710 | 108729 | AGACAGTCGCTTCCACTTGT | 55 | 3536 |
| 1318702 | 4856 | 4875 | 106911 | 106930 | AACATTGACACCACCACCTC | 79 | 3537 |
| 1318704 | 2798 | 2817 | 62222 | 62241 | TTTTCTGCTTTTGCCTCCAA | 22† | 3538 |
| 1318707 | 14 | 33 | 999 | 1018 | AGCGGCCGTCCATCTTGGAC | 96 | 3539 |
| 1318709 | N/A | N/A | 1624 | 1643 | CCCCAACAAGGCTCTGCCTC | 78 | 3540 |
| 1318711 | 5693 | 5712 | 126209 | 126228 | ACTTCTGCCCACCAGCGGTA | 83 | 3541 |
| 1318712 | N/A | N/A | 115061 | 115080 | TGTGTATCACCTTCCTCACT | 73 | 3542 |
| 1318714 | 4 | 23 | 989 | 1008 | CATCTTGGACCCGTCCCGGC | 92 | 3543 |
| 1318716 | 3220 | 3239 | 71458 | 71477 | ACAAAGCTTCACAGCATCCA | 60 | 3544 |
| 1318722 | 4590 | 4609 | 103667 | 103686 | CTGGCCCACTTCAATGTATT | 89 | 3545 |
| 1318728 | 7614 | 7633 | N/A | N/A | CTCTGTGTCTTCTTCTGGTG | 86 | 3546 |
| 1318730 | 384 | 403 | 1363 | 1382 | CGGCTCCTCAGCCACAGCCG | 86 | 3547 |
| 1318735 | 4516 | 4535 | 101369 | 101388 | AATTAACCCGTAACTGAACC | 58 | 3548 |
| 1318736 | 6764 | 6783 | N/A | N/A | TCCCCAAACAGATCATTCAA | 88 | 3549 |
| 1318741 | 4836 | 4855 | 106891 | 106910 | TTTTTGGGTTTCAAGCTCTT | 48 | 3550 |
| 1318745 | 4808 | 4827 | 106863 | 106882 | TCAGCTTTATTTGTTCCTCT | 25 | 3551 |

Example 4: Dose-Dependent Inhibition of Human HTT in HepG2 Cells by Modified Oligonucleotides Modified oligonucleotides selected from the examples above were tested at various doses in HepG2 cells. Cultured HepG2 cells at a density of 20,000 cells per well were treated by electroporation with various concentrations of modified oligonucleotide as specified in the tables below. After a treatment period of approximately 24 hours, total RNA was isolated from the cells and HTT RNA levels were measured by quantitative real-time RTPCR. Human HTT primer-probe set RTS2617 (described herein above) was used to measure RNA levels as described above. HTT RNA levels were normalized to total RNA content, as measured by RIBOGREEN®. Reduction of HTT RNA is presented in the tables below as percent HTT RNA, relative to the amount of HTT RNA in untreated control cells (% UTC).

The half maximal inhibitory concentration (IC₅₀) of each modified oligonucleotide was calculated using a linear regression on a log/linear plot of the data in Excel and is also presented in the tables below.

TABLE 48
Dose-dependent reduction of human HTT RNA
in HepG2 cells by modified oligonucleotides

| Compound | HTT RNA (% UTC) | | | | | IC50 |
|---|---|---|---|---|---|---|
| No. | 62 nM | 185 nM | 556 nM | 1667 nM | 5000 nM | (μM) |
| 387916 | 100 | 94 | 90 | 58 | 34 | 2.87 |
| 646210 | 83 | 80 | 70 | 50 | 29 | 1.47 |
| 646211 | 85 | 70 | 51 | 34 | 12 | 0.57 |
| 646214 | 103 | 99 | 77 | 57 | 29 | 2.10 |
| 646215 | 92 | 94 | 74 | 68 | 36 | 3.48 |
| 646218 | 105 | 81 | 54 | 33 | 16 | 0.82 |
| 646219 | 86 | 88 | 68 | 48 | 21 | 1.23 |
| 646221 | 104 | 86 | 69 | 42 | 33 | 1.48 |
| 646222 | 102 | 97 | 71 | 46 | 32 | 1.71 |
| 646259 | 94 | 85 | 77 | 55 | 27 | 1.80 |
| 646264 | 96 | 88 | 76 | 47 | 41 | 2.27 |
| 646265 | 98 | 103 | 71 | 43 | 21 | 1.32 |
| 646284 | 93 | 89 | 69 | 57 | 35 | 2.20 |
| 646311 | 94 | 100 | 61 | 47 | 26 | 1.38 |
| 646344 | 92 | 69 | 57 | 30 | 14 | 0.64 |

TABLE 49
Dose-dependent reduction of human HTT RNA
in HepG2 cells by modified oligonucleotides

| Compound | HTT RNA (% UTC) | | | | | IC50 |
|---|---|---|---|---|---|---|
| No. | 62 nM | 185 nM | 556 nM | 1667 nM | 5000 nM | (μM) |
| 387916 | 103 | 98 | 89 | 42 | 28 | 1.82 |
| 646287 | 112 | 106 | 100 | 80 | 56 | >5 |
| 646301 | 99 | 76 | 37 | 28 | 13 | 0.57 |
| 646313 | 97 | 93 | 63 | 52 | 22 | 1.32 |
| 646342 | 114 | 98 | 74 | 52 | 26 | 1.72 |
| 646356 | 92 | 87 | 62 | 46 | 29 | 1.33 |
| 646364 | 98 | 93 | 74 | 40 | 18 | 1.18 |
| 646366 | 91 | 90 | 72 | 60 | 23 | 1.72 |
| 646370 | 94 | 102 | 72 | 38 | 20 | 1.23 |
| 646376 | 94 | 83 | 54 | 26 | 12 | 0.67 |
| 646384 | 97 | 97 | 81 | 50 | 43 | 2.99 |
| 646398 | 104 | 99 | 90 | 52 | 32 | 2.41 |
| 646401 | 95 | 81 | 69 | 36 | 14 | 0.89 |
| 646411 | 95 | 81 | 56 | 32 | 13 | 0.74 |
| 646412 | 105 | 84 | 52 | 25 | 12 | 0.71 |

### Example 5: Dose-Dependent Inhibition of Human HTT in A431 Cells by Modified Oligonucleotides

Modified oligonucleotides selected from the examples above were tested at various doses in A431 cells. Cultured A431 cells at a density of 10,000 cells per well were treated by free uptake with various concentrations of modified oligonucleotide as specified in the tables below. After a treatment period of approximately 48 hours, total RNA was isolated from the cells and HTT RNA levels were measured by quantitative real-time RTPCR. Human HTT primer-probe set RTS36485 (described herein above) was used to measure RNA levels as described above. HTT RNA levels were normalized to total RNA content, as measured by RIBOGREEN®. Reduction of HTT RNA is presented in the tables below as percent HTT RNA, relative to the amount of HTT RNA in untreated control cells (% UTC).

The half maximal inhibitory concentration (IC₅₀) of each modified oligonucleotide was calculated using a linear regression on a log/linear plot of the data in Excel and is also presented in the tables below.

TABLE 50
Dose-dependent reduction of human HTT RNA
in A431 cells by modified oligonucleotides

| Compound | HTT RNA (% UTC) | | | | IC50 |
|---|---|---|---|---|---|
| No. | 234 nM | 938 nM | 3750 nM | 15000 nM | (μM) |
| 387916 | 38 | 16 | 7 | 3 | <0.23 |
| 953845 | 102 | 83 | 59 | 58 | >15 |
| 953941 | 94 | 87 | 61 | 54 | >15 |
| 953957 | 127 | 117 | 115 | 116 | >15 |
| 953966 | 78 | 64 | 54 | 49 | 9.37 |
| 954021 | 78 | 57 | 43 | 32 | 2.36 |
| 954022 | 100 | 87 | 75 | 65 | >15 |
| 954030 | 74 | 55 | 41 | 39 | 2.42 |
| 954047 | 66 | 32 | 17 | 13 | 0.44 |
| 954085 | 113 | 105 | 92 | 81 | >15 |
| 954101 | 92 | 84 | 71 | 57 | >15 |
| 954118 | 88 | 73 | 62 | 49 | 13.74 |
| 954205 | 93 | 79 | 45 | 47 | 6.83 |
| 954238 | 72 | 66 | 44 | 31 | 2.52 |
| 954294 | 88 | 89 | 69 | 60 | >15 |
| 954317 | 99 | 89 | 61 | 47 | 11.37 |
| 954365 | 76 | 52 | 39 | 33 | 1.89 |
| 954366 | 92 | 74 | 54 | 48 | 8.77 |
| 954374 | 89 | 65 | 44 | 39 | 3.99 |

TABLE 51
Dose-dependent reduction of human HTT RNA
in A431 cells by modified oligonucleotides

| | HTT RNA (% UTC) | | | | IC50 |
|---|---|---|---|---|---|
| Compound No. | 234 nM | 938 nM | 3750 nM | 15000 nM | (μM) |
| 387916 | 37 | 14 | 6 | 3 | <0.23 |
| 953799 | 109 | 119 | 107 | 119 | >15 |
| 953871 | 112 | 101 | 83 | 74 | >15 |
| 953896 | 74 | 59 | 50 | 38 | 3.45 |
| 953905 | 111 | 87 | 61 | 33 | 6.44 |
| 953991 | 104 | 90 | 79 | 65 | >15 |
| 953993 | 102 | 83 | 84 | 72 | >15 |
| 954023 | 87 | 60 | 49 | 38 | 4.05 |
| 954033 | 94 | 80 | 58 | 58 | >15 |
| 954129 | 108 | 117 | 100 | 111 | >15 |
| 954135 | 121 | 110 | 117 | 107 | >15 |
| 954272 | 87 | 73 | 56 | 41 | 6.78 |
| 954288 | 102 | 90 | 73 | 62 | >15 |
| 954304 | 86 | 63 | 45 | 21 | 2.39 |
| 954313 | 104 | 94 | 76 | 58 | >15 |
| 954335 | 88 | 65 | 52 | 33 | 3.86 |
| 954343 | 90 | 63 | 44 | 36 | 3.62 |
| 954391 | 83 | 76 | 57 | 48 | 11.41 |
| 954392 | 88 | 84 | 74 | 73 | >15 |

TABLE 52
Dose-dependent reduction of human HTT RNA
in A431 cells by modified oligonucleotides

| | HTT RNA (% UTC) | | | | IC50 |
|---|---|---|---|---|---|
| Compound No. | 234 nM | 938 nM | 3750 nM | 15000 nM | (μM) |
| 387916 | 41 | 16 | 7 | 2 | <0.23 |
| 953906 | 71 | 74 | 65 | 49 | >15 |
| 953921 | 66 | 93 | 78 | 62 | >15 |
| 953954 | 75 | 62 | 52 | 36 | 3.76 |
| 953978 | 102 | 98 | 89 | 81 | >15 |
| 954027 | 77 | 63 | 35 | 24 | 1.81 |
| 954042 | 80 | 43 | 35 | 21 | 1.28 |
| 954058 | 102 | 87 | 75 | 56 | >15 |
| 954066 | 99 | 63 | 51 | 31 | 3.77 |
| 954081 | 96 | 95 | 66 | 47 | 13.91 |
| 954123 | 88 | 67 | 49 | 39 | 4.63 |
| 954185 | 106 | 102 | 110 | 114 | >15 |
| 954194 | 91 | 84 | 60 | 46 | 10.83 |
| 954266 | 69 | 64 | 51 | 44 | 5.69 |
| 954273 | 87 | 81 | 61 | 51 | >15 |
| 954274 | 79 | 66 | 52 | 49 | 8.19 |
| 954290 | 82 | 51 | 44 | 34 | 2.39 |
| 954329 | 86 | 91 | 82 | 78 | >15 |
| 954353 | 97 | 116 | 88 | 85 | >15 |

TABLE 53
Dose-dependent reduction of human HTT RNA
in A431 cells by modified oligonucleotides

| | HTT RNA (% UTC) | | | | IC50 |
|---|---|---|---|---|---|
| Compound No. | 234 nM | 938 nM | 3750 nM | 15000 nM | (μM) |
| 387916 | 39 | 14 | 5 | 2 | <0.23 |
| 388817 | 100 | 93 | 81 | 82 | >15 |
| 953827 | 87 | 87 | 65 | 69 | >15 |
| 953979 | 86 | 89 | 72 | 65 | >15 |
| 953980 | 94 | 81 | 77 | 56 | >15 |
| 954028 | 77 | 100 | 72 | 54 | >15 |
| 954035 | 100 | 88 | 74 | 59 | >15 |
| 954043 | 78 | 61 | 40 | 28 | 2.14 |
| 954044 | 68 | 49 | 32 | 27 | 1.03 |
| 954068 | 72 | 58 | 36 | 17 | 1.36 |
| 954204 | 110 | 93 | 77 | 57 | >15 |
| 954219 | 113 | 106 | 126 | 101 | >15 |
| 954299 | 95 | 79 | 75 | 101 | >15 |
| 954339 | 75 | 82 | 66 | 53 | >15 |
| 954364 | 65 | 45 | 41 | 28 | 1.00 |
| 954372 | 87 | 71 | 78 | 65 | >15 |
| 954395 | 102 | 106 | 78 | 91 | >15 |
| 954411 | 77 | 66 | 52 | 63 | >15 |
| 954412 | 104 | 98 | 77 | 68 | >15 |

Modified oligonucleotides selected from the examples above were tested at various doses in A431 cells. Cultured A431 cells at a density of 10,000 cells per well were treated by free uptake with various concentrations of modified oligonucleotide as specified in the tables below. After a treatment period of approximately 48 hours, total RNA was isolated from the cells and HTT RNA levels were measured by quantitative real-time RTPCR. Human HTT primer-probe set RTS36478 (described herein above) was used to measure RNA levels as described above. HTT RNA levels were normalized to total RNA content, as measured by RIBOGREEN®. Reduction of HTT RNA is presented in the tables below as percent HTT RNA, relative to the amount of HTT RNA in untreated control cells (% UTC).

The half maximal inhibitory concentration (IC₅₀) of each modified oligonucleotide was calculated using a linear regression on a log/linear plot of the data in Excel and is also presented in the tables below. N.D. in the tables below refers to instances where the value was Not Defined.

TABLE 54
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 85 | 61 | 39 | 24 | 0.59 |
| 1314541 | 60 | 38 | 22 | 12 | 0.11 |
| 1314544 | 71 | 41 | 26 | 14 | 0.20 |
| 1314723 | 57 | 37 | 19 | 10 | 0.09 |
| 1314943 | 73 | 44 | 27 | 17 | 0.24 |
| 1315039 | 67 | 45 | 28 | 16 | 0.20 |
| 1315041 | 70 | 47 | 33 | 19 | 0.26 |
| 1315074 | 32 | 15 | 9 | 9 | <0.06 |
| 1315123 | 71 | 42 | 30 | 15 | 0.23 |
| 1315615 | 78 | 63 | 41 | 29 | 0.63 |
| 1315692 | 56 | 31 | 20 | 11 | 0.07 |
| 1315698 | 71 | 47 | 29 | 18 | 0.25 |
| 1315847 | 73 | 53 | 34 | 21 | 0.35 |
| 1316080 | 99 | 71 | 49 | 29 | 1.02 |
| 1316096 | 81 | 55 | 39 | 25 | 0.50 |
| 1316234 | 79 | 55 | 36 | 18 | 0.41 |
| 1316312 | 47 | 26 | 16 | 10 | <0.06 |
| 1316500 | 84 | 60 | 38 | 21 | 0.53 |
| 1316679 | 81 | 55 | 34 | 18 | 0.41 |

TABLE 55
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 90 | 64 | 40 | 24 | 0.67 |
| 1314789 | 63 | 42 | 23 | 11 | 0.15 |
| 1314848 | 78 | 50 | 28 | 16 | 0.31 |
| 1315014 | 69 | 43 | 25 | 16 | 0.20 |
| 1315137 | 66 | 45 | 24 | 11 | 0.18 |
| 1315143 | 68 | 37 | 17 | 9 | 0.15 |
| 1315207 | 88 | 66 | 38 | 21 | 0.61 |
| 1315294 | 65 | 40 | 23 | 13 | 0.15 |
| 1315566 | 73 | 41 | 26 | 19 | 0.24 |
| 1315702 | 57 | 32 | 20 | 12 | 0.08 |
| 1315752 | 74 | 49 | 32 | 16 | 0.30 |
| 1315874 | 87 | 63 | 34 | 17 | 0.50 |
| 1316169 | 69 | 51 | 27 | 14 | 0.25 |
| 1316218 | 73 | 54 | 36 | 18 | 0.35 |
| 1316589 | 72 | 46 | 28 | 16 | 0.25 |
| 1316616 | 85 | 64 | 42 | 20 | 0.59 |
| 1316620 | 76 | 59 | 37 | 25 | 0.47 |
| 1316972 | 81 | 55 | 33 | 20 | 0.41 |
| 1317178 | 75 | 53 | 33 | 22 | 0.35 |

TABLE 56
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 72 | 63 | 40 | 25 | 0.50 |
| 1314696 | 84 | 54 | 30 | 31 | 0.43 |
| 1314810 | 42 | 25 | 14 | 10 | <0.06 |
| 1314894 | 96 | 85 | 77 | 35 | 2.75 |
| 1314930 | 75 | 51 | 38 | 23 | 0.38 |
| 1314999 | 55 | 35 | 21 | 13 | 0.08 |
| 1315027 | 77 | 50 | 30 | 20 | 0.33 |
| 1315155 | 65 | 44 | 36 | 24 | 0.22 |
| 1315174 | 74 | 46 | 32 | 15 | 0.27 |
| 1315556 | 64 | 42 | 38 | 24 | 0.21 |
| 1315578 | 62 | 39 | 22 | 11 | 0.13 |
| 1315808 | 100 | 75 | 57 | 40 | 1.71 |
| 1316170 | 74 | 54 | 33 | 21 | 0.36 |
| 1316535 | 71 | 52 | 44 | 34 | 0.51 |
| 1316563 | 46 | 26 | 15 | 9 | <0.06 |
| 1316725 | 73 | 50 | 31 | 17 | 0.29 |
| 1316768 | 112 | 61 | 43 | 30 | 0.91 |
| 1316803 | 78 | 58 | 37 | 30 | 0.54 |
| 1316893 | 101 | 86 | 81 | 58 | >4 |

TABLE 57
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 100 | 64 | 45 | 26 | 0.83 |
| 1314562 | 90 | 67 | 67 | 34 | 1.62 |
| 1314685 | 72 | 47 | 26 | 23 | 0.26 |
| 1314813 | 64 | 41 | 25 | 15 | 0.15 |
| 1314888 | 81 | 66 | 41 | 20 | 0.58 |
| 1315056 | 64 | 43 | 29 | 13 | 0.17 |
| 1315482 | 90 | 47 | 27 | 15 | 0.37 |
| 1315558 | 96 | 104 | 85 | 77 | >4 |
| 1315781 | 96 | 65 | 45 | 27 | 0.82 |
| 1315803 | 79 | 62 | 43 | 26 | 0.59 |
| 1316274 | 74 | 56 | 40 | 26 | 0.47 |
| 1316324 | 85 | 62 | 45 | 31 | 0.77 |
| 1316938 | 95 | 73 | 62 | 58 | >4 |
| 1317061 | 82 | 48 | 38 | 19 | 0.40 |
| 1317115 | 75 | 53 | 37 | 23 | 0.39 |
| 1317128 | 79 | 59 | 31 | 16 | 0.40 |
| 1317163 | 87 | 65 | 47 | 25 | 0.76 |
| 1317190 | 106 | 95 | 88 | 79 | >4 |
| 1317205 | 75 | 47 | 27 | 13 | 0.27 |

TABLE 58
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 86 | 49 | 33 | 18 | 0.40 |
| 1314548 | 80 | 58 | 28 | 11 | 0.36 |
| 1314650 | 59 | 31 | 23 | 11 | 0.09 |
| 1314744 | 61 | 42 | 25 | 15 | 0.14 |
| 1314824 | 68 | 49 | 31 | 18 | 0.25 |
| 1314964 | 79 | 57 | 35 | 20 | 0.43 |
| 1314982 | 87 | 47 | 29 | 20 | 0.39 |
| 1315234 | 74 | 52 | 29 | 18 | 0.31 |
| 1315321 | 46 | 23 | 13 | 9 | <0.06 |
| 1315447 | 69 | 43 | 30 | 23 | 0.23 |
| 1315636 | 77 | 48 | 31 | 16 | 0.31 |
| 1315877 | 72 | 45 | 23 | 10 | 0.22 |
| 1315886 | 93 | 72 | 64 | 63 | >4 |
| 1315921 | 38 | 16 | 8 | 4 | <0.06 |
| 1316310 | 75 | 50 | 31 | 16 | 0.31 |
| 1316551 | 84 | 43 | 28 | 14 | 0.31 |
| 1316772 | 93 | 63 | 37 | 21 | 0.61 |
| 1317204 | 72 | 48 | 29 | 15 | 0.26 |
| 1317227 | 73 | 44 | 27 | 15 | 0.24 |

TABLE 59
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 77 | 65 | 42 | 23 | 0.56 |
| 1314487 | 54 | 35 | 18 | 10 | 0.07 |
| 1314570 | 71 | 47 | 36 | 20 | 0.29 |
| 1314596 | 78 | 61 | 33 | 22 | 0.45 |
| 1314899 | 79 | 54 | 30 | 20 | 0.38 |
| 1314904 | 60 | 35 | 24 | 15 | 0.11 |
| 1315562 | 69 | 50 | 30 | 21 | 0.26 |
| 1315672 | 55 | 34 | 18 | 11 | 0.08 |
| 1315733 | 76 | 48 | 32 | 23 | 0.33 |
| 1315747 | 67 | 45 | 35 | 19 | 0.24 |
| 1315754 | 70 | 45 | 29 | 14 | 0.23 |
| 1315964 | 44 | 24 | 16 | 8 | <0.06 |
| 1316061 | 70 | 49 | 31 | 26 | 0.29 |
| 1316098 | 72 | 49 | 32 | 19 | 0.28 |
| 1316154 | 66 | 47 | 30 | 15 | 0.22 |
| 1316254 | 89 | 52 | 33 | 23 | 0.48 |
| 1316604 | 47 | 28 | 14 | 19 | <0.06 |
| 1316716 | 65 | 45 | 27 | 20 | 0.19 |
| 1316965 | 78 | 55 | 32 | 18 | 0.38 |

TABLE 60
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 85 | 107 | 51 | 26 | 1.30 |
| 1314791 | 106 | 66 | 46 | 34 | 1.07 |
| 1314822 | 77 | 79 | 41 | 22 | 0.76 |
| 1314849 | 42 | 24 | 12 | 8 | <0.06 |
| 1315621 | 105 | 60 | 38 | 20 | 0.66 |
| 1315723 | 67 | 46 | 29 | N.D. | 0.20 |
| 1315867 | 52 | 36 | 22 | 12 | 0.07 |
| 1315881 | 90 | 55 | 46 | 29 | 0.74 |
| 1315883 | 70 | 55 | N.D. | 19 | 0.34 |
| 1315911 | 87 | 62 | 46 | 28 | 0.75 |
| 1316106 | 72 | 53 | 36 | 22 | 0.35 |
| 1316346 | 140 | 59 | 38 | 21 | 0.85 |
| 1316392 | 63 | 42 | 18 | 7 | 0.14 |
| 1316414 | 68 | 39 | 17 | 6 | 0.16 |
| 1316704 | 79 | 60 | 37 | 19 | 0.46 |
| 1316835 | 85 | 59 | 44 | 29 | 0.68 |
| 1317084 | 66 | 52 | 37 | 22 | 0.29 |
| 1317090 | 48 | 32 | 24 | 12 | <0.06 |
| 1317136 | 76 | 39 | 20 | 8 | 0.21 |

TABLE 61
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 78 | 58 | 41 | 18 | 0.46 |
| 1314606 | 80 | 60 | 36 | 22 | 0.48 |
| 1314678 | 81 | 60 | 41 | 39 | 0.83 |
| 1314828 | 72 | 49 | 29 | 19 | 0.28 |
| 1314829 | 58 | 29 | 16 | 9 | 0.07 |
| 1314935 | 59 | 44 | 26 | 16 | 0.14 |
| 1315083 | 48 | 28 | 16 | 11 | <0.06 |
| 1315309 | 75 | 60 | 36 | 22 | 0.44 |
| 1315347 | 56 | 36 | 20 | 10 | 0.09 |
| 1315501 | 59 | 36 | 26 | 13 | 0.11 |
| 1315512 | 79 | 44 | 33 | 17 | 0.31 |
| 1315652 | 61 | 32 | 20 | 8 | 0.10 |
| 1315746 | 71 | 43 | 26 | 20 | 0.23 |
| 1315885 | 88 | 69 | 45 | 24 | 0.76 |
| 1316435 | 47 | 23 | 13 | 8 | <0.06 |
| 1316639 | 38 | 23 | 14 | 9 | <0.06 |
| 1316788 | 79 | 49 | 38 | 20 | 0.38 |
| 1316846 | 74 | 57 | 29 | 22 | 0.36 |
| 1317164 | 72 | 52 | 39 | 17 | 0.34 |

TABLE 62
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 91 | 64 | 31 | 20 | 0.54 |
| 1314511 | 86 | 41 | 23 | 17 | 0.30 |
| 1314540 | 75 | 42 | 27 | 13 | 0.24 |
| 1314603 | 73 | 47 | 33 | 23 | 0.33 |
| 1315317 | 48 | 26 | 14 | 7 | <0.06 |
| 1315408 | 62 | 41 | 26 | 14 | 0.14 |
| 1315499 | 95 | 77 | 69 | 52 | >4 |
| 1315536 | 73 | 43 | 26 | 14 | 0.23 |
| 1315537 | 85 | 52 | 28 | 16 | 0.38 |
| 1315584 | 75 | 60 | 35 | 18 | 0.40 |
| 1315794 | 75 | 37 | 23 | 13 | 0.20 |
| 1315950 | 55 | 32 | 14 | 6 | 0.07 |
| 1316400 | 66 | 39 | 21 | 12 | 0.15 |
| 1316621 | 69 | 39 | 26 | 8 | 0.19 |
| 1316672 | 77 | 51 | 28 | 12 | 0.30 |
| 1316766 | 65 | 34 | 24 | 17 | 0.13 |
| 1316908 | 70 | 46 | 24 | 19 | 0.22 |
| 1317017 | 63 | 33 | 17 | 9 | 0.11 |
| 1317031 | 63 | 30 | 20 | 10 | 0.10 |

TABLE 63
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 83 | 58 | 44 | 24 | 0.59 |
| 1314708 | 52 | 31 | 18 | 15 | <0.06 |
| 1314762 | 45 | 20 | 9 | 6 | <0.06 |
| 1314827 | 81 | 58 | 41 | 24 | 0.55 |
| 1314891 | 74 | 57 | 35 | 22 | 0.40 |
| 1314907 | 71 | 34 | 18 | 8 | 0.16 |
| 1314918 | 45 | 22 | 12 | 6 | <0.06 |
| 1314920 | 88 | 64 | 43 | 25 | 0.68 |
| 1314967 | 33 | 14 | 6 | 4 | <0.06 |
| 1315340 | 53 | 22 | 14 | 6 | <0.06 |
| 1315412 | 80 | 53 | 36 | 22 | 0.42 |
| 1315678 | 90 | 44 | 24 | 11 | 0.33 |
| 1315749 | 71 | 49 | 31 | 18 | 0.28 |
| 1316062 | 57 | 30 | 14 | 8 | 0.07 |
| 1316162 | 97 | 80 | 58 | 34 | 1.54 |
| 1316544 | 57 | 35 | 19 | 10 | 0.09 |
| 1316722 | 90 | 73 | 49 | 27 | 0.94 |
| 1316775 | 81 | 60 | 41 | 21 | 0.52 |
| 1317050 | 77 | 49 | 34 | 23 | 0.35 |

TABLE 64
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 83 | 61 | 38 | 23 | 0.56 |
| 1314845 | 61 | 44 | 25 | 13 | 0.15 |
| 1314916 | 90 | 81 | 31 | 16 | 0.63 |
| 1314928 | 85 | 68 | 53 | 34 | 1.11 |
| 1315077 | 68 | 44 | 18 | 20 | 0.18 |
| 1315147 | 74 | 45 | 31 | 15 | 0.26 |
| 1315341 | 54 | 27 | 16 | 9 | <0.06 |
| 1315425 | 64 | 42 | 28 | 17 | 0.16 |
| 1315498 | 81 | 53 | 38 | 23 | 0.46 |
| 1315722 | 86 | 58 | 39 | 22 | 0.54 |
| 1315812 | 70 | 52 | 32 | 23 | 0.30 |
| 1315879 | 81 | 61 | 47 | 26 | 0.66 |
| 1315897 | 98 | 66 | 40 | 23 | 0.73 |
| 1315974 | 65 | 44 | 33 | 27 | 0.20 |
| 1316232 | 83 | 52 | 35 | 21 | 0.42 |
| 1316421 | 85 | 66 | 49 | 27 | 0.81 |
| 1316508 | 99 | 64 | 41 | 26 | 0.76 |
| 1316762 | 73 | 60 | 37 | 27 | 0.46 |
| 1316926 | 55 | 31 | 21 | 22 | <0.06 |

TABLE 65
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 90 | 56 | 37 | 21 | 0.53 |
| 1315878 | 67 | 42 | 27 | 15 | 0.18 |
| 1316143 | 64 | 48 | 31 | 21 | 0.21 |
| 1316972 | 69 | 47 | 32 | 19 | 0.25 |
| 1317142 | 73 | 38 | 26 | 15 | 0.21 |
| 1318569 | 72 | 49 | 31 | 19 | 0.29 |
| 1318594 | 70 | 52 | 42 | 31 | 0.43 |
| 1318605 | 80 | 46 | 29 | 19 | 0.32 |
| 1318615 | 61 | 39 | 21 | 13 | 0.12 |
| 1318620 | 61 | 35 | 28 | 15 | 0.12 |
| 1318641 | 75 | 50 | 39 | 21 | 0.37 |
| 1318695 | 68 | 39 | 23 | 15 | 0.17 |
| 1318698 | 83 | 47 | 24 | 16 | 0.31 |
| 1318708 | 57 | 30 | 19 | 13 | 0.07 |
| 1318715 | 79 | 51 | 33 | 19 | 0.36 |
| 1318726 | 66 | 41 | 27 | 17 | 0.17 |
| 1318742 | 63 | 38 | 25 | 15 | 0.13 |
| 1318745 | 66 | 41 | 26 | 16 | 0.17 |
| 1318747 | 64 | 41 | 32 | 17 | 0.17 |

TABLE 66
Dose-dependent reduction of human HTT RNA
in A431 cells by modified oligonucleotides

| | HTT RNA (% UTC) | | | | IC50 |
|---|---|---|---|---|---|
| Compound No. | 63 nM | 250 nM | 1000 nM | 4000 nM | (μM) |
| 388241 | 66 | 58 | 43 | 32 | 0.50 |
| 1314755 | 95 | 61 | 39 | 27 | 0.70 |
| 1314833 | 56 | 53 | 47 | 22 | 0.25 |
| 1314867 | 71 | 50 | 25 | 17 | 0.25 |
| 1314905 | 38 | 22 | 15 | 6 | <0.06 |
| 1315119 | 73 | 51 | 24 | 20 | 0.27 |
| 1315141 | 74 | 46 | 28 | 22 | 0.28 |
| 1315214 | 48 | 22 | 16 | 9 | <0.06 |
| 1315282 | 125 | 120 | 112 | 76 | >4 |
| 1315343 | 66 | 37 | 25 | 9 | 0.15 |
| 1315405 | 56 | 44 | 22 | 17 | 0.11 |
| 1315604 | 65 | 42 | 38 | 27 | 0.22 |
| 1315813 | 77 | 67 | 38 | 13 | 0.46 |
| 1316229 | 100 | 72 | 49 | 29 | 1.03 |
| 1316506 | 68 | 35 | 20 | 10 | 0.15 |
| 1316802 | 70 | 40 | 24 | 13 | 0.18 |
| 1316984 | 74 | 63 | 59 | 33 | 1.08 |

### Example 6: Design of Modified Oligonucleotides Complementary to Human HTT Nucleic Acid

Modified oligonucleotides complementary to a human HTT nucleic acid were designed, as described in the tables below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

The modified oligonucleotides in the table below are 17 nucleosides in length, wherein the sugar motif for the gapmers is (from 5′ to 3′): eeeeddddddddkkeee; wherein each “d” represents a 2′-β-D-deoxyribosyl sugar moiety, each “e” represents a 2′-MOE sugar moiety, and each “k” represents a cEt sugar moiety. The modified oligonucleotides have an internucleoside linkage motif of (from 5′ to 3′): soosssssssssooss; wherein each “s” represents a phosphorothioate internucleoside linkage, and each “o” represents a phosphodiester internucleoside linkage. Each cytosine residue is a 5-methylcytosine.

TABLE 67
Modified oligonucleotides with a mixed MOE/cEt sugar motif and mixed PO/PS
internucleoside linkages complementary to human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1419386 | ACAGCTTTTATTTCCAT | N/A | N/A | 126800 | 126816 | 3552 |
| 1419387 | ACTCATTTCCACCTTCA | 5430 | 5446 | 115308 | 115324 | 3553 |
| 1419388 | AGAGGTCATCTTCGGTC | N/A | N/A | 118733 | 118749 | 3554 |
| 1419389 | AGCTTTTATTTCCATAC | N/A | N/A | 126798 | 126814 | 3555 |
| 1419390 | ATCTTGTTTTACCACCC | N/A | N/A | 35334 | 35350 | 3556 |
| 1419391 | ATTACTTTTTACAATGG | N/A | N/A | 62838 | 62854 | 3557 |
| 1419392 | CACAGCTTTTATTTCCA | N/A | N/A | 126801 | 126817 | 3558 |
| 1419393 | CACTCATTTCCACCTTC | 5431 | 5447 | 115309 | 115325 | 3559 |
| 1419394 | CAGCTTTTATTTCCATA | N/A | N/A | 126799 | 126815 | 3560 |
| 1419395 | CATCTTGTTTTACCACC | N/A | N/A | 35335 | 35351 | 3561 |
| 1419396 | CATTACTTTTTACAATG | N/A | N/A | 62839 | 62855 | 3562 |
| 1419397 | CATTTCCACCTTCAGCT | 5427 | 5443 | 115305 | 115321 | 3563 |
| 1419398 | CTAGAGGTCATCTTCGG | N/A | N/A | 118735 | 118751 | 3564 |
| 1419399 | CTCATTTCCACCTTCAG | 5429 | 5445 | 115307 | 115323 | 3565 |
| 1419400 | CTTTGATTTTTACCAGT | N/A | N/A | 60027 | 60043 | 3566 |
| 1419401 | GAGGTCATCTTCGGTCA | N/A | N/A | 118732 | 118748 | 3567 |
| 1419402 | GCATCTTGTTTTACCAC | N/A | N/A | 35336 | 35352 | 3568 |
| 1419403 | GCATTACTTTTTACAAT | N/A | N/A | 62840 | 62856 | 3569 |
| 1419404 | GCTGCTCACTCATTTCC | 5437 | 5453 | 115315 | 115331 | 3570 |
| 1419405 | GCTTTGATTTTTACCAG | N/A | N/A | 60028 | 60044 | 3571 |
| 1419406 | GCTTTTATTTCCATACA | N/A | N/A | 126797 | 126813 | 3572 |
| 1419407 | GGTCACAGCTTTTATTT | N/A | N/A | 126804 | 126820 | 3573 |
| 1419408 | GGTTAGTTTTCCTTTTA | N/A | N/A | 117880 | 117896 | 3574 |
| 1419409 | GTCACAGCTTTTATTTC | N/A | N/A | 126803 | 126819 | 3575 |
| 1419410 | GTTAGTTTTCCTTTTAT | N/A | N/A | 117879 | 117895 | 3576 |
| 1419412 | TAGAGGTCATCTTCGGT | N/A | N/A | 118734 | 118750 | 3577 |
| 1419413 | TAGTTTTCCTTTTATAT | N/A | N/A | 117877 | 117893 | 3578 |
| 1419414 | TCACAGCTTTTATTTCC | N/A | N/A | 126802 | 126818 | 3579 |
| 1419415 | TCACTCATTTCCACCTT | 5432 | 5448 | 115310 | 115326 | 3580 |
| 1419416 | TCATTTCCACCTTCAGC | 5428 | 5444 | 115306 | 115322 | 3581 |
| 1419417 | TCTTGTTTTACCACCCA | N/A | N/A | 35333 | 35349 | 3582 |
| 1419418 | TGCTGCTCACTCATTTC | 5438 | 5454 | 115316 | 115332 | 3583 |
| 1419419 | TGGTCACAGCTTTTATT | N/A | N/A | 126805 | 126821 | 3584 |
| 1419420 | TGTTGCTGCTCACTCAT | 5441 | 5457 | 115319 | 115335 | 3585 |
| 1419421 | TTACTTTTTACAATGGA | N/A | N/A | 62837 | 62853 | 3586 |
| 1419422 | TTAGTTTTCCTTTTATA | N/A | N/A | 117878 | 117894 | 3587 |
| 1419423 | TTGATTTTTACCAGTTA | N/A | N/A | 60025 | 60041 | 3588 |
| 1419424 | TTGCTGCTCACTCATTT | 5439 | 5455 | 115317 | 115333 | 3589 |
| 1419425 | TTTGATTTTTACCAGTT | N/A | N/A | 60026 | 60042 | 3590 |

The modified oligonucleotide in the table below is a 5-10-5 MOE gapmer. The gapmer is 20 nucleosides in length, wherein the sugar motif for the gapmer is (from 5′ to 3′): eeeeeddddddddddeeeee; wherein each “d” represents a 2′-β-D-deoxyribosyl sugar moiety, and each “e” represents a 2′-MOE sugar moiety. The gapmer has an internucleoside linkage motif of (from 5′ to 3′): sooosssssssssssooos; wherein each “s” represents a phosphorothioate internucleoside linkage, and each “o” represents a phosphodiester internucleoside linkage. Each cytosine residue is a 5-methylcytosine.

TABLE 68
5-10-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 444652 | TTTCTCTATTGCACATTCCA | 5809 | 5828 | 130392 | 130411 | 3591 |

The modified oligonucleotides in the table below are 5-10-5 MOE gapmers. The gapmers are 20 nucleosides in length, wherein the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. The gapmers have an internucleoside linkage motif of (from 5′ to 3′): sooosssssssssssooss; wherein each “s” represents a phosphorothioate internucleoside linkage, and each “o” represents a phosphodiester internucleoside linkage. Each cytosine residue is a 5-methylcytosine.

TABLE 69
5-10-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1391291 | ACACAGCCTGCTAGAGGTCA | N/A | N/A | 118742 | 118761 | 3592 |
| 1391292 | CTGCTCACTCATTTCCACCT | 5433 | 5452 | 115311 | 115330 | 3593 |
| 1391293 | CTCACTCATTTCCACCTTCA | 5430 | 5449 | 115308 | 115327 | 3594 |
| 1391294 | ACAGCCTGCTAGAGGTCATC | N/A | N/A | 118740 | 118759 | 3595 |
| 1391295 | CTTTGATTTTTACCAGTTAA | N/A | N/A | 60024 | 60043 | 3596 |
| 1391296 | TCATTTCCACCTTCAGCTGT | 5425 | 5444 | 115303 | 115322 | 3597 |
| 1391297 | TTGGGGGTGGTCACAGCTTT | N/A | N/A | 126809 | 126828 | 3598 |
| 1391298 | TTTTGCTTTGATTTTTACCA | N/A | N/A | 60029 | 60048 | 3599 |
| 1391299 | CCACCTTCAGCTGTTTTGTA | 5419 | 5438 | 115297 | 115316 | 3600 |
| 1391301 | TGCTGCTCACTCATTTCCAC | 5435 | 5454 | 115313 | 115332 | 3601 |
| 1391302 | GTTTTTTGCTTTGATTTTTA | N/A | N/A | 60032 | 60051 | 3602 |
| 1391303 | TTTGCTTTGATTTTTACCAG | N/A | N/A | 60028 | 60047 | 3603 |
| 1391304 | TTCCACCTTCAGCTGTTTTG | 5421 | 5440 | 115299 | 115318 | 3604 |
| 1391305 | TTTTTGCTTTGATTTTTACC | N/A | N/A | 60030 | 60049 | 3605 |
| 1391306 | GCCTGCTAGAGGTCATCTTC | N/A | N/A | 118737 | 118756 | 3606 |
| 1391307 | TTGTAGGTTTTTTGCTTTGA | N/A | N/A | 60038 | 60057 | 3607 |
| 1391308 | TGGTCACAGCTTTTATTTCC | N/A | N/A | 126802 | 126821 | 3608 |
| 1391309 | TGTTTTACCACCCATGCTGG | N/A | N/A | 35327 | 35346 | 3609 |
| 1391310 | GTGCATTACTTTTTACAATG | N/A | N/A | 62839 | 62858 | 3610 |
| 1391311 | GGGGTGGTCACAGCTTTTAT | N/A | N/A | 126806 | 126825 | 3611 |
| 1391312 | TAGAGGTCATCTTCGGTCAT | N/A | N/A | 118731 | 118750 | 3612 |
| 1391313 | GTTGGGGGTGGTCACAGCTT | N/A | N/A | 126810 | 126829 | 3613 |
| 1391314 | TATGTTGCTGCTCACTCATT | 5440 | 5459 | 115318 | 115337 | 3614 |
| 1391315 | TGCTTTGATTTTTACCAGTT | N/A | N/A | 60026 | 60045 | 3615 |
| 1391316 | ATTTCCACCTTCAGCTGTTT | 5423 | 5442 | 115301 | 115320 | 3616 |
| 1391318 | GAGGTCATCTTCGGTCATGT | N/A | N/A | 118729 | 118748 | 3617 |
| 1391319 | TCTTGTTTTACCACCCATGC | N/A | N/A | 35330 | 35349 | 3618 |
| 1391320 | CACAGCTTTTATTTCCATAC | N/A | N/A | 126798 | 126817 | 3619 |
| 1391321 | TACTTTTTACAATGGACAGT | N/A | N/A | 62833 | 62852 | 3620 |
| 1391322 | AGGTCATCTTCGGTCATGTG | N/A | N/A | 118728 | 118747 | 3621 |
| 1391323 | ATTACTTTTTACAATGGACA | N/A | N/A | 62835 | 62854 | 3622 |
| 1391324 | TGCATTACTTTTTACAATGG | N/A | N/A | 62838 | 62857 | 3623 |
| 1391325 | GGCATCTTGTTTTACCACCC | N/A | N/A | 35334 | 35353 | 3624 |
| 1391326 | TTGCTTTGATTTTTACCAGT | N/A | N/A | 60027 | 60046 | 3625 |
| 1391327 | ATCTTGTTTTACCACCCATG | N/A | N/A | 35331 | 35350 | 3626 |
| 1391328 | TTACTTTTTACAATGGACAG | N/A | N/A | 62834 | 62853 | 3627 |
| 1391329 | TGGGGGTGGTCACAGCTTTT | N/A | N/A | 126808 | 126827 | 3628 |
| 1391330 | CTGCTAGAGGTCATCTTCGG | N/A | N/A | 118735 | 118754 | 3629 |
| 1391332 | GCTGCTCACTCATTTCCACC | 5434 | 5453 | 115312 | 115331 | 3630 |
| 1391333 | GTTGCTGCTCACTCATTTCC | 5437 | 5456 | 115315 | 115334 | 3631 |
| 1391334 | AGCCTGCTAGAGGTCATCTT | N/A | N/A | 118738 | 118757 | 3632 |
| 1391335 | TGGCATCTTGTTTTACCACC | N/A | N/A | 35335 | 35354 | 3633 |
| 1391336 | AAGTGCATTACTTTTTACAA | N/A | N/A | 62841 | 62860 | 3634 |
| 1391337 | GGTGGTCACAGCTTTTATTT | N/A | N/A | 126804 | 126823 | 3635 |
| 1391338 | ATGGCATCTTGTTTTACCAC | N/A | N/A | 35336 | 35355 | 3636 |
| 1391339 | CTCATTTCCACCTTCAGCTG | 5426 | 5445 | 115304 | 115323 | 3637 |
| 1391340 | TGTAGGTTTTTTGCTTTGAT | N/A | N/A | 60037 | 60056 | 3638 |
| 1391341 | GTTTTACCACCCATGCTGGA | N/A | N/A | 35326 | 35345 | 3639 |
| 1391342 | CCTGCTAGAGGTCATCTTCG | N/A | N/A | 118736 | 118755 | 3640 |
| 1391343 | TAGGTTTTTTGCTTTGATTT | N/A | N/A | 60035 | 60054 | 3641 |
| 1391344 | TTTTTTGCTTTGATTTTTAC | N/A | N/A | 60031 | 60050 | 3642 |
| 1391345 | CAGCCTGCTAGAGGTCATCT | N/A | N/A | 118739 | 118758 | 3643 |
| 1391346 | GGGGGTGGTCACAGCTTTTA | N/A | N/A | 126807 | 126826 | 3644 |
| 1391347 | GTAGGTTTTTTGCTTTGATT | N/A | N/A | 60036 | 60055 | 3645 |
| 1391348 | ACAGCTTTTATTTCCATACA | N/A | N/A | 126797 | 126816 | 3646 |
| 1391349 | CACAGCCTGCTAGAGGTCAT | N/A | N/A | 118741 | 118760 | 3647 |
| 1391351 | GCTCACTCATTTCCACCTTC | 5431 | 5450 | 115309 | 115328 | 3648 |
| 1391352 | GTGGTCACAGCTTTTATTTC | N/A | N/A | 126803 | 126822 | 3649 |
| 1391353 | TGTTGCTGCTCACTCATTTC | 5438 | 5457 | 115316 | 115335 | 3650 |
| 1391355 | TTGTTTTACCACCCATGCTG | N/A | N/A | 35328 | 35347 | 3651 |
| 1391356 | ATGTTGCTGCTCACTCATTT | 5439 | 5458 | 115317 | 115336 | 3652 |
| 1391357 | TTTCCACCTTCAGCTGTTTT | 5422 | 5441 | 115300 | 115319 | 3653 |
| 1391358 | AGAGGTCATCTTCGGTCATG | N/A | N/A | 118730 | 118749 | 3654 |
| 1391359 | GGTTTTTTGCTTTGATTTTT | N/A | N/A | 60033 | 60052 | 3655 |
| 1391360 | AGGTTTTTTGCTTTGATTTT | N/A | N/A | 60034 | 60053 | 3656 |
| 1391361 | GCTAGAGGTCATCTTCGGTC | N/A | N/A | 118733 | 118752 | 3657 |
| 1391362 | GGTCATCTTCGGTCATGTGA | N/A | N/A | 118727 | 118746 | 3658 |
| 1391363 | TGCTAGAGGTCATCTTCGGT | N/A | N/A | 118734 | 118753 | 3659 |
| 1391364 | TCACTCATTTCCACCTTCAG | 5429 | 5448 | 115307 | 115326 | 3660 |
| 1391365 | CATTTCCACCTTCAGCTGTT | 5424 | 5443 | 115302 | 115321 | 3661 |
| 1391366 | TCCACCTTCAGCTGTTTTGT | 5420 | 5439 | 115298 | 115317 | 3662 |
| 1391367 | GGGTGGTCACAGCTTTTATT | N/A | N/A | 126805 | 126824 | 3663 |
| 1391368 | AGTGCATTACTTTTTACAAT | N/A | N/A | 62840 | 62859 | 3664 |
| 1394315 | GTTAGTTTTCCTTTTATATT | N/A | N/A | 117876 | 117895 | 3665 |
| 1394316 | ACTTGGTTAGTTTTCCTTTT | N/A | N/A | 117881 | 117900 | 3666 |
| 1394317 | TCCTTTTATATTCATCAGAA | N/A | N/A | 117868 | 117887 | 3667 |
| 1394318 | TTCCTTTTATATTCATCAGA | N/A | N/A | 117869 | 117888 | 3668 |
| 1394320 | TTAGTTTTCCTTTTATATTC | N/A | N/A | 117875 | 117894 | 3669 |
| 1394321 | TAACTTGGTTAGTTTTCCTT | N/A | N/A | 117883 | 117902 | 3670 |
| 1394323 | AACTTGGTTAGTTTTCCTTT | N/A | N/A | 117882 | 117901 | 3671 |
| 1394325 | TTGGTTAGTTTTCCTTTTAT | N/A | N/A | 117879 | 117898 | 3672 |
| 1394326 | TAGTTTTCCTTTTATATTCA | N/A | N/A | 117874 | 117893 | 3673 |
| 1394331 | CTTGGTTAGTTTTCCTTTTA | N/A | N/A | 117880 | 117899 | 3674 |
| 1394335 | AGTTTTCCTTTTATATTCAT | N/A | N/A | 117873 | 117892 | 3675 |
| 1394336 | TTTCCTTTTATATTCATCAG | N/A | N/A | 117870 | 117889 | 3676 |
| 1456397 | ATATTTTCTCAACTTTAAAC | N/A | N/A | 119644 | 119663 | 3677 |
| 1456398 | CATATTTTCTCAACTTTAAA | N/A | N/A | 119645 | 119664 | 3678 |
| 1456399 | CCATATTTTCTCAACTTTAA | N/A | N/A | 119646 | 119665 | 3679 |
| 1456400 | AGCCATATTTTCTCAACTTT | N/A | N/A | 119648 | 119667 | 3680 |
| 1456401 | CAAGCCATATTTTCTCAACT | N/A | N/A | 119650 | 119669 | 3681 |
| 1456402 | ACAAGCCATATTTTCTCAAC | N/A | N/A | 119651 | 119670 | 3682 |

The modified oligonucleotides in the table below are 5-10-5 MOE gapmers. The gapmers are 20 nucleosides in length, wherein the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. The gapmers have an internucleoside linkage motif of (from 5′ to 3′): sossssssssssssssoss; wherein each “s” represents a phosphorothioate internucleoside linkage, and each “o” represents a phosphodiester internucleoside linkage. Each cytosine residue is a 5-methylcytosine.

TABLE 70
5-10-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1394439 | GGTTAGTTTTCCTTTTATAT | N/A | N/A | 117877 | 117896 | 2770 |
| 1394440 | GCATTACTTTTTACAATGGA | N/A | N/A | 62837 | 62856 | 3029 |
| 1394441 | ACTCATTTCCACCTTCAGCT | 5427 | 5446 | 115305 | 115324 | 2471 |
| 1394442 | GCATCTTGTTTTACCACCCA | N/A | N/A | 35333 | 35352 | 1208 |
| 1394443 | GCTTTGATTTTTACCAGTTA | N/A | N/A | 60025 | 60044 | 1502 |
| 1394444 | GTCACAGCTTTTATTTCCAT | N/A | N/A | 126800 | 126819 | 1652 |
| 1394445 | CTAGAGGTCATCTTCGGTCA | N/A | N/A | 118732 | 118751 | 2412 |

The modified oligonucleotides in the table below are 5-10-5 MOE gapmers. The gapmers are 20 nucleosides in length, wherein the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. The gapmers have an internucleoside linkage motif of (from 5′ to 3′): ssoosssssssssssooss; wherein each “s” represents a phosphorothioate internucleoside linkage, and each “o” represents a phosphodiester internucleoside linkage. Each cytosine residue is a 5-methylcytosine.

TABLE 71
5-10-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1394446 | CTAGAGGTCATCTTCGGTCA | N/A | N/A | 118732 | 118751 | 2412 |
| 1394447 | GGTTAGTTTTCCTTTTATAT | N/A | N/A | 117877 | 117896 | 2770 |
| 1394448 | GCTTTGATTTTTACCAGTTA | N/A | N/A | 60025 | 60044 | 1502 |
| 1394449 | ACTCATTTCCACCTTCAGCT | 5427 | 5446 | 115305 | 115324 | 2471 |
| 1394450 | GTCACAGCTTTTATTTCCAT | N/A | N/A | 126800 | 126819 | 1652 |
| 1394451 | GCATTACTTTTTACAATGGA | N/A | N/A | 62837 | 62856 | 3029 |
| 1394452 | GCATCTTGTTTTACCACCCA | N/A | N/A | 35333 | 35352 | 1208 |

The modified oligonucleotides in the table below are 5-8-5 MOE gapmers. The gapmers are 18 nucleosides in length, wherein the sugar motif for the gapmers is (from 5′ to 3′): eeeeeddddddddeeeee; wherein each “d” represents a 2′-β-D-deoxyribosyl sugar moiety, and each “e” represents a 2′-MOE sugar moiety. The gapmers have an internucleoside linkage motif of (from 5′ to 3′): sooosssssssssooss; wherein each “s” represents a phosphorothioate internucleoside linkage, and each “o” represents a phosphodiester internucleoside linkage. Each cytosine residue is a 5-methylcytosine.

TABLE 72
5-8-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1394418 | TCACAGCTTTTATTTCCA | N/A | N/A | 126801 | 126818 | 3683 |
| 1394419 | TCATTTCCACCTTCAGCT | 5427 | 5444 | 115305 | 115322 | 3684 |
| 1394420 | ATCTTGTTTTACCACCCA | N/A | N/A | 35333 | 35350 | 3685 |
| 1394421 | GGTTAGTTTTCCTTTTAT | N/A | N/A | 117879 | 117896 | 3686 |
| 1394422 | GCATTACTTTTTACAATG | N/A | N/A | 62839 | 62856 | 3687 |
| 1394423 | GTCACAGCTTTTATTTCC | N/A | N/A | 126802 | 126819 | 3688 |
| 1394424 | CTCATTTCCACCTTCAGC | 5428 | 5445 | 115306 | 115323 | 3689 |
| 1394425 | CACAGCTTTTATTTCCAT | N/A | N/A | 126800 | 126817 | 3690 |
| 1394427 | CATCTTGTTTTACCACCC | N/A | N/A | 35334 | 35351 | 3691 |
| 1394428 | ATTACTTTTTACAATGGA | N/A | N/A | 62837 | 62854 | 3692 |
| 1394429 | AGAGGTCATCTTCGGTCA | N/A | N/A | 118732 | 118749 | 3693 |
| 1394430 | TTAGTTTTCCTTTTATAT | N/A | N/A | 117877 | 117894 | 3694 |
| 1394431 | TAGAGGTCATCTTCGGTC | N/A | N/A | 118733 | 118750 | 3695 |
| 1394432 | TTTGATTTTTACCAGTTA | N/A | N/A | 60025 | 60042 | 3696 |
| 1394433 | CATTACTTTTTACAATGG | N/A | N/A | 62838 | 62855 | 3697 |
| 1394434 | GTTAGTTTTCCTTTTATA | N/A | N/A | 117878 | 117895 | 3698 |
| 1394437 | GCATCTTGTTTTACCACC | N/A | N/A | 35335 | 35352 | 3699 |
| 1419360 | GGTCACAGCTTTTATTTC | N/A | N/A | 126803 | 126820 | 3700 |
| 1419361 | ACAGCTTTTATTTCCATA | N/A | N/A | 126799 | 126816 | 3701 |
| 1419362 | CAGCTTTTATTTCCATAC | N/A | N/A | 126798 | 126815 | 3702 |
| 1419365 | TGGTCACAGCTTTTATTT | N/A | N/A | 126804 | 126821 | 3703 |
| 1419370 | AGCTTTTATTTCCATACA | N/A | N/A | 126797 | 126814 | 3704 |
| 1456411 | CCATATTTTCTCAACTTT | N/A | N/A | 119648 | 119665 | 3705 |
| 1456412 | GCCATATTTTCTCAACTT | N/A | N/A | 119649 | 119666 | 3706 |
| 1456413 | CATATTTTCTCAACTTTA | N/A | N/A | 119647 | 119664 | 3707 |

The modified oligonucleotides in the table below are 5-8-5 MOE gapmers. The gapmers are 18 nucleosides in length, wherein the sugar motif for the gapmers is (from 5′ to 3′): eeeeeddddddddeeeee; wherein each “d” represents a 2′-β-D-deoxyribosyl sugar moiety, and each “e” represents a 2′-MOE sugar moiety. The gapmers have an internucleoside linkage motif of (from 5′ to 3′): sossssssssssssoss; wherein each “s” represents a phosphorothioate internucleoside linkage, and each “o” represents a phosphodiester internucleoside linkage. Each cytosine residue is a 5-methylcytosine.

TABLE 73
5-8-5 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1419374 | GGTCACAGCTTTTATTTC | N/A | N/A | 126803 | 126820 | 3700 |
| 1419375 | ACAGCTTTTATTTCCATA | N/A | N/A | 126799 | 126816 | 3701 |
| 1419376 | CAGCTTTTATTTCCATAC | N/A | N/A | 126798 | 126815 | 3702 |
| 1419379 | TGGTCACAGCTTTTATTT | N/A | N/A | 126804 | 126821 | 3703 |
| 1419380 | CACAGCTTTTATTTCCAT | N/A | N/A | 126800 | 126817 | 3690 |
| 1419383 | GTCACAGCTTTTATTTCC | N/A | N/A | 126802 | 126819 | 3688 |
| 1419384 | AGCTTTTATTTCCATACA | N/A | N/A | 126797 | 126814 | 3704 |

The modified oligonucleotides in the table below are 6-10-4 MOE gapmers. The gapmers are 20 nucleosides in length, wherein the sugar motif for the gapmers is (from 5′ to 3′): eeeeeeddddddddddeeee; wherein each “d” represents a 2′-β-D-deoxyribosyl sugar moiety, and each “e” represents a 2′-MOE sugar moiety. The gapmers have an internucleoside linkage motif of (from 5′ to 3′): sooooossssssssssoss; wherein each “s” represents a phosphorothioate internucleoside linkage, and each “o” represents a phosphodiester internucleoside linkage. Each cytosine residue is a 5-methylcytosine.

TABLE 74
6-10-4 MOE gapmers with mixed PO/PS internucleoside linkages complementary to
human HTT

| | | SEQ | SEQ | SEQ ID | SEQ ID | |
|---|---|---|---|---|---|---|
| | | ID No: | ID No: | No: 2 | No: 2 | |
| Compound | | 1 Start | 1 Stop | Start | Stop | SEQ |
| Number | Sequence (5′ to 3′) | Site | Site | Site | Site | ID NO. |
| 1394343 | ACTCATTTCCACCTTCAGCT | 5427 | 5446 | 115305 | 115324 | 2471 |
| 1394344 | GCATCTTGTTTTACCACCCA | N/A | N/A | 35333 | 35352 | 1208 |
| 1394345 | GTCACAGCTTTTATTTCCAT | N/A | N/A | 126800 | 126819 | 1652 |
| 1394346 | GCATTACTTTTTACAATGGA | N/A | N/A | 62837 | 62856 | 3029 |
| 1394347 | GCTTTGATTTTTACCAGTTA | N/A | N/A | 60025 | 60044 | 1502 |
| 1394348 | CTAGAGGTCATCTTCGGTCA | N/A | N/A | 118732 | 118751 | 2412 |
| 1394349 | GGTTAGTTTTCCTTTTATAT | N/A | N/A | 117877 | 117896 | 2770 |
| 1394357 | GGCATCTTGTTTTACCACCC | N/A | N/A | 35334 | 35353 | 3624 |
| 1394358 | TCTTGTTTTACCACCCATGC | N/A | N/A | 35330 | 35349 | 3618 |
| 1394359 | TGGCATCTTGTTTTACCACC | N/A | N/A | 35335 | 35354 | 3633 |
| 1394360 | ATCTTGTTTTACCACCCATG | N/A | N/A | 35331 | 35350 | 3626 |
| 1394361 | ATGGCATCTTGTTTTACCAC | N/A | N/A | 35336 | 35355 | 3636 |
| 1394367 | GGGTGGTCACAGCTTTTATT | N/A | N/A | 126805 | 126824 | 3663 |
| 1394368 | GGTGGTCACAGCTTTTATTT | N/A | N/A | 126804 | 126823 | 3635 |
| 1394369 | TTTGCTTTGATTTTTACCAG | N/A | N/A | 60028 | 60047 | 3603 |
| 1394370 | TAGAGGTCATCTTCGGTCAT | N/A | N/A | 118731 | 118750 | 3612 |
| 1394371 | CACAGCTTTTATTTCCATAC | N/A | N/A | 126798 | 126817 | 3619 |
| 1394372 | CTGCTAGAGGTCATCTTCGG | N/A | N/A | 118735 | 118754 | 3629 |
| 1394373 | GCTCACTCATTTCCACCTTC | 5431 | 5450 | 115309 | 115328 | 3648 |
| 1394374 | GAGGTCATCTTCGGTCATGT | N/A | N/A | 118729 | 118748 | 3617 |
| 1394375 | TGCATTACTTTTTACAATGG | N/A | N/A | 62838 | 62857 | 3623 |
| 1394376 | GCTAGAGGTCATCTTCGGTC | N/A | N/A | 118733 | 118752 | 3657 |
| 1394377 | CTCATTTCCACCTTCAGCTG | 5426 | 5445 | 115304 | 115323 | 3637 |
| 1394378 | ACAGCTTTTATTTCCATACA | N/A | N/A | 126797 | 126816 | 3646 |
| 1394379 | TGGTCACAGCTTTTATTTCC | N/A | N/A | 126802 | 126821 | 3608 |
| 1394380 | TGCTAGAGGTCATCTTCGGT | N/A | N/A | 118734 | 118753 | 3659 |
| 1394381 | AGTGCATTACTTTTTACAAT | N/A | N/A | 62840 | 62859 | 3664 |
| 1394382 | CTCACTCATTTCCACCTTCA | 5430 | 5449 | 115308 | 115327 | 3594 |
| 1394383 | AGAGGTCATCTTCGGTCATG | N/A | N/A | 118730 | 118749 | 3654 |
| 1394384 | ATTACTTTTTACAATGGACA | N/A | N/A | 62835 | 62854 | 3622 |
| 1394385 | TACTTTTTACAATGGACAGT | N/A | N/A | 62833 | 62852 | 3620 |
| 1394386 | TCATTTCCACCTTCAGCTGT | 5425 | 5444 | 115303 | 115322 | 3597 |
| 1394387 | GTGGTCACAGCTTTTATTTC | N/A | N/A | 126803 | 126822 | 3649 |
| 1394388 | TTTTTGCTTTGATTTTTACC | N/A | N/A | 60030 | 60049 | 3605 |
| 1394389 | TTTTGCTTTGATTTTTACCA | N/A | N/A | 60029 | 60048 | 3599 |
| 1394390 | TGCTTTGATTTTTACCAGTT | N/A | N/A | 60026 | 60045 | 3615 |
| 1394391 | CTTTGATTTTTACCAGTTAA | N/A | N/A | 60024 | 60043 | 3596 |
| 1394392 | TCACTCATTTCCACCTTCAG | 5429 | 5448 | 115307 | 115326 | 3660 |
| 1394393 | GTGCATTACTTTTTACAATG | N/A | N/A | 62839 | 62858 | 3610 |
| 1394394 | CATTTCCACCTTCAGCTGTT | 5424 | 5443 | 115302 | 115321 | 3661 |
| 1394395 | TTACTTTTTACAATGGACAG | N/A | N/A | 62834 | 62853 | 3627 |
| 1394396 | TTGCTTTGATTTTTACCAGT | N/A | N/A | 60027 | 60046 | 3625 |
| 1394397 | ACTTGGTTAGTTTTCCTTTT | N/A | N/A | 117881 | 117900 | 3666 |
| 1394398 | GTTAGTTTTCCTTTTATATT | N/A | N/A | 117876 | 117895 | 3665 |
| 1394399 | TAGTTTTCCTTTTATATTCA | N/A | N/A | 117874 | 117893 | 3673 |
| 1394400 | TTAGTTTTCCTTTTATATTC | N/A | N/A | 117875 | 117894 | 3669 |
| 1394401 | CTTGGTTAGTTTTCCTTTTA | N/A | N/A | 117880 | 117899 | 3674 |
| 1394402 | TTGGTTAGTTTTCCTTTTAT | N/A | N/A | 117879 | 117898 | 3672 |
| 1456403 | ATATTTTCTCAACTTTAAAC | N/A | N/A | 119644 | 119663 | 3677 |
| 1456404 | CATATTTTCTCAACTTTAAA | N/A | N/A | 119645 | 119664 | 3678 |
| 1456405 | CCATATTTTCTCAACTTTAA | N/A | N/A | 119646 | 119665 | 3679 |
| 1456406 | GCCATATTTTCTCAACTTTA | N/A | N/A | 119647 | 119666 | 940 |
| 1456407 | AGCCATATTTTCTCAACTTT | N/A | N/A | 119648 | 119667 | 3680 |
| 1456408 | AAGCCATATTTTCTCAACTT | N/A | N/A | 119649 | 119668 | 919 |
| 1456409 | CAAGCCATATTTTCTCAACT | N/A | N/A | 119650 | 119669 | 3681 |
| 1456410 | ACAAGCCATATTTTCTCAAC | N/A | N/A | 119651 | 119670 | 3682 |

### Example 7: Tolerability of Modified Oligonucleotides Complementary to Human HTT in Wild-Type Mice, 3 Hour Study

Modified oligonucleotides described above were tested in wild-type female C57/Bl6 mice to assess the tolerability of the oligonucleotides. Wild-type female C57/Bl6 mice each received a single ICV dose of modified oligonucleotide at 700 μg. Each treatment group consisted of 4 mice. A group of 4 mice received PBS as a negative control for each experiment. Each experiment is identified in separate tables below. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a sub-score of 0 if it met the criteria and 1 if it did not (the functional observational battery score or FOB). After all 7 criteria were evaluated, the scores were summed for each mouse and averaged within each treatment group. The results are presented in the tables below.

TABLE 75
Tolerability scores in mice (n = 4) at 700 μg dose

| Compound No. | 3 hr. FOB |
|---|---|
| PBS | 0.00 |
| 1314967 | 1.00 |
| 1314979 | 2.00 |
| 1315343 | 1.00 |
| 1315578 | 1.00 |
| 1316218 | 1.50 |
| 1316639 | 1.00 |
| 1317084 | 0.00 |

TABLE 76
Tolerability scores in mice (n = 4) at 700 μg dose

| Compound No. | 3 hr. FOB |
|---|---|
| PBS | 0.00 |
| 1314723 | 0.00 |
| 1315073 | 0.00 |
| 1315119 | 4.50 |
| 1315147 | 6.75 |
| 1315214 | 0.00 |
| 1315813 | 0.00 |
| 1391301 | 0.00 |
| 1391310 | 1.25 |
| 1391320 | 0.00 |
| 1394347 | 0.50 |
| 1394371 | 0.00 |
| 1394375 | 6.50 |
| 1394378 | 0.00 |
| 1394401 | 4.25 |
| 1394402 | 0.00 |
| 1394418 | 0.00 |
| 1394421 | 1.00 |
| 1394440 | 5.25 |
| 1394444 | 1.25 |
| 1419386 | 0.00 |

TABLE 77
Tolerability scores in mice (n = 4) at 700 μg dose

| Compound No. | 3 hr. FOB |
|---|---|
| PBS | 0.00 |
| 1314833 | 3.00 |
| 1391348 | 1.00 |
| 1394393 | 3.00 |

### Example 8: Tolerability of Modified Oligonucleotides Complementary to Human HTT in Rats, 3-Hour Study

Modified oligonucleotides described above were tested in rats to assess the tolerability of the oligonucleotides. Sprague Dawley rats each received a single intrathecal (IT) dose of 3 mg of modified oligonucleotide listed in the tables below. Each treatment group consisted of 3-4 rats. A group of 3-4 rats received PBS as a negative control. Each experiment is identified in separate tables below. At 3 hours post-injection, movement in 7 different parts of the body were evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). After each of the 7 body parts were evaluated, the sub-scores were summed for each rat and then averaged for each group. For example, if a rat's tail, head, and all other evaluated body parts were moving 3 hours after the 3 mg IT dose, it would get a summed score of 0. If another rat was not moving its tail 3 hours after the 3 mg IT dose but all other evaluated body parts were moving, it would receive a score of 1. Results are presented as the average score for each treatment group.

TABLE 78
Tolerability scores in rats (n = 4) at 3 mg dose

| Compound No. | 3 hr. FOB |
|---|---|
| PBS | 0.25 |
| 388241 | 2.75 |
| 1315343 | 0.50 |
| 1315578 | 0.50 |
| 1316218 | 1.50 |
| 1316639 | 2.25 |
| 1317084 | 0.25 |

TABLE 79
Tolerability scores in rats (n = 4) at 3 mg dose

| Compound No. | 3 hr. FOB |
|---|---|
| PBS | 0.00 |
| 1314979 | 2.50 |
| 1391333 | 2.00 |
| 1391353 | 4.00 |
| 1394371 | 0.00 |
| 1394378 | 0.00 |
| 1394379 | 2.00 |
| 1394392 | 2.00 |

TABLE 80
Tolerability scores in rats (n = 4) at 3 mg dose

| Compound No. | 3 hr. FOB |
|---|---|
| PBS | 0.25 |
| 1314967 | 2.25 |
| 1315073 | 1.00 |
| 1315214 | 3.00‡ |
| 1315813 | 1.50‡ |
| 1316392 | 0.50 |
| 1318641 | 3.00 |
| 1391293 | 0.25 |
| 1391320 | 0.00 |
| 1394375 | 3.25 |
| 1394401 | 2.50 |
| 1394402 | 1.75 |
| 1394439 | 4.00 |
| 1394440 | 3.50 |

‡indicates that fewer than 4 samples were available

TABLE 81
Tolerability scores in rats (n = 3) at 3 mg dose

| Compound No. | 3 hr. FOB |
|---|---|
| PBS | 0.00 |
| 1314833 | 0.33 |
| 1316061 | 0.67 |
| 1316846 | 0.33 |
| 1391301 | 2.33 |
| 1391310 | 0.67 |
| 1391348 | 0.00 |
| 1391351 | 3.00 |
| 1391364 | 2.00 |
| 1394336 | 0.33 |
| 1394346 | 1.33 |
| 1394347 | 3.00‡ |
| 1394348 | 2.67 |
| 1394393 | 2.67 |
| 1394443 | 2.67 |
| 1394444 | 5.00 |
| 1394445 | 3.00 |
| 1394446 | 1.67 |
| 1394450 | 1.33 |

‡indicates that fewer than 3 samples were available

TABLE 82
Tolerability scores in rats (n = 3) at 3 mg dose

| Compound No. | 3 hr. FOB |
|---|---|
| PBS | 0.00 |
| 1314723 | 0.00 |
| 1315077 | 1.33 |
| 1315119 | 1.00 |
| 1315147 | 0.00 |
| 1315321 | 2.00 |
| 1315833 | 3.00‡ |
| 1315921 | 3.33 |
| 1318605 | 4.00 |
| 1391314 | 4.00 |
| 1391356 | 4.00 |
| 1394418 | 0.00 |
| 1394421 | 1.67 |
| 1394441 | 0.00 |
| 1419386 | 0.00 |
| 1419392 | 0.33 |

‡indicates that fewer than 3 samples were available

TABLE 83
Tolerability scores in rats (n = 3) at 3 mg dose

| Compound No. | 3 hr. FOB |
|---|---|
| PBS | 0.00 |
| 1419387 | 0.00 |
| 1419404 | 0.00 |
| 1419415 | 0.33 |
| 1419420 | 2.67 |

TABLE 84
Tolerability scores in rats (n = 3) at 3 mg dose

| Compound No. | 3 hr. FOB |
|---|---|
| PBS | 0.00 |
| 1316689 | 1.00 |
| 1456397 | 0.33 |
| 1456398 | 0.00 |
| 1456399 | 0.33 |
| 1456400 | 1.00 |
| 1456401 | 0.00 |
| 1456402 | 0.00 |
| 1456403 | 0.00 |
| 1456404 | 0.00 |
| 1456405 | 0.00 |
| 1456406 | 0.67 |
| 1456407 | 1.00 |
| 1456408 | 1.00 |
| 1456409 | 0.00 |
| 1456410 | 0.00 |
| 1456411 | 1.33 |
| 1456412 | 2.00 |
| 1456413 | 3.33 |

### Example 9: Activity of Modified Oligonucleotides Complementary to Human HTT in Transgenic Mice

Transgenic mice expressing human HTT (The Jackson Laboratory, Stock No: 008197) were used to test activity of modified oligonucleotides described above.

### Treatment

Transgenic mice were divided into groups of 3-4 mice each. Each mouse received a single ICV bolus of 200-300 μg of modified oligonucleotide as indicated in the tables below. A group of 3-4 mice received PBS as a negative control.

### RNA Analysis

Two weeks post treatment, mice were sacrificed, and RNA was extracted from cortical brain tissue and spinal cord for RTPCR analysis to measure amount of HTT RNA using human primer probe set RTS2617 (described herein above). Results are presented as percent human HTT relative to PBS control, normalized to mouse cyclophilin A. Mouse cyclophilin A RNA was amplified using mouse prime probe set m_cyclo24 (forward sequence TCGCCGCTTGCTGCA, designated herein as SEQ ID NO: 20; reverse sequence ATCGGCCGTGATGTCGA, designated herein as SEQ ID NO: 21; probe sequence CCATGGTCAACCCCACCGTGTTC, designated herein as SEQ ID NO: 22).

Each experiment is identified in separate tables below. N.D. in the tables below refers to instances where the value was Not Defined.

TABLE 85
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314810 | 47 | 52 |
| 1314849 | 54 | 52 |
| 1314935 | 24 | 43 |
| 1314999 | 32 | 44 |
| 1315234 | 64 | 72 |
| 1315340 | 57 | 57 |
| 1315621 | 44 | 46 |
| 1315781 | 61 | 75 |
| 1316535 | 69 | 34 |
| 1316768 | 50 | 36 |
| 1316788 | 60 | 31 |
| 1317090 | 20 | 25 |
| 1317204 | 49‡ | 73‡ |
| 1317227 | 41 | 42 |

‡indicates that fewer than 4 samples were available

TABLE 86
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314540 | 62 | 44 |
| 1314541 | 51 | 22 |
| 1314544 | 73 | 40 |
| 1314606 | 78 | 34 |
| 1314708 | 74 | 40 |
| 1314723 | 35 | 19 |
| 1314891 | 74 | 31 |
| 1314967 | 35 | 5 |
| 1314979 | 46‡ | 8‡ |
| 1315041 | 54 | 25 |
| 1315089 | 49 | 20 |
| 1315143 | 49 | 42 |
| 1315321 | 53 | 18 |
| 1315343 | 32 | 9 |
| 1315405 | 75 | 35 |
| 1315578 | 46 | 10 |
| 1315652 | 67 | 28 |
| 1315698 | 47‡ | 44‡ |
| 1315702 | 48‡ | 21‡ |
| 1315797 | 65 | 29 |
| 1315877 | 42 | 25 |
| 1316169 | 44 | 25 |
| 1316218 | 27 | 9 |
| 1316235 | 64 | 34 |
| 1316392 | 34 | 14 |
| 1316435 | 83 | 33 |
| 1316762 | 81 | 50 |
| 1316766 | 91 | 57 |
| 1316803 | 53 | 22 |
| 1316846 | 39 | 16 |
| 1317084 | 29 | 11 |
| 1317136 | 48 | 29 |
| 1317229 | 32 | 20 |

‡indicates that fewer than 4 samples were available

TABLE 87
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314685 | 60 | 23 |
| 1314789 | 59 | 39 |
| 1314813 | 125 | 74 |
| 1314824 | 87 | 51 |
| 1314845 | 51 | 28 |
| 1314848 | 57 | 25 |
| 1314916 | 75 | 37 |
| 1314943 | 64 | 52 |
| 1315119 | 39 | 19 |
| 1315294 | 92‡ | 68‡ |
| 1315309 | 36 | 31 |
| 1315408 | 72 | 45 |
| 1315584 | 84 | 45 |
| 1315672 | 54 | 21 |
| 1315722 | 60 | 45 |
| 1315974 | 78 | 45 |
| 1316062 | 54 | 43 |
| 1316154 | 59 | 74 |
| 1316185 | 134 | 94 |
| 1316254 | 97 | 42 |
| 1316310 | 66‡ | 20‡ |
| 1316324 | 68 | 54 |
| 1316414 | 57 | 28 |
| 1316604 | 96 | 69 |
| 1316614 | 63 | 34 |
| 1316716 | 74 | 65 |
| 1316775 | 48 | 45 |
| 1316858 | 61 | 88 |
| 1316936 | 39 | 54 |
| 1317017 | 28 | 33 |
| 1317031 | 36 | 27 |
| 1317128 | 63 | 58 |
| 1317205 | 57 | 39 |
| 1318725 | 99 | 86 |

‡indicates that fewer than 4 samples were available

TABLE 88
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 388241 | 44 | 20 |
| 1314635 | 56 | 68 |
| 1314647 | 66 | 68 |
| 1314833 | 29 | 16 |
| 1315027 | 41 | 48 |
| 1315039 | 35 | 47 |
| 1315214 | 26 | 12 |
| 1315501 | 68 | 61 |
| 1315813 | 29 | 14 |
| 1315833 | 51 | 19 |
| 1316545 | 56 | 30 |
| 1316620 | 48 | 35 |
| 1316722 | 45 | 29 |
| 1317164 | 41 | 20 |
| 1318569 | 38 | 28 |
| 1318621 | 54 | 42 |
| 1318675 | 42 | 27 |

TABLE 89
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314696 | 22 | 44 |
| 1314904 | 30 | 38 |
| 1315141 | 59 | 84 |
| 1315878 | 67 | 36 |
| 1315964 | 53 | 65 |
| 1316117 | 43 | 61 |
| 1316143 | 78 | 53 |
| 1316449 | 31 | 35 |
| 1316972 | 34 | 21 |
| 1318566 | 92 | 46 |
| 1318573 | 42 | 31 |
| 1318690 | 50 | 30 |
| 1318691 | 86 | 87 |
| 1318695 | 41 | 44 |
| 1318745 | 53 | 45 |
| 1318747 | 40 | 28 |

TABLE 90
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314526 | 121 | 159 |
| 1314549 | 74 | 56 |
| 1314870 | 107 | 107 |
| 1314905 | 54 | 100 |
| 1314966 | 59 | 79 |
| 1315056 | 46 | 51 |
| 1315074 | 28 | 43 |
| 1315083 | 60 | 35 |
| 1315137 | 68 | 122 |
| 1315341 | 20 | 39 |
| 1315425 | 83 | 165 |
| 1315817 | 49 | 63 |
| 1315867 | 32 | 49 |
| 1316312 | 33 | 46 |
| 1316563 | 42 | 41 |
| 1316621 | 33 | 40 |
| 1316802 | 30 | 23 |

TABLE 91
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 388241 | 56 | 44 |
| 1314603 | 52 | 31 |
| 1314762 | 53 | 42 |
| 1314829 | 20 | 20 |
| 1314918 | 27 | 31 |
| 1314955 | 52 | 54 |
| 1315077 | 20 | 19 |
| 1315347 | 27 | 22 |
| 1315544 | 115 | 77 |
| 1315692 | 78 | 50 |
| 1315747 | 66 | 44 |
| 1315883 | 47 | 50 |
| 1315921 | 27 | 19 |
| 1315950 | 47 | 35 |
| 1316506 | 63 | 50 |
| 1316544 | 32 | 41 |
| 1316551 | 35 | 21 |
| 1316552 | 80 | 58 |
| 1316639 | 14 | 10 |
| 1316725 | 34 | 25 |
| 1317175 | 63 | N.D. |
| 1318657 | 88 | N.D. |
| 1318685 | 52 | N.D. |

TABLE 92
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314511 | 70 | 65 |
| 1315178 | 53 | 27 |
| 1315556 | 57 | 41 |
| 1316023 | 59 | 32 |
| 1316926 | 50 | 38 |
| 1317142 | 51 | 21 |
| 1318592 | 86 | 56 |
| 1318594 | 104 | 61 |
| 1318605 | 41 | 19 |
| 1318620 | 57 | 27 |
| 1318629 | 62 | 52 |
| 1318632 | 44 | 30 |
| 1318641 | 33 | 14 |
| 1318701 | 67 | 25 |
| 1318741 | 93 | 49 |

TABLE 93
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1318708 | 24 | 66 |
| 1314771 | 49 | 49 |
| 1314828 | 67 | 47 |
| 1314920 | 34 | 27 |
| 1315482 | 45 | 38 |
| 1316232 | 48 | 49 |
| 1316400 | 26 | 28 |
| 1317236 | 34 | 35 |
| 1318652 | 64 | 47 |
| 1314487 | 50 | 61 |
| 1314650 | 51 | 37 |
| 1314678 | 47 | 24 |
| 1314815 | 53 | 49 |
| 1314996 | 48 | 48 |
| 1315014 | 29 | 32 |
| 1315123 | 35 | 27 |
| 1315147 | 28 | 18 |

TABLE 94
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 388241 | 73 | 56 |
| 1315373 | 81 | 105 |
| 1315537 | 45 | 53 |
| 1315723 | 41 | 87 |
| 1315749 | 25 | 49 |
| 1315800 | 78 | 65 |
| 1315808 | 48 | 75 |
| 1315865 | 42 | 69 |
| 1315911 | 46 | 64 |
| 1316051 | 56 | 71 |
| 1316096 | 51 | 92 |
| 1316679 | 43 | 74 |
| 1316745 | 44 | 44 |
| 1317163 | 40‡ | 102 |
| 1318586 | 39 | 42 |
| 1318623 | 86 | 63 |
| 1318715 | 90 | 77 |
| 1318742 | 83 | 67 |

‡indicates that fewer than 4 samples were available

TABLE 95
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314592 | 29 | 40 |
| 1316811 | 40 | 61 |
| 1391295 | 45 | 62 |
| 1391298 | 51 | 37 |
| 1391303 | 46 | 50 |
| 1391305 | 47 | 46 |
| 1391309 | 70 | 92 |
| 1391315 | 44 | 43 |
| 1391319 | 37 | 44 |
| 1391325 | 28 | 33 |
| 1391326 | 54 | 59 |
| 1391327 | 37 | 36 |
| 1391335 | 32 | 29 |
| 1391338 | 45 | 55 |
| 1391341 | 61 | 119 |
| 1391355 | 74 | 70 |
| 1394344 | 21 | 21 |
| 1394347 | 17 | 17 |
| 1394357 | 29 | 23 |
| 1394358 | 50 | 53 |
| 1394359 | 18 | 30 |
| 1394360 | 40 | 38 |
| 1394361 | 29 | 38 |
| 1394369 | 32 | 43 |
| 1394388 | 40 | 31 |

TABLE 96
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314907 | 31 | 27 |
| 1391293 | 36 | 14 |
| 1391296 | 51 | 31 |
| 1391299 | 42 | 45 |
| 1391304 | 47 | 49 |
| 1391310 | 26 | 17 |
| 1391316 | 56 | 35 |
| 1391321 | 52 | 51 |
| 1391323 | 64 | 43 |
| 1391324 | 39 | 37 |
| 1391336 | 52 | 54 |
| 1391339 | 48 | 23 |
| 1391351 | 18 | 17 |
| 1391357 | 72 | 50 |
| 1391364 | 32 | 15 |
| 1391365 | 57 | 24 |
| 1391366 | 95 | 31 |
| 1391368 | 50 | 27 |
| 1394346 | 16 | 10 |
| 1394375 | 35 | 11 |
| 1394381 | 39 | 133 |
| 1394384 | 74 | 47 |
| 1394385 | 54 | 96 |

TABLE 97
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1391292 | 25 | 32 |
| 1391301 | 38 | 16 |
| 1391306 | 87 | 70 |
| 1391312 | 54 | 39 |
| 1391314 | 89 | 19 |
| 1391318 | 65 | 73 |
| 1391322 | 66 | 69 |
| 1391330 | 75 | 90 |
| 1391332 | 27 | 23 |
| 1391333 | 15 | 3 |
| 1391334 | 84 | 97 |
| 1391342 | 58 | 52 |
| 1391353 | 14 | 5 |
| 1391356 | 20 | 19 |
| 1391358 | 65 | 71 |
| 1391361 | 57 | 54 |
| 1391362 | 46 | 48 |
| 1391363 | 81 | 96 |
| 1394343 | 50 | 46 |
| 1394373 | 23 | 23 |
| 1394377 | 56 | 45 |
| 1394382 | 45 | 20 |
| 1394386 | 66 | 42 |

TABLE 98
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1315073 | 25 | 11 |
| 1316061 | 27 | 18 |
| 1391291 | 81 | 112 |
| 1391294 | 86 | 87 |
| 1391328 | 56 | 36 |
| 1391345 | 82 | 83 |
| 1391349 | 60 | 77 |
| 1394348 | 31 | 23 |
| 1394390 | 36 | 24 |
| 1394391 | 39 | 24 |
| 1394392 | 27 | 10 |
| 1394393 | 31 | 18 |
| 1394394 | 58 | 40 |
| 1394395 | 132 | 55 |
| 1394396 | 51 | 24 |
| 1394440 | 39 | 13 |
| 1394441 | 55 | 19 |
| 1394442 | 30 | 24 |
| 1394443 | 42 | 25 |
| 1394448 | 83 | 37 |
| 1394449 | 32 | 24 |
| 1394451 | 33 | 26 |
| 1394452 | 29 | 22 |

TABLE 99
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1315670 | 40 | 39 |
| 1391308 | 28 | 31 |
| 1391320 | 23 | 14 |
| 1391337 | 74 | 67 |
| 1391348 | 30 | 16 |
| 1391352 | 70 | 48 |
| 1391367 | 73 | 32 |
| 1394345 | 17 | 25 |
| 1394367 | 78 | 42 |
| 1394368 | 54 | 26 |
| 1394370 | 56 | 28 |
| 1394371 | 26 | 10 |
| 1394372 | 81 | 34 |
| 1394374 | 92 | 36 |
| 1394376 | 54 | 31 |
| 1394378 | 28 | 10 |
| 1394379 | 27 | 6 |
| 1394380 | 62 | 23 |
| 1394383 | 66 | 30 |
| 1394387 | 61 | 23 |
| 1394444 | 61 | 15 |
| 1394445 | 74 | 29 |
| 1394446 | 74 | 26 |

TABLE 100
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1394315 | 51 | 29 |
| 1394316 | 47 | 21 |
| 1394317 | 56 | 45 |
| 1394318 | 41 | 23 |
| 1394320 | 86 | 78 |
| 1394321 | 68 | 74 |
| 1394323 | 58 | 96 |
| 1394325 | 76 | 58 |
| 1394326 | 49 | 54 |
| 1394331 | 50 | 44 |
| 1394335 | 86 | 41 |
| 1394336 | 48 | 14 |
| 1394349 | 25 | 37 |
| 1394397 | 36 | 27 |
| 1394398 | 56 | 77 |
| 1394399 | 46 | 55 |
| 1394400 | 43 | 50 |
| 1394401 | 33 | 14 |
| 1394402 | 22 | 8 |
| 1394439 | 20 | 10 |
| 1394447 | 34 | 41 |
| 1394450 | 38 | 25 |

TABLE 101
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1314899 | 66 | 43 |
| 1315156 | 76 | 38 |
| 1315412 | 78 | 47 |
| 1315754 | 49 | 31 |
| 1315794 | 75 | 54 |
| 1316106 | 67 | 44 |
| 1316193 | 68 | 29 |
| 1316421 | 79 | 57 |
| 1316500 | 68 | 59 |
| 1316984 | 74 | 57 |

TABLE 102
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1394429 | 82 | 80 |
| 1394430 | 67 | 78 |
| 1394431 | 67 | 59 |
| 1394432 | 90 | 77 |
| 1394433 | 78 | 77 |
| 1394434 | 82 | 80 |
| 1394437 | 34 | 34 |
| 1316648 | 44 | 21 |

TABLE 103
Reduction of human HTT RNA in transgenic mice (n = 4)
treated with 200 μg of modified oligonucleotide

HTT RNA (% control)

| Compound ID | Spinal Cord | Cortex |
|---|---|---|
| PBS | 100 | 100 |
| 1316776 | 47 | 27 |
| 1391297 | 137 | 82 |
| 1391302 | 112 | 64 |
| 1391307 | 64 | 31 |
| 1391311 | 86 | 45 |
| 1391313 | 123 | 84 |
| 1391329 | 126 | 64 |
| 1391340 | 85 | 24 |
| 1391343 | 65 | 48 |
| 1391344 | 123 | 51 |
| 1391346 | 145 | 52 |
| 1391347 | 81‡ | 72 |
| 1391359 | 83 | 40 |
| 1391360 | 124 | 58 |
| 1394389 | 58 | 23 |
| 1394418 | 49 | 19 |
| 1394419 | 86 | 37 |
| 1394420 | 96 | 50 |
| 1394421 | 43 | 17 |
| 1394422 | 180 | 64 |
| 1394423 | 40 | 25 |
| 1394424 | 59 | 37 |
| 1394425 | 32 | 22 |
| 1394427 | 25 | 37 |
| 1394428 | 72 | 80 |

‡indicates that fewer than 4 samples were available

TABLE 104
Reduction of human HTT RNA in transgenic mice (n = 3)
treated with 200 μg of modified oligonucleotide

HTT RNA (% control)

| Compound ID | Spinal Cord | Cortex |
|---|---|---|
| PBS | 100 | 100 |
| 388241 | 86§ | 36§ |
| 1419360 | 39‡ | 24‡ |
| 1419361 | 90 | 61 |
| 1419362 | 108 | 39 |
| 1419365 | 136‡ | 58‡ |
| 1419370 | 67 | 57 |
| 1419374 | 164 | 84 |
| 1419375 | 115 | 41 |
| 1419376 | 82 | 23 |
| 1419379 | 100 | 52 |
| 1419380 | 102 | 39 |
| 1419383 | 128 | 34 |
| 1419384 | 133 | 56 |
| 1419386 | 29 | 12 |
| 1419389 | 82 | 39 |
| 1419392 | 84 | 10 |
| 1419394 | 133 | 29 |
| 1419406 | 73 | 56 |
| 1419407 | 157 | 136 |
| 1419409 | 182 | 132 |
| 1419414 | 135 | 47 |
| 1419419 | 134 | 71 |

‡indicates that fewer than 3 samples were available
§indicates that 4 samples were available

TABLE 105
Reduction of human HTT RNA in transgenic mice (n = 3)
treated with 200 μg of modified oligonucleotide

HTT RNA (% control)

| Compound ID | Spinal Cord | Cortex |
|---|---|---|
| PBS | 100 | 100 |
| 1419387 | 32 | 18 |
| 1419390 | 75 | 49 |
| 1419391 | 90 | 81 |
| 1419395 | 69 | 90 |
| 1419396 | 109 | 97 |
| 1419397 | 47 | 30 |
| 1419399 | 72 | 22 |
| 1419400 | 70 | 68 |
| 1419402 | 57 | 57 |
| 1419403 | 82 | 75 |
| 1419405 | 59 | 52 |
| 1419416 | 56 | 25 |
| 1419417 | 56 | 56 |
| 1419421 | 103 | 96 |
| 1419423 | 97 | 89 |
| 1419425 | 83 | 94 |

TABLE 106
Reduction of human HTT RNA in transgenic mice (n = 3)
treated with 200 μg of modified oligonucleotide

HTT RNA (% control)

| Compound ID | Spinal Cord | Cortex |
|---|---|---|
| PBS | 100 | 100 |
| 1419388 | 50 | 44 |
| 1419393 | 45 | 25 |
| 1419398 | 93 | 67 |
| 1419401 | 62 | 75 |
| 1419404 | 15 | 12 |
| 1419408 | 120 | 54 |
| 1419410 | 127 | 47 |
| 1419412 | 75 | 47 |
| 1419413 | 132 | 51 |
| 1419415 | 28 | 17 |
| 1419418 | 52 | 28 |
| 1419420 | 12 | 6 |
| 1419422 | 124 | 73 |
| 1419424 | 41 | 50 |

TABLE 107
Reduction of human HTT RNA in transgenic mice (n = 4)
treated with 300 μg of modified oligonucleotide

HTT RNA (% control)

| Compound ID | Spinal Cord | Cortex |
|---|---|---|
| PBS | 100 | 100 |
| 1316689 | 45 | 27 |
| 1456397 | 107 | 90 |
| 1456398 | 101 | 107 |
| 1456399 | 61‡ | 18‡ |
| 1456400 | 42 | 12 |
| 1456401 | 49 | 23 |
| 1456402 | 62 | 31 |
| 1456403 | 79 | 60 |
| 1456404 | 58‡ | 51‡ |
| 1456405 | 47 | 14 |
| 1456406 | 20 | 6 |
| 1456407 | 29 | 13 |
| 1456408 | 32 | 18 |
| 1456409 | 42‡ | 17‡ |
| 1456410 | 42‡ | 28‡ |
| 1456411 | 58 | 25 |
| 1456412 | 40 | 22 |
| 1456413 | 67 | 47 |

‡indicates that fewer than 4 samples were available

### Example 10: Potency of Modified Oligonucleotides Complementary to Human HTT RNA in Transgenic Mice

Modified oligonucleotides described above were tested in human HTT transgenic mice (described herein above).

### Treatment

Human HTT transgenic mice were divided into groups of 4 mice each. Each mouse received a single ICV bolus of modified oligonucleotide at the doses indicated in tables below. A group of 3-4 mice received PBS as a negative control.

### RNA Analysis

Two weeks post treatment, mice were sacrificed, and RNA was extracted from the spinal cord and cortex for quantitative real-time RTPCR analysis of RNA expression of HTT using primer probe set RTS2617 (described herein above). Results are presented as percent human HTT RNA relative to PBS control, adjusted to mouse cyclophilin A (described herein above).

Dose response and tissue concentration response data were analyzed using Microsoft Excel (v14.4) and GraphPad Prism software (v 8.2.0, San Diego, CA). ED₅₀ values were calculated from log transformed dose and individual animal HTT RNA levels using the built in GraphPad formula “log(agonist) vs. response—Find ECanything”, with the following constraints: bottom >0, top=100, and F=50 for ED50.

Each experiment is identified in separate tables below. As shown in the tables below, treatment with modified oligonucleotides resulted in dose-responsive reduction of HTT RNA in comparison to the PBS control. N.D. in the tables below refers to instances where the value was Not Defined.

TABLE 108
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 388241 | 10 | 85 | 185.4 | 62 | 31.1 |
| | 30 | 85 | | 56 | |
| | 100 | 55 | | 30 | |
| | 300 | 44 | | 26 | |
| 444652 | 10 | 89 | 277.5 | 133 | 186.1 |
| | 30 | 95 | | 71 | |
| | 100 | 56 | | 54 | |
| | 300 | 53 | | 48 | |

TABLE 109
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1314967 | 3 | 82 | 45.5 | 118 | 108.5 |
| | 10 | 61 | | 95 | |
| | 30 | 61 | | 87 | |
| | 100 | 36 | | 52 | |
| | 300 | 31 | | 20 | |
| 1314979 | 3 | 147 | 73.6 | 82 | 119.2 |
| | 10 | 133 | | 73 | |
| | 30 | 98 | | 78 | |
| | 100 | 97 | | 51 | |
| | 300 | 103 | | 35 | |
| 1315343 | 3 | 163 | 124.7 | 95 | 49.1 |
| | 10 | 102 | | 67 | |
| | 30 | 80 | | 65 | |
| | 100 | 49 | | 37 | |
| | 300 | 35 | | 19 | |
| 1315578 | 3 | 113 | 156.7 | 110 | 70.6 |
| | 10 | 58 | | 103 | |
| | 30 | 96 | | 69 | |
| | 100 | 69 | | 28 | |
| | 300 | 26 | | 34 | |
| 1316218 | 3 | 68 | 106.0 | 130 | 34.3 |
| | 10 | 111‡ | | 99 | |
| | 30 | 71 | | 42 | |
| | 100 | 33 | | 25 | |
| | 300 | 26‡ | | 22‡ | |
| 1394379 | 3 | 111 | 73.6 | 121 | 37.9 |
| | 10 | 84 | | 97 | |
| | 30 | 56 | | 55 | |
| | 100 | 54 | | 20 | |
| | 300 | 18 | | 5 | |

‡indicates that fewer than 4 samples were available

TABLE 110
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 388241 | 3 | 60 | 16.9 | 75 | 44.4 |
| | 10 | 61 | | 87 | |
| | 30 | 50 | | 51 | |
| | 100 | 24‡ | | 34‡ | |
| | 300 | 28 | | 24 | |
| 444652 | 3 | 57 | 41.1 | 75 | 47.5 |
| | 10 | 82 | | 77 | |
| | 30 | 47 | | 41 | |
| | 100 | 40 | | 52 | |
| | 300 | 34 | | 27 | |
| 627246 | 3 | 61 | 13.1 | 77 | 74.3 |
| | 10 | 42 | | 69 | |
| | 30 | 50 | | 57 | |
| | 100 | 46 | | 43 | |
| | 300 | 30 | | 41 | |

‡indicates that fewer than 4 samples were available

TABLE 111
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 627246 | 3 | 75 | N.D. | 84 | 166.9 |
| | 10 | 68 | | 89 | |
| | 30 | 81‡ | | 81‡ | |
| | 100 | 61 | | 52 | |
| | 300 | 42 | | 46 | |
| | 700 | 35 | | 26 | |
| 1314723 | 3 | 90 | 44.6 | 69 | 12.2 |
| | 10 | 80 | | 56 | |
| | 30 | 55 | | 38 | |
| | 100 | 27 | | 16 | |
| | 300 | 28 | | 6 | |
| | 700 | 13 | | 4 | |
| 1315073 | 3 | 108 | 177.1 | 87 | 36.1 |
| | 10 | 186 | | 67 | |
| | 30 | 123 | | 62 | |
| | 100 | 45 | | 31 | |
| | 300 | 34 | | 9 | |
| | 700 | 31 | | 8 | |

‡indicates that fewer than 4 samples were available

TABLE 112
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 388241 | 3 | 141 | 438.0 | 109 | 107.8 |
| | 10 | 87 | | 83 | |
| | 30 | 84 | | 82 | |
| | 100 | 70 | | 47 | |
| | 300 | 58 | | 28 | |
| | 700 | 45‡ | | 18‡ | |
| 1316218 | 3 | 102 | 157.9 | 63 | 30.7 |
| | 10 | 118 | | 83 | |
| | 30 | 52 | | 52 | |
| | 100 | 44 | | 30 | |
| | 300 | 48 | | 17 | |
| | 700 | 34 | | 13 | |
| 1391320 | 3 | 231 | 352.6 | 82 | 40.2 |
| | 10 | 177 | | 82 | |
| | 30 | 136 | | 55 | |
| | 100 | 99 | | 31 | |
| | 300 | 43 | | 17 | |
| | 700 | 36 | | 6 | |
| 1394347 | 3 | 69 | 1475.0 | 79 | 19.1 |
| | 10 | 97 | | 64 | |
| | 30 | 62 | | 39 | |
| | 100 | 42 | | 20 | |
| | 300 | 60 | | 16 | |
| | 700 | 65‡ | | 15‡ | |

‡indicates that fewer than 4 samples were available

TABLE 113
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1315813 | 3 | 83 | 63.3 | 85 | 38.4 |
| | 10 | 71 | | 76 | |
| | 30 | 67 | | 50 | |
| | 100 | 44 | | 28‡ | |
| | 300 | 28 | | 28 | |
| | 700 | 18 | | 9 | |
| 1391301 | 3 | 70 | 14.0 | 40 | 2.2 |
| | 10 | 45 | | 42 | |
| | 30 | 43 | | 37 | |
| | 100 | 33 | | 12 | |
| | 300 | 28 | | 12 | |
| | 700 | 16‡ | | 8‡ | |
| 1394371 | 3 | 63 | 24.5 | 50 | 6.7 |
| | 10 | 74 | | 62 | |
| | 30 | 49 | | 27 | |
| | 100 | 26 | | 16 | |
| | 300 | 26 | | 13 | |
| | 700 | 15 | | 6 | |
| 1394375 | 3 | 68 | 34.2 | 107 | 40.5 |
| | 10 | 65 | | 73 | |
| | 30 | 44 | | 59 | |
| | 100 | 34 | | 18 | |
| | 300 | 44 | | 26 | |
| | 700 | 29 | | 12 | |
| 1394378 | 3 | 73 | 12.8 | 96 | 26.7 |
| | 10 | 42 | | 66 | |
| | 30 | 38 | | 37 | |
| | 100 | 35 | | 36 | |
| | 300 | 25 | | 13 | |
| | 700 | 14 | | 6 | |
| 1394401 | 3 | 91 | 24.5 | 134 | 64.8 |
| | 10 | 45 | | 105 | |
| | 30 | 45 | | 47 | |
| | 100 | 28 | | 42 | |
| | 300 | 36 | | 23 | |
| | 700 | 17 | | 11 | |

‡indicates that fewer than 4 samples were available

TABLE 114
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1315214 | 3 | 136 | 111.4 | 40 | 2.4 |
| | 10 | 107 | | 35 | |
| | 30 | 75 | | 18 | |
| | 100 | 49 | | 20 | |
| | 300 | 30 | | 9 | |
| | 700 | N.D. | | N.D. | |
| 1394401 | 3 | 103 | 182.9 | 85 | 40.8 |
| | 10 | 94 | | 65 | |
| | 30 | 86 | | 56 | |
| | 100 | 57 | | 48 | |
| | 300 | 39 | | 11 | |
| | 700 | 28 | | 12 | |
| 1394402 | 3 | 72 | 55.4 | 61 | 8.4 |
| | 10 | 85 | | 39 | |
| | 30 | 60 | | 51 | |
| | 100 | 31 | | 23 | |
| | 300 | 31 | | 9 | |
| | 700 | 25 | | 7 | |
| 1394440 | 3 | 86 | 43.8 | 74 | 18.5 |
| | 10 | 70 | | 62 | |
| | 30 | 58 | | 44 | |
| | 100 | 36 | | 23 | |
| | 300 | 23 | | 16 | |
| | 700 | 15 | | 9 | |
| 1394444 | 3 | 145 | 244.3 | 37 | N.D. |
| | 10 | 145 | | 43 | |
| | 30 | 78 | | 38 | |
| | 100 | 55 | | 27 | |
| | 300 | 42 | | 15 | |
| | 700 | 41 | | 9 | |

TABLE 115
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1315578 | 3 | 114 | 111.4 | 116 | 70.0 |
| | 10 | 85 | | 80 | |
| | 30 | 76‡ | | 72‡ | |
| | 100 | 56 | | 40 | |
| | 300 | 49 | | 19 | |
| | 700 | 39 | | 10 | |

‡indicates that fewer than 4 samples were available

TABLE 116
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1314833 | 3 | 55 | 2.3 | 56 | 4.1 |
| | 10 | 26 | | 39 | |
| | 30 | 33‡ | | 26‡ | |
| | 100 | 17 | | 18 | |
| | 300 | 18 | | 17 | |
| | 700 | 16 | | 9 | |
| 1315921 | 3 | 77 | 17.2 | 116 | 46.8 |
| | 10 | 41 | | 57 | |
| | 30 | 49 | | 67 | |
| | 100 | 35 | | 22 | |
| | 300 | 24 | | 33 | |
| | 700 | 13‡ | | 9‡ | |
| 1394346 | 3 | 57 | 2.5 | 51 | 4.7 |
| | 10 | 21 | | 35 | |
| | 30 | 24 | | 56 | |
| | 100 | 20 | | 15 | |
| | 300 | 12 | | 11 | |
| | 700 | 11 | | 8 | |
| 1394393 | 3 | 84 | 21.4 | 125 | 55.0 |
| | 10 | 50 | | 65 | |
| | 30 | 44 | | 52 | |
| | 100 | 30 | | 44 | |
| | 300 | 25 | | 25 | |
| | 700 | 18‡ | | 11‡ | |

‡indicates that fewer than 4 samples were available

TABLE 117
Reduction of human HTT RNA in transgenic mice

| | | Spinal Cord | | Cortex | |
|---|---|---|---|---|---|
| | Dose | HTT RNA | ED₅₀ | HTT RNA | ED₅₀ |
| Compound ID | (μg) | (% control) | (μg) | (% control) | (μg) |
| PBS | N/A | 100 | N/A | 100 | N/A |
| 1315077 | 3 | 120 | 116.5 | 293 | 136.6 |
| | 10 | 67 | | 100 | |
| | 30 | 60 | | 104 | |
| | 100 | 42 | | 53 | |
| | 300 | 36 | | 21 | |
| | 700 | 43 | | 26 | |
| 1315119 | 3 | 43 | N.D. | 149 | 358.3 |
| | 10 | 31 | | 177 | |
| | 30 | 29 | | 173 | |
| | 100 | 22 | | 131 | |
| | 300 | 33 | | 58 | |
| | 700 | 19 | | 21‡ | |
| 1391348 | 3 | 95 | 359.2 | 264 | 507.1 |
| | 10 | 88 | | 122 | |
| | 30 | 88 | | 119 | |
| | 100 | 61‡ | | 111‡ | |
| | 300 | 53 | | 67 | |
| | 700 | 42 | | 38 | |
| 1394418 | 3 | 127 | 143.0 | 62 | 42.7 |
| | 10 | 76 | | 65 | |
| | 30 | 82 | | 57 | |
| | 100 | 73 | | 29 | |
| | 300 | 17 | | 55 | |
| | 700 | 16‡ | | 22‡ | |

‡indicates that fewer than 4 samples were available

### Example 11: Activity of Modified Oligonucleotides Complementary to Human HTT RNA in Transgenic Mice, Multiple Doses

Modified oligonucleotides described above were tested in human HTT transgenic mice (described herein above).

Human HTT transgenic mice were divided into groups of 4 mice each. Each mouse received a single ICV bolus of modified oligonucleotide at the various doses indicated in the table below. A group of 4 mice received PBS as a negative control.

Two weeks post treatment, mice were sacrificed, and RNA was extracted from cortical brain tissue and spinal cord for quantitative real-time RT-PCR analysis to measure the amount of HTT RNA using human primer probe set RTS2617 (described herein above) which detects total HTT RNA levels. HTT RNA levels were normalized to mouse cyclophilin A. Mouse cyclophilin A was amplified using primer probe set m_cyclo24 (described herein above). Results are presented as percent human HTT RNA relative to the amount of HTT RNA in PBS treated animals, (% control). ED50s were calculated in Prism using nonlinear fit with variable slope (four parameter), top constrained to 100% (or 1), bottom constrained to 0. Y=Bottom+(Top−Bottom)/(1+(IC50/X)\hat{ }HillSlope).

TABLE 118
Reduction of human HTT RNA in transgenic mice

| Compound | Dose | HTT (% control) | | | |
|---|---|---|---|---|---|
| No. | (ug) | Cortex | ED50 (ug) | Spinal Cord | ED50 (ug) |
| PBS | — | 100 | — | 100 | — |
| 443139 | 3 | 106 | 96 | 117 | 288 |
| | 10 | 87‡ | | 98‡ | |
| | 30 | 65 | | 84 | |
| | 100 | 50 | | 65 | |
| | 300 | 31 | | 54 | |
| 1394371 | 3 | 100 | 43 | 76 | 16 |
| | 10 | 74 | | 59 | |
| | 30 | 67 | | 36 | |
| | 100 | 25 | | 25 | |
| | 300 | 9 | | 16 | |

‡Indicates less than 4 samples were available

1.-56. (canceled)

57. An oligomeric compound comprising a modified oligonucleotide according to the following chemical notation:
(SEQ ID NO: 3619)
^{m}CₑₛAₑₒ^{m}CₑₒAₑₒGₑₒ^{m}CₑₒT_{ds}T_{ds}T_{ds}T_{ds}A_{ds}T_{ds}T_{ds}T_{ds}^{m}C_{ds}^{m}C_{ds}AₑₒTₑₛAₑₛ^{m}Cₑ,
wherein
A=an adenine,
^{m}C=a 5-methylcytosine,
G=a guanine,
T=a thymine,
e=a 2′-O-methoxyethylribose modified sugar,
d=a 2′-deoxyribose sugar moiety,
s=a phosphorothioate internucleoside linkage, and
o=a phosphodiester internucleoside linkage.

58. (canceled)

59. (canceled)

60. The oligomeric compound of claim 57, wherein the modified oligonucleotide is a pharmaceutically acceptable salt.

61. The oligomeric compound of claim 60, which is a pharmaceutically acceptable salt comprising one or more cations selected from sodium, potassium, calcium, and magnesium.

62. The oligomeric compound of claim 57, wherein the oligomeric compound is a singled-stranded oligomeric compound.

63. A modified oligonucleotide according to the following chemical structure: 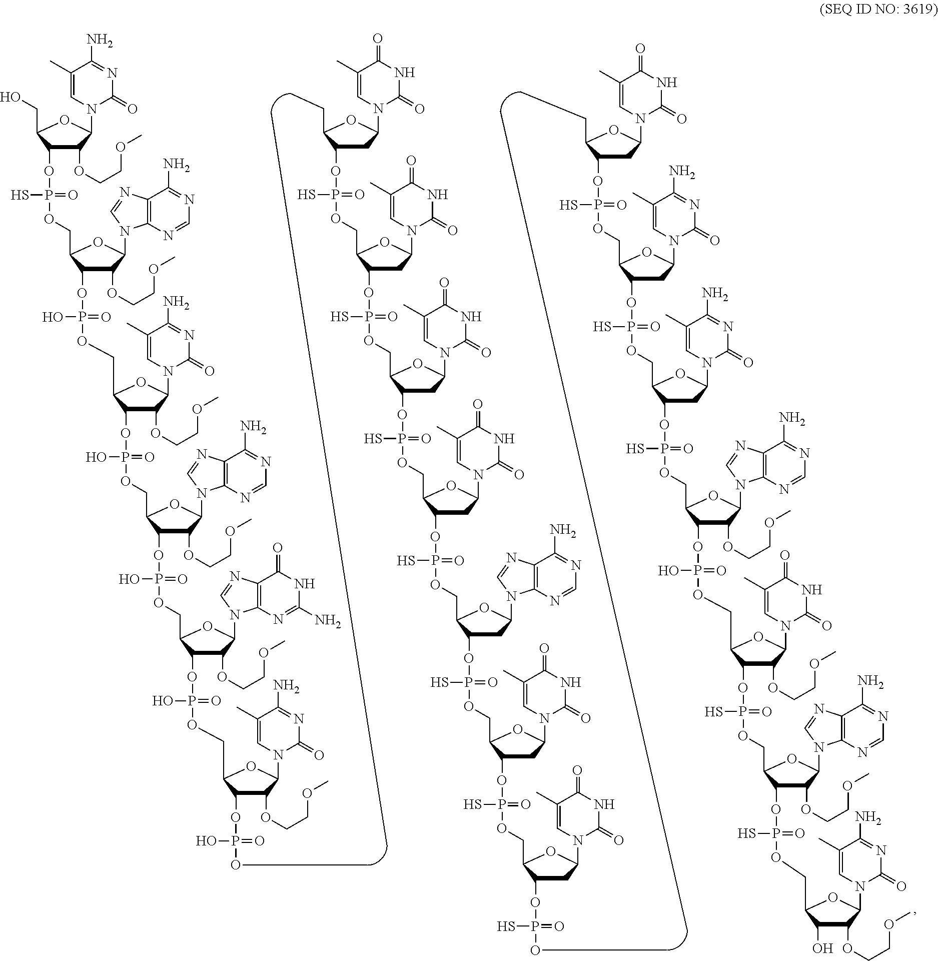
or a pharmaceutically acceptable salt thereof.

64. The modified oligonucleotide of claim 63, which is a pharmaceutically acceptable salt comprising one or more cations selected from sodium, potassium, calcium, and magnesium.

65. A modified oligonucleotide according to the following chemical structure:

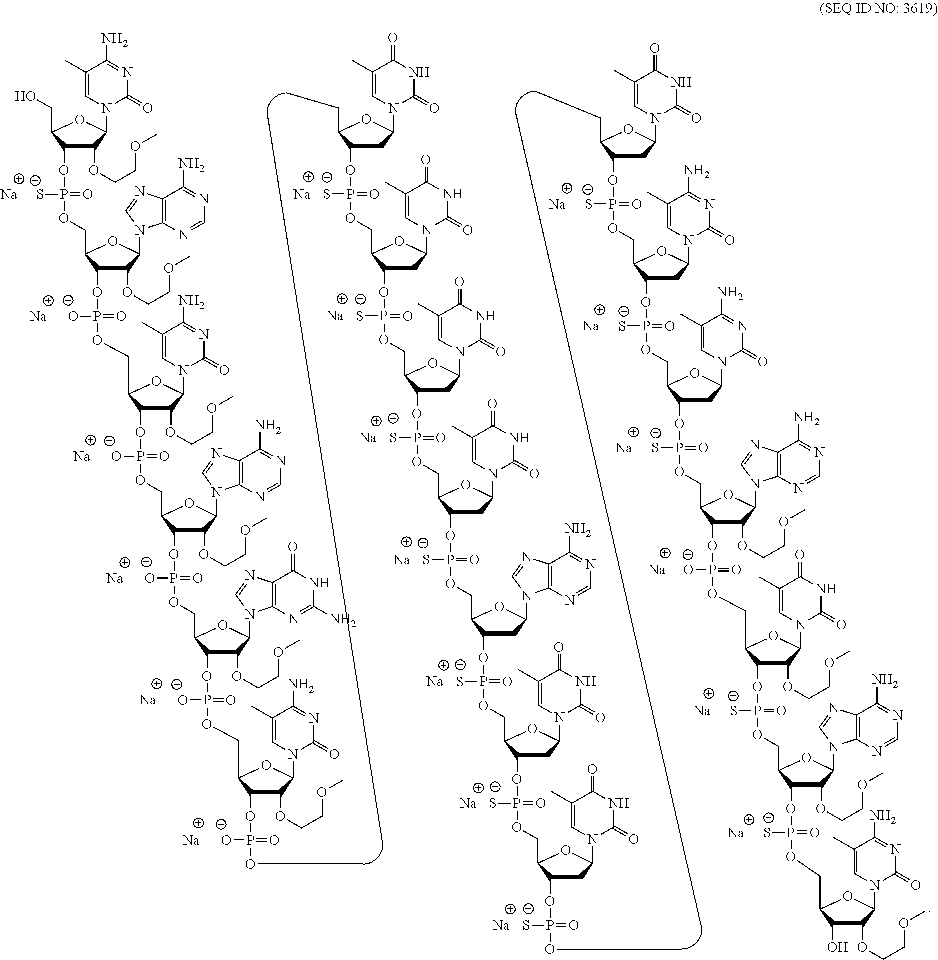

66.-79. (canceled)

80. A population of oligomeric compounds of claim 57, wherein all of the phosphorothioate internucleoside linkages of the modified oligonucleotide are stereorandom.

81.-106. (canceled)

107. A pharmaceutical composition comprising the oligomeric compound of claim 57 and a pharmaceutically acceptable carrier or diluent.

108. The pharmaceutical composition of claim 107, wherein the pharmaceutically acceptable diluent comprises phosphate buffered saline (PBS) or artificial cerebrospinal fluid (aCSF).

109. The pharmaceutical composition of claim 108, wherein the pharmaceutical composition consists essentially of the oligomeric compound and PBS.

110. The pharmaceutical composition of claim 108, wherein the pharmaceutical composition consists essentially of the oligomeric compound and aCSF.

111.-128. (canceled)

129. A pharmaceutical composition comprising the modified oligonucleotide of claim 63; and a pharmaceutically acceptable carrier or diluent.

130. The pharmaceutical composition of claim 129, wherein the pharmaceutically acceptable diluent comprises phosphate buffered saline (PBS) or artificial cerebrospinal fluid (aCSF).

131. The pharmaceutical composition of claim 130, wherein the pharmaceutical composition consists essentially of the modified oligonucleotide and PBS.

132. The pharmaceutical composition of claim 130, wherein the pharmaceutical composition consists essentially of the modified oligonucleotide and aCSF.

133. A pharmaceutical composition comprising the modified oligonucleotide of claim 64; and a pharmaceutically acceptable carrier or diluent.

134. The pharmaceutical composition of claim 133, wherein the pharmaceutically acceptable diluent comprises phosphate buffered saline (PBS) or artificial cerebrospinal fluid (aCSF).

135. The pharmaceutical composition of claim 134, wherein the pharmaceutical composition consists essentially of the modified oligonucleotide and PBS.

136. The pharmaceutical composition of claim 134, wherein the pharmaceutical composition consists essentially of the modified oligonucleotide and aCSF.

137. A pharmaceutical composition comprising the modified oligonucleotide of claim 65; and a pharmaceutically acceptable carrier or diluent.

138. The pharmaceutical composition of claim 137, wherein the pharmaceutically acceptable diluent comprises phosphate buffered saline (PBS) or artificial cerebrospinal fluid (aCSF).

139. The pharmaceutical composition of claim 138, wherein the pharmaceutical composition consists essentially of the modified oligonucleotide and PBS.

140. The pharmaceutical composition of claim 138, wherein the pharmaceutical composition consists essentially of the modified oligonucleotide and aCSF.

141. A population of modified oligonucleotides of claim 63, wherein all of the phosphorothioate internucleoside linkages of the modified oligonucleotide are stereorandom.

142. A population of modified oligonucleotides of claim 64, wherein all of the phosphorothioate internucleoside linkages of the modified oligonucleotide are stereorandom.

143. A population of modified oligonucleotides of claim 65, wherein all of the phosphorothioate internucleoside linkages of the modified oligonucleotide are stereorandom.

144. A pharmaceutical composition comprising the population of oligomeric compounds of claim 80; and a pharmaceutically acceptable carrier or diluent.

145. The pharmaceutical composition of claim 144, wherein the pharmaceutically acceptable diluent comprises phosphate buffered saline (PBS) or artificial cerebrospinal fluid (aCSF).

146. A pharmaceutical composition comprising the population of modified oligonucleotides of claim 141; and a pharmaceutically acceptable carrier or diluent.

147. The pharmaceutical composition of claim 146, wherein the pharmaceutically acceptable diluent comprises phosphate buffered saline (PBS) or artificial cerebrospinal fluid (aCSF).

148. A pharmaceutical composition comprising the population of modified oligonucleotides of claim 142; and a pharmaceutically acceptable carrier or diluent.

149. The pharmaceutical composition of claim 148, wherein the pharmaceutically acceptable diluent comprises phosphate buffered saline (PBS) or artificial cerebrospinal fluid (aCSF).

150. A pharmaceutical composition comprising the population of modified oligonucleotides of claim 143; and a pharmaceutically acceptable carrier or diluent.

151. The pharmaceutical composition of claim 150, wherein the pharmaceutically acceptable diluent comprises phosphate buffered saline (PBS) or artificial cerebrospinal fluid (aCSF).
